(19) United States
(12) Patent Application Publication
(10) Pub. No.: US 2026/0272882 A1
(43) Pub. Date: Sep. 17, 2026

(54) ORAL DOSAGE FORMS OF ELRAGLUSIB

(71) Applicant: Actuate Therapeutics, Inc., Fort Worth, TX (US)

(72) Inventors: Richard KENLEY, Poway, CA (US); Terrence Dahl, Sunnyvale, CA (US); Daniel M. Schmitt, Grayslake, IL (US); Scott Bone, Bend, OR (US)

(21) Appl. No.: 19/669,218

(22) Filed: May 6, 2026

Related U.S. Application Data
(63) Continuation of application No. 18/864,338, filed on Jun. 27, 2023.
(60) Provisional application No. 63/355,718, filed on Jun. 27, 2022.

(51) Int. Cl. A61K 31/407; A61K 9/20; A61K 47/10; A61K 47/26; A61K 47/32; A61K 47/38

(52) U.S. Cl. CPC A61K 31/407; A61K 9/2009; A61K 9/2013; A61K 9/2018; A61K 9/2027; A61K 9/2054; A61K 47/10; A61K 47/26; A61K 47/32; A61K 47/38

(57) ABSTRACT

The present invention relates to solid dispersions comprising amorphous 3-(5-fluorobenzofuran-3-yl)-4-(5-methyl-5H-[1,3]dioxolo[4,5-f]indol-7-yl)-1H-pyrrole-2,5-dione and a stabilizing polymer; liquid solutions comprising 3-(5-fluorobenzofuran-3-yl)-4-(5-methyl-5H-[1,3]dioxolo[4,5-f]indol-7-yl)-1H-pyrrole-2,5-dione; liquid suspensions comprising 3-(5-fluorobenzofuran-3-yl)-4-(5-methyl-5H-[1,3]dioxolo[4,5-f]indol-7-yl)-1H-pyrrole-2,5-dione; pharmaceutical compositions containing these compositions, and uses thereof for treating disease.

### CROSS-REFERENCE TO RELATED APPLICATION

This application is a continuation application of U.S. application Ser. No. 18/864,338, filed on Nov. 8, 2024, which is a 371 U.S. National Stage of International Application No. PCT/US2023/069158, filed on Jun. 27, 2023, which claims the benefit of U.S. Provisional Application No. 63/355,718, filed on Jun. 27, 2022, the entirety of which is incorporated by reference herein.

### TECHNICAL FIELD

The present disclosure relates to oral dosage forms of 3-(5-fluorobenzofuran-3-yl)-4-(5-methyl-5H-[1,3]dioxolo[4,5-f]indol-7-yl)-1H-pyrrole-2,5-dione, also known as elraglusib.

### BACKGROUND

3-(5-fluorobenzofuran-3-yl)-4-(5-methyl-5H-[1,3]dioxolo[4,5-f]indol-7-yl)-1H-pyrrole-2,5-dione, also referred herein as 9-ING-41 or elraglusib, is a small molecule of empirical formula C₂₂H₁₃N₂O₅F having the chemical structure:

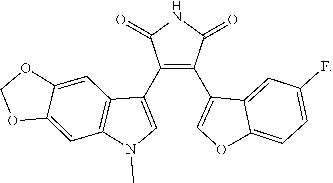 [SMILES: Cn1cc(C2=C(c3coc4ccc(F)cc34)C(=O)NC2=O)c2cc3c(cc21)OCO3]

The structure, properties, and/or biological activity of elraglusib are set forth in U.S. Pat. No. 8,207,216; Gaisina et al., From a Natural Product Lead to the Identification of Potent and Selective Benzofuran-3-yl-(indol-3-yl) maleimides as Glycogen Synthase Kinase 3β Inhibitors That Suppress Proliferation and Survival of Pancreatic Cancer Cells, J. Med. Chem. 2009, 52, 1853-1863; and Hilliard, et al., Glycogen Synthase Kinase 3β Inhibitors Induce Apoptosis in Ovarian Cancer Cells and Inhibit In-Vivo Tumor Growth, Anti-Cancer Drugs 2011, 22, 978-985.

Elraglusib is a glycogen synthase kinase-3 beta (GSK-3β) inhibitor being investigated for use as a single agent and for use in combination with other chemotherapy in patients with refractory hematologic malignancies or solid tumors. GSK-3β is a constitutively active serine-threonine kinase. Increased expression and aberrant function of GSK-3β have been implicated in the biology of numerous diseases, including cancer, inflammation, fibrosis, and neurodegeneration.

The water solubility of elraglusib is extremely low, which has limited its use in clinical practice to administration by slow intravenous infusion. An intravenous infusion of elraglusib comprises a 10 mg/mL solution of elraglusib API in a co-solvent vehicle that is subsequently added to a sterile intravenous diluent such as 5% w/v dextrose injection. This infusion regimen is burdensome to caregivers and patients alike, requiring once- or twice-weekly dosing and long (≥4 hour) infusion times. Thus, there is a need for liquid or solid oral dosage formulations of elraglusib that can be administered.

### SUMMARY OF THF INVENTION

The present disclosure provides novel solid amorphous dispersions, liquid solutions, and liquid suspensions of elraglusib that are suitable for oral administration.

The present disclosure also provides novel amorphous solid dispersions (“ASDs”), tablets, liquid solutions, and liquid suspensions of elraglusib that are suitable for oral administration.

In some aspects, the disclosure provides ASDs comprising amorphous 3-(5-fluorobenzofuran-3-yl)-4-(5-methyl-5H-[1,3]dioxolo[4,5-f]indol-7-yl)-1H-pyrrole-2,5-dione and a stabilizing polymer.

In some aspects, the disclosure provides ASDs incorporated into tablet dosage forms.

In some aspects, the disclosure provides liquid solutions comprising 3-(5-fluorobenzofuran-3-yl)-4-(5-methyl-5H-[1,3]dioxolo[4,5-f]indol-7-yl)-1H-pyrrole-2,5-dione (elraglusib); an emulsifier that is a polyethylene glycol, mustard lecithin, soy lecithin, egg lecithin, monoglyceride, diglyceride, polysorbate, stearoyl lactylate, sorbitan ester, polyglycerol ester, or sucrose ester; a pharmaceutically acceptable alcohol; and a surfactant that is sodium stearate, 4-(5-dodecyl)benzenesulfonate, sodium lauryl sulfate, docusate sodium, phosphatidylcholine, benzalkonium chloride, polyoxyethylene sorbitan fatty acid esters, polyoxyethylene 15 hydroxy stearate, polyoxyethylene castor oil derivatives, polyoxyethylene stearates, sorbitan fatty acid esters, polyoxyethylene alkyl ethers, and polyoxyethylene nonylphenol ether.

In other aspects, the disclosure provides liquid suspensions comprising: elraglusib; an emulsifier that is a polyethylene glycol, mustard lecithin, soy lecithin, egg lecithin, monoglyceride, diglyceride, polysorbate, stearoyl lactylate, sorbitan ester, polyglycerol ester, or sucrose ester; a pharmaceutically acceptable alcohol; a surfactant that is sodium stearate, 4-(5-dodecyl)benzenesulfonate, sodium lauryl sulfate, docusate sodium, phosphatidylcholine, benzalkonium chloride, polyoxyethylene sorbitan fatty acid esters, polyoxyethylene 15 hydroxy stearate, polyoxyethylene castor oil derivatives, polyoxyethylene stearates, sorbitan fatty acid esters, polyoxyethylene alkyl ethers, and polyoxyethylene nonylphenol ether; and a pharmaceutically acceptable diluent.

The disclosure also provides methods of making these novel ASDs, tablets, liquid solutions, and liquid suspensions of elraglusib and methods of using these novel ASDs, tablets, liquid solutions, and liquid suspensions of elraglusib for treating disease.

The disclosure also provides method of using the disclosed novel ASDs, tablets, liquid solutions, and liquid suspensions of elraglusib.

### BRIEF DESCRIPTION OF THF DRAWINGS

FIG. 1 shows X-ray powder diffraction (XRPD) patterns of amorphous elraglusib solid dispersions (ASDs) post solubility in simulated gastric fluid (SGF).

FIG. 2 shows XRPD of ASDs post solubility in Fasted-State Simulated Intestinal Fluid (FaSSIF).

FIG. 3 shows XRPD of ASDs post solubility in Fed-State Simulated Intestinal Fluid (FeSSIF).

FIG. 4 shows XRPD overlay of various ASDs after 2 weeks of exposure to 40° C. 33% RH.

FIG. 5 shows an XRPD overlay of various ASDs after 3 weeks of exposure to 40° C. 75% RH.

FIG. 6 shows an XRPD overlay of ASD stability samples after 9 week exposure to 40° C./33% RH.

FIG. 7 shows an XRPD overlay of ASD stability samples after 9 week exposure to 40° C./75% RH.

FIG. 8 shows an XRPD of “Elra:PVAP”, a specific ASD made with Phthalavin (polyvinyl acetate phthalate or PVAP) having 50% w/w elraglusib-loading and that was prepared by spray-drying from tetrahydrofuran (THF) solvent.

FIG. 9 shows a polarized light microscopy (PLM) image of Elra:PVAP made by spray drying from THF.

FIG. 10 shows TGA/DSC curves of Elra:PVAP made by spray drying from THF-(no pin hole in DSC pan).

FIGS. 11A and B shows TGA and mDSC after additional drying of Elra:PVAP made by spray drying from THF (A) no pin hole in DSC pan and (B) with pin hole in DSC pan.

FIG. 12 shows HPLC chromatogram of Elra:PVAP made by spray drying from THF.

FIG. 13A-C shows particle size distribution (PSD) analysis of Elra:PVAP—(A) run 1, (B) run 2, and (C) run 3.

FIG. 14 shows XRPD of “Elra:CAP”, a specific ASD made with cellulose acetate phthalate (CAP) having 50% w/w elraglusib-loading and that was prepared by spray drying from THF solvent.

FIG. 15 shows a PLM image of Elra:CAP spray-dried using THF.

FIG. 16 shows TGA/DSC curves Elra:CAP made by spray-drying from THF.

FIGS. 17A and B shows TGA and mDSC after additional drying of Elra:CAP made by spray-drying from THF (A) no pin hole in DSC pan and (B) with pin hole in DSC pan.

FIG. 18 shows HPLC chromatogram of Elra:CAP made by spray-drying from THF.

FIG. 19A-C shows PSD analysis of Elra:CAP spray dried from THF-(A) run 1, (B) run 2, and (C) run 3.

FIG. 20 shows XRPD of solids recovered after kinetic solubility analysis for Elra:PVAP and Elra:CAP made by spray-drying from THF.

FIGS. 21A and B shows XRPD of Elra:CAP made by spray-drying from THF after (A) 6 weeks and (B) 12 weeks at various storage conditions.

FIG. 22 shows TGA and mDSC of Elra:PVAP without pinhole in DSC pan after exposure to (A) at 5° C. sample for 6 weeks, (B) 40° C. 33% RH sample for 6 weeks, (C) 40° C. 75% RH sample for 6 weeks, (D) at 5° C. sample for 12 weeks, (E) 40° C. 33% RH sample for 12 weeks, and (F) 40° C. 75% RH sample for 12 weeks.

FIG. 23 shows TGA and mDSC of Elra:CAP without pinhole in DSC pan after exposure to (A) at 5° C. sample for 6 weeks, (B) 40° C. 33% RH sample for 6 weeks, (C) 40° C. 75% RH sample for 6 weeks, (D) at 5° C. sample for 12 weeks, (E) 40° C. 33% RH sample for 12 weeks, and (F) 40° C. 75% RH sample for 12 weeks.

FIG. 24 shows ssNMR overlay of API reference, Phthalavin, and CAP focusing on baseline resolved peak at 91.5 ppm for API.

FIG. 25 shows ssNMR overlay of API reference, Phthalavin, and CAP where first derivative was applied to each spectrum.

FIG. 26 shows ssNMR overlay of first derivative spectrum of 6014415-13-A5 5° C. 12 weeks, API reference, and Phthalavin.

FIG. 27 shows ssNMR overlay of first derivative spectrum of 6014415-13-A5 40° C./75% RH 12 weeks, API reference, and Phthalavin.

FIG. 28 shows ssNMR overlay of first derivative spectrum of 6014415-14-A1 5° C. 12 weeks, API reference, and CAP.

FIG. 29 shows ssNMR overlay of first derivative spectrum of 6014415-14-A1 40° C./75% RH 12 weeks, API reference, and CAP

FIG. 30 shows elraglusib plasma concentration vs. time profile for the pharmacokinetic studies outlined in Example 3.

FIG. 31 shows the XRPD of Elra:CAP made from spray-drying from THFASD material batch 35044-004 showing monomodal distribution characteristic of an amorphous material. See Example 2B.

FIG. 32 shows SEM images of ASD material showing hollow sphere, collapsed sphere, and shattered sphere morphology (batch 35044-004). See Example 2B.

FIG. 33 shows SEM images showing elraglusib particles prior to milling (top) and after milling (bottom). See Example 2B.

FIG. 34 shows PSD of elraglusib pre-milling (left) and post-milling (right) with change in distribution See Example 2B.

FIG. 35 shows XRPD of elraglusib pre-milling (top) and post-milling (bottom) with consistent profiles. See Example 2B.

FIG. 36 shows dissolution profiles of elraglusib dosage forms in 6% w/w CTAB (hexadecyltrimethylammonium bromide) in 0.7 M sodium chloride with paddle speed 75 rpm vs 100 rpm showing varying final dissolution. See Example 2B.

FIG. 37 shows dissolution profiles in 1% and 6% w/w CTAB in 0.7 M sodium chloride showing improved dissolution profile for ASD material. See Example 2B.

FIG. 38 shows elraglusib plasma concentration vs. time profile for the pharmacokinetic studies outlined in Example 3. The IV and Oral Solution profiles represent the mean of studies 2 and 3.

FIG. 39 shows the DSC of the elraglusib API. TA-Q2000 Differential Scanning calorimetric analyzer for DSC.

FIG. 40 shows the TGA of the elraglusib API. TA-Q500 Thermogravimetric analyzer for TGA.

FIG. 41 shows the Elraglusib Plasma Concentration after Administration by IV Infusion, Orally in a Fasted State, and Orally in a Fed State. See Example 5.

FIG. 42 shows the relationships among the elraglusib exposure metrics of Cmax (top panel) and AUC∞ (bottom panel) for the 3 Treatments for Individual Subjects (PK Evaluable Population). See Example 5.

FIG. 43 shows the relative oral bioavailability based on Cmax and AUC∞ of elraglusib in the fasted state compared to an IV infusion as a function of elraglusib dose (PK evaluable population). See Example 5.

FIG. 44 shows the relative oral bioavailability based on Cmax and AUC∞ of elraglusib in the fed state compared to an IV infusion as a function of elraglusib dose (PK evaluable population). See Example 5.

FIG. 45 shows the relative oral bioavailability based on Cmax and AUC∞ of elraglusib in the fed state compared to the fasted state as a function of elraglusib dose (PK evaluable population). See Example 5.

FIG. 46 shows SEM images of ASD prototypes showing mixed morphologies of intact, collapsed, and shattered spheres. See Example 6.

FIG. 47 shows the XRPD of ASD lots compared to native elraglusib showing no detectable crystal peaks in the dispersions. See Example 6.

FIG. 48 shows biorelevant dissolution for Elraglusib ASD prototypes in FaSSIF pH 6.5. (Note: X denotes the “free” drug concentration) See Example 6.

FIG. 49 shows Biorelevant dissolution profiles for elraglusib ASD prototypes in FeSSIF pH 5.0 (Note: X denotes the “free” drug concentration). See Example 6.

FIG. 50 shows compression profiles for elraglusib:CAP and elraglusib:EL100 ASD formulations showing increased compressibility of the elraglusib:EL100 formulation. See Example 6.

FIG. 51 shows a visual comparison of elraglusib:EL100 and elraglusib:CAP ASD tablets. See Example 6.

FIG. 52 shows dissolution overlay of tablet formulations (Note: Data shown is normalized to the percent dissolved at 120 minutes due to the percent dissolved exceeding 110% and a high variability between vessels (>4.2% RSD)). See Example 6.

FIG. 53 shows SEM images for the 5 ASD sublots described in Example 7.

FIG. 54 shows PXRD testing of the 5 ASD sublots described in Example 7. Each sublot exhibits an amorphous halo with no evidence of crystallinity.

FIG. 55 shows the dissolution profile of the composite lot of lead ASD described in Example 7 (non-sink biorelevant; conditions: 50 ml beaker, 30 mL FaSSIF pH=6.5) compared to that of previous batch of ASD.

FIG. 56 shows Sieve Particle Size Analysis of Granules for the Elraglusib Lead Tablet Formulation (Data Ref: BBS0002-062). See Example 8

FIG. 57 shows non-sink dissolution testing results performed on the lead tablet formulation compared to the ingoing ASD. See Example 8.

FIG. 58 shows the process used for blend-delump-blend of intragranular blends (Ref: BBS0002-019). See Example 6.

FIG. 59 shows the compactability of elraglusib pre-granulated blends for prototype formulations (Ref: BBS0002-019). See Example 6.

FIG. 60 shows the process used for final blending of the elraglusib prototype formulations (Ref: BBS0002-019). See Example 6.

FIG. 61 shows non-sink dissolution of elraglusib prototype formulations compared to ingoing ASD (Ref: BBS0002-024). See Example 6.

FIG. 62 shows non-sink dissolution of elraglusib prototype formulations compared to ingoing ASD. See Example 6.

FIG. 63 shows non-sink dissolution of elraglusib prototype formulations compared to ingoing ASD (Ref: BBS0002-044). See Example 6.

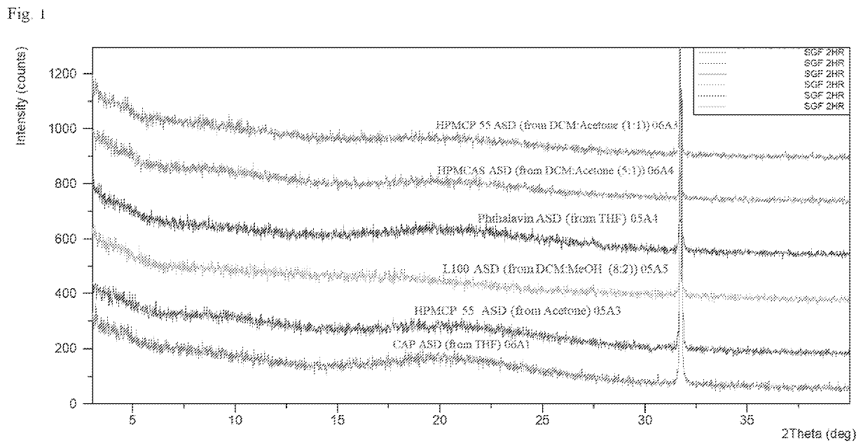

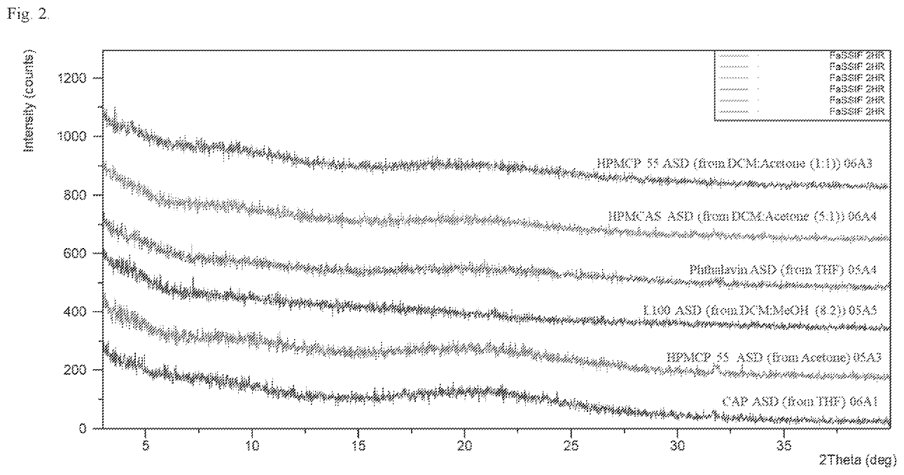

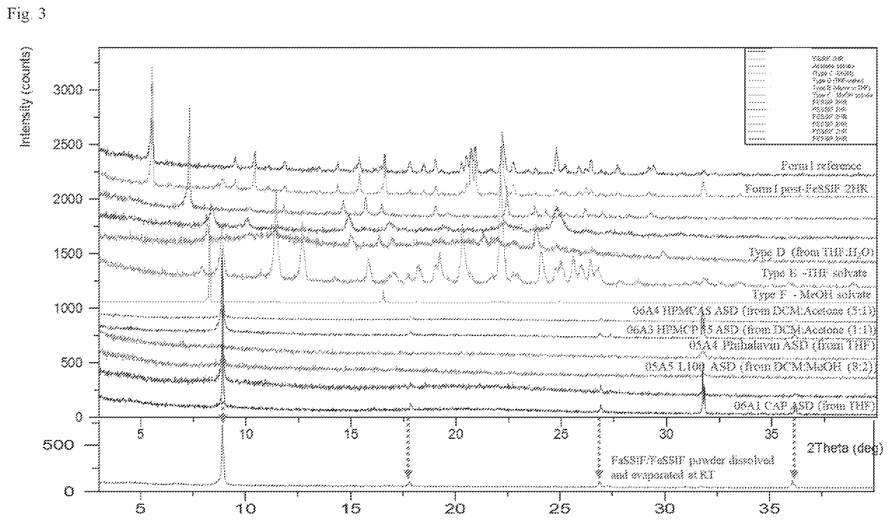

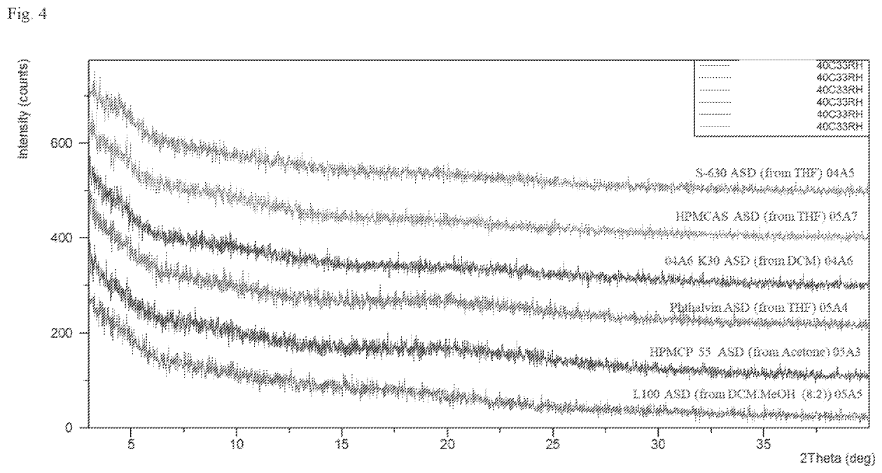

### DETAILED DESCRIPTION OF ILLUSTRATIVE EMBODIMENTS

The disclosure may be more fully appreciated by reference to the following description, including the following definitions and examples. Certain features of the disclosed compositions and methods which are described herein in the context of separate aspects, may also be provided in combination in a single aspect. Alternatively, various features of the disclosed compositions and methods that are, for brevity, described in the context of a single aspect, may also be provided separately or in any subcombination.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

The term “solid dispersions” as applied to a material that comprises an amorphous active ingredient are referred to herein as amorphous solid dispersions, or “ASDs.”

The term “SDD” refers to spray dried dispersion, i.e., a solid dispersion made by spray-drying. An ASD that is made by spray-drying is one type of SDD.

As used in the specification including the appended claims, the singular forms “a,” “an,” and “the” include the plural, and reference to a particular numerical value includes at least that particular value, unless the context clearly dictates otherwise.

When a range of values is expressed, an exemplary embodiment includes from the one particular value and/or to the other particular value. All ranges are inclusive and combinable. Further, reference to values stated in ranges includes each and every value within that range. When values are expressed as approximations, by use of the preposition “about,” it will be understood that the particular value forms another embodiment. The term “about” as used herein when referring to a measurable value such as an amount, a temporal duration, and the like, is meant to encompass reasonable variations of the value, such as, for example, ±10% from the specified value. For example, the phrase “about 50%” can include ±10% of 50, or from 45% to 55%, inclusive of 50%.

It is to be appreciated that certain features of the disclosure which are, for clarity, described herein in the context of separate embodiments, may also be provided in combination in a single embodiment. Conversely, various features of the disclosure that are, for brevity, described in the context of a single embodiment, may also be provided separately or in any subcombination.

As used herein, whether by itself or in conjunction with another term or terms, “pharmaceutically acceptable” indicates that the designated entity such as, for example, a pharmaceutically acceptable excipient, is generally chemically and/or physically compatible with other ingredients in a composition, and/or is generally physiologically compatible with the recipient thereof.

As used herein, whether by themselves or in conjunction with another term or terms, “subject(s),” “individual(s),” and “patient(s)”, refer to mammals, including humans. The term human(s) refers to and includes, a human child, adolescent, or adult.

As used herein, whether by itself or in conjunction with another term or terms, it should be understood that the phrases “method of treating” and “method of treatment” may be used interchangeably with the phrase “for use in the treatment of” a particular disease.

As used herein, whether by themselves or in conjunction with another term or terms, “treats,” “treating,” “treated,” and “treatment,” refer to and include ameliorative, palliative, and/or curative uses and results, or any combination thereof. In other embodiments, the methods described herein can be used prophylactically, that is, preventatively. It should be understood that “prophylaxis” or a prophylactic use or result do not refer to nor require absolute or total prevention (i.e., a 100% preventative or protective use or result). As used herein, prophylaxis or a prophylactic (preventative) use or result refers to uses and results in which administration of a compound or composition diminishes or reduces the severity of a particular condition, symptom, disorder, or disease described herein; diminishes or reduces the likelihood of experiencing a particular condition, symptom, disorder, or disease described herein; or delays the onset or relapse (reoccurrence) of a particular condition, symptom, disorder, or disease described herein; or any combination of the foregoing.

As used herein, whether used alone or in conjunction with another term or terms, “therapeutic” and “therapeutically effective amount” refer to an amount of a compound or composition that (a) treats a particular condition, symptom, disorder, or disease described herein; (b) attenuates, ameliorates, or eliminates one or more symptoms of a particular condition, disorder, or disease described herein; (c) delays the onset or relapse (reoccurrence) of a particular condition, symptom, disorder, or disease described herein; (d) prevents the onset of a particular condition, symptom, disorder, or disease described herein. It should be understood that the terms “therapeutic” and “therapeutically effective” encompass any one of the aforementioned effects (a)-(d), either alone or in combination with any of the others (a)-(d).

“Pharmaceutically acceptable salt” refers to a salt of a compound of the disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols. Pharmaceutically acceptable excipients will be known to those of skill in the art, and include, for example, excipients described in treatises such as, for example, Paul J. Sheskey, et al. (eds), Handbook of Pharmaceutical Excipients, Pharmaceutical Press; 9th Revised edition (Oct. 20, 2020).

A “solvate” refers to a physical association of a compound of the disclosure with one or more solvent molecules.

As used herein, the term “isomers” refers to compounds which have identical chemical constitution, but differ with regard to the arrangement of the atoms or groups in space, e.g., enantiomers, diastereomers, tautomers.

The term “administering” means either directly administering a compound or composition of the present invention, or administering a prodrug, derivative or analog which will form an equivalent amount of the active compound or substance within the body.

The terms “Glycogen Synthase Kinase-3 Beta” and “GSK-3β” are used interchangeably and according to their common, ordinary meaning and refer to proteins of the same or similar names and functional fragments and homologs thereof. The term includes any recombinant or naturally occurring form of, or variants thereof that maintain GSK-3β activity (e.g., within at least 30%, 40%, 50%, 60%, 70%, 80%, 90%, 95%, or 100% activity compared to GSK-3β).

### Amorphous Solid Dispersions of Elraglusib

In some aspects, the disclosure is directed to a solid dispersion comprising amorphous 3-(5-fluorobenzofuran-3-yl)-4-(5-methyl-5H-[1,3]dioxolo[4,5-f]indol-7-yl)-1H-pyrrole-2,5-dione (i.e., elraglusib) and a stabilizing polymer.

As used herein, the term “solid dispersion” refers to a solid material in which an active pharmaceutical ingredient is dispersed in another material. In some embodiments, the solid dispersion is homogeneous such that the amorphous elraglusib is homogeneously dispersed throughout the polymer thereby.

In some embodiments, the dispersion itself has a single glass transition temperature (Tg) which demonstrates that the dispersion is homogeneous.

Tg as used herein is the characteristic temperature where a glassy material, upon gradual heating, undergoes a relatively rapid (e.g., 10 to 100 seconds) physical change from a glass state to a fluid state. The Tg of an amorphous material such as a polymer, drug or dispersion can be measured by several techniques, including by a dynamic mechanical analyzer (DMA), a dilatometer, dielectric analyzer, and by a differential scanning calorimeter (DSC). The exact values measured by each technique can vary somewhat but usually fall within 10° to 30° C. of each other. Regardless of the technique used, when an amorphous dispersion exhibits a single Tg, this indicates that the dispersion is substantially homogenous.

As used herein, “stabilizing polymer” refers to a polymer that maintains the 3-(5-fluorobenzofuran-3-yl)-4-(5-methyl-5H-[1,3]dioxolo[4,5-f]indol-7-yl)-1H-pyrrole-2,5-dione (i.e., elraglusib) in amorphous form.

In some embodiments, the stabilizing polymer is N-vinyl-2-pyrrolidone-vinyl acetate copolymer (copovidone), cellulose acetate phthalate (CAP), Hypromellose: hydroxypropyl methylcellulose, hydroxypropyl methylcellulose phthalate, hydroxypropyl methylcellulose acetate succinate, polyvinyl acetate phthalate (Phthalavin or PVAP), polyvinylpyrrolidone, poly(methylmethacrylate-co-methacrylic acid), or a miscible mixture thereof. A miscible mixture of these polymers is one in which the mixture of polymers forms a single phase.

In some embodiments, the stabilizing polymer is N-vinyl-2-pyrrolidone-vinyl acetate copolymer (copovidone).

In some embodiments, the stabilizing polymer is cellulose acetate phthalate (CAP).

In some embodiments, the stabilizing polymer is hydroxypropyl methylcellulose phthalate.

In some embodiments, the stabilizing polymer is hydroxypropyl methylcellulose acetate succinate.

In some embodiments, the stabilizing polymer is polyvinyl acetate phthalate (Phthalavin or PVAP).

In some embodiments, the stabilizing polymer is polyvinylpyrrolidone.

In some embodiments, the stabilizing polymer is hydroxypropyl methylcellulose phthalate, hydroxypropyl methylcellulose acetate succinate, polyvinyl acetate phthalate, polyvinylpyrrolidone, or a mixture thereof.

In some embodiments, the stabilizing polymer is a methacrylic acid polymer.

In some embodiments, the stabilizing polymer is a methacrylic acid copolymer with an alkyl methacrylate.

In some embodiments, the stabilizing polymer is a poly(methyl methacrylate-co-methacrylic acid), also referred to as methacrylic acid-methyl methacrylate copolymer or poly(methacrylic acid-co-methyl methacrylate); such as, for example, Eudragit® L100 polymer (“EL100” or “L100” herein).

In some embodiments, the stabilizing polymer is a poly(methyl methacrylate-co-methacrylic acid) (1:1).

In some embodiments, the stabilizing polymer is Eudragit® L100.

In some embodiments, the stabilizing polymer is Eudragit® L100-55 polymer.

In some embodiments, the stabilizing polymer is soluble at a pH above 5.

In some embodiments, the stabilizing polymer is soluble at a pH above 5.5.

In some embodiments, the stabilizing polymer is soluble at a pH above 6.

In some aspects, the 3-(5-fluorobenzofuran-3-yl)-4-(5-methyl-5H-[1,3]dioxolo[4,5-f]indol-7-yl)-1H-pyrrole-2,5-dione is present in an amount of from about 10% to about 70% by weight relative to the weight of the stabilizing polymer.

In some aspects, the 3-(5-fluorobenzofuran-3-yl)-4-(5-methyl-5H-[1,3]dioxolo[4,5-f]indol-7-yl)-1H-pyrrole-2,5-dione is present in an amount of from about 10% to about 60% by weight relative to the weight of the stabilizing polymer.

In some aspects, the 3-(5-fluorobenzofuran-3-yl)-4-(5-methyl-5H-[1,3]dioxolo[4,5-f]indol-7-yl)-1H-pyrrole-2,5-dione is present in an amount of from about 10% to about 50% by weight relative to the weight of the stabilizing polymer.

In some embodiments, the 3-(5-fluorobenzofuran-3-yl)-4-(5-methyl-5H-[1,3]dioxolo[4,5-f]indol-7-yl)-1H-pyrrole-2,5-dione is present in an amount of from about 20% to about 60%, or from about 25% to about 50%, by weight relative to the weight of the stabilizing polymer.

In some embodiments, the 3-(5-fluorobenzofuran-3-yl)-4-(5-methyl-5H-[1,3]dioxolo[4,5-f]indol-7-yl)-1H-pyrrole-2,5-dione is present in an amount of about 50% by weight relative to the weight of the stabilizing polymer.

In other embodiments, the 3-(5-fluorobenzofuran-3-yl)-4-(5-methyl-5H-[1,3]dioxolo[4,5-f]indol-7-yl)-1H-pyrrole-2,5-dione is present in an amount of from about 20% to about 60% by weight relative to the weight of the stabilizing polymer, such as, for example, any one of about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60% by weight relative to the weight of the stabilizing polymer.

In some embodiments, the 3-(5-fluorobenzofuran-3-yl)-4-(5-methyl-5H-[1,3]dioxolo[4,5-f]indol-7-yl)-1H-pyrrole-2,5-dione is present in an amount of from about 25% to about 50% by weight relative to the weight of the stabilizing polymer, such as, for example, any one of about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50% by weight relative to the weight of the stabilizing polymer.

In some embodiments the weight ratio of amorphous 3-(5-fluorobenzofuran-3-yl)-4-(5-methyl-5H-[1,3]dioxolo[4,5-f]indol-7-yl)-1H-pyrrole-2,5-dione to stabilizing polymer ranges from about 30:70 to about 50:50, such as, for example, any one of 30:70, 31:69, 32:68, 33:67, 34:66, 35:65, 36:64, 37:63, 38:62, 39:61, 40:60, 41:59, 42:58, 43:57, 44:56, 45:55, 46:54, 47:53, 48:52, 49:51, or 50:50.

In some embodiments, the ASD comprises about 40-50% by weight elraglusib and about 50-60% by weight of cellulose acetate phthalate (CAP).

In some embodiments, the ASD comprises about 50% by weight elraglusib and about 50% by weight of cellulose acetate phthalate (CAP).

In some embodiments, the ASD comprises about 40-50% by weight elraglusib and about 50-60% by weight of poly(methyl methacrylate-co-methacrylic acid).

In some embodiments, the ASD comprises about 50% by weight elraglusib and about 50% by weight of poly(methyl methacrylate-co-methacrylic acid).

In some embodiments, the ASD comprises about 40-50% by weight elraglusib and about 50-60% by weight of Eudragit® L00.

In some embodiments, the ASD comprises about 50% by weight elraglusib and about 50% by weight of Eudragit® L00.

In some embodiments the solid dispersion has a single glass transition temperature.

In some embodiments, the solid dispersion is stable for at least 48 hours when exposed to 60° C. and 75% relative humidity, such as for example, at least 48 hours, at least 72 hours, at least 96 hours, or at least 120 hours. As used herein, the term “stable” means that elraglusib in the solid dispersion remains amorphous as measured by XRPD or other analytical techniques.

In other embodiments, the solid dispersion is stable for at least 4 weeks when exposed to 40° C. and 75% relative humidity, such as for example, at least 4 weeks, at least 8 weeks, or at least 12 weeks.

In other embodiments, the solid dispersion is stable for at least 12 weeks when exposed to 40° C. and 75% relative humidity.

In some aspects, oral administration of a solid dispersion of the disclosure to a patient results in an elraglusib AUC∞ that is at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 80%, at least 91%, at least 92%, at least 93%, at least 94%, at least 95%, at least 96%, or at least 97%, of the elraglusib AUC∞ resulting from IV administration to the patient of an equivalent dose (on a mg/kg basis) of elraglusib.

In some aspects, oral administration of a solid dispersion of the disclosure to a patient results in an elraglusib AUC∞ that is at least 68% of the elraglusib AUC∞ resulting from IV administration to the patient of an equivalent dose (on a mg/kg basis) of elraglusib.

In some aspects, oral administration of a solid dispersion of the disclosure to a patient results in an elraglusib AUC∞ that is at least 97% of the elraglusib AUC∞ resulting from IV administration to the patient of an equivalent dose (on a mg/kg basis) of elraglusib.

In some aspects, the disclosure is directed to processes for preparing the solid dispersions of the disclosure.

In some embodiments, dispersions are formed by “solvent processing” in which elraglusib and a polymer are dissolved in a common solvent. “Common” here means that the solvent, which can be a mixture of compounds, will simultaneously dissolve the drug and the polymer(s). After both the elraglusib and the polymer have been dissolved, the solvent is rapidly removed by evaporation or by mixing with a non-solvent. Exemplary processes are spray-drying, and precipitation by rapid mixing of the polymer and drug solution with CO₂, water, or some other non-solvent. In some embodiments, removal of the solvent results in a solid dispersion which is homogeneous. As described previously, in such homogeneous dispersions, the elraglusib is dispersed homogeneously throughout the polymer and can be thought of as a solid solution of elraglusib in the polymer(s).

The solvent may be removed through the process of spray-drying. The term spray-drying is used conventionally and broadly refers to processes involving breaking up liquid mixtures into small droplets (atomization) and rapidly removing solvent from the mixture in a container (spray-drying apparatus) where there is a strong driving force for evaporation of solvent from the droplets. The strong driving force for solvent evaporation is generally provided by maintaining the partial pressure of solvent in the spray-drying apparatus well below the vapor pressure of the solvent at the temperature of the drying droplets. This is accomplished by either (1) maintaining the pressure in the spray-drying apparatus at a partial vacuum (e.g., 0.01 to 0.50 atm); (2) mixing the liquid droplets with a warm drying gas; or (3) both. In addition, at least a portion of the heat required for evaporation of solvent may be provided by heating the spray solution.

Solvents suitable for spray-drying can be any organic compound in which elraglusib and polymer are mutually soluble such that the total dissolved solids is at least 8% by weight of the solution. In some embodiments, the solvent is also volatile with a boiling point of 150° C. or less. In addition, the solvent should have relatively low toxicity and be removed from the dispersion to a level that is acceptable according to The International Committee on Harmonization (ICH) guidelines. Removal of solvent to this level may require a processing step such as tray-drying subsequent to the spray-drying. Solvents include alcohols such as methanol, ethanol, n-propanol, isopropanol, and butanol; ketones such as acetone, methyl ethyl ketone and methyl isobutyl ketone; esters such as ethyl acetate and propylacetate; ethers such as tetrahydrofuran (THF), and various other solvents such as acetonitrile, methylene chloride, toluene, and 1,1,1-trichloroethane. Lower volatility solvents such as dimethyl acetamide or dimethylsulfoxide can also be used. Mixtures of solvents, such as 50% methanol and 50% acetone, can also be used, as can mixtures with water as long as the polymer and elraglusib are sufficiently soluble and chemically stable to make the spray-drying process practicable. Generally, due to the hydrophobic nature of elraglusib, non-aqueous solvents, or mixtures of a non-aqueous solvent and water are used. In some embodiments, non-aqueous solvents comprise less than or equal to about 10 wt. % water. In other embodiments, non-aqueous solvents comprise less than about 10 wt. % water; in some embodiments, less than 1 wt. % water.

In some embodiments, solvents for spray drying elraglusib/polymer solutions are THF, acetone, ethanol, methanol, mixtures thereof, or mixtures with water.

In some embodiments solvents for spray-drying elraglusib/CAP solutions are acetone/water mixtures.

In some embodiments solvents for spray-drying elraglusib/CAP solutions are mixtures of 90-95% by weight acetone and 5-10% by weight of water.

In some embodiments solvents for spray-drying elraglusib/CAP solutions are mixtures of 90% by weight acetone and 10% by weight of water.

In some embodiments solvents for spray-drying elraglusib/CAP solutions are mixtures of 95% by weight acetone and 5% by weight of water.

In some embodiments solvents for spray-drying elraglusib/poly(methyl methacrylate-co-methacrylic acid) solutions are acetone mixtures with water.

In some embodiments solvents for spray-drying elraglusib/poly(methyl methacrylate-co-methacrylic acid) solutions are mixtures of 90-95% by weight acetone and 5-10% by weight of water.

In some embodiments solvents for spray-drying elraglusib/poly(methyl methacrylate-co-methacrylic acid) solutions are mixtures of 90% by weight acetone and 10% by weight of water.

In some embodiments solvents for spray-drying elraglusib/poly(methyl methacrylate-co-methacrylic acid) solutions are mixtures of 95% by weight acetone and 5% by weight of water.

In some embodiments solvents for spray-drying elraglusib/EL100 solutions are acetone mixtures with water.

In some embodiments solvents for spray-drying elraglusib/EL100 solutions are mixtures of 90-95% by weight acetone and 5-10% by weight of water.

In some embodiments solvents for spray-drying elraglusib/EL100 solutions are mixtures of 90% by weight acetone and 10% by weight of water.

In some embodiments solvents for spray-drying elraglusib/EL100 solutions are mixtures of 95% by weight acetone and 5% by weight of water.

Generally, the temperature and flow rate of the drying gas is chosen so that the polymer/drug-solution droplets are dry enough by the time they reach the wall of the apparatus that they are essentially solid, and so that they form a fine powder and do not stick to the apparatus wall. The actual length of time to achieve this level of dryness depends on the size of the droplets. The large surface-to-volume ratio of the droplets and the large driving force for evaporation of solvent leads to actual drying times of a few seconds or less, and more typically less than 0.1 second. This rapid drying is often critical to the particles maintaining a uniform, homogeneous dispersion instead of separating into drug-rich and polymer-rich phases.

Spray-drying processes and spray-drying equipment are described generally in Perry's Chemical Engineers Handbook, Sixth Edition (R. H. Perry, D. W. Green, J. O. Maloney, eds.) McGraw-Hill Book Co. 1984, pages 2054 to 2057. More details on spray-drying processes and equipment are reviewed by Marshall “Atomization and Spray-Drying.” 50 Chem. Eng. Prog. Monogr. Series 2 (1954).

In some embodiments, the process for preparing the solid dispersion comprises the steps of: (a) dissolving 3-(5-fluorobenzofuran-3-yl)-4-(5-methyl-5H-[1,3]dioxolo[4,5-f]indol-7-yl)-1H-pyrrole-2,5-dione and the stabilizing polymer in a suitable solvent to form a solution; and (b) removing the solvent by evaporation to form the solid dispersion.

Suitable solvents for dissolving 3-(5-fluorobenzofuran-3-yl)-4-(5-methyl-5H-[1,3]dioxolo[4,5-f]indol-7-yl)-1H-pyrrole-2,5-dione and the stabilizing polymer in the processes of the disclosure include dichloromethane, tetrahydrofuran, aqueous tetrahydrofuran, acetone, aqueous acetone, methanol, ethanol, ethyl acetate, and mixtures thereof.

In some embodiments, a suitable solvent for dissolving elraglusib and the stabilizing polymer in the processes of the disclosure is aqueous acetone.

In some embodiments, a suitable solvent for dissolving elraglusib and the stabilizing polymer in the processes of the disclosure is aqueous acetone comprising about 90% acetone by weight and about 10% water by weight.

In some embodiments, a suitable solvent for dissolving elraglusib and the stabilizing polymer in the processes of the disclosure is aqueous acetone comprising about 95% acetone by weight and about 5% water by weight.

In some embodiments, stabilizing polymers suitable for use in the processes for preparing the solid dispersions of the disclosure include N-vinyl-2-pyrrolidone-vinyl acetate copolymer (copovidone), cellulose acetate phthalate (CAP), hydroxypropyl methylcellulose phthalate, hydroxypropyl methylcellulose acetate succinate, polyvinyl acetate phthalate (Phthalavin), polyvinylpyrrolidone, or a miscible mixture thereof. A miscible mixture of these polymers is one in which the mixture of polymers forms a single phase.

In some embodiments, stabilizing polymers suitable for use in the processes for preparing the solid dispersions of the disclosure include N-vinyl-2-pyrrolidone-vinyl acetate copolymer (copovidone), cellulose acetate phthalate (CAP), hydroxypropyl methylcellulose phthalate, hydroxypropyl methylcellulose acetate succinate, polyvinyl acetate phthalate (Phthalavin), polyvinylpyrrolidone, poly(methyl methacrylate-co-methacrylic acid) exemplified by Eudragit® L100 polymer (“EL100”), or a miscible mixture thereof. A miscible mixture of these polymers is one in which the mixture of polymers forms a single phase.

In some embodiments, a stabilizing polymer suitable for use in the processes for preparing the solid dispersions of the disclosure is a poly(methyl methacrylate-co-methacrylic acid).

In some embodiments, a stabilizing polymer suitable for use in the processes for preparing the solid dispersions of the disclosure is Eudragit® L100 polymer (“EL100”).

In some embodiments, a stabilizing polymer suitable for use in the processes for preparing the solid dispersions of the disclosure is cellulose acetate phthalate (CAP).

In some embodiments of the disclosed processes, the solvent is removed by evaporation. The solvent may be removed by any evaporation technique that results in preparation of a solid dispersion of the disclosure, i.e., a solid dispersion containing amorphous elraglusib.

In some embodiments the solvent is evaporated by spray-drying.

In some embodiments the solvent is evaporated under reduced pressure, e.g., partial vacuum.

In some embodiments the solvent is evaporated using an inert gas. For example, in some aspects, the solvent is evaporated by passing the solution under a stream of inert gas. In some embodiments, the inert gas comprises nitrogen. In other embodiments, the inert gas comprises argon.

### Oral Liquid Suspensions and Liquid Solutions of Elraglusib

### Oral Liquid Solutions

In other aspects, the disclosure provides liquid solutions comprising 3-(5-fluorobenzofuran-3-yl)-4-(5-methyl-5H-[1,3]dioxolo[4,5-f]indol-7-yl)-1H-pyrrole-2,5-dione (elraglusib); an emulsifier that is a polyethylene glycol, mustard lecithin, soy lecithin, egg lecithin, monoglyceride, diglyceride, polysorbate, stearoyl lactylate, sorbitan ester, polyglycerol ester, or sucrose ester; a pharmaceutically acceptable alcohol; and a surfactant that is sodium stearate, 4-(5-dodecyl)benzenesulfonate, sodium lauryl sulfate, docusate sodium, phosphatidylcholine, benzalkonium chloride, polyoxyethylene sorbitan fatty acid esters, polyoxyethylene 15 hydroxy stearate, polyoxyethylene castor oil derivatives, polyoxyethylene stearates, sorbitan fatty acid esters, polyoxyethylene alkyl ethers, and polyoxyethylene nonylphenol ether.

In some embodiments, the disclosure provides liquid solutions comprising about 4.0-6.0 wt. % 3-(5-fluorobenzofuran-3-yl)-4-(5-methyl-5H-[1,3]dioxolo[4,5-f]indol-7-yl)-1H-pyrrole-2,5-dione; about 75-95 wt. % of an emulsifier that is a polyethylene glycol, mustard lecithin, soy lecithin, egg lecithin, monoglyceride, diglyceride, polysorbate, stearoyl lactylate, sorbitan ester, polyglycerol ester, or sucrose ester; about 2-17% wt. % of a pharmaceutically acceptable alcohol; about 0.5-10% wt. % of a surfactant that is sodium stearate, 4-(5-dodecyl)benzenesulfonate, sodium lauryl sulfate, docusate sodium, phosphatidylcholine, benzalkonium chloride, polyoxyethylene sorbitan fatty acid esters, polyoxyethylene 15 hydroxy stearate, polyoxyethylene castor oil derivatives, polyoxyethylene stearates, sorbitan fatty acid esters, polyoxyethylene alkyl ethers, and polyoxyethylene nonylphenol ether.

In some aspects, the liquid solution comprises about 4.0-6.0 wt. % 3-(5-fluorobenzofuran-3-yl)-4-(5-methyl-5H-[1,3]dioxolo[4,5-f]indol-7-yl)-1H-pyrrole-2,5-dione, such as, for example, one of about 4.0%, about 4.1%, about 4.2%, about 4.3%, about 4.4%, about 4.5%, about 4.6%, about 4.7%, about 4.8%, about 4.9%, about 5.0%, about 5.1%, about 5.2%, about 5.3%, about 5.4%, about 5.5%, about 5.6%, about 5.7%, about 5.8%, about 5.9%, or about 6.0%.

In some embodiments, the emulsifier is a polyethylene glycol (PEG).

In other embodiments, the emulsifier is a polyethylene glycol having a molecular weight of 100-1000 Da.

In other embodiments, the emulsifier is PEG 400.

In some embodiments, the emulsifier is a mustard lecithin.

In some embodiments, the emulsifier is a soy lecithin.

In some embodiments, the emulsifier is an egg lecithin.

In some embodiments, the emulsifier is a monoglyceride.

In some embodiments, the emulsifier is a diglyceride.

In some embodiments, the emulsifier is a polysorbate.

In some embodiments, the emulsifier is a stearoyl lactylate.

In some embodiments, the emulsifier is a sorbitan ester.

In some embodiments, the emulsifier is a polyglycerol ester.

In some embodiments, the emulsifier is a sucrose ester.

In some aspects, the emulsifier is present in an amount of about 75-95 wt. %, such as, for example, one of about 75 wt. %, about 76 wt. %, about 77 wt. %, about 78 wt. %, about 79 wt. %, about 80 wt. %, about 81 wt. %, about 82 wt. %, about 83 wt. %, about 84 wt. %, about 85 wt. %, about 86 wt. %, about 87 wt. %, about 88 wt. %, about 89 wt. %, about 90 wt. %, about 91 wt. %, about 92 wt. %, about 93 wt. %, about 94 wt. %, or about 95 wt. %.

Pharmaceutically acceptable alcohols are alcohols that are not toxic when orally administered to a human in the amount present in the composition. Alcohols that are not toxic are those that are identified as Generally Recognized as Safe (“GRAS”) by the U.S. Food and Drug Administration. An exemplary pharmaceutically acceptable alcohol is ethanol.

In some embodiments, the pharmaceutically acceptable alcohol is ethanol.

In some aspects, the pharmaceutically acceptable alcohol is present in an amount of about 2-17% wt. %, such as, for example, one of about 2 wt. %, about 3 wt. %, about 4 wt. %, about 5 wt. %, about 6 wt. %, about 7 wt. %, about 8 wt. %, about 9 wt. %, about 10 wt. %, about 11 wt. %, about 12 wt. %, about 13 wt. %, about 14 wt. %, about 15 wt. %, about 16 wt. %, or about 17 wt. %.

In some embodiments, the surfactant is sodium stearate.

In some embodiments, the surfactant is 4-(5-dodecyl)benzenesulfonate.

In some embodiments, the surfactant is sodium lauryl sulfate.

In some embodiments, the surfactant is docusate sodium.

In some embodiments, the surfactant is phosphatidylcholine.

In some embodiments, the surfactant is benzalkonium chloride.

In some embodiments, the surfactant is polyoxyethylene sorbitan fatty acid esters.

In some embodiments, the surfactant is polysorbate 80 (Polyoxyethylenesorbitan monooleate).

In some embodiments, the surfactant is polyoxyethylene 15 hydroxy stearate.

In some embodiments, the surfactant is a polyoxyethylene castor oil derivative.

In some embodiments, the surfactant is a polyoxyethylene stearate.

In some embodiments, the surfactant is a sorbitan fatty acid ester.

In some embodiments, the surfactant is a polyoxyethylene alkyl ethers.

In some embodiments, the surfactant is a polyoxyethylene nonylphenol ether.

In some aspects, the surfactant is present in an amount of about 0.5-10% wt. % such as, for example, one of: about 0.5 wt. %, about 0.6 wt. %, about 0.7 wt. %, about 0.8 wt. %, about 0.9 wt. %, about 1.0 wt. %, about 1.5 wt. %, about 2 wt. %, about 2.5 wt. %, about 3 wt. %, about 3.5 wt. %, about 4 wt. %, about 4.5 wt. %, about 5 wt. %, about 5.5 wt. %, about 6 wt. %, about 6.5 wt. %, about 7 wt. %, about 7.5 wt. %, about 8 wt. %, about 8.5 wt. %, about 9 wt. %, about 9.5 wt. %, or about 10 wt. %.

In some embodiments, the disclosure is directed to a liquid solution comprising about 4.3-5.5 wt. % 3-(5-fluorobenzofuran-3-yl)-4-(5-methyl-5H-[1,3]dioxolo[4,5-f]indol-7-yl)-1H-pyrrole-2,5-dione; about 75-95 wt. % polyethylene glycol 400, about 1-16 wt. % ethanol, about 0.5-8.5 wt. % polysorbate 80, and about 0-5 wt. % water.

In some embodiments, the disclosure is directed to a liquid solution comprising about 4.5-5.5 wt. % 3-(5-fluorobenzofuran-3-yl)-4-(5-methyl-5H-[1,3]dioxolo[4,5-f]indol-7-yl)-1H-pyrrole-2,5-dione; about 75-95 wt. % polyethylene glycol 400, about 1-16 wt. % ethanol, about 0.5-8.5 wt. % polysorbate 80, and about 0-5 wt. % water.

In some embodiments, the liquid solution comprises about 4.7-4.8 wt. % elraglusib, about 76-91 wt. % polyethylene glycol 400, about 4.7-14.3 wt. % ethanol, about 0.5-1 wt. % polysorbate 80.

In other embodiments, the liquid solution comprises about 4.7-4.8 wt. % elraglusib, about 89-90 wt. % polyethylene glycol 400, about 4.7-4.8 wt. % ethanol, and about 0.9-1 wt. % polysorbate 80.

In other embodiments, the liquid solution comprises about 4.2-4.4 wt. % elraglusib, about 89-90 wt. % polyethylene glycol 400, about 4.7-4.8 wt. % ethanol, and about 0.9-1 wt. % polysorbate 80.

In some embodiments the concentration of elraglusib in the liquid solution is at least 45 mg/mL, such as, for example, one of 45 mg/mL, 46 mg/mL, 47 mg/mL, 48 mg/mL, 49 mg/mL, 50 mg/mL, 51 mg/mL, 52 mg/mL, 53 mg/mL, and the like.

In some embodiments the concentration of elraglusib in the liquid solution is about 50 mg/mL.

In some embodiments the concentration of elraglusib in the liquid solution is at least 50 mg/mL.

In some embodiments, the liquid solution contains less than 3% total related substances as measured by high performance liquid chromatography (HPLC) area %. As used herein, the term “related substance” refers to a compound, present as an impurity, that is structurally related to elraglusib.

As used herein, HPLC area % is measured using the HPLC method set forth herein in Table 5.

In some embodiments, the solution contains less than 2% total related substances as measured by HPLC area %.

In some embodiments, the solution contains less than 1% total related substances as measured by HPLC area %.

In some embodiments, the elraglusib in the solution has a purity greater than 97% as measured by HPLC area %.

In some embodiments, the elraglusib in the solution has a purity greater than 98% as measured by HPLC area %.

In some embodiments, the elraglusib in the solution has a purity greater than 99% as measured by HPLC area %.

In some embodiments, the solution contains less than 2%, as measured by HPLC area %, of a related substance having a relative retention time of 0.96 or 0.97 relative to the retention time of elraglusib.

In some embodiments, the solution contains less than 1%, of a related substance having a relative retention time of 0.96 or 0.97 relative to the retention time of elraglusib.

In some embodiments, the solution contains less than 0.5%, of a related substance having a relative retention time of 0.96 or 0.97 relative to the retention time of elraglusib.

In some embodiments, the solution contains less than 0.2%, as measured by high performance liquid chromatography (HPLC) area %, of a related substance having a relative retention time of 0.96 or 0.97 relative to the retention time of elraglusib.

In some embodiments, the related substance is one of the following compounds:

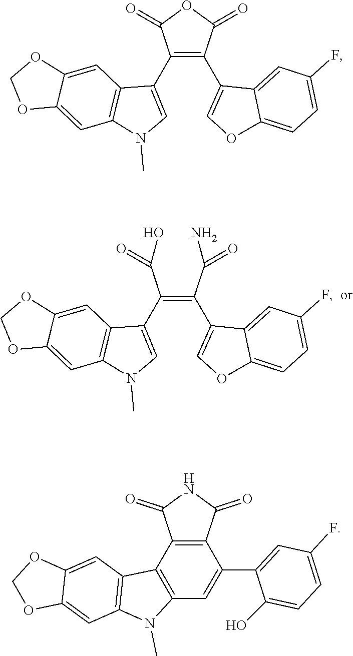

In some aspects, administration of an oral solution of the disclosure to a patient results in an elraglusib AUC∞ that is at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, or at least 80%, of the elraglusib AUC∞ resulting from IV administration to the patient of an equivalent dose (on a mg/kg basis) of elraglusib.

In some embodiments, administration of an oral solution of the disclosure to a patient results in an elraglusib AUC∞ that is at least 13% of the elraglusib AUC∞ resulting from IV administration to the patient of an equivalent dose (on a mg/kg basis) of elraglusib.

In some embodiments, administration of an oral solution of the disclosure to a patient results in an elraglusib AUC∞ that is at least 17% of the elraglusib AUC∞ resulting from IV administration to the patient of an equivalent dose (on a mg/kg basis) of elraglusib.

In some embodiments, administration of an oral solution of the disclosure to a patient results in an elraglusib AUC∞ that is at least 20% of the elraglusib AUC∞ resulting from IV administration to the patient of an equivalent dose (on a mg/kg basis) of elraglusib.

In some embodiments, administration of an oral solution of the disclosure to a patient results in an elraglusib AUC∞ that is at least 40% of the elraglusib AUC∞ resulting from IV administration to the patient of an equivalent dose (on a mg/kg basis) of elraglusib.

In some embodiments, administration of an oral solution of the disclosure to a patient results in an elraglusib AUC∞ that is at least 60% of the elraglusib AUC∞ resulting from IV administration to the patient of an equivalent dose (on a mg/kg basis) of elraglusib.

In some embodiments, administration of an oral solution of the disclosure to a patient results in an elraglusib AUC∞ that is at least 80% of the elraglusib AUC∞ resulting from IV administration to the patient of an equivalent dose (on a mg/kg basis) of elraglusib.

In some aspects of the disclosure, administration of an oral solution of the disclosure to a fed patient results in an elraglusib AUC∞ that is at least 2.4 times, at least 2.5 times, at least 2.6 times, at least 2.7 times, at least 2.8 times, at least 2.9 times, at least 3.0 times, at least 3.1 times, at least 3.2 times, at least 3.3 times, at least 3.4 times, at least 3.5 times, at least 3.6 times, at least 3.7 times, or at least 3.8 times, of the elraglusib AUC∞ resulting from administration of administration of the oral solution of the disclosure to a fasted patient.

### Oral Liquid Suspensions

In some aspects, the disclosure provides liquid suspensions comprising: elraglusib; an emulsifier that is a polyethylene glycol, mustard lecithin, soy lecithin, egg lecithin, monoglyceride, diglyceride, polysorbate, stearoyl lactylate, sorbitan ester, polyglycerol ester, or sucrose ester; a pharmaceutically acceptable alcohol; a surfactant that is sodium stearate, 4-(5-dodecyl)benzenesulfonate, sodium lauryl sulfate, docusate sodium, phosphatidylcholine, benzalkonium chloride, polyoxyethylene sorbitan fatty acid esters, polyoxyethylene 15 hydroxy stearate, polyoxyethylene castor oil derivatives, polyoxyethylene stearates, sorbitan fatty acid esters, polyoxyethylene alkyl ethers, and polyoxyethylene nonylphenol ether; and a pharmaceutically acceptable diluent.

In some embodiments, the disclosure is directed to a liquid suspension comprising about 0.04-0.6 wt. % elraglusib, about 0.8 to 10 wt. % of an emulsifier that is a polyethylene glycol, mustard lecithin, soy lecithin, egg lecithin, monoglyceride, diglyceride, polysorbate, stearoyl lactylate, sorbitan ester, polyglycerol ester, or sucrose ester; about 0.04-1.7 wt. % of a pharmaceutically acceptable alcohol, about 0.009-1 wt. % a surfactant that is sodium stearate, 4-(5-dodecyl)benzenesulfonate, sodium lauryl sulfate, docusate sodium, phosphatidylcholine, benzalkonium chloride, polyoxyethylene sorbitan fatty acid esters, polyoxyethylene 15 hydroxy stearate, polyoxyethylene castor oil derivatives, polyoxyethylene stearates, sorbitan fatty acid esters, polyoxyethylene alkyl ethers, and polyoxyethylene nonylphenol ether; and about 87 to 99 wt % of a pharmaceutically acceptable diluent.

In some aspects, the disclosure is directed to a liquid suspension comprising about 0.08-0.6 wt. % elraglusib, about 1.5 to 10 wt. % of an emulsifier that is a polyethylene glycol, mustard lecithin, soy lecithin, egg lecithin, monoglyceride, diglyceride, polysorbate, stearoyl lactylate, sorbitan ester, polyglycerol ester, or sucrose ester; about 0.08-1.7 wt. % of a pharmaceutically acceptable alcohol, about 0.01-1 wt. % a surfactant that is sodium stearate, 4-(5-dodecyl)benzenesulfonate, sodium lauryl sulfate, docusate sodium, phosphatidylcholine, benzalkonium chloride, polyoxyethylene sorbitan fatty acid esters, polyoxyethylene 15 hydroxy stearate, polyoxyethylene castor oil derivatives, polyoxyethylene stearates, sorbitan fatty acid esters, polyoxyethylene alkyl ethers, and polyoxyethylene nonylphenol ether; and about 87 to 99 wt % of a pharmaceutically acceptable diluent.

In some aspects, the disclosure is directed to a liquid suspension comprising about 0.15-0.6 wt. % elraglusib, about 3 to 10 wt. % of an emulsifier that is a polyethylene glycol, mustard lecithin, soy lecithin, egg lecithin, monoglyceride, diglyceride, polysorbate, stearoyl lactylate, sorbitan ester, polyglycerol ester, or sucrose ester; about 0.15-1.7 wt. % of a pharmaceutically acceptable alcohol, about 0.03-1 wt. % a surfactant that is sodium stearate, 4-(5-dodecyl)benzenesulfonate, sodium lauryl sulfate, docusate sodium, phosphatidylcholine, benzalkonium chloride, polyoxyethylene sorbitan fatty acid esters, polyoxyethylene 15 hydroxy stearate, polyoxyethylene castor oil derivatives, polyoxyethylene stearates, sorbitan fatty acid esters, polyoxyethylene alkyl ethers, and polyoxyethylene nonylphenol ether; and about 87 to 99 wt % of a pharmaceutically acceptable diluent.

In some aspects, the disclosure is directed to a liquid suspension comprising about 0.1-0.6 wt. % elraglusib, about 1.5 to 10 wt. % of an emulsifier that is a polyethylene glycol, mustard lecithin, soy lecithin, egg lecithin, monoglyceride, diglyceride, polysorbate, stearoyl lactylate, sorbitan ester, polyglycerol ester, or sucrose ester; about 0.1-1.7 wt. % of a pharmaceutically acceptable alcohol, about 0.01-1 wt. % a surfactant that is sodium stearate, 4-(5-dodecyl)benzenesulfonate, sodium lauryl sulfate, docusate sodium, phosphatidylcholine, benzalkonium chloride, polyoxyethylene sorbitan fatty acid esters, polyoxyethylene 15 hydroxy stearate, polyoxyethylene castor oil derivatives, polyoxyethylene stearates, sorbitan fatty acid esters, polyoxyethylene alkyl ethers, and polyoxyethylene nonylphenol ether; and about 87 to 98 wt % of a pharmaceutically acceptable diluent.

In some aspects, the liquid suspension comprises about 0.0.4-0.6 wt. % 3-(5-fluorobenzofuran-3-yl)-4-(5-methyl-5H-[1,3]dioxolo[4,5-f]indol-7-yl)-1H-pyrrole-2,5-dione, such as, for example, one of about 0.04%, about 0.05%, about 0.06%, about 0.07%, about 0.08%, about 0.09%, about 0.1%, about 0.15%, about 0.2%, about 0.25%, about 0.3%, about 0.35%, about 0.4%, about 0.45%, about 0.5%, about 0.55%, or about 0.6%.

In some embodiments of the liquid suspensions, the emulsifier is a polyethylene glycol (PEG).

In other embodiments of the liquid suspensions, the emulsifier is a polyethylene glycol having a molecular weight of 100-1000 Da.

In other embodiments of the liquid suspensions, the emulsifier is PEG 400.

In some embodiments, the emulsifier is a mustard lecithin.

In some embodiments, the emulsifier is a soy lecithin.

In some embodiments, the emulsifier is an egg lecithin.

In some embodiments, the emulsifier is a monoglyceride.

In some embodiments, the emulsifier is a diglyceride.

In some embodiments, the emulsifier is a polysorbate.

In some embodiments, the emulsifier is a stearoyl lactylate.

In some embodiments, the emulsifier is a sorbitan ester.

In some embodiments, the emulsifier is a polyglycerol ester.

In some embodiments, the emulsifier is a sucrose ester.

In some aspects, the emulsifier is present in the liquid suspensions in an amount of about 0.8-10 wt. %, such as, for example, one of about 0.8 wt. %, about 0.9 wt. %, about 1.0 wt. %, about 1.1 wt. %, about 1.2 wt. %, about 1.3 wt. %, about 1.4 wt. %, about 1.5 wt. %, about 2 wt. %, about 2.5 wt. %, about 3 wt. %, about 3.5 wt. %, about 4 wt. %, about 4.5 wt. %, about 5 wt. %, about 5.5 wt. %, about 6 wt. %, about 6.5 wt. %, about 7 wt. %, about 7.5 wt. %, about 8 wt. %, about 8.5 wt. %, about 9 wt. %, about 9.5 wt. %, or about 10 wt. %.

In some embodiments of the liquid suspensions, the pharmaceutically acceptable alcohol is ethanol.

In some aspects, the pharmaceutically acceptable alcohol is present in an amount of about 0.04-1.7 wt. %, such as, for example, one of about 0.04 wt. %, about 0.05 wt. %, about 0.06 wt. %, about 0.07 wt. %, about 0.08 wt. %, about 0.09 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, or about 1.7 wt. %.

In some embodiments, the surfactant in the liquid suspensions is sodium stearate.

In some embodiments, the surfactant is 4-(5-dodecyl)benzenesulfonate.

In some embodiments, the surfactant is sodium lauryl sulfate.

In some embodiments, the surfactant is docusate sodium.

In some embodiments, the surfactant is phosphatidylcholine.

In some embodiments, the surfactant is benzalkonium chloride.

In some embodiments, the surfactant is polyoxyethylene sorbitan fatty acid esters.

In some embodiments, the surfactant is polysorbate 80 (Polyoxyethylenesorbitan monooleate).

In some embodiments, the surfactant is polyoxyethylene 15 hydroxy stearate.

In some embodiments, the surfactant is a polyoxyethylene castor oil derivative.

In some embodiments, the surfactant is a polyoxyethylene stearate.

In some embodiments, the surfactant is a sorbitan fatty acid ester.

In some embodiments, the surfactant is a polyoxyethylene alkyl ethers.

In some embodiments, the surfactant is a polyoxyethylene nonylphenol ether.

In some aspects, the surfactant is present in the liquid suspensions in an amount of about 0.009-1% wt. % such as, for example, one of: about 0.009 wt. %, about 0.01 wt. %, about 0.05 wt. %, about 0.1 wt. %, about 0.15 wt. %, about 0.2 wt. %, about 0.25 wt. %, about 0.3 wt. %, about 0.35 wt. %, about 0.4 wt. %, about 0.45 wt. %, about 0.5 wt. %, about 0.55 wt. %, about 0.6 wt. %, about 0.65 wt. %, about 0.7 wt. %, about 0.75 wt. %, about 0.8 wt. %, about 0.85 wt. %, about 0.9 wt. %, about 0.95 wt. %, or about 1 wt. %.

In some aspects, the oral suspensions of the disclosure comprise about 87-99 wt % of a pharmaceutically acceptable diluent, such as, for example, one of 87 wt %, 88 wt %, 89 wt %, 90 wt %, 91 wt %, 92 wt %, 93 wt %, 94 wt %, 95 wt %, 96 wt %, 97 wt %, 98 wt %, or 99 wt %. As used herein, a pharmaceutically acceptable diluent is a liquid which can be safely ingested by a human. Examples of pharmaceutically acceptable diluents include water and aqueous solutions, such as saline, electrolyte solutions, sugar solutions, flavoring solutions, and the like.

In some embodiments, the pharmaceutically acceptable diluent is water.

In other embodiments, the pharmaceutically acceptable diluent is dextrose (5%) in water (also known as D5W).

In some embodiments, the disclosure is directed to a suspension comprising: about 0.04-0.05 wt. % elraglusib, about 0.8-1.0 wt. % polyethylene glycol 400, about 0.04-0.05 wt. % ethanol, about 0.009-0.05 wt. % polysorbate 80, and about 98-99 wt. % of Dextrose (5%) in water solution.

In some embodiments, the disclosure is directed to a suspension comprising: about 0.08-0.1 wt. % elraglusib, about 1.5-2.0 wt. % polyethylene glycol 400, about 0.08-0.1 wt. % ethanol, about 0.018-0.1 wt. % polysorbate 80, and about 97-99 wt. % of Dextrose (5%) in water solution.

In some embodiments, the disclosure is directed to a suspension comprising: about 0.15-0.2 wt. % elraglusib, about 3-4 wt. % polyethylene glycol 400, about 0.15-0.2 wt. % ethanol, about 0.03-0.2 wt. % polysorbate 80, and about 96-97 wt. % of D5W.

In some embodiments, the disclosure is directed to a suspension comprising: about 0.1-0.4 wt. % elraglusib, about 2-8 wt. % polyethylene glycol 400, about 0.1-0.5 wt. % ethanol, about 0.01-0.05 wt. % polysorbate 80, and about 98 wt. % of D5W.

In some embodiments, the suspension comprises about 0.1-0.5 wt. % elraglusib, about 2.6-9.4 wt. % Polyethylene glycol 400, about 0.1-0.5 wt. % ethanol, and about 0.03-0.1 wt. % polysorbate 80; and about 97 wt. % of Dextrose (5%) in water solution.

In some embodiments, the suspension comprises about 0.15-0.6 wt. % elraglusib, about 2.7-9.5 wt. % Polyethylene glycol 400, about 0.5-1.7 wt. % ethanol, and about 0.01 wt. % polysorbate 80; and about 96 wt. % of D5W.

In some embodiments, the concentration of elraglusib in the suspension is at least 0.5 mg/mL.

In some embodiments, the concentration of elraglusib in the suspension is at least 1 mg/mL.

In some embodiments, the concentration of elraglusib in the suspension is at least 2 mg/mL.

In some aspects, the liquid suspension has particles having a diameter in the range of 100-1000 nm.

In some aspects, oral administration of a liquid solution or liquid suspension of the disclosure to a subject results elraglusib plasma concentrations ranging from 1000 to 10000 ng/mL.

In some aspects, administration of an oral suspension of the disclosure to a patient results in an elraglusib AUC∞ that is at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, or at least 80%, of the elraglusib AUC∞ resulting from IV administration to the patient of an equivalent dose (on a mg/kg basis) of elraglusib.

In some embodiments, administration of an oral suspension of the disclosure to a patient results in an elraglusib AUC∞ that is at least 13% of the elraglusib AUC∞ resulting from IV administration to the patient of an equivalent dose (on a mg/kg basis) of elraglusib.

In some embodiments, administration of an oral suspension of the disclosure to a patient results in an elraglusib AUC∞ that is at least 17% of the elraglusib AUC∞ resulting from IV administration to the patient of an equivalent dose (on a mg/kg basis) of elraglusib.

In some embodiments, administration of an oral suspension of the disclosure to a patient results in an elraglusib AUC∞ that is at least 20% of the elraglusib AUC∞ resulting from IV administration to the patient of an equivalent dose (on a mg/kg basis) of elraglusib.

In some embodiments, administration of an oral suspension of the disclosure to a patient results in an elraglusib AUC∞ that is at least 40% of the elraglusib AUC∞ resulting from IV administration to the patient of an equivalent dose (on a mg/kg basis) of elraglusib.

In some embodiments, administration of an oral suspension of the disclosure to a patient results in an elraglusib AUC∞ that is at least 60% of the elraglusib AUC∞ resulting from IV administration to the patient of an equivalent dose (on a mg/kg basis) of elraglusib.

In some embodiments, administration of an oral suspension of the disclosure to a patient results in an elraglusib AUC∞ that is at least 80% of the elraglusib AUC∞ resulting from IV administration to the patient of an equivalent dose (on a mg/kg basis) of elraglusib.

In some aspects of the disclosure, administration of an oral suspension of the disclosure to a fed patient results in an elraglusib AUC∞ that is at least 2.4 times, at least 2.5 times, at least 2.6 times, at least 2.7 times, at least 2.8 times, at least 2.9 times, at least 3.0 times, at least 3.1 times, at least 3.2 times, at least 3.3 times, at least 3.4 times, at least 3.5 times, at least 3.6 times, at least 3.7 times, or at least 3.8 times, of the elraglusib AUC∞ resulting from administration of administration of the oral solution of the disclosure to a fasted patient.

### Pharmaceutical Compositions for Oral Administration

The pharmaceutical compositions of the disclosure are typically formulated to provide a therapeutically effective amount of elraglusib as the active ingredient. In some embodiments, the pharmaceutical compositions contain elraglusib and one or more pharmaceutically acceptable excipients or carriers, including inert solid diluents and fillers, liquid diluents, including sterile aqueous solution and various organic solvents, permeation enhancers, solubilizers, adjuvants, surfactants, and the like.

In some aspects, the disclosure provides pharmaceutical compositions for oral administration comprising an elraglusib solid dispersion as disclosed herein, a elraglusib liquid suspension as disclosed herein, or liquid solution as disclosed herein.

In some aspects, the disclosure provides pharmaceutical compositions for oral administration comprising an elraglusib ASD as disclosed herein, an elraglusib liquid suspension as disclosed herein, or an elraglusib liquid solution as disclosed herein.

In some embodiments, the pharmaceutical composition consists of an elraglusib amorphous solid dispersion as disclosed herein, an elraglusib liquid solution as disclosed herein, or a elraglusib liquid suspension as disclosed herein.

In some aspects, pharmaceutical compositions of the disclosure can be administered alone or in combination with one or more other agents, which are also typically administered in the form of pharmaceutical compositions. Where desired, the elraglusib and the other agent(s) may be mixed into a preparation or both components may be formulated into separate preparations to use them in combination separately or at the same time.

In some embodiments, the disclosure provides a solid pharmaceutical composition for oral administration containing: (i) an effective amount of elraglusib; optionally (ii) an effective amount of a second agent; and (iii) a pharmaceutical excipient suitable for oral administration. In some embodiments, the composition further contains: (iv) an effective amount of a third agent.

In some aspects, the disclosure is directed to pharmaceutical compositions comprising a therapeutically effective amount of an amorphous solid dispersion of the disclosure and a pharmaceutically acceptable excipient.

In some embodiments, the pharmaceutical composition is in a form suitable for oral (e.g., peroral) administration such as, for example tablets, capsules, caplets, reconstitutable powders, elixirs, liquids, colloidal or other types of suspensions, beads, beadlets, granules, microparticles, nanoparticles, and combinations thereof.

In some embodiments, the pharmaceutical composition is a tablet.

In other embodiments, the pharmaceutical composition is a capsule.

In some embodiments, the amount of elraglusib in the pharmaceutical composition is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g (or a number in the range defined by and including any two numbers above).

In some embodiments, the amount of elraglusib is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g (or a number in the range defined by and including any two numbers above).

In some embodiments, the amount of elraglusib is in the range of 0.0001-10 g, 0.0005-9 g, 0.001-8 g, 0.005-7 g, 0.01-6 g, 0.05-5 g, 0.1-4 g, 0.5-4 g, or 1-3 g.

In some embodiments in the treatment of adult humans, dosages from 0.01 to 1000 mg, from 0.5 to 100 mg, and from 1 to 50 mg per day are examples of dosages that may be used. The exact dosage will depend upon the form in which the compound is administered, the subject to be treated, the body weight of the subject to be treated, and the preference and experience of the attending physician.

In some aspects, the liquid solution or liquid suspensions of the disclosure are orally administered. In such embodiments, the liquid solution or liquid suspensions may further comprise a sweetening or flavoring agent, a coloring agent or dyes, an emulsifying and/or suspending agent, and diluents as water, ethanol, propylene glycol, glycerin, polysorbates and combinations thereof.

In other aspects, the solid dispersions of the disclosure are orally administered.

Pharmaceutical compositions comprising the solid dispersions of the disclosure can be formulated as discrete dosage forms, such as capsules, cachets, or tablets, liquids or aerosol sprays each containing a predetermined amount of elraglusib as a powder or as granules; or a suspension in an aqueous or non-aqueous liquid, an oil-in-water emulsion, or a water-in-oil liquid emulsion. Such dosage forms can be prepared by any of the methods of pharmacy. In general, the compositions are prepared by uniformly and intimately admixing the active ingredient with liquid carriers or finely divided solid carriers or both, and then, if necessary, shaping the product into the desired dosage form. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

### Capsules

In some aspects, the disclosure is directed to pharmaceutical dosage forms that are capsules comprising an elraglusib ASD of the disclosure.

In some embodiments, the disclosure is directed to pharmaceutical dosage forms that are capsules comprising an elraglusib ASD of the disclosure.

In some embodiments, the capsules comprise an elraglusib ASD of the disclosure, and one or more pharmaceutically acceptable excipients.

In some embodiments, the capsules comprise an elraglusib ASD of the disclosure, and one or more pharmaceutically acceptable excipients wherein the weight ratio of ASD to pharmaceutically acceptable excipients is about 19:1.

In some embodiments, the capsules comprise an elraglusib ASD of the disclosure, and croscarmellose sodium.

In some embodiments, the capsules comprise an elraglusib ASD of the disclosure, and silica.

In some embodiments, the capsules comprise an elraglusib ASD of the disclosure, and a blend of croscarmellose sodium and silica.

In some embodiments, the capsules comprise an elraglusib ASD of the disclosure, and a blend of croscarmellose sodium and silica in a weight ratio of croscarmellose sodium:silica (90:10).

In some embodiments, the capsules comprise an elraglusib ASD of the disclosure, and a blend of croscarmellose sodium and silica wherein the weight ratio of ASD to blend of croscarmellose sodium and silica is about 19:1.

In some embodiments, the ASD in the capsules of the disclosure is Elra:PVAP.

In some embodiments, the ASD in the capsules of the disclosure is Elra:CAP.

In some embodiments, the ASD in the capsules of the disclosure is Elra:poly(methyl methacrylate-co-methacrylic acid) (i.e., amorphous elraglusib with poly(methyl methacrylate-co-methacrylic acid)).

In some embodiments, the ASD in the capsules of the disclosure is Elra:EL100 (i.e., amorphous elraglusib with Eudragit® EL100 polymer).

In some embodiments, the capsules of the disclosure comprise a hard gelatin capsule.

In other embodiments, the capsules of the disclosure comprise a soft gelatin capsule.

### Tablets

In some aspects, the disclosure is directed to pharmaceutical dosage forms that are tablets comprising an elraglusib ASD of the disclosure.

In some embodiments, the tablets of the disclosure comprise:
(i) an ASD comprising elraglusib and a stabilizing polymer;
(ii) a binder;
(iii) a filler;
(iv) a disintegrant; and
(v) a lubricant.

In some embodiments, the tablets of the disclosure comprise:
(i) about 40-60% by weight of an ASD comprising elraglusib and a stabilizing polymer;
(ii) about 19-27% by weight of a binder;
(iii) about 10-25% by weight of a filler;
(iv) about 4-9% by weight of a disintegrant; and
(v) about 1-3% of a lubricant.

In some embodiments, the tablets of the disclosure comprise:
(i) about 50% by weight of an ASD comprising elraglusib and a stabilizing polymer;
(ii) about 19.5% by weight of a binder;
(iii) about 19.5% by weight of a filler;
(iv) about 9% by weight of a disintegrant; and
(v) about 2% of a lubricant.

In some embodiments of the tablets of the disclosure, the ASD comprises elraglusib and poly(methyl methacrylate-co-methacrylic acid).

In some embodiments of the tablets of the disclosure, the ASD comprises about 50% by weight elraglusib and about 50% by weight poly(methyl methacrylate-co-methacrylic acid).

In some embodiments of the tablets of the disclosure, the ASD comprises elraglusib and EL100.

In some embodiments of the tablets of the disclosure, the ASD comprises about 50% by weight elraglusib and about 50% by weight EL100.

In some embodiments of the tablets of the disclosure, the ASD comprises elraglusib and CAP.

In some embodiments of the tablets of the disclosure, the ASD comprises about 50% by weight elraglusib and about 50% by weight CAP.

In some embodiments of the tablets of the disclosure, the binder is microcrystalline cellulose.

In some embodiments of the tablets of the disclosure, the filler is mannitol.

In some embodiments of the tablets of the disclosure, the disintegrant is croscarmellose sodium or a polyvinyl pyrrolidone.

In some embodiments of the tablets of the disclosure, the disintegrant is croscarmellose sodium.

In some embodiments of the tablets of the disclosure, the lubricant is one or more of a syloid silica or magnesium stearate.

In some embodiments of the tablets of the disclosure, the lubricant comprises a syloid silica and magnesium stearate.

In some embodiments, the tablets of the disclosure comprise:
(i) an ASD comprising about 50% by weight elraglusib and about 50% by weight poly(methyl methacrylate-co-methacrylic acid);
(ii) microcrystalline cellulose;
(iii) mannitol;
(iv) croscarmellose sodium;
(v) silicon dioxide; and
(vi) magnesium stearate.

In some embodiments, the tablets of the disclosure comprise:
(i) about 50% by weight of an ASD comprising about 50% by weight elraglusib and about 50% by weight poly(methyl methacrylate-co-methacrylic acid);
(ii) about 19.5% by weight microcrystalline cellulose;
(iii) about 19.5% by weight mannitol;
(iv) about 9% by weight croscarmellose sodium;
(v) about 1% by weight silicon dioxides; and
(vi) about 1% magnesium stearate

In some embodiments, the tablets of the disclosure comprise:
(i) 50% by weight of an ASD comprising about 50% by weight elraglusib and about 50% by weight poly(methyl methacrylate-co-methacrylic acid);
(ii) 19.5% by weight microcrystalline cellulose;
(iii) 19.5% by weight mannitol;
(iv) 9% by weight croscarmellose sodium;
(v) 1% by weight silicon dioxide; and
(vi) 1% magnesium stearate

In some embodiments, the tablets of the disclosure comprise:
(i) an ASD comprising about 50% by weight elraglusib and about 50% by weight EL100;
(ii) microcrystalline cellulose;
(iii) mannitol;
(iv) croscarmellose sodium;
(v) silicon dioxide; and
(vi) magnesium stearate.

In some embodiments, the tablets of the disclosure comprise:
(i) about 50% by weight of an ASD comprising about 50% by weight elraglusib and about 50% by weight EL100;
(ii) about 19.5% by weight microcrystalline cellulose;
(iii) about 19.5% by weight mannitol;
(iv) about 9% by weight croscarmellose sodium;
(v) about 1% by weight silicon dioxides; and
(vi) about 1% magnesium stearate

In some embodiments, the tablets of the disclosure comprise:
(i) 50% by weight of an ASD comprising about 50% by weight elraglusib and about 50% by weight EL100;
(ii) 19.5% by weight microcrystalline cellulose;
(iii) 19.5% by weight mannitol;
(iv) 9% by weight croscarmellose sodium;
(v) 1% by weight silicon dioxide; and
(vi) 1% magnesium stearate

In some embodiments, the tablets of the disclosure comprise:
(i) about 42% by weight of an ASD comprising elraglusib and a stabilizing polymer;
(ii) about 27% by weight of a binder;
(iii) about 25% by weight of a filler;
(iv) about 4% by weight of a disintegrant; and
(v) about 2% of a lubricant.

In some embodiments, the tablets of the disclosure comprise:
(i) an ASD comprising about 50% by weight elraglusib and about 50% by weight poly(methyl methacrylate-co-methacrylic acid);
(ii) microcrystalline cellulose;
(iii) mannitol;
(iv) croscarmellose sodium;
(v) silicon dioxide; and
(vi) magnesium stearate.

In some embodiments, the tablets of the disclosure comprise:
(i) about 42% by weight of an ASD comprising about 50% by weight elraglusib and about 50% by weight poly(methyl methacrylate-co-methacrylic acid);
(ii) about 27% by weight microcrystalline cellulose;
(iii) about 25% by weight mannitol;
(iv) about 4% by weight croscarmellose sodium;
(v) about 0.5% by weight silicon dioxide; and
(vi) about 1.5% magnesium stearate

In some embodiments, the tablets of the disclosure comprise:
(i) an ASD comprising about 50% by weight elraglusib and about 50% by weight EL100;
(ii) microcrystalline cellulose;
(iii) mannitol;
(iv) croscarmellose sodium;
(v) silicon dioxide; and
(vi) magnesium stearate.

In some embodiments, the tablets of the disclosure comprise:
(i) about 42% by weight of an ASD comprising about 50% by weight elraglusib and about 50% by weight EL100;
(ii) about 27% by weight microcrystalline cellulose;
(iii) about 25% by weight mannitol;
(iv) about 4% by weight croscarmellose sodium;
(v) about 0.5% by weight silicon dioxide; and
(vi) about 1.5% magnesium stearate

In some embodiments, the tablets of the disclosure comprise:
(i) an ASD comprising about 50% by weight elraglusib and about 50% by weight CAP;
(ii) microcrystalline cellulose;
(iii) mannitol;
(iv) croscarmellose sodium;
(v) silicon dioxide; and
(vi) magnesium stearate.

In some embodiments, the tablets of the disclosure comprise:
(i) about 42% by weight of an ASD comprising about 50% by weight elraglusib and about 50% by weight CAP;
(ii) about 27% by weight microcrystalline cellulose;
(iii) about 25% by weight mannitol;
(iv) about 4% by weight croscarmellose sodium;
(v) about 0.5% by weight silicon dioxide; and
(vi) about 1.5% magnesium stearate

In some embodiments, the tablets of the disclosure comprise:
(i) about 50% by weight of an ASD comprising elraglusib and a stabilizing polymer;
(ii) about 22% by weight of a binder;
(iii) about 22% by weight of a filler;
(iv) about 5% by weight of a disintegrant; and
(v) about 2% of a lubricant.

In some embodiments, the tablets of the disclosure comprise:
(i) about 50% by weight of an ASD comprising about 50% by weight elraglusib and about 50% by weight EL100;
(ii) about 21.5% by weight microcrystalline cellulose;
(iii) about 21.5% by weight mannitol;
(iv) about 5% by weight croscarmellose sodium;
(v) about 1% by weight silicon dioxide; and
(vi) about 1% by weight magnesium stearate.

In some embodiments, the tablets of the disclosure comprise:
(i) about 50% by weight of an ASD comprising about 50% by weight elraglusib and about 50% by weight poly(methyl methacrylate-co-methacrylic acid);
(ii) about 21.5% by weight microcrystalline cellulose;
(iii) about 21.5% by weight mannitol;
(iv) about 5% by weight croscarmellose sodium;
(v) about 1% by weight silicon dioxide; and
(vi) about 1% by weight magnesium stearate.

In some embodiments, the tablets of the disclosure comprise:
(i) about 55.6% by weight of an ASD comprising elraglusib and a stabilizing polymer;
(ii) about 24.9% by weight of a binder;
(iii) about 12.5% by weight of a filler;
(iv) about 5% by weight of a disintegrant; and
(v) about 2% of a lubricant.

In some embodiments, the tablets of the disclosure comprise:
(i) about 55.6% by weight of an ASD comprising about 50% by weight elraglusib and about 50% by weight poly(methyl methacrylate-co-methacrylic acid);
(ii) about 21.5% by weight microcrystalline cellulose;
(iii) about 12.5% by weight mannitol;
(iv) about 5% by weight croscarmellose sodium;
(v) about 1% by weight silicon dioxide; and
(vi) about 1% by weight magnesium stearate.

In some embodiments, the tablets of the disclosure comprise:
(i) about 55.6% by weight of an ASD comprising about 50% by weight elraglusib and about 50% by weight EL100;
(ii) about 21.5% by weight microcrystalline cellulose;
(iii) about 12.5% by weight mannitol;
(iv) about 5% by weight croscarmellose sodium;
(v) about 1% by weight silicon dioxide; and
(vi) about 1% by weight magnesium stearate.

In some embodiments, the tablets of the disclosure comprise:
(i) about 58.8% by weight of an ASD comprising elraglusib and a stabilizing polymer;
(ii) about 20.8% by weight of a binder;
(iii) about 10.4% by weight of a filler;
(iv) about 8% by weight of a disintegrant; and
(v) about 2% of a lubricant.

In some embodiments, the tablets of the disclosure comprise:
(i) about 58.8% by weight of an ASD comprising about 50% by weight elraglusib and about 50% by weight poly(methyl methacrylate-co-methacrylic acid);
(ii) about 20.8% by weight microcrystalline cellulose;
(iii) about 10.4% by weight mannitol;
(iv) about 8% by weight croscarmellose sodium;
(v) about 1% by weight silicon dioxide; and
(vi) about 1% by weight magnesium stearate.

In some embodiments, the tablets of the disclosure comprise:
(i) about 58.8% by weight of an ASD comprising about 50% by weight elraglusib and about 50% by weight EL100;
(ii) about 20.8% by weight microcrystalline cellulose;
(iii) about 10.4% by weight mannitol;
(iv) about 8% by weight croscarmellose sodium;
(v) about 1% by weight silicon dioxide; and
(vi) about 1% by weight magnesium stearate.

In some aspects, oral administration of a tablet of the disclosure to a patient results in an elraglusib AUC∞ that is at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, or at least 80%, of the elraglusib AUC∞ resulting from IV administration to the patient of an equivalent dose (on a mg/kg basis) of elraglusib.

In some aspects, oral administration of a solid dispersion of the disclosure to a patient results in an elraglusib AUC∞ that is at least 68% of the elraglusib AUC∞ resulting from IV administration to the patient of an equivalent dose (on a mg/kg basis) of elraglusib.

In some embodiments, the pharmaceutical compositions of the disclosure are anhydrous. Anhydrous pharmaceutical compositions of the disclosure can be prepared using anhydrous or low moisture containing ingredients and low moisture or low humidity conditions. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

Elraglusib can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions and solutions) or aerosols; or carriers such as starches, sugars, microcrystalline cellulose, diluents, granulating agents, lubricants, binders, polymers, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions of the disclosure include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pregelatinized starch, hydroxypropyl cellulose, hydroxypropyl methyl cellulose (hypromellose), microcrystalline cellulose, and mixtures thereof.

Examples of suitable fillers for use in the pharmaceutical compositions disclosed herein include, but are not limited to, talc, calcium carbonate (e.g., granules or powder), microcrystalline cellulose, powdered cellulose, dextrates, kaolin, mannitol, silicic acid, sorbitol, starch, pregelatinized starch, and mixtures thereof.

Disintegrants may be used in the pharmaceutical compositions to provide tablets that disintegrate when exposed to an aqueous environment. The amount of disintegrant used may vary based upon the type of formulation and mode of administration, and may be readily discernible to those of ordinary skill in the art. In some embodiments, about 0.5 to about 15 weight percent of disintegrant, or about 1 to about 5 weight percent of disintegrant, may be used in the pharmaceutical composition. Disintegrants that can be used to form pharmaceutical compositions of the disclosure include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pregelatinized starch, other starches, clays, other algins, other celluloses, gums or mixtures thereof.

Lubricants which can be used to form pharmaceutical compositions of the disclosure include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a colloid silicon dioxide gel, a coagulated aerosol of synthetic silica, or mixtures thereof. A lubricant can optionally be added, in an amount of, for example, less than about 1 weight percent of the pharmaceutical composition.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions of the disclosure include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions.

Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and diacetylated tartaric acid esters of mono- and diglycerides; succinylated mono- and diglycerides; citric acid esters of mono- and diglycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10-oleate, Tween 40, Tween 60, Tween 80, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a subject using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids.

Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

In some embodiments, the elraglusib composition is administered in multiple doses. Dosing may be about once, twice, three times, four times, five times, six times, or more than six times per day. Dosing may be about once a month, once every two weeks, once a week, twice a week, or once every other day. In another embodiment elraglusib composition and another agent are administered together about once per day to about 6 times per day. In another embodiment the administration of a elraglusib composition and an agent continues for less than about 7 days. In yet another embodiment the administration continues for more than about 6, 10, 14, 28 days, two months, six months, or one year. In some cases, continuous dosing is achieved and maintained as long as necessary.

Administration of an elraglusib composition may continue as long as necessary. In some embodiments, the elraglusib composition is administered for more than 1, 2, 3, 4, 5, 6, 7, 14, or 28 days. In some embodiments, the elraglusib composition is administered for less than 28, 14, 7, 6, 5, 4, 3, 2, or 1 day. In some embodiments, the elraglusib composition is administered for more than 1, 2, 3, 4, 5, 6, 12, 18, or 24 months. In some embodiments, the elraglusib composition is administered chronically on an ongoing basis, for as long as necessary, e.g., for the treatment of a chronic disease or disorder.

An effective amount of the elraglusib composition may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

In some aspects, the methods of the disclosure result in plasma concentrations of elraglusib that exhibit a food effect. The term “food effect,” as used herein, means that the extent and/or rate of drug absorption depends on whether the subject is fed or fasted at the time that the drug is administered.

The term “fed” as used herein, means that the subject has eaten food immediately prior to and/or immediately after ingesting elraglusib or a pharmaceutically acceptable salt thereof. For example, in some embodiments, “fed” means that the subject has eaten food 30 minutes or less prior to ingesting the elraglusib or a pharmaceutically acceptable salt thereof.

The term “fasted” as used herein, means that the subject has not eaten food in the 8 hours immediately prior to, or within 4 hours immediately after, ingesting the elraglusib or a pharmaceutically acceptable salt thereof.

In some embodiments, oral administration of an elraglusib pharmaceutical composition of the disclosure to a subject results in a plasma elraglusib Cmax (fed):Cmax (fasted) ratio that is about 2.5-5, for example, about 2.5, about 2.6, about 2.7, about 2.8, about 2.9, about 3.0, about 3.1, about 3.2, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, or about 5.0.

In some embodiments, oral administration of an elraglusib pharmaceutical composition of the disclosure to a subject results in plasma elraglusib Cmax (fed):Cmax (fasted) ratio that is 2.5 or greater, such as, for example, 2.5, 2.6, 2.7, 2.8, 2.9, 3.0, 3.1, 3.2, 3.4, 3.5, 3.6, 3.7, 3.8, 3.9, 4.0, 4.1, 4.2, 4.3, 4.4, 4.5, 4.6, 4.7, 4.8, 4.9, or 5.0.

In some embodiments, oral administration of an elraglusib pharmaceutical composition of the disclosure to a subject results in plasma elraglusib Cmax (fed) that is at least 250% of the corresponding plasma elraglusib Cmax (fasted) such as, for example, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, and the like.

As used herein, the term “Cmax (fasted)”, refers to the peak concentration in the plasma a following oral administration of an elraglusib pharmaceutical composition of the disclosure to a subject that is fasted. As used herein, the term “Cmax (fed)”, refers to the peak concentration in the plasma following oral administration of an elraglusib pharmaceutical composition of the disclosure to a subject that is fed. The peak concentration of elraglusib in the subject's plasma samples can be determined using an appropriate pharmacokinetic model applied to a plasma concentration vs. time profile determined using standard analytical methods. Appropriate pharmacokinetic models for determining Cmax are known to those of ordinary skill in the art.

In some embodiments of the disclosed methods, the Cmax (fed):Cmax (fasted) ratio is calculated using the arithmetic mean of the individual Cmax (fed) values calculated in a population of subjects and the arithmetic mean of the individual Cmax (fasted) values calculated in a population of subjects.

In some embodiments of the disclosed methods, the Cmax (fed):Cmax (fasted) ratio is calculated using the geometric mean of the individual Cmax (fed) values calculated in a population of subjects and the geometric mean of the individual Cmax (fasted) values calculated in a population of subjects.

In some embodiments, oral administration of an elraglusib pharmaceutical composition of the disclosure to a subject results in plasma elraglusib AUC∞ (fed):AUC∞ (fasted) ratio that is about 2.4 to 3.6, for example about 2.4, 2.5, 2.6, 2.7, 2.8, 2.9, 3.0, 3.1, 3.2, 3.3, 3.4, 3.5, or 3.6.

In some embodiments, oral administration of an elraglusib pharmaceutical composition of the disclosure to a subject results in plasma elraglusib AUC∞ (fed):AUC∞ (fasted) ratio that is 2.4 or greater, such as, for example, 2.4, 2.5, 2.6, 2.7, 2.8, 2.9, 3.0, 3.1, 3.2, 3.3, 3.4, 3.5, 3.6, and the like.

In some embodiments, oral administration of an elraglusib pharmaceutical composition of the disclosure to a subject results in plasma elraglusib AUC∞ (fed) that is at least 240% of the corresponding plasma elraglusib AUC∞ (fasted) such as, for example, 240%, 245%, 250%, 255%, 260%, 265%, 270%, 275%, 280%, 285%, 290%, 295%, 300%, 305%, 310%, 315%, 320%, 325%, 330%, 335%, 340%, 345%, 350%, and the like.

As used herein, the term “AUC∞ (fasted)”, refers to the area under the plasma concentration-time curve from extrapolation of AUCo to ∞ following oral administration of an elraglusib pharmaceutical composition of the disclosure to a subject that is fasted. As used herein, the term “AUC∞ (fed)”, refers to the area under the plasma concentration-time curve from extrapolation of AUCo to ∞ following oral administration of an elraglusib pharmaceutical composition of the disclosure to a subject that is fed. Methods for determining concentration-time curves and AUC are known to those of ordinary skill in the art.

In some embodiments of the disclosed methods, the AUC∞ (fed):AUC∞ (fasted) ratio is calculated using the arithmetic mean of the individual AUC∞ (fed) values calculated in a population of subjects and the arithmetic mean of the individual AUC∞ (fasted) values calculated in a population of subjects.

In other embodiments of the disclosed methods, the AUC∞ (fed):AUC∞ (fasted) ratio is calculated using the geometric mean of the individual AUC∞ (fed) values calculated in a population of subjects and the geometric mean of the individual AUC∞ (fasted) values calculated in a population of subjects.

### Methods of Use

In some aspects, the disclosure is directed to methods for treating a disease or disorder in a subject in need thereof, the methods comprising orally administering to the subject a solid dispersion according to any embodiment disclosed herein or a liquid solution or liquid suspension according to any embodiment disclosed herein.

In some embodiments, the disclosure is directed to methods for treating a disease or disorder in a subject in need thereof, the method comprising orally administering to the subject a solid dispersion of the disclosure.

In some embodiments, the disclosure is directed to methods for treating a disease or disorder in a subject in need thereof, the method comprising orally administering to the subject a solution or suspension of the disclosure.

In some embodiments of such methods, the solution or suspension is orally administered as a pharmaceutical composition.

In other embodiments of such methods, the solid dispersion is orally administered as pharmaceutical composition.

In some embodiments, an effective amount of elraglusib for use in the methods of treatment of the disclosure is based on the weight and condition of the patient and the disease being treated. In some embodiments, an effective amount of elraglusib is about from 0.1 mg/kg to 20 mg/kg, for example, about 0.1 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg.

In some embodiments, the disease or disorder is cancer.

In some embodiments, the cancer is brain cancer, lung cancer, breast cancer, ovarian cancer, bladder cancer, neuroblastoma, renal cancer, pancreatic cancer, sarcomas, or glioblastoma.

In some embodiments, the cancer is glioblastoma.

In other embodiments, the disease or disorder is a lymphoproliferative disorder.

In some embodiments, the lymphoproliferative disorder is a malignant B-cell lymphoproliferative disorder, such as, for example, Diffuse large B-cell lymphoma, acute lymphocytic leukemia, lymphoid blastic phase Chronic Myeloid Leukemia, Chronic lymphocytic leukemia/Small lymphocytic lymphoma, Extranodal marginal zone B-cell lymphomas, Mucosa-associated lymphoid tissue lymphomas, Follicular lymphoma, Mantle cell lymphoma, Nodal marginal zone B-cell lymphoma, Burkitt lymphoma, Hairy cell leukemia, Primary central nervous system lymphoma, Splenic marginal zone B-cell lymphoma, Waldenstrom's macroglobulinemia/Lymphoplasmacytic lymphoma, Multiple myeloma, Plasma cells dyscrasias, Plasma cell neoplasms, Primary mediastinal B-cell lymphoma, Hodgkin Disease, or Castelman's Disease.

In some embodiments, the malignant B-cell lymphoproliferative disorder is Diffuse large B-cell lymphoma.

In other embodiments, the Diffuse large B-cell lymphoma is Double-Hit lymphoma.

In other embodiments, the malignant lymphoproliferative disorder is a malignant T-cell lymphoproliferative disorder, such as, for example, T-cell leukemia/lymphoma, Extranodal natural killer/T-cell lymphoma, Cutaneous T-cell lymphoma, Enteropathy-type T-cell lymphoma, Angioimmunoblastic T-cell lymphoma, Anaplastic large T/null-cell lymphoma, Subcutaneous panniculitis-like T-cell lymphoma, T-cell acute lymphocytic leukemia, T-cell large granular lymphocyte leukemia, Lymphoid blastic phase Chronic Myeloid Leukemia, post-transplantation lymphoproliferative syndromes, human T-cell leukemia virus type 1-positive (HTLV-1⁺) adult T-cell leukemia/lymphoma (ATL), T-cell prolymphocytic leukemia (T-PLL), or unspecified T-cell lymphoma.

In other embodiments, the disease or disorder is traumatic brain injury.

In other embodiments, the disease or disorder is idiopathic pulmonary fibrosis.

In other embodiments, the disease or disorder is pleural fibrosis.

The disclosure is also directed to the following aspects:
Aspect 1. A solid dispersion comprising amorphous 3-(5-fluorobenzofuran-3-yl)-4-(5-methyl-5H-[1,3]dioxolo[4,5-f]indol-7-yl)-1H-pyrrole-2,5-dione and a stabilizing polymer.
Aspect 2. The solid dispersion according to aspect 1, wherein said stabilizing polymer is N-vinyl-2-pyrrolidone-vinyl acetate copolymer (copovidone), cellulose acetate phthalate, hydroxypropyl methylcellulose phthalate, hydroxypropyl methylcellulose acetate succinate, polyvinyl acetate phthalate, polyvinylpyrrolidone, or a miscible mixture thereof.
Aspect 3. The solid dispersion according to aspect 2, wherein said stabilizing polymer is N-vinyl-2-pyrrolidone-vinyl acetate copolymer (copovidone).
Aspect 4. The solid dispersion according to aspect 2, wherein said stabilizing polymer is cellulose acetate phthalate.
Aspect 5. The solid dispersion according to aspect 2, wherein said stabilizing polymer is hydroxypropyl methylcellulose phthalate.
Aspect 6. The solid dispersion according to aspect 2, wherein said stabilizing polymer is hydroxypropyl methylcellulose acetate succinate.
Aspect 7. The solid dispersion according to aspect 2, wherein said stabilizing polymer is polyvinyl acetate phthalate.
Aspect 8. The solid dispersion according to aspect 2, wherein said stabilizing polymer is polyvinylpyrrolidone.
Aspect 9. The solid dispersion according to any one of the preceding aspects, wherein the 3-(5-fluorobenzofuran-3-yl)-4-(5-methyl-5H-[1,3]dioxolo[4,5-f]indol-7-yl)-1H-pyrrole-2,5-dione is present in an amount of from about 10% to about 70% by weight relative to the weight of the stabilizing polymer.
Aspect 10. The solid dispersion according to any one of the preceding aspects, wherein the 3-(5-fluorobenzofuran-3-yl)-4-(5-methyl-5H-[1,3]dioxolo[4,5-f]indol-7-yl)-1H-pyrrole-2,5-dione is present in an amount of from about 20% to about 60% by weight relative to the weight of the stabilizing polymer.
Aspect 11. The solid dispersion according to any one of the preceding aspects, wherein the 3-(5-fluorobenzofuran-3-yl)-4-(5-methyl-5H-[1,3]dioxolo[4,5-f]indol-7-yl)-1H-pyrrole-2,5-dione is present in an amount of from about 25% to about 50% by weight relative to the weight of the stabilizing polymer.
Aspect 12. The solid dispersion according to any one of the preceding aspects, wherein the weight ratio of amorphous 3-(5-fluorobenzofuran-3-yl)-4-(5-methyl-5H-[1,3]dioxolo[4,5-f]indol-7-yl)-1H-pyrrole-2,5-dione to stabilizing polymer ranges from about 30:70 to about 50:50.
Aspect 13. The solid dispersion according to any one of the preceding aspects, wherein said solid dispersion has a single glass transition temperature.
Aspect 14. The solid dispersion according to any one of the preceding aspects, wherein said solid dispersion is stable for at least 48 hours at 60° C. and 75% relative humidity.
Aspect 15. The solid dispersion according to any one of the preceding aspects, wherein said solid dispersion is stable for at least 4 weeks at 40° C. and 75% relative humidity.
Aspect 16. The solid dispersion according to any one of the preceding aspects, wherein said solid dispersion is stable for at least 12 weeks at 40° C. and 75% relative humidity.
Aspect 17. A pharmaceutical composition comprising a therapeutically effective amount of a solid dispersion according to any one of the preceding aspects and a pharmaceutically acceptable excipient.
Aspect 18. A process of preparing the solid dispersion according to any one of aspects 1 to 17 comprising the steps of: a) dissolving 3-(5-fluorobenzofuran-3-yl)-4-(5-methyl-5H-[1,3]dioxolo[4,5-f]indol-7-yl)-1H-pyrrole-2,5-dione and a stabilizing polymer in a solvent to form a solution; and b) removing the solvent by evaporation to form the solid dispersion.
Aspect 19. The process according to aspect 18, wherein the solvent is dichloromethane, tetrahydrofuran, aqueous tetrahydrofuran, acetone, aqueous acetone, methanol, ethanol, ethyl acetate, and mixtures thereof.
Aspect 20. The process according to any one of aspects 18-19, wherein the solvent is evaporated by spray-drying.
Aspect 21. The process according to any one of aspects 18-19, wherein the solvent is evaporated under reduced pressure.
Aspect 22. The process according to any one of aspects 18-19, wherein the solvent is evaporated using an inert gas.
Aspect 23. A liquid solution comprising 3-(5-fluorobenzofuran-3-yl)-4-(5-methyl-5H-[1,3]dioxolo[4,5-f]indol-7-yl)-1H-pyrrole-2,5-dione (elraglusib); an emulsifier that is a polyethylene glycol, mustard lecithin, soy lecithin, egg lecithin, monoglyceride, diglyceride, polysorbate, stearoyl lactylate, sorbitan ester, polyglycerol ester, or sucrose ester; a pharmaceutically acceptable alcohol; and a surfactant that is sodium stearate, 4-(5-dodecyl)benzenesulfonate, sodium lauryl sulfate, docusate sodium, phosphatidylcholine, benzalkonium chloride, polyoxyethylene sorbitan fatty acid esters, polyoxyethylene 15 hydroxy stearate, polyoxyethylene castor oil derivatives, polyoxyethylene stearates, sorbitan fatty acid esters, polyoxyethylene alkyl ethers, and polyoxyethylene nonylphenol ether.
Aspect 24. The liquid solution of aspect 23, comprising about 4.0-6.0 wt. % 3-(5-fluorobenzofuran-3-yl)-4-(5-methyl-5H-[1,3]dioxolo[4,5-f]indol-7-yl)-1H-pyrrole-2,5-dione (elraglusib); about 75-95 wt. % of an emulsifier that is a polyethylene glycol, mustard lecithin, soy lecithin, egg lecithin, monoglyceride, diglyceride, polysorbate, stearoyl lactylate, sorbitan ester, polyglycerol ester, or sucrose ester; about 2-17% wt. % of a pharmaceutically acceptable alcohol; and about 0.5-10% wt. % of a surfactant that is sodium stearate, 4-(5-dodecyl)benzenesulfonate, sodium lauryl sulfate, docusate sodium, phosphatidylcholine, benzalkonium chloride, polyoxyethylene sorbitan fatty acid esters, polyoxyethylene 15 hydroxy stearate, polyoxyethylene castor oil derivatives, polyoxyethylene stearates, sorbitan fatty acid esters, polyoxyethylene alkyl ethers, and polyoxyethylene nonylphenol ether.
Aspect 25. The liquid solution of aspect 23 or aspect 24, wherein the emulsifier is a polyethylene glycol (PEG).
Aspect 26. The liquid solution of aspect 25, wherein the polyethylene glycol has a molecular weight of 100-1000 Da.
Aspect 27. The liquid solution of aspect 25 or aspect 26, wherein the emulsifier is PEG 400.
Aspect 28. The liquid solution of any one of aspects 23-27, wherein the pharmaceutically acceptable alcohol is ethanol.
Aspect 29. The liquid solution of any one of aspects 23-28, wherein the surfactant is polysorbate 80 (Polyoxyethylenesorbitan monooleate).
Aspect 30. The liquid solution of any one of aspects 23-29, comprising about 4.3-5.5 wt. % elraglusib.
Aspect 31. The liquid solution of any one of aspects 23 to 30, wherein the concentration of elraglusib in the solution is at least 45 mg/mL.
Aspect 32. The liquid solution of any one of aspects 23 to 30, wherein the concentration of elraglusib in the solution is at least 50 mg/mL.
Aspect 33. The solution of any one of aspects 23 to 32, wherein the elraglusib purity is greater than 97% as measured by HPLC area %.
Aspect 34. The solution of aspect 33, wherein the elraglusib purity is greater than 98% as measured by HPLC area %.
Aspect 35. The solution of aspect 34, wherein the elraglusib purity is greater than 99% as measured by HPLC area %.
Aspect 36. The solution of any one of aspects 23 to 35, wherein the solution contains less than 2%, as measured by HPLC area %, of an impurity having a relative retention time of 0.97 or 0.96, relative to the retention time of elraglusib.
Aspect 37. The solution of any one of aspects 23 to 36, wherein the solution contains less than 1%, as measured by HPLC area %, of an impurity having a relative retention time of 0.97 or of 0.96 relative to the retention time of elraglusib.
Aspect 38. A liquid suspension comprising: elraglusib; an emulsifier that is a polyethylene glycol, mustard lecithin, soy lecithin, egg lecithin, monoglyceride, diglyceride, polysorbate, stearoyl lactylate, sorbitan ester, polyglycerol ester, or sucrose ester; a pharmaceutically acceptable alcohol; a surfactant that is sodium stearate, 4-(5-dodecyl)benzenesulfonate, sodium lauryl sulfate, docusate sodium, phosphatidylcholine, benzalkonium chloride, polyoxyethylene sorbitan fatty acid esters, polyoxyethylene 15 hydroxy stearate, polyoxyethylene castor oil derivatives, polyoxyethylene stearates, sorbitan fatty acid esters, polyoxyethylene alkyl ethers, and polyoxyethylene nonylphenol ether; and a pharmaceutically acceptable diluent.
Aspect 39. The liquid suspension of aspect 38, comprising: about 0.04-0.6 wt. % elraglusib, about 0.8 to 10 wt. % of an emulsifier that is a polyethylene glycol, mustard lecithin, soy lecithin, egg lecithin, monoglyceride, diglyceride, polysorbate, stearoyl lactylate, sorbitan ester, polyglycerol ester, or sucrose ester; about 0.04-1.7 wt. % of a pharmaceutically acceptable alcohol, about 0.009-1 wt. % a surfactant that is sodium stearate, 4-(5-dodecyl)benzenesulfonate, sodium lauryl sulfate, docusate sodium, phosphatidylcholine, benzalkonium chloride, polyoxyethylene sorbitan fatty acid esters, polyoxyethylene 15 hydroxy stearate, polyoxyethylene castor oil derivatives, polyoxyethylene stearates, sorbitan fatty acid esters, polyoxyethylene alkyl ethers, and polyoxyethylene nonylphenol ether; and about 87 to 98 wt % of a pharmaceutically acceptable diluent.
Aspect 40. The liquid suspension of aspect 38 or aspect 39, wherein the emulsifier is a polyethylene glycol (PEG).
Aspect 41. The liquid suspension of aspect 40, wherein the polyethylene glycol has a molecular weight of 100-1000 Da.
Aspect 42. The liquid suspension of aspect 40 or aspect 41, wherein the emulsifier is PEG 400.
Aspect 43. The liquid suspension of any one of aspects 38-42, wherein the pharmaceutically acceptable alcohol is ethanol.
Aspect 44. The liquid suspension of any one of aspects 38-43, wherein the surfactant is polysorbate 80 (Polyoxyethylenesorbitan monooleate).
Aspect 45. The liquid suspension of any one of aspects 38-44, comprising about 0.04 wt. % elraglusib.
Aspect 46. The liquid suspension of any one of aspects 38-44, comprising about 0.1 wt. % elraglusib.
Aspect 47. The liquid suspension of any one of aspects 38-44, comprising about 0.2 wt. % elraglusib.
Aspect 48. The liquid suspension of any one of aspects 38-44, comprising about 0.5 wt. % elraglusib.
Aspect 49. The solution or suspension of any one of aspects 38 to 48, wherein the concentration of elraglusib in the suspension is at least 0.4 mg/mL.
Aspect 50. The solution or suspension of any one of aspects 38 to 48, wherein the concentration of elraglusib in the suspension is at least 0.5 mg/mL.
Aspect 51. The liquid suspension of any one of aspects 38-50, wherein the pharmaceutically acceptable diluent is water, saline solution, an electrolyte solution, a sugar solution, or a flavoring solution.
Aspect 52. The liquid suspension of aspect 51, wherein the pharmaceutically acceptable diluent is dextrose (5%) in water (D5W).
Aspect 53. The suspension of any one of aspects 38 to 52, wherein the elraglusib purity is greater than 97% as measured by HPLC area %.
Aspect 54. The suspension of any one of aspects 38 to 52, wherein the elraglusib purity is greater than 98% as measured by HPLC area %.
Aspect 55. The suspension of any one of aspects 38 to 52, wherein the elraglusib purity is greater than 99% as measured by HPLC area %.
Aspect 56. The suspension of any one of aspects 38 to 52, wherein the suspension contains less than 2%, as measured by HPLC area %, of an impurity having a relative retention time of 0.97 or 0.96, relative to the retention time of elraglusib.
Aspect 57. The suspension of any one of aspects 38 to 52, wherein the suspension contains less than 1%, as measured by HPLC area %, of an impurity having a relative retention time of 0.97 or of 0.96 relative to the retention time of elraglusib.
Aspect 58. A method of achieving an elraglusib plasma concentration ranging from 1000 to 10000 ng/ml in a human, the method comprising orally administering to the human a solid dispersion, liquid solution, liquid suspension, or a pharmaceutical composition, of the disclosure.
Aspect 59. A method of treating a disease or disorder in a subject in need thereof, said method comprising orally administering to the subject the solid dispersion of any one of aspects 1 to 16, the pharmaceutical composition of aspect 17, the solution of any one of aspects 23-37, or the suspension of any one of aspects 38-57.
Aspect 60. The method according to aspect 59, wherein the disease or disorder is cancer.
Aspect 61. The method according to aspect 60 wherein the cancer is brain cancer, lung cancer, breast cancer, ovarian cancer, bladder cancer, neuroblastoma, renal cancer, pancreatic cancer, or glioblastoma.
Aspect 62. The method according to aspect 60 wherein the cancer is the cancer is glioblastoma.
Aspect 63. The method according to aspect 59, wherein the disease or disorder is a lymphoproliferative disorder.
Aspect 64. The method according to aspect 63, wherein the lymphoproliferative disorder is a malignant lymphoproliferative disorder.
Aspect 65. The method according to aspect 64, wherein the malignant lymphoproliferative disorder is a malignant B-cell lymphoproliferative disorder.
Aspect 66. The method according to aspect 65, wherein the malignant B-cell lymphoproliferative disorder is Diffuse large B-cell lymphoma, acute lymphocytic leukemia, lymphoid blastic phase Chronic Myeloid Leukemia, Chronic lymphocytic leukemia/Small lymphocytic lymphoma, Extranodal marginal zone B-cell lymphomas, Mucosa-associated lymphoid tissue lymphomas, Follicular lymphoma, Mantle cell lymphoma, Nodal marginal zone B-cell lymphoma, Burkitt lymphoma, Hairy cell leukemia, Primary central nervous system lymphoma, Splenic marginal zone B-cell lymphoma, Waldenstrom's macroglobulinemia/Lymphoplasmacytic lymphoma, Multiple myeloma, Plasma cells dyscrasias, Plasma cell neoplasms, Primary mediastinal B-cell lymphoma, Hodgkin Disease, or Castelman's Disease.
Aspect 67. The method according to aspect 66, wherein the malignant B-cell lymphoproliferative disorder is Diffuse large B-cell lymphoma.
Aspect 68. The method according to aspect 67, wherein the Diffuse large B-cell lymphoma is Double-Hit lymphoma.
Aspect 69. The method according to aspect 63, wherein the lymphoproliferative disorder is a malignant T-cell lymphoproliferative disorder.
Aspect 70. The method according to aspect 69, wherein the malignant T-cell lymphoproliferative disorder is T-cell leukemia/lymphoma, Extranodal natural killer/T-cell lymphoma, Cutaneous T-cell lymphoma, Enteropathy-type T-cell lymphoma, Angioimmunoblastic T-cell lymphoma, Anaplastic large T/null-cell lymphoma, Subcutaneous panniculitis-like T-cell lymphoma, T-cell acute lymphocytic leukemia, T-cell large granular lymphocyte leukemia, Lymphoid blastic phase Chronic Myeloid Leukemia, post-transplantation lymphoproliferative syndromes, human T-cell leukemia virus type 1-positive (HTLV-1⁺) adult T-cell leukemia/lymphoma (ATL), T-cell prolymphocytic leukemia (T-PLL), or unspecified T-cell lymphoma.
Aspect 71. The method according to aspect 59, wherein the disease or disorder is traumatic brain injury.
Aspect 72. The method according to aspect 59, wherein the disease or disorder is idiopathic pulmonary fibrosis.
Aspect 73. The method according to aspect 59, wherein the disease or disorder is pleural fibrosis.

Having described the disclosure with reference to certain preferred embodiments, other embodiments will become apparent to one skilled in the art from consideration of the specification. The disclosure is further illustrated by reference to the following examples. It will be apparent to those skilled in the art that many modifications, both to materials and methods, may be practiced without departing from the scope of the disclosure.

### EXAMPLES

### Example 1—Preparation of an Oral Liquid

An elraglusib liquid dosage form that can be administered directly by oral ingestion or that can be diluted into dextrose 5% in water to form a liquid suspension suitable for oral administration was prepared. Table 1-1 shows the batch formula.

TABLE 1-1
Batch Formula for 5 L Scale

| Ingredient | kg | % w/w | kg/L | Liter |
|---|---|---|---|---|
| Polyethylene Glycol 400, | 5.04 | 89.9 | 1.04 | 4.46 |
| NF | | | | |
| Ethanol, 200 Proof | 0.268 | 4.8 | 0.06 | 0.34 |
| (100%), USP/EP/ACS | | | | |
| Polysorbate 80, NF | 0.0537 | 0.958 | 0.01 | 0.05 |
| Elraglusib powder | 0.2421 | 4.3 | 0.05 | N/A |
| Totals | 5.60 | 100 | 1.16 | 4.85 |

*Total elraglusib weighed (247.0 g) times Assay value for the elraglusib lot (0.98) = 0.2421 kg

Day 1: 5.040 kg of PEG400 was weighed out in a dispensing booth into a 6 L flask. The solution was covered and transferred to a biosafety cabinet. Elraglusib powder was weighed out in the dispensing booth in four separate measurements: 64.42 g, 69.89 g, 76.76 g, and 35.97 g. The total weight was 247.04 g. Each portion was transferred to the biosafety cabinet and sequentially added to the flask containing PEG400 and allowed to stir overnight protected from light.

Day 2: A layer of elraglusib was on the bottom of the flask. A combination of a shaker plate and stir plate was used to loosen the bottom layer of the flask. The flask was then placed on a shaker plate and allowed to mix overnight protected from light.

Day 3: The elraglusib appeared to be in solution. A 57-mL aliquot of Polysorbate 80 (Tween80) was added to the solution using a serological pipette and placed on a shaker plate for 60 minutes. After the 60 minutes, 270.05 g of dehydrated ethanol was added to the solution and placed on a shaker plate to mix for an additional 60 minutes.

Prefiltration of the Bulk Samples: A 20 mL sample was pulled from the top, middle, and bottom of the pre-filtration bulk material on day 3 using a new serological pipette. The samples were stored in 20 mL amber vials at room temperature protected from light in a cardboard box.

Filtration of the Bulk Solution: The solution was transferred to a laminar flow hood and filtered into a clean dry 6 L flask via a peristaltic pump using a 4-in 0.2 μm Opticap XL4 Durapore clarifying filter.

Vial Fill of the Filtered Bulk Solution: The amber 20-mL vials (molded glass) were filled with 11±0.1 mL of filtered bulk solution in a laminar flow hood using a calibrated repeater pipette with a 50 mL tip. The vials were sealed with FluroTec rubber stoppers and metal crimps with white flip-off seals. The vials were stored in cardboard boxes at ambient conditions protected from light.

Characterization of a 50 mg/mL solution for oral suspension batch is summarized in Table 1-2.

TABLE 1-2
Elraglusib Solution, 50 mg/mL Batch Analysis

| ASSAY | SPECIFICATION | RESULT |
|---|---|---|
| Appearance | Clear, very light amber-red to | As described |
| | dark amber-red solution, | |
| | essentially free of visible | |
| | particulates | |
| Potency | 90.0% to 110.0% | Beginning (n = 3) 96.0% |
| | | Middle (n = 3 96.5% |
| | | End (n = 3) 96.3% |
| Individual Impurities | Each Individual Impurity ≤ 1.5% | Beginning Unknown RRT 0.96: 0.2% |
| | | (n = 3) 9-ING-41 Anhydride: 0.8% |
| | | Middle Unknown RRT 0.96: 0.2% |
| | | (n = 3) 9-ING-41 Anhydride: 0.8% |
| | | End Unknown RRT 0.96: 0.2% |
| | | (n = 3) 9-ING-41 Anhydride: 0.8% |
| Total Impurities | Total Impurities ≤ 3.0% | Beginning (n = 3) 1.0% |
| | | Middle (n = 3) 1.0% |
| | | End (n = 3) 1.0% |
| Deliverable Volume | Mean ≥ 10 mL | Mean volume (n = 30): 10.02 mL |
| | Each Individual Unit ≥ 9.5 mL | Each Individual Unit was ≥9.5 mL |
| Viscosity | Record Result | Mean Viscosity (n = 3) 84.5 cP1 |
| | | (2.55% RSD) |
| Density | Record Result | 1.1097 g/mL |
| Antimicrobial | Meets Requirements of USP, 51. | Meets requirements |
| Effectiveness | | |
| (USP <51>) | | |
| Bacterial Endotoxin | ≤5 EU/mL | <3.5 EU/mL |
| (USP <85>) | | |
| Microbial Enumeration | TAMC ≤ 10 CFU/100 mL | TAMC < 1 CFU/100 mL |
| (USP <61>) | TYMC ≤ 10 CFU/100 mL | TYMC < 1 CFU/100 mL |

### Formulation

The starting point for elraglusib oral solution formulation development was a elraglusib 10 mg/mL IV Injection drug product comprising 10 mg/mL elraglusib dissolved in a Vehicle of PEG400:EtOH:Tween80, 75:17:8% w/w. Development of an elraglusib oral solution drug product was aimed at:
increasing elraglusib concentration (thereby lowering the dose volume required);
maintaining the same excipients as the elraglusib 10 mg/mL Injection drug product;
lowering the EtOH concentration (thereby decreasing the ethanol dose administered to patients);
lowering the Tween 80 concentration (thereby decreasing the Tween 80 dose administered to patients);
achieving >50% bioavailability by oral administration versus intravenous (IV) administration.

An initial study prepared 50 mg/mL elraglusib solutions in 12 Vehicle Variants. Table 1-3 shows the Vehicle Variant compositions.

TABLE 1-3
Vehicle Variants Evaluated for 9-ING-41
Oral Formulation Development

| | Compositions | | | | | |
|---|---|---|---|---|---|---|
| Variant | PEG400 | API | EtOH | Tween80 | Water | EtOH + |
| # | (g) | (g) | (g) | (g) | (g) | Tween80 |
| 1 | 75 | 5 | 17 | 8 | 0 | 25 |
| 2 | 79 | 5 | 17 | 4 | 0 | 21 |
| 3 | 83 | 5 | 17 | 0 | 0 | 17 |
| 4 | 82 | 5 | 1 | 8 | 0 | 18 |
| 5 | 87 | 5 | 5 | 8 | 0 | 13 |
| 6 | 84 | 5 | 15 | 1 | 0 | 16 |
| 7 | 94 | 5 | 5 | 1 | 0 | 6.0 |
| 8 | 80 | 5 | 15 | 0 | 5 | 15 |
| 9 | 85 | 5 | 10 | 0 | 5 | 10 |
| 10 | 90 | 5 | 5 | 0 | 5 | 5.0 |
| 11 | 85 | 5 | 15 | 0 | 0 | 15 |
| 12 | 95 | 5 | 5 | 0 | 0 | 5.0 |

Each Vehicle Variant was made to 50 mg/mL with elraglusib and was tested (with and without syringe filtration) with respect to: (1) elraglusib main peak Area %, (2) related substance RRT 0.96 Area %, and (3) related Substance Area % RRT 0.97. RRT is the HPLC Relative Retention Time, i.e., the retention time of the substance relative to the retention time of elraglusib.
elraglusib remained soluble at 50 mg/mL with no precipitation or haziness and no visible particulates evident.
elraglusib concentration averaged 99.0±2.9% of 50 mg/mL in all non-filtered Vehicle Variants.
elraglusib concentration averaged 98.6±2.7% of 50 mg/mL in all syringe-filtered Vehicle Variants.
Variants 7 through 12 (Table 1-3) were made within the 24-hr mixing of API into PEG400 and exhibited more consistent values at main peak (i.e., elraglusib peak) Area % value=99% and with 97-102% recovery.
Related Substance RRT 0.96 area % values were higher (>1.2%) in Variants 1 through 6, than in in Variants 7 through 12 (<0.5%).

Related Substance RRT 0.96 levels appear to increase with increasing EtOH % or Tween80%, or both in the Vehicle Variants.

To assess the application of these 12 Vehicle Variants made to 50 mg/ml elraglusib for producing a liquid oral suspension drug product, two in vitro studies were undertaken. One study determined elraglusib solubility at ambient temperature after diluting each of the 12 Vehicle Variants to 0.50-, 1.0-, and 2.0-mg/mL elraglusib concentrations into each of two potential oral solution diluents, namely: 5% Dextrose Injection (D5W) and Pedialyte® (PED).

All 12 Vehicle Variants made to 50 mg/mL elraglusib produced precipitate when diluted to 0.50-, 1.0- and 2.0-mg/mL concentrations into PED. Vehicle Variants 1 through 5 produced frank precipitate when diluted into D5W, but Vehicle Variants 6 through 12 produced hazy, fine dispersions that persisted for at least 4 hours after dilution. For 50 mg/mL elraglusib in Vehicle Variant 7 diluted to 2 mg/mL in D5W, Dynamic Light Scattering (DLS) analysis indicated mean particle diameter values at approximately 200 nm. HPLC analysis using the method shown in Table 1-5 indicated a potential correlation between high Vehicle Variant EtOH and/or Tween 80 content and formation of related substance RRT 0.97 at elevated levels.

Another study determined the concentrations of soluble and soluble plus particulate elraglusib in each of three biorelevant dissolution media (FaSSGF, FaSSIF, and FeSSIF) after adding 1.0-, and 2.0-mg/mL dilutions in D5W from the 50-mg/mL Vehicle Variant 7 formulation. When either the 1.0-mg/mL (700 mL) or 2.0-mg/mL (350 mL) D5W suspensions were brought to 900 mL total volume with biorelevant dissolution media, a suspension containing both soluble elraglusib and elraglusib particulates resulted. The concentrations of soluble elraglusib were somewhat higher in FeSSIF medium (~50% of theoretical [9-ING-41]) than either FaSSGF medium (~20% of theoretical [9-ING-41]) or FaSSIF medium (~10% of theoretical [9-ING-41]).

Elraglusib at 50 mg/mL in Vehicle Variant 7 (PEG400:EtOH:Tween80 94:5:1% w/w) was further developed as an oral solution dosage form. The specifications for the 50 mg/ml oral solution are summarized in Table 1-4.

TABLE 1-4
9-ING-41 50-mg/mL Oral Solution Specifications

| Quality Attribute | Method | Acceptance Criteria |
|---|---|---|
| Appearance | Visual Inspection | Clear, red solution essentially |
| | | free of visible foreign matter |
| Identity | UV-vis Spectrum | Sample spectrum matches |
| | | reference standard spectrum |
| Identity | RP-HPLC (438 nm) | Sample retention time |
| | | matches reference standard |
| | | retention time |
| Assay | | 95.0% to 105.0% of Label |
| | | Claim |
| 9-ING-41% Purity | | ≥97.0% |
| Individual Impurities | | Each Individual Impurity ≤ 1.5% |
| Total Impurities | | Total Impurities ≤ 3.0% |
| Deliverable Volume | Volumetric, USP <698> | Meets Requirements |
| Alcohol Content | Gas Chromatography, USP | Report Result |
| | <611>, Method IIA | |
| Density | Gravimetric USP <698> | Report Result |
| Anti-microbial Effectiveness, USP | Membrane Filtration | Meets Requirements |
| <51> | USP <51> | |
| Microbial Enumeration, | Membrane Filtration | TAMC ≤ 1000 CFU/mL |
| | USP <61>, USP <62> | TYMC ≤ 100 CFU/mL |
| | | Absence of E. Coli in 1 mL |

HPLC was used for testing identify, assay purity, and related substances. Table 1-5 shows the HPLC method chromatographic operating parameters.

TABLE 1-5
HPLC Method Chromatographic Operating Parameters

| Parameter | Condition | | |
|---|---|---|---|
| Column | Waters Atlantis T3, 150 × 4.6 mm, 3 μm | | |
| Flow Rate | 1.0 mL/min | | |
| Run Time | 27 minutes | | |
| Sample Temp | 5° C. | | |
| Column Temp | 30° C. | | |
| Injection Volume | 20 μL | | |
| Detection | UV 438 nm | | |
| 9-ING-41 Retention Time | Approximately 11.2 min | | |
| Detector Peak Width | 0.05 min (1 s) or smaller | | |
| Slit Width | 4 nm | | |
| Gradient | Minutes | % A | % B |
| | 0 | 70 | 30 |
| | 15 | 10 | 90 |
| | 20 | 10 | 90 |
| | 20.1 | 70 | 30 |
| | 27 | 70 | 30 |

| | |
|---|---|
| Diluent | Acetonitrile |
| Mobile Phase A | 0.02% w/w TFA in water |
| Mobile Phase B | 0.02% w/w TFA in Acetonitrile |
| Column Care | Flush the column with 50% acetonitrile and |
| | 50% water at 1.0 mL/min for 30 minutes. |
| | Store the column in 100% acetonitrile |

### Characterization of Drug Product Impurities

HPLC indicated two related substances, namely:
Related substance eluting at Relative Retention Time RRT 0.96, and
elraglusib anhydride analog eluting at Relative Retention Time RRT 1.21

### Example 2A—Preparation of an Amorphous Solid Dispersion (ASD)

### Characterization of Elraglusib Starting Material

The starting elraglusib was characterized using XRPD, PLM, TGA, and DSC. XRPD showed the material to be crystalline. A PLM image showed plate-like birefringent particles characteristic of a semi-crystalline solid. The TGA plot showed a weight loss of 0.91% indicating the anhydrous nature of the material, while the DSC plot shows an endothermic peak corresponding to a melting point at 227.1° C. (onset). These data plots indicate that elraglusib is a crystalline solid of Form I. Methods of preparing elraglusib Form I are known in the art. See U.S. Pat. No. 11,136,334.

Elraglusib has a Tg of ~111° C. (384.15 K) and a Tm of ~228° C. (501.15 K) giving it a Tm (K)/Tg (K) ratio of ~1.3; the c Log P value is ~4.3. To maintain the API in an amorphous state, the Tg of the ASD is preferably above 80° C. The elraglusib:CAP ASD and the elraglusib:EL100 ASD both feature Tg~106° C.

The HPLC reference standard was a Standard Stock Solution of 0.2 mg/mL elraglusib in acetonitrile. The method acquisition included 438-nm detection. HPLC analysis detected both API and a related substance peak at relative retention time RRT=0.97.

### Amorphous Solid Dispersion (ASD) Screening and Characterization

### Solubility Analysis of Elraglusib and Polymers

The approximate solubility of both elraglusib (Table 2A-1) and the stabilizing polymers (Table 2A-2) of interest was individually studied in solvents commonly used for spray-drying. In addition, a more quantitative solubility value of elraglusib was also obtained by high performance liquid chromatography (HPLC) in presence of organic solvents with or without surfactants (Table 2A-3). Tetrahydrofuran (THF) or THF-containing solvent mixtures showed highest solubility for the elraglusib among the organic solvents tested. XRPD analysis of the solids recovered from some solubility experiments at 24 hours showed that the samples containing THF solvent converted to the THF solvate, while samples having only water and surfactant did not demonstrate any form change. An approximate solubility check was performed for the polymers to ensure that a sufficient amount can dissolve to produce 50% w/w drug load as per the solubility of API in that specific solvent. The result of the study showed that except for hypromellose acetate succinate (HPMCAS) in dichloromethane:acetone (5:1), all other polymers and solvent combinations tested can be used to prepare 1:1 mixture of API and the polymer (Table 2A-4).

TABLE 2A-1
Approximate solubility determination of
API in presence of various solvents

| API | | Approximate Solubility |
|---|---|---|
| (elraglusib) | Solvent | Range (mg/mL) |
| API | Tetrahydrofuran (THF) | S > 64.0 |
| API | Acetone | 28.3 < S < 85.0 |
| API | Dichloromethane (DCM) | 6.2 < S < 20.7 |
| API | DCM:Methanol (MeOH) | 4.2 < S < 14.0 |
| API | DCM:Ethanol (EtOH | 11.0 < S < 33.0 |
| API | DCM:Acetone | 18.0 < S < 54.0 |
| API | Ethyl Acetate | 9.7 < S < 29.0 |
| API | THF:Water (85:15) | S > 98.0 |
| API | Acetone:Water (85:15) | 18.0 < S < 54.0 |

TABLE 2A-2
Approximate solubility determination of various polymers in different solvents

| | | Approximate Solubility |
|---|---|---|
| Polymer | Solvent | Range (mg/mL) |
| Plasdone S-630 (copovidone) | Tetrahydrofuran (THF) | 93.0 < S < 186.0 |
| Plasdone S-630 (copovidone) | Acetone | S > 190.0 |
| Plasdone S-630 (copovidone) | Dichloromethane (DCM) | S > 192.0 |
| HP 55 (HPMC Phthalate) | DCM:Ethanol (EtOH) | S > 38.0 |
| HP 55 (HPMC Phthalate) | DCM: Acetone | 60.0 < S < 120.0 |
| HP 55 (HPMC Phthalate) | Ethyl Acetate | S < 4.7 |
| HP 55 (HPMC Phthalate) | Acetone: Water (75:25) | 24.0 < S < 48.0 |
| AQOAT ® AS-HG | THF | 41.0 < S < 82.0 |
| (Hypromellose acetate | | |
| succinate/HPMC AS) | | |
| AQOAT ® AS-HG | Acetone | 63.0 < S < 126.0 |
| (Hypromellose acetate | | |
| succinate/HPMC AS) | | |
| AQOAT ® AS-HG | DCM | 43.0 < S < 86.0 |
| (Hypromellose acetate | | |
| succinate/HPMC AS) | | |
| Methocel E4M | THF | S < 7.1 |
| (Hypromellose/HPMC)) | | |
| Methocel E4M | THF:H2O (3:1) | 6.9 < S < 23.0 |
| (Hypromellose/HPMC)) | | |
| Methocel E4M | Acetone:water (85:15) | 1.8 < S < 6.0 |
| (Hypromellose/HPMC)) | | |
| Methocel E4M | DCM:EtOH | S < 4.9 |
| (Hypromellose/HPMC)) | | |
| Kollidone 25 (Povidone) | Acetone:water (75:25) | 6.9 < S < 23.0 |
| Kollidone 25 (Povidone) | THF | S < 2.3 |
| Kollidone 25 (Povidone) | Acetone | S < 4.5 |
| Kollidone 25 (Povidone) | Ethyl Acetate | S < 4.8 |
| “EL 100” | THE | 16.3 < S < 49.0 |
| “EL 100” | DCM | S < 2.8 |
| “EL 100” | DCM:MeOH (75:25) | 9.3 < S < 28.0 |
| “EL 100” | Acetone:water (97:3) | 38.0 < S < 76.0 |
| Kollidone 30 (povidone) | THF | S < 7.1 |
| Kollidone 30 (povidone) | THF:water (85:15) | S > 126.0 |
| Kollidone 30 (povidone) | DCM | S > 126.0 |
| Kollidone 30 (povidone) | Acetone | S < 3.2 |
| Kollidone 30 (povidone) | Acetone:water (75:25) | S < 2.8 |
| Phthalavin (polyvinyl acetate | THF | S > 122.0 |
| phthalate) or “PVAP” | | |
| PVAP | DCM | S < 2.8 |
| PVAP | Acetone | 2.5 < S < 8.3 |
| PVAP | DCM:MeOH | S > 50.0 |
| Methocel K4M | DCM | S < 3.9 |
| (Hypromellose/HPMC) | | |
| Methocel K4M | THF | S < 3.6 |
| (Hypromellose/HPMC) | | |
| Methocel K4M | Acetone | S < 2.9 |
| (Hypromellose/HPMC) | | |
| Methocel K4M | THF:water (70:30) | 3.5 < S < 11.7 |
| (Hypromellose/HPMC) | | |
| Eastman C-A-P (cellulose | Acetone | S > 72 |
| acetate phthalate) or “CAP” | | |
| “CAP” | THF | S > 98 |
| “CAP” | DCM | S < 3.6 |

TABLE 2A-3
Quantitative solubility determination (by HPLC) for
API in presence of organic solvents and surfactants

| | Surfactant | Solubility (n = 2) | XRPD Profile After |
|---|---|---|---|
| Solvent* | (10% w/v) | (mg/mL) # | Solubility Experiment |
| DCM | Not used | 15.09 | Form I + one peak ~9o 2θ |
| Acetone | Not used | 13.03 | Acetone Solvate |
| THF (with | Not used | 25.28 | Type E (THF solvate) |
| stabilizer BHT) | | | |
| DCM:EtOH | Not used | 16.43 | Type C (EtOH solvate) |
| (1:1) | | | |
| DCM:Acetone | Not used | 19.82 | Acetone Solvate |
| (1:1) | | | |
| DCM:Acetone | Not used | 24.46 | Acetone Solvate |
| (5:1) | | | |
| DCM:MeOH | Not used | 11.78 | Type F (MeOH solvate) |
| (8:2) | | | |
| THF | Not used | 52.7 | THF solvate |
| THF | Tween 80 | 49.8 | THE solvate |
| THF | TPGS | 46.8 | THE solvate |
| THF | Kolliphor HS 15 | 47.6 | THE solvate |
| THF | Kolliphor EL | 48.0 | THF solvate |
| THF | Poloxamer P188 | 49.8 | THE solvate |
| THF | Labrasol | 52.3 | THF solvate |
| THF:H₂O | Not used | 61.2 | THF solvate |
| (90:10) | | | |
| THF:H₂O | Labrasol | 54.8 | THE solvate |
| (90:10) | | | |
| H₂O | Labrasol | 0.16 | Form I |
| H₂O | Tween 80 | 0.24 | Form I |
| H₂O | Kolliphor EL | 0.12 | Form I |

TABLE 2A-4
Determining visual solubilization of polymer at maximum
concentration of API in that organic solvent

| | | Solubility | |
|---|---|---|---|
| Polymer | Solvent | (mg/mL) | Observation |
| HPMCP55 | Acetone | >13 | Dissolved - clear |
| CAP | THF | >25 | Dissolved - clear |
| PVAP | THF | >122 | Dissolved - clear |
| HPMCP55 | DCM:Acetone (1:1) | 60.0 < S < 120.0 | Dissolved - clear |
| HPMCAS | DCM:Acetone (5:1) | >24 | Dissolved - Hazy |
| “EL100” | DCM:MeOH (8:2) | >12 | Dissolved - clear |

### ASD Screening

ASD screening was conducted using elraglusib starting material and various polymers. A total of 24 screening experiments were performed to prepare ASDs, among them 5 experiments by solvent air blow drying at 50% w/w drug loading, 4 experiments by rotavap at 25% w/w drug loading, and 15 experiments by rotovap at 50% w/w drug loading. Initially, the ASDs were obtained by rapid evaporation of the solvent, which was achieved by air blow drying. In these experiments, a solution containing 150 mg API and 150 mg of polymer to achieve a 50% w/w drug loading was prepared in 4 ml of the desired solvent. The solution was sonicated for 5 min, followed by visual analysis to make sure it is clear and then the solution was exposed to an air stream for fast evaporation. Obtained solids were analyzed by XRPD.

The characterization results are summarized in Table 2A-5.

TABLE 2A-5
ASD screening by air blow drying at 50% w/w drug loading

| | | | | | Drug | | |
|---|---|---|---|---|---|---|---|
| | | | | | loading | | |
| API wt. | | Weight | | Volume | (calculated | Method of | Solid |
| (mg) | Polymer | (mg) | Solvent | (mL) | %) | drying | Form |
| 150 | Plasdone S- | 150 | Acetone | 4 | 50 | Air blow | Amorphous |
| | 630 | | | | | | |
| | (copovidone) | | | | | | |
| 150 | HPMCAS HG | 150 | Acetone | 4 | 50 | Air blow | Acetone |
| | | | | | | | Solvate |
| 150 | HPMCP 55 | 150 | Acetone | 4 | 50 | Air blow | Acetone |
| | | | | | | | Solvate |
| 150 | Eudragit ® | 150 | Acetone:H₂O | 4 | 50 | Air blow | Acetone |
| | L100 | | (97:3) | | | | Solvate |
| 150 | CAP | 150 | Acetone | 4 | 50 | Air blow | Acetone |
| | | | | | | | Solvate |

The results showed that only Plasdone S-630 polymer gave amorphous material while all other produced crystalline acetone solvate.

To successfully obtain more amorphous hits rotavap was used with only 25% drug loading. To prepare ASDs by rotavap, a solution of API and polymer (having 25% API) was prepared in the desired solvent. The solution was sonicated for 5 min, followed by visual analysis to make sure it is clear and then the solution was kept in a Rotavap for 15 min at 50 mbar vacuum. The solids obtained were kept in vacuum oven at 30° C. over the weekend. The samples were analyzed by XRPD, TGA and DSC for glass transition temperature Tg. All results are summarized in Table 2A-6.

TABLE 2A-6
ASD screening using rotary evaporator at 25% drug loading

| | | | | | Drug | | |
|---|---|---|---|---|---|---|---|
| API | | | | | loading | Method | |
| wt. | | Weight | | Volume | (calculated | of | Solid |
| (mg) | Polymer | (mg) | Solvent | (mL) | %) | drying | Form |
| 62 | HPMCP | 200 | Acetone | 4 | 25 | Rotavap | Amorphous |
| | 55 | | | | | | |
| 62 | HPMCAS | 200 | Acetone | 4 | 25 | Rotavap | Amorphous |
| | HG | | | | | | |
| 62 | Eudragit ® | 200 | Acetone:H2O | 4 | 25 | Rotavap | Acetone |
| | L100 | | (97:3) | | | | Solvate |
| 62 | CAP | 200 | Acetone | 4 | 25 | Rotavap | Amorphous |

Screening with 50% drug loading using a rotavap was performed. For this screening, a higher amount (e.g., more dilute) of solvent, ≥6 mL (compared to 4 mL as used previously) was used to dissolve the 150 mg of elraglusib and 150 mg of the polymer, to prevent any risk of material precipitation in solution before performing the rotavap or spray drying experiments. Results of these screening and formulation characterization are summarized in Table 2A-7.

TABLE 2A-7
Summary of ASD screening by rotavap at 50% elraglusib loading and their characterization

| | | | | | | wt. loss | | Tg |
|---|---|---|---|---|---|---|---|---|
| | | | | | | (% w/w) | Tg (° C.) | (° C.) |
| | | | V | DL | Solid | at 110° | (no pin | (pin |
| # | Polymer | Solvent | (mL) | (%) | Form | C. | hole) | hole) |
| 1 | Plasdone S- | THF | 6 | 50 | Amorphous | 0.36 (at | 57.4 | 53.9 |
| | 630 | | | | | 90° C.) | | |
| | (copovidone) | | | | | | | |
| 2 | Plasdone S- | DCM | 6 | 50 | Amorphous | 1.21 | 59.0 | 98.6* |
| | 630 (copovidone) | | | | | | | |
| 3 | CAP | THF | 6 | 50 | Amorphous | 2.15 | 92.8* | 120.8 |
| 4 | CAP | Acetone | 6 | 50 | Amorphous | 0.89 | 97.4 | 116.5 |
| 5 | Methocel | THF:Water | 8 | 50 | Form I | NA | NA | NA |
| | K4M | (7:3) | | | | | | |
| | (Hypromellose/HPMC) | | | | | | | |
| 6 | HPMCP 55 | Acetone | 6 | 50 | Amorphous | 0.84 | 49.2 | 53.6 |
| 7 | HPMCP 55 | DCM:EtOH | 8 | 50 | Solvate | NA | NA | NA |
| | | (1:1) | | | | | | |
| 8 | HPMCP 55 | DCM:Acetone | 6 | 50 | Amorphous | 1.79 | 126.5 | 115.4* |
| | | (1:1) | | | | | | |
| 90 | HPMCAS | THF | 6 | 50 | Amorphous | 2.62 | 85.4 | 83.1* |
| 10 | HPMCAS | DCM:MeOH | 10 | 50 | Solvate | NA | NA | NA |
| | | (7:3) | | | | | | |
| 11 | HPMCAS | DCM:Acetone | 6 | 50 | Amorphous | 1.44 | 44.6 | 56.6 |
| | | (5:1) | | | | | | |
| 12 | Eudragit ® | DCM:MeOH | 8 | 50 | Amorphous | 1.16 | 84.4 | 122 |
| | L100 (“EL100”) | (8:2) | | | | | | |
| 13 | Povidone | THF:water | 16 | 50 | Type D | NA | NA | NA |
| | K30 | (4:3) | | | | | | |
| 14 | Povidone | DCM | 6 | 50 | Amorphous | 0.68 (at | 84.0 | 89.0 |
| | K30 | | | | | 90° C.) | | |
| 15 | Phthalavin | THF | 6 | 50 | Amorphous | 3.15 | * | 74.9* |
| | (polyvinyl | | | | | | | |
| | acetate | | | | | | | |
| | phthalate/PVAP) | | | | | | | |

*distinct Tg like pattern not observed, tentative Tg

These experiments demonstrated that most of the experiments yielded amorphous material except for hypromellose (HPMC) K4M from THF:Water (7:3), HPMCP 55 from DCM:EtOH (1:1), HPMCAS from DCM:MeOH (7:3), and povidone K30 from THF:Water (4:3), which showed crystalline material. Therefore, a total of 11 amorphous formulations at 50% drug loading were obtained. To determine the Tg and the total residual solvent/moisture content of these ASDs, the ASDs were characterized by mDSC and TGA, respectively. The mDSC was carried out in both hermetically sealed pan without and with pin hole, to determine Tg in presence of residual solvent (i.e., wet Tg) and Tg of the solid after the solvent was removed (i.e., dry Tg), respectively. Results pertaining to mDSC and TGA analysis are summarized in Table 2A-7.

### Kinetic Solubility in Biorelevant Media

Kinetic solubility of six elraglusib ASD compositions was measured in biorelevant media at 37° C. The three biorelevant media were: SGF, FaSSIF, and FeSSIF. These were prepared using procedure and FaSSIF/FeSSIF/FaSSGF Powder obtained from Biorelevant.com (https://biorelevant.com/learning_center/how-make-fassif/). A suspension of each ASD was prepared in SGF, FaSSIF, and FeSSIF and each was allowed to stir at 400 rpm. An aliquot of the supernatant was taken out at 1 and 2 hours, syringe filtered using a cellulose acetate filter (0.22 μM pore size) and analyzed by HPLC. The solubilities of each ASD in SGF, FaSSIF, and FeSSIF are presented in Table 2A-8, Table 2A-9, and Table 2A-10, respectively. The solubility assessment showed higher solubility in FaSSIF and FeSSIF compared to SGF for all ASDs. In addition, at 2 hours the remaining solids were analyzed by XRPD. Data showed that all the ASDs remained amorphous post-solubility analysis. XRPD of some of the sample recovered from SGF and FaSSIF showed presence of peak ~32° 2θ, which corresponds to residual NaCl (from buffer) being crystallized on ASD solids. In addition, data also showed that most of the ASDs post FeSSIF 2-hour solubility have peaks, ~9° 2θ, 17.5° 2θ, 26° 2θ, and ~36° 2θ. These peaks correspond to residual FeSSIF buffer crystallizing on the ASD solids upon drying. (See Tables 2A-8, -9, and -10).

TABLE 2A-8
Summary of solubility results for ASDs in SGF

| | | | 1 hr | 2 hr | |
|---|---|---|---|---|---|
| | | | solubility | solubility | |
| | | | in SGF | in SGF | |
| # | Polymer | Solvent | (mg/mL) | (mg/mL) | XRPD after SGF |
| 4 | HPMCAS | DCM:Acetone | <0.01 | <0.01 | Amorphous + one NaCl |
| | | (5:1) | | | peak (~32 o 2θ)* |
| 6 | HPMCP | DCM:Acetone | <0.01 | <0.01 | Amorphous + one NaCl |
| | 55 | (1:1) | | | peak (~32 o 2θ)* |
| 7 | EL100 | DCM:MeOH | <0.01 | <0.01 | Amorphous + one NaCl |
| | | (8:2) | | | peak (~32 o 2θ)* |
| 9 | CAP | THF | <0.01 | <0.01 | Amorphous + one NaCl |
| | | | | | peak (~32 o 2θ)* |
| 10 | PVAP | THF | <0.01 | <0.01 | Amorphous + one NaCl |
| | | | | | peak (~32 o 2θ)* |
| 12 | HPMCP | Acetone | <0.01 | <0.01 | Amorphous + one NaCl |
| | 55 | | | | peak (~32 o 2θ)* |

*NaCl peak = corresponds to residual NaCl being crystallized on ASD solids recovered after solubility study.

TABLE 2A-9
Summary of solubility results for ASDs in FaSSIF

| | | | 1 hr | 2 hr | |
|---|---|---|---|---|---|
| | | | solubility in | solubility in | |
| | | | FaSSIF | FaSSIF | XRPD after |
| # | Polymer | Solvent | (mg/mL) | (mg/mL) | FaSSIF |
| 4 | HPMCAS | DCM:Acetone | 0.032 | 0.061 | Amorphous + one |
| | | (5:1) | | | small NaCl peak |
| | | | | | (~32 o 2θ)* |
| 6 | HPMCP | DCM:Acetone | 0.029 | 0.048 | Amorphous |
| | 55 | (1:1) | | | |
| 7 | EL100 | DCM:MeOH | 0.02 | 0.044 | Amorphous |
| | | (8:2) | | | |
| 9 | CAP | THF | 0.017 | 0.042 | Amorphous + one |
| | | | | | small NaCl peak |
| | | | | | (~32 o 2θ)* |
| 10 | PVAP | THF | 0.016 | 0.036 | Amorphous + one |
| | | | | | small NaCl peak |
| | | | | | (~32 o 2θ)* |
| 12 | HPMCP | Acetone | 0.003 | 0.014 | Amorphous |
| | 55 | | | | |

*NaCl peak = corresponds to residual NaCl being crystallized on ASD solids recovered after solubility study.

TABLE 2A-10
Summary of solubility results for ASDs in FeSSIF

| | | 1 hr | 2 hr | |
|---|---|---|---|---|
| | | solubility in | solubility in | |
| | | FeSSIF | FeSSIF | |
| | | (mg/mL) | (mg/mL) | |
| Polymer | Solvent | (n = 2) | (n = 2) | XRPD after FeSSIF |
| PVAP | THF | 0.206 | 0.152 | Amorphous + one small |
| | | | | NaCl peak (~32° 2θ) |
| HPMCP | DCM:Acetone | 0.022 | 0.113 | Amorphous + four |
| 55 | (1:1) | | | FeSSIF peaks* |
| EL100 | DCM:MeOH | 0.026 | 0.056 | Amorphous + four |
| | (8:2) | | | FeSSIF peaks* |
| CAP | THF | 0.028 | 0.034 | Amorphous + four |
| | | | | FeSSIF peaks* |
| HPMCAS | DCM:Acetone | 0.018 | 0.025 | Amorphous + four |
| | (5:1) | | | FeSSIF peaks* |
| HPMCP | Acetone | 0.022 | 0.025 | Amorphous + four |
| 55 | | | | FeSSIF peaks* |
| NA | NA | 0.005 | 0.006 | Form I + one peak ~9° |
| | | | | 2θ* |

### ASD Stability Analysis

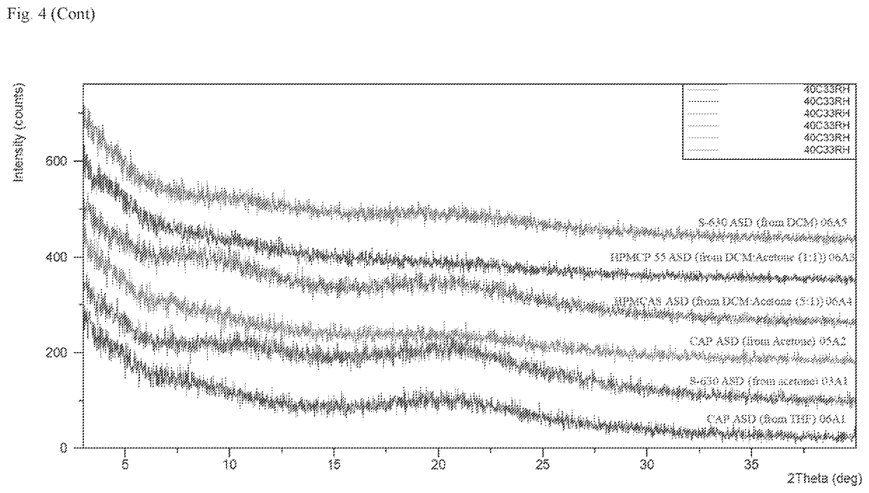

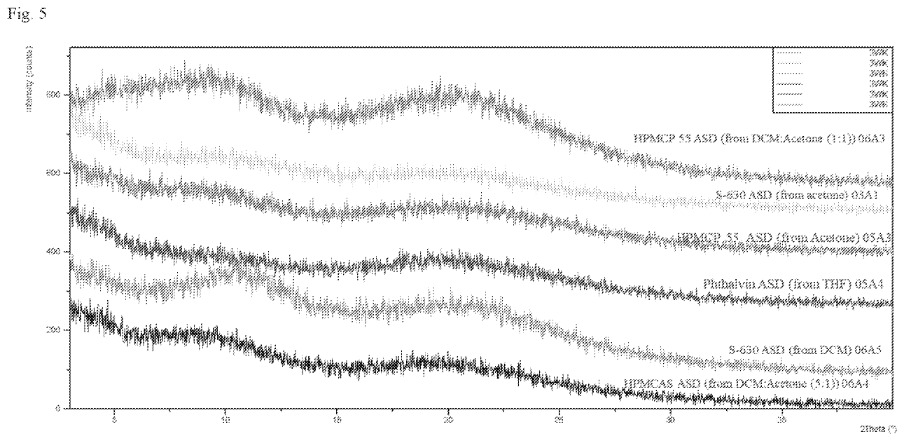

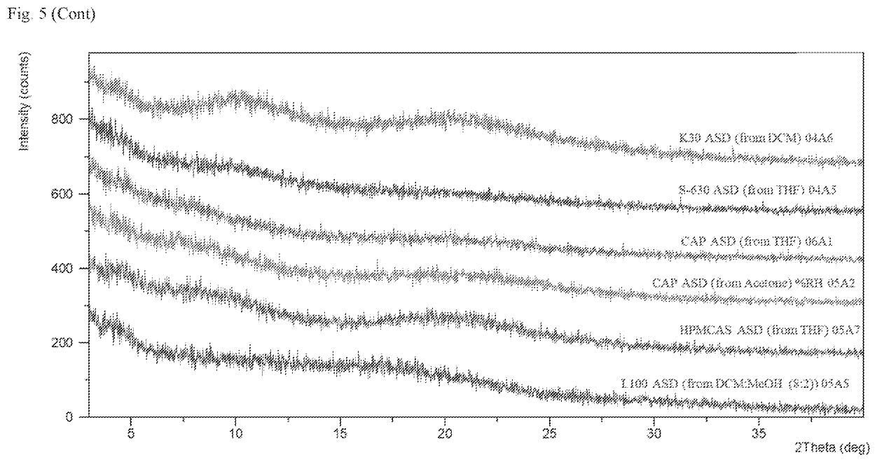

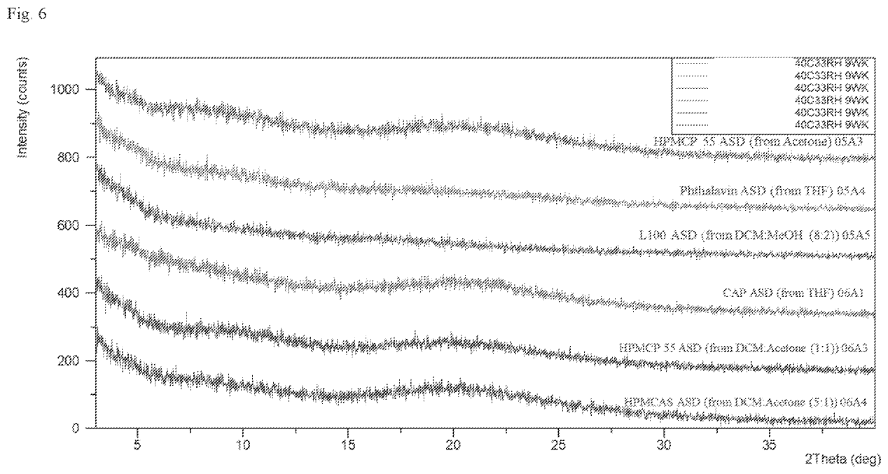

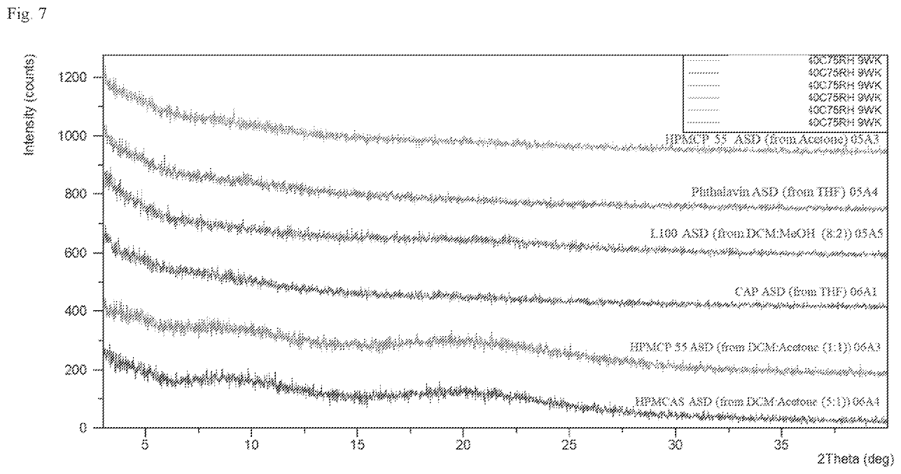

The six selected ASDs were stored and studied at the accelerated conditions of 40° C./33% RH and 40° C./75% RH to monitor the stability of the ASD at different timed intervals by XRPD, mDSC, and TGA. All six ASDs stored at 40° C./33% RH for two weeks remained amorphous as per XRPD data. See FIG. 4. Similarly, all six ASDs stored at 40° C./75% RH for three weeks also remained amorphous by XRPD. See FIG. 5. All six ASDs (stored at both 40° C./33% RH and 40° C./75% RH) were re-analyzed by XRPD again at nine-week time point and were found to be amorphous. See FIGS. 6, 7. In addition to determining the Tg values and the total residual solvent/moisture content of the ASD formulations, six ASDs after exposure to the accelerated conditions, ASDs were characterized by mDSC and TGA. The mDSC for stability samples was carried in hermetically sealed pan without any pin hole, to determine Tg in presence of residual solvent or moisture (i.e., wet Tg). Results pertaining to mDSC and TGA analysis are summarized in Table 2A-11.

TABLE 2A-11
Summary of characterization of ASDs after exposure to accelerated stability conditions

| | | | | TGA | | | | |
|---|---|---|---|---|---|---|---|---|
| | | | 3-week | wt. | Tg | 2-week | | |
| | | | stability | loss | (no | stability | wt. loss | Tg |
| | | | (40° | (% w/w) | pin | (40° | (% w/w) | (° C.) |
| | | | C./75% | at | hole) | C./33% | at 110° | (no pin |
| # | Polymer | Solvent | RH) | 110° C. | ° C. | RH) | C. | hole) |
| 1 | Hypromellose | DCM:Acetone | Amorphous | 0.62 | 71.5 | Amorphous | 0.02 | 71.3 |
| | Acetate | (5:1) | | | | | | |
| | Succinate | | | | | | | |
| | (HPMCAS) | | | | | | | |
| 2 | PVAP | THF | Amorphous | 1.27 | 50 | Amorphous | 1.09 | 42.3 |
| 3 | Hypromellose | DCM:Acetone | Amorphous | 1.37 | 58.4 | Amorphous | 0.51 | 54.1 |
| | Phthalate | (1:1) | | | | | | |
| | (HPMCP) 55 | | | | | | | |
| 4 | CAP | THF | Amorphous | 1.52 | 49.2 | Amorphous | 0.55 | 46.6* |
| 5 | HPMCP 55 | Acetone | Amorphous | 1.53 | 59.2 | Amorphous | 0.31 | 57.8 |
| 6 | EL100 | DCM:MeOH | Amorphous | 2.09 | 76.4 | Amorphous | 0.31 | 98.2 |
| | | (8:2) | | | | | | |
| 7 | Plasdone S- | THF | Amorphous | NA | NA | Amorphous | NA | NA |
| | 630 | | | | | | | |
| | (copovidone) | | | | | | | |
| 8 | Plasdone S- | DCM | Amorphous | NA | NA | Amorphous | NA | NA |
| | 630 | | | | | | | |
| | (copovidone) | | | | | | | |
| 9 | Plasdone S- | Acetone** | Amorphous | NA | NA | Amorphous | NA | NA |
| | 630 | | | | | | | |
| | (copovidone) | | | | | | | |
| 10 | CAP | Acetone | Amorphous | NA | NA | Amorphous | NA | NA |
| 11 | HPMCAS | THF | Amorphous | NA | NA | Amorphous | NA | NA |
| 12 | Povidone | DCM | Amorphous | NA | NA | Amorphous | NA | NA |
| | K30 | | | | | | | |

*distinct Tg like pattern not observed, tentative Tg;
NA = data not available,
mDSC/TGA was performed for shortlisted six ASDs, while other ASDs just analyzed by XRPD.

Elra:PVAP (50% Amorphous Elraglusib in PVAP) Scaled-Up Preparation by Spray-Drying from THF

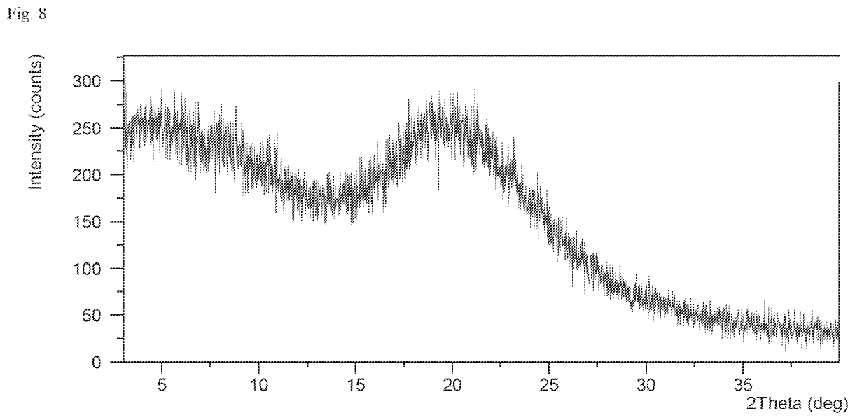

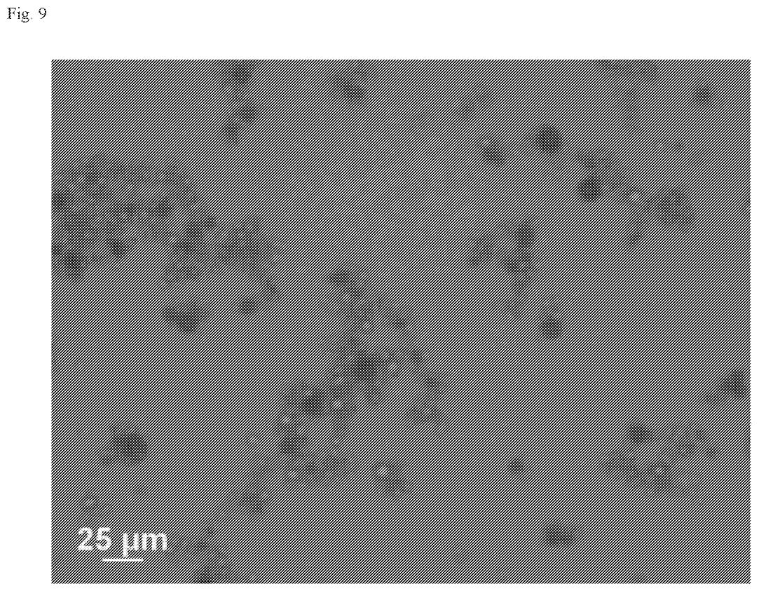

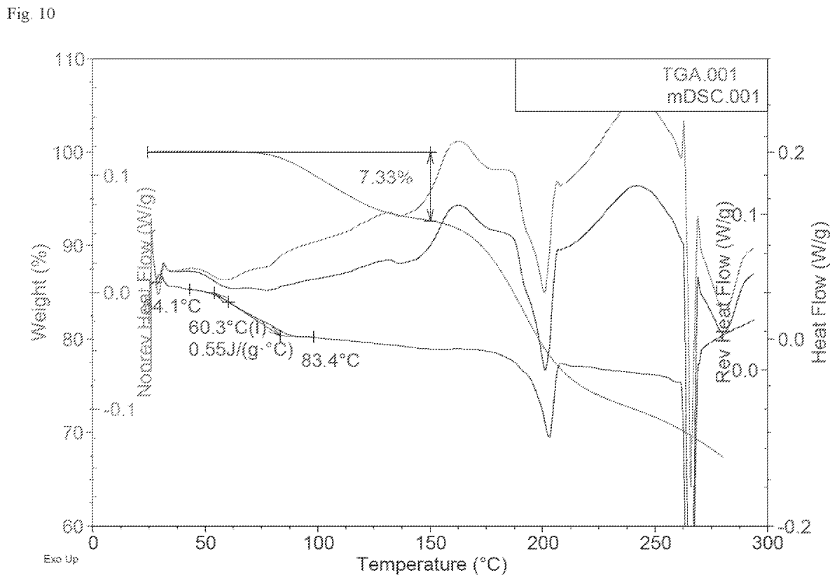

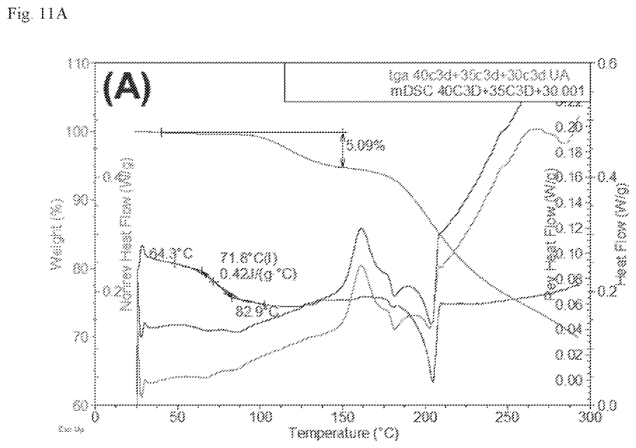

The Elra:PVAP ASD (comprising 50% w/w amorphous elraglusib in PVAP polymer) was prepared by dissolving 24 gm of elraglusib and 24 gm of PVAP in 600 mL of THF followed by spray drying using a Buchi™ B-290 spray dryer equipped with B-295 inert loop. The parameters used for spray-drying are listed in Table 2A-12. About 26 gm of Elra:PVAP was obtained, which was dried in vacuum oven at RT for two days. The XRPD of the Elra:PVAP, shown in FIG. 8, shows that material is amorphous. The PLM image, shown in FIG. 9, shows that the material consists of aggregates of spherical non-birefringent particles. The mDSC showed that the material has Tg of 60.3° C., and the TGA showed wight loss of 7.33% up to 150° C. (see FIG. 10). The material was subjected to further drying and was intermittently monitored for residual solvent by TGA; the results are presented in Table 2A-13. TGA shows that the material dried for additional 9 days in a vacuum oven at temperatures between 30° C. and 40° C. lost 5.09% of its weight up to 150° C. mDSC of this additionally dried material was carried out to determine Tg of the material in a DSC pan without pin hole and in a DSC pan with pin hole and found to be 71.8° C. and 77.8° C., respectively (FIG. 11). KF (Karl Fisher water analysis) showed presence of 3.0% water (at KF oven temperature of 200° C.).

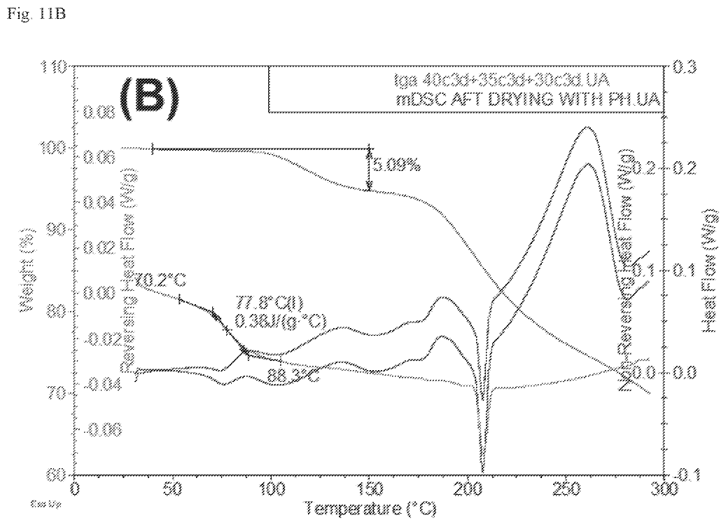

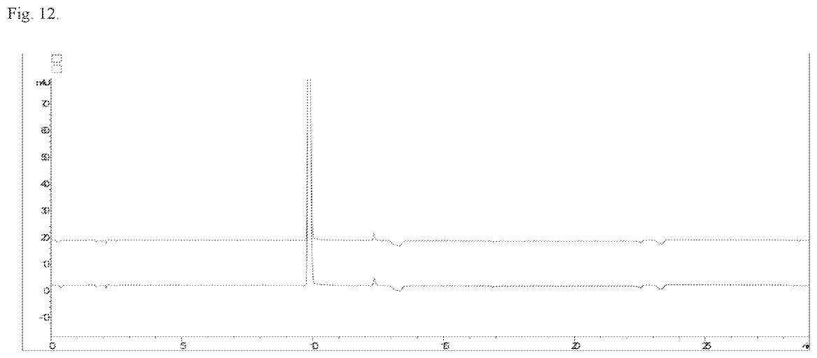

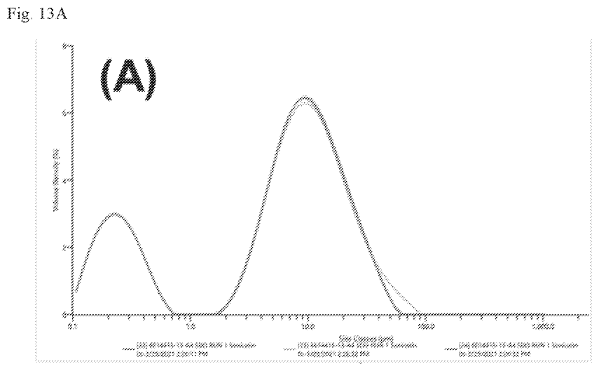

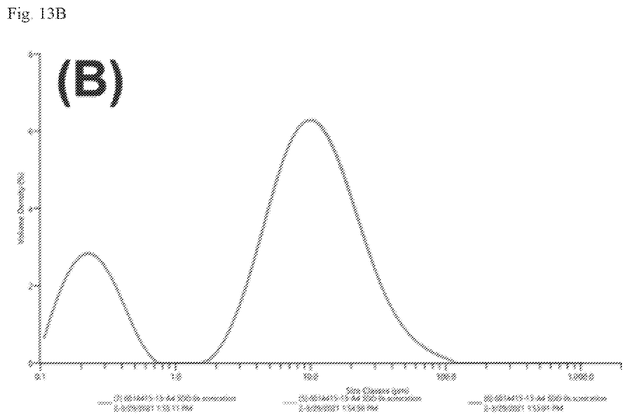

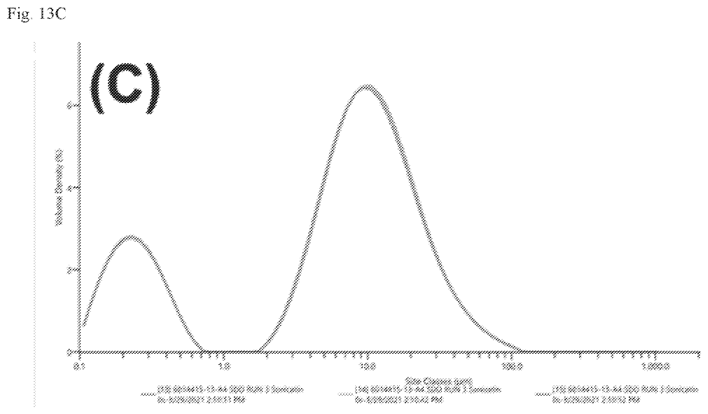

Drug loading was assessed by HPLC in triplicate by dissolving about 25 mg of Elra:PVAP in 100 mL of THF:H₂O:ACN (20:40:40). The obtained drug loading was further corrected by considering TGA weight loss for the material used for HPLC assay. The corrected drug loading was found to be 46.42% w/w (Table 2A-14) and HPLC chromatogram (monitored at 438 nm) showed presence of only API peak and known (elraglusib anhydride) related substance peak at RRT of 1.23 (FIG. 12). PSD (particle size distribution) of the sample was performed using Malvern Mastersizer instrument. About 50 mg of sample was dispersed in 10 mL of 0.25% Lecithin in Isopar-G, followed by PSD analysis in triplicate without any sonication. Data showed that D10, D50, and D90 particle size distributions were, respectively is 0.22, 7.94, and 24.97 μm. (Table 2A-15). PSD profiles for this material were distinctly bimodal as shown in FIG. 13.

TABLE 2A-12
Spray-Drying Operating Parameters
used for preparation of Elra:PVAP

| Item | Parameter | |
|---|---|---|
| Inlet | 100° | C. |
| Outlet | 55-60° | C. |
| Aspirator | 97% | |
| Pump | 30% | |
| Drying gas flow rate | 50 | cfm |

| | |
|---|---|
| Nozzle Clean | 1 |

TABLE 2A-13
Summary of the drying and characterization of Elra:PVAP

| Tg (° C.) | | | | | Tg | Tg | | |
|---|---|---|---|---|---|---|---|---|
| (no pin | Wt. loss (% w/w) at 150° C. by TGA | | | | (° C.) | (° C.) | KF | Experimental |
| hole) | | +3 d at | +3 d at | +3 d at | (no | (with | (Oven | Drug |
| Before | 2 d at RT | 40° C. | 35° C. | 30° C. | pin | pin | T = | loading* |
| drying | Vacuum | Vacuum | Vacuum | Vacuum | hole) | hole) | 200° C.) | (n = 3) |
| 60.3 | 7.33 | 5.18 | 5.03 | 5.09 | 71.8 | 77.8 | 3.0 | 46.42 |

TABLE 2A-14
Summary of drug loading determination of Elra:PVAP

| | | Average | |
|---|---|---|---|
| | Drug | Drug | |
| | Loading | Loading | Corrected average Drug |
| Sample# | (% w/w) | (% w/w) | Loading (% w/w) |
| 1 | 44.13 | 44.17 | 46.42* |
| 2 | 43.95 | | |
| 3 | 44.42 | | |

*Drug loading was corrected after considering 4.86% weight loss.

TABLE 2A-15
Summary of PSD analysis of Elra:PVAP

| | Run #1 | Run #2 | Run #3 | Average |
|---|---|---|---|---|
| D10 (μm) | 0.215 | 0.220 | 0.223 | 0.22 |
| D50 (μm) | 7.64 | 8.06 | 8.12 | 7.94 |
| D90 (μm) | 23.6 | 25.6 | 25.7 | 24.97 |

Elra:CAP (50% Amorphous Elraglusib in CAP) Scaled-Up Preparation by Spray-Drying from THF

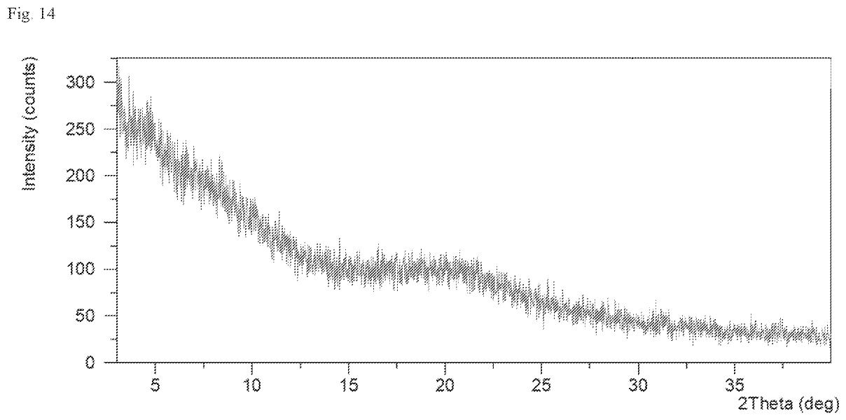

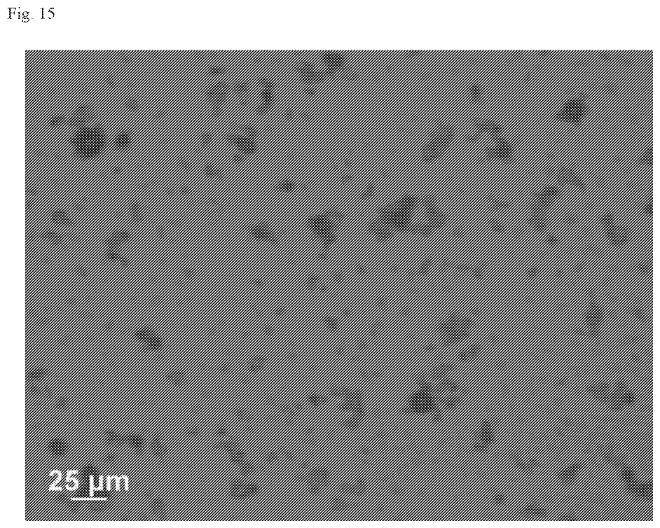

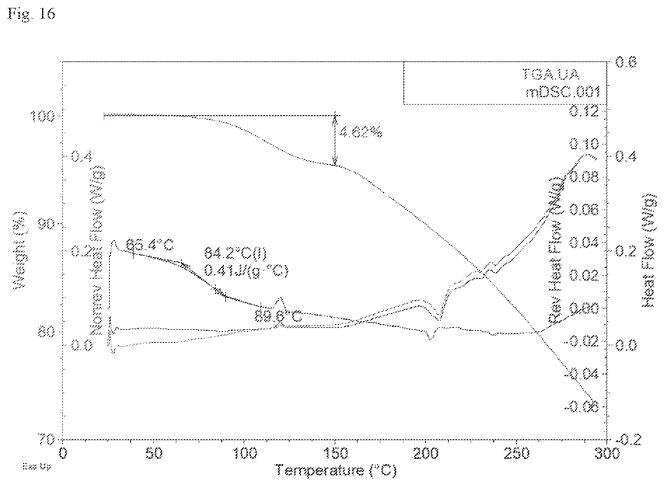

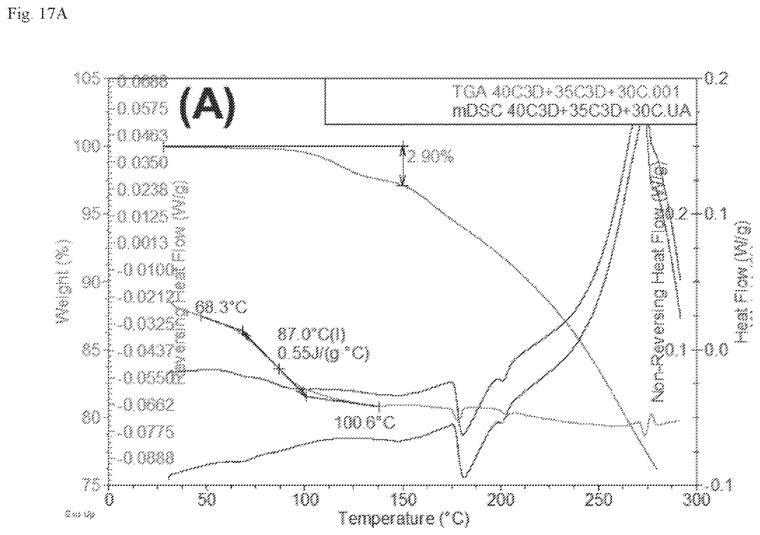

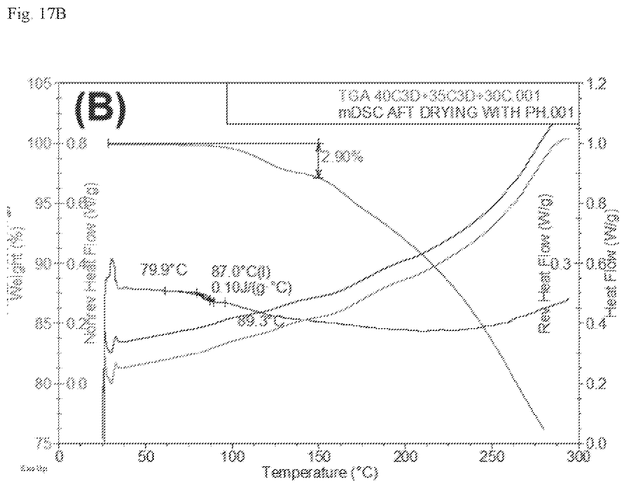

The Elra:CAP ASD (comprising 50% w/w amorphous elraglusib in CAP polymer) was prepared by dissolving 24 gm of elraglusib and 24 gm of CAP in 600 mL of THF followed by spray drying using a Buchi™ B-290 spray-dryer equipped with B-295 inert loop. The parameters used for spray drying are listed in Table 2A-16. About 29 gm of ASD was obtained, which was dried in vacuum oven at room temperature for two days. XRPD of the Elra:CAP ASD, shown in FIG. 14, showed that material was amorphous. A PLM image is displayed in FIG. 15 and shows that the material has aggregates of irregularly shaped non-birefringent particles. An mDSC showed that the material has Tg of 84.2° C., while TGA showed a weight loss of 4.62% until 150° C. (FIG. 16). Further drying with intermittent monitoring for residual solvent by TGA was performed, as shown in Table 2A-17. The TGA of the additionally dried material showed 2.90% weight loss to 150° C., while mDSC of the additionally dried material was also conducted to determine Tg of the material in DSC pan without pin hole and with pin hole and found to be 87.0° C. and 87.0° C., respectively (FIG. 17). KF showed presence of 2.9% water (at KF oven temperature of 200° C.).

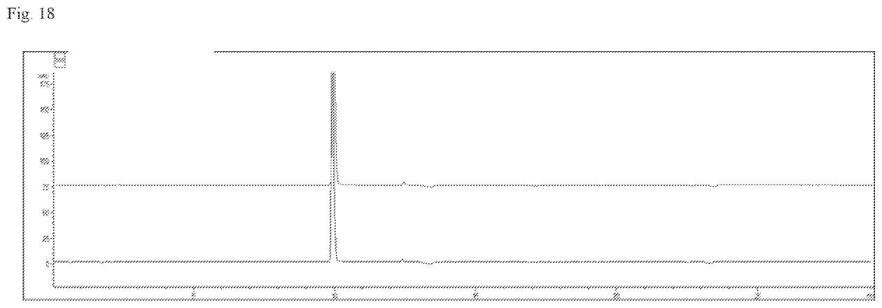

The drug loading was assessed by HPLC in triplicate by dissolving approximately 25 mg of the Elra:CAP ASD in 100 mL of THF:H₂O:ACN (20:40:40). The obtained drug loading was further corrected by considering TGA weight loss for the material used for HPLC assay. The Elra:CAP ASD, had a corrected drug loading of 46.98% w/w (Table 2A-18), the HPLC chromatograms showed only the API peak and the known related substance peak (elraglusib anhydride) at RRT 1.23 (FIG. 18).

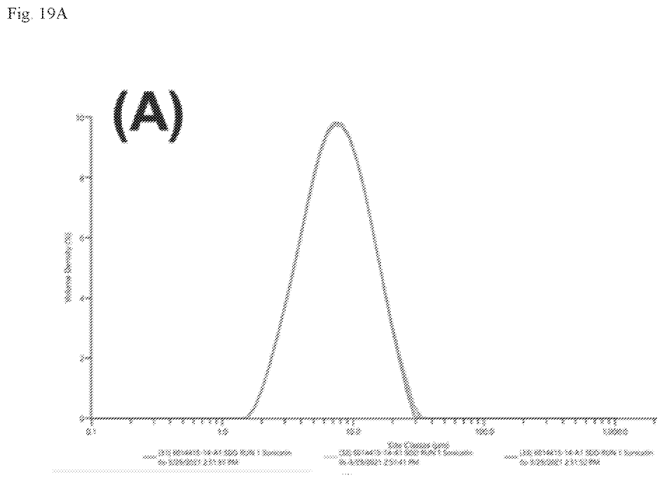

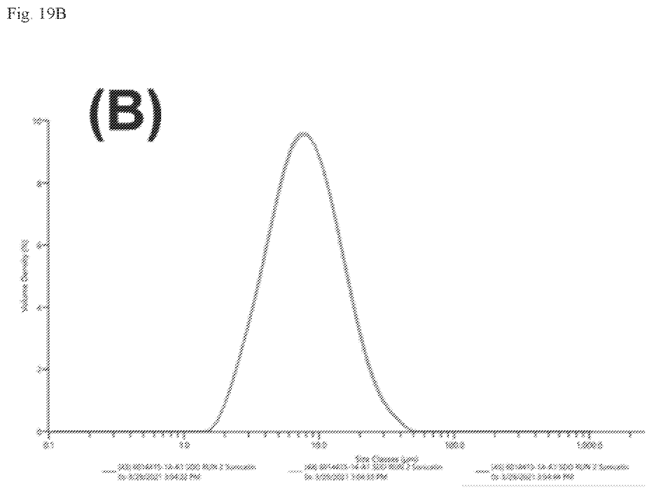

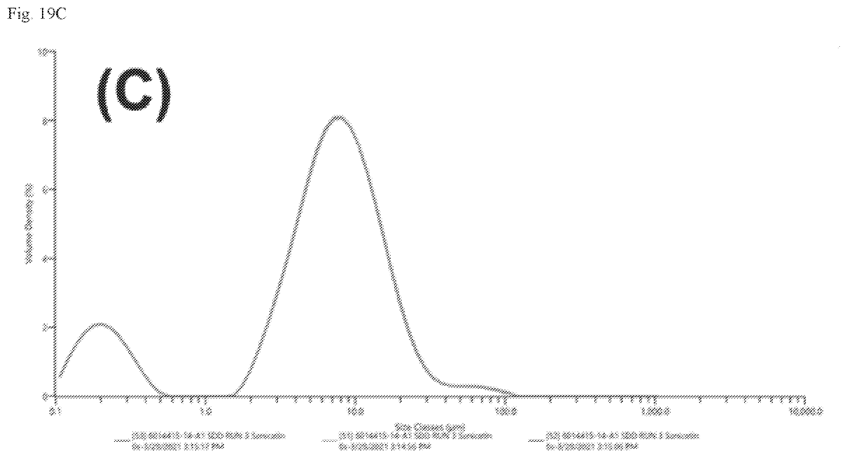

Particle Size Distribution (PSD) of the sample was also performed using Malvern Mastersizer instrument. About 50 mg of sample was dispersed in 10 mL of 0.25% Lecithin in Isopar-G, followed by PSD analysis in triplicate without any application of sonication. The average D10, D50, and D90, particle size distributions, respectively, are 2.38, 7.34, and 16.73 μm. (Table 2A-19). PSD profiles for this material showed either mono-modal or bimodal distribution of particles as shown in FIG. 19.

TABLE 2A-16
Spray Drying Operating parameters used
for preparation of Elra:CAP from THF

| Item | Parameter | |
|---|---|---|
| Inlet | 100° | C. |
| Outlet | 60° | C. |
| Aspirator | 97% | |
| Pump | 30% | |
| Drying gas flow rate | 50 | cfm |

| | |
|---|---|
| Nozzle Clean | 1 |

TABLE 2A-17
Summary of the drying and characterization of Elra:CAP

| | | | | | | Tg (° C.) | | |
|---|---|---|---|---|---|---|---|---|
| Tg (° C.) | | | | | Tg (° C.) | (with | | |
| (no pin | Wt. loss (% w/w) at 150° C. | | | | (no pin | pin | KF | Experimental |
| hole) | | +3 d at | +3 d at | +3 d at | hole) | hole) | (Oven | Drug |
| Before | 2 d at RT | 40° C. | 35° C. | 30° C. | After | After | T = 200° | loading* |
| drying | Vacuum | Vacuum | Vacuum | Vacuum | drying | drying | C.) | (n = 3) |
| 84.2 | 4.62 | 3.18 | 2.75 | 2.90 | 87.0 | 87.0 | 2.9 | 46.98 |

TABLE 2A-18
Summary of drug loading determination of Elra:CAP

| | Drug | Average Drug | Corrected average |
|---|---|---|---|
| | Loading | Loading | Drug Loading |
| Sample# | (% w/w) | (% w/w) | (% w/w) |
| 1 | 44.86 | 45.62 | 46.98* |
| 2 | 46.01 | | |
| 3 | 45.98 | | |

TABLE 2A-19
Summary of PSD analysis of Elra:CAP

| | Run #1 | Run #2 | Run #3 | Average |
|---|---|---|---|---|
| D10 (μm) | 3.4 | 3.48 | 0.258 | 2.38 |
| D50 (μm) | 7.45 | 7.72 | 6.86 | 7.34 |
| D90 (μm) | 15.8 | 17.3 | 17.1 | 16.73 |

### Elra:PVAP and Elra:CAP (Scaled Up) Kinetic Solubility Study

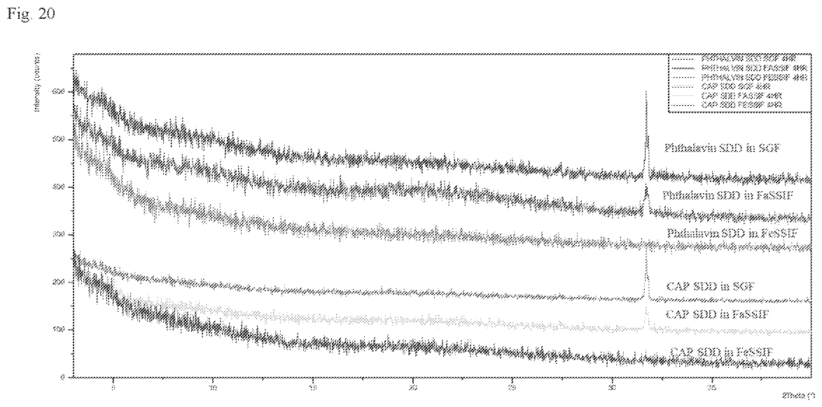

The kinetic solubility profiles of the Elra:PVAP ASD and Elra:CAP ASD were measured in biorelevant media at 37° C. Suspensions of each ASD were prepared in three biorelevant media, namely: SGF, FaSSIF, and FeSSIF. These suspensions were stirred at 400 rpm. An aliquot of the supernatant was withdrawn at 5 min, 10 min, 30 min, and 4 hours, syringe-filtered using a cellulose acetate filter (0.22 μM pore size) and analyzed by HPLC. The solubility profiles of these ASDs in SGF, FaSSIF, and FeSSIF are presented in Table 2A-20. The solubility assessment showed that both ASDs have higher solubility in FaSSIF and FeSSIF compared to SGF and Elra:PVAP has higher solubilities compared to Elra:CAP under similar conditions. In addition, XRPD of solid recovered after solubility analysis at 4 hours showed that both of the ASDs remained amorphous after dissolution (FIG. 20). XRPD of the sample recovered from SGF and FaSSIF showed presence of a peak ~32° 20, which corresponds to residual NaCl (from buffer) being crystallized.

TABLE 2A-20
Summary of kinetic solubility analysis for Elra:CAP and Elra:PVAP ASDs

| | | | | | | XRPD of solid |
|---|---|---|---|---|---|---|
| | | Solubility (mg/mL) (n = 2) | | | | recovered after |
| Polymer | Media | 5 min | 10 min | 30 min | 4 hr | solubility (@t = 4 hr) |
| Elra:PVAP | SGF | <LOD | <LOD | <LOD | <LOD | Amorphous + one |
| | | | | | | NaCl peak (~32° |
| | | | | | | 2θ)* |
| | FaSSIF | 1.62 | 5.49 | 5.91 | 12.25 | Amorphous + one |
| | | | | | | NaCl peak (~32° |
| | | | | | | 2θ)* |
| | FeSSIF | 2.42 | 2.53 | 2.99 | 4.52 | Amorphous |
| Elra:CAP | SGF | <LOD | <LOD | <LOD | <LOD | Amorphous + one |
| | | | | | | NaCl peak (~32° |
| | | | | | | 2θ)* |
| | FaSSIF | 1.01 | 1.29 | 0.97 | 1.81 | Amorphous + one |
| | | | | | | NaCl peak (~32° |
| | | | | | | 2θ)* |
| | FeSSIF | 1.35 | 1.46 | 1.54 | 1.71 | Amorphous |

*NaCl peak = corresponds to residual NaCl being crystallized on ASD solids recovered after solubility study.
LOD = 0.006 mg/mL

### Elra:PVAP and Elra:CAP ASD Stability Study

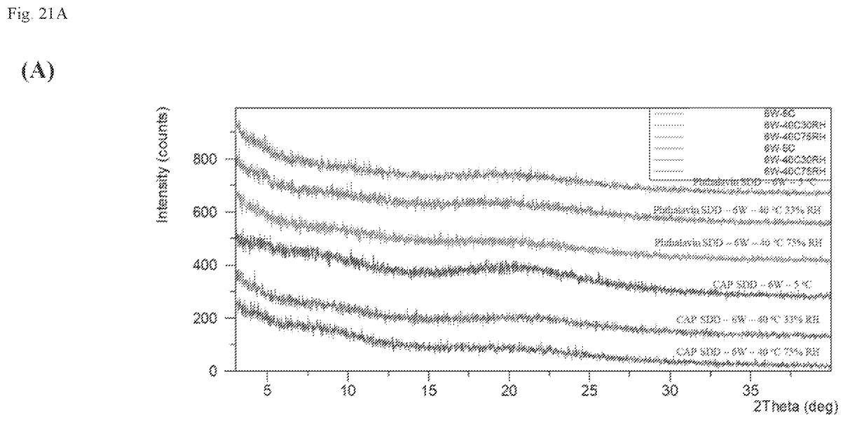

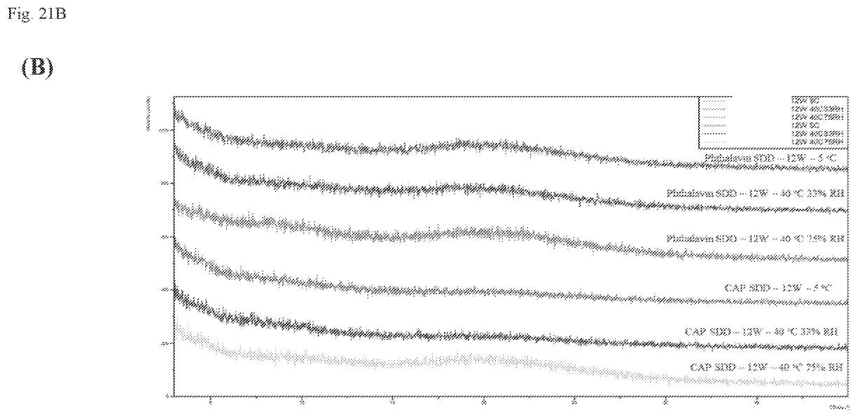

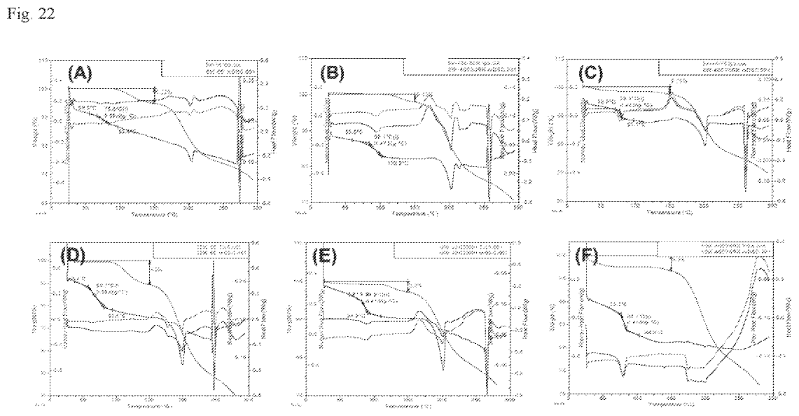

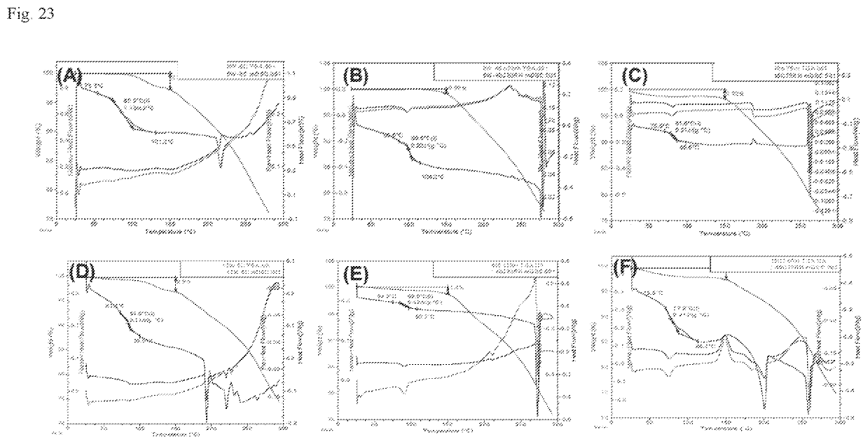

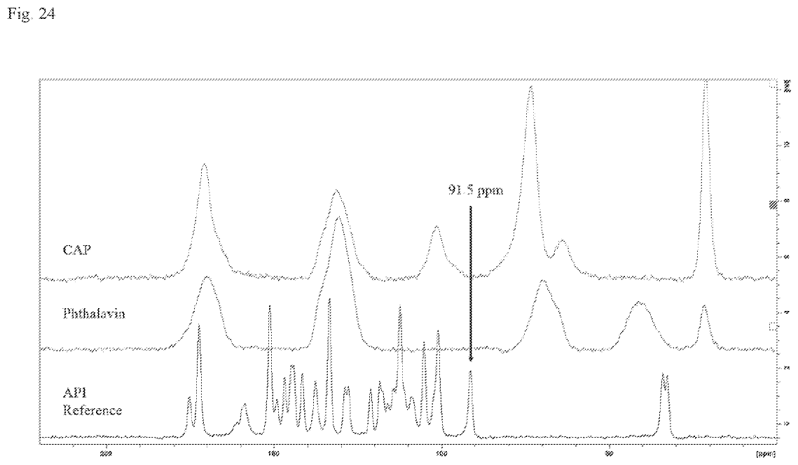

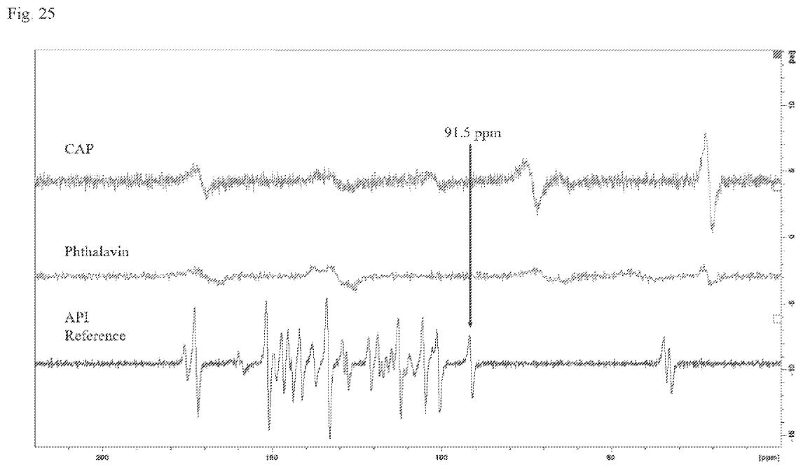

To check susceptibility of both Elra:PVAP and Elra:CAP ASDs to crystallization under accelerated stability conditions, each ASD was stored at 5° C., 40° C./33% RH and 40° C./75% RH and monitored at 6-week and 12-week time point by XRPD, mDSC, TGA, and HPLC. Both ASDs stored remained amorphous up to 12 weeks storage under the above-mentioned conditions (FIG. 21). The mDSC of the stability samples was carried out in a hermetically sealed pan without any pin hole, to determine the Tg in the presence of residual solvent or moisture (i.e., wet Tg). Results pertaining to mDSC and TGA analysis are summarized in Table 2A-21 and presented in FIG. 22 and FIG. 23. Both ASDs stored at 40° C./33% RH for six weeks showed lowest weight loss and highest Tg compared to other storage conditions, which could be result of partial drying of the sample at 40° C./33% RH (compared to starting material's initial storage condition of 25° C. and 60-70% RH). In most of these conditions, the Tg of both ASDs was within 10% of the initial Tg value, except for Elra:CAP, which showed ~11.5% decrease in Tg value compared to initial Tg.

HPLC analysis of both ASDs stored at all three conditions did not show any degradation when monitored at 438 nm, it only showed peaks for the known impurity at RRT ~1.26. The HPLC samples monitored at 438 nm and their respective % area purity profiles are presented in Table 2A-22 and Table 2A-23. Data showed that % area purity remain about 99% in all the initial (t=0) and stability samples. An HPLC chromatogram was also obtained using HPLC monitored at 210 nm for a sample stored for 12 weeks at different conditions. The results showed that % area purity remain about 99% in all the stability samples (Table 2A-24 and 2A-25). These results collectively showed that both ASD samples are physically and chemically stable at 40° C./33% RH and 40° C./75% RH up to 12 weeks.

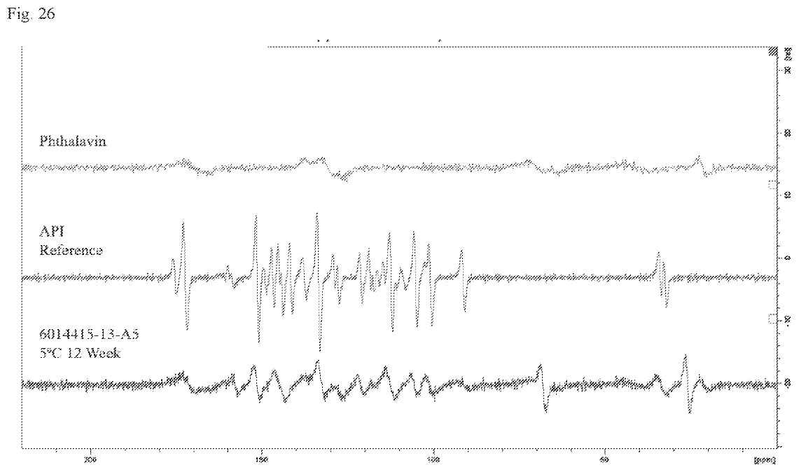

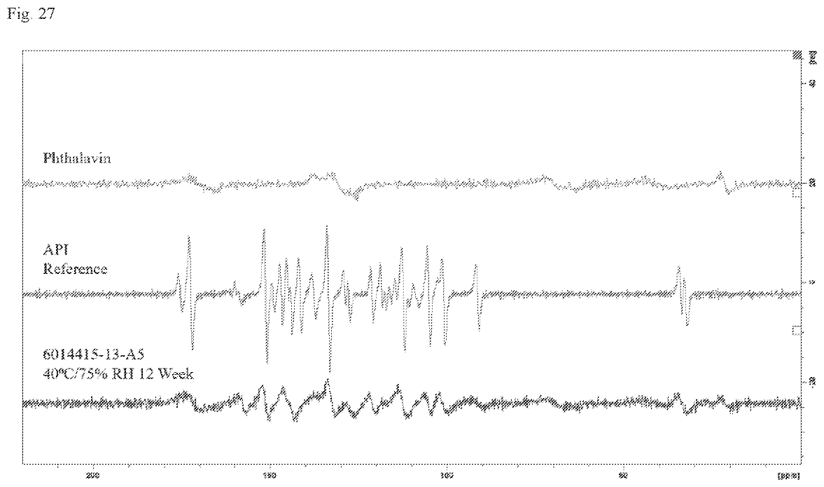

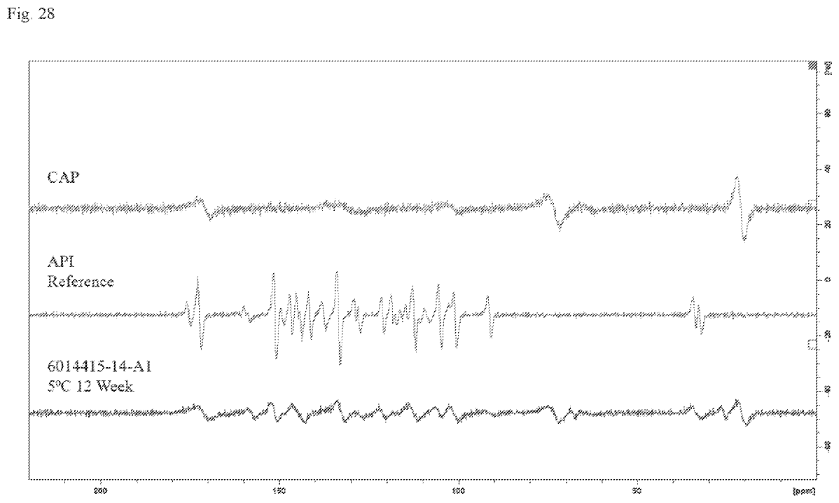

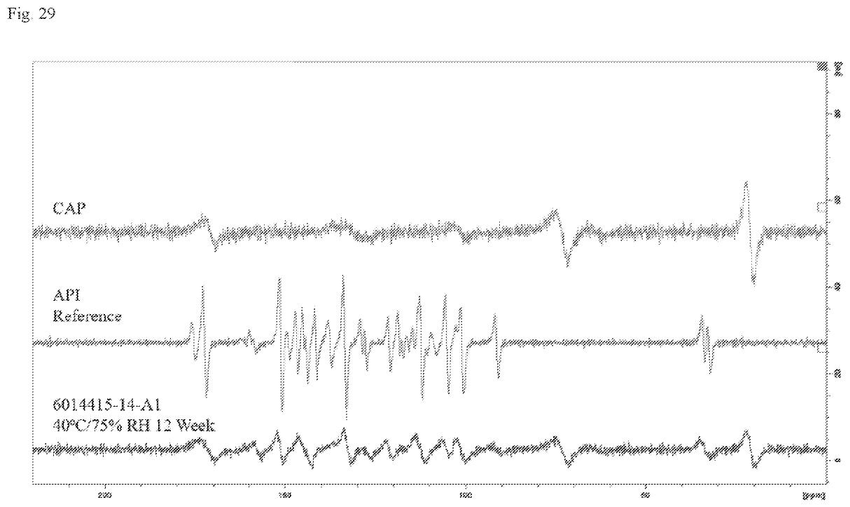

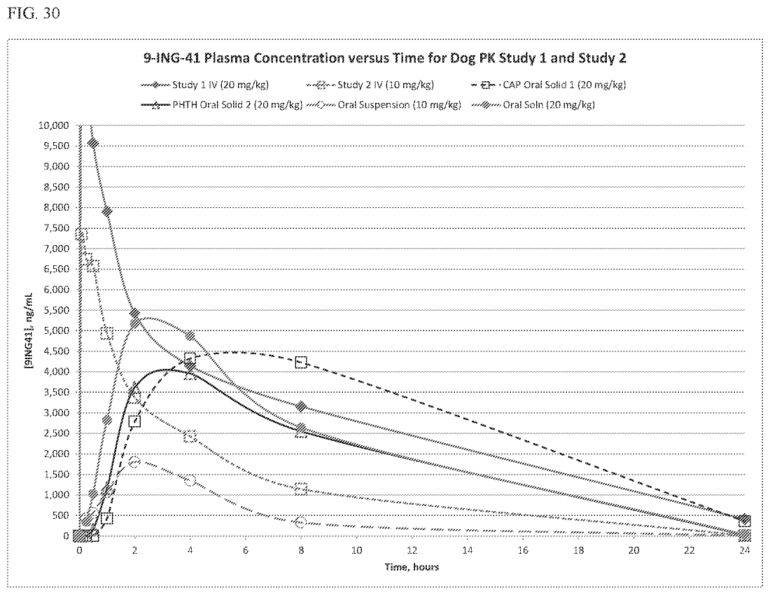

Overlay of elraglusib, Elra:CAP, and Elra:PVAP ASDs by ssNMR displayed a baseline resolved peak of the API at 87 ppm indicating a suitable location for monitoring the stability of ASD batches for crystallizing under accelerated stability conditions (FIG. 24). The first derivative was applied to the stability sample spectra (FIGS. 25-29). For pure amorphous samples, ssNMR produces gaussian curves that when applied to first derivative become a flat baseline. Crystalline material produces Laplacian curves by ssNMR which can be clearly observed after applying the first derivative.

Signal to noise ratio of API reference was 8.18. In all four stability samples, the signal to noise ratio for the first derivative peak at 87 ppm remained <2. Results are summarized in Table 2A-26.

The results of the CPMAS ssNMR showed no detectable crystallinity in any of the ASDs tested after their exposure to both the stability conditions.

TABLE 2A-21
Summary of characterization of stability samples of Elra:CAP and Elra:PVAP

| | | | Wt. loss (% w/w) at 150° C. | | | | Tg (° C.) (no pin hole) | | | |
|---|---|---|---|---|---|---|---|---|---|---|
| | Stability | | | | 40° | 40° | | | 40° | 40° |
| | time | | | | C./ | C./ | | | C./ | C./ |
| | point | | | 5° | 33% | 75% | | 5° | 33% | 75% |
| ASD | (weeks) | XRPD | Initial | C. | RH | RH | Initial | C. | RH | RH |
| Elra:PVAP | 6 | Amorphous | 5.09 | 5.22 | 2.15 | 2.73 | 71.8 | 75.0 | 98.1 | 78.4 |
| | 12 | Amorphous | 5.09 | 5.3 | 2.9 | 3.3 | 71.8 | 66.7 | 84.2 | 82.1 |
| Elra:CAP | 6 | Amorphous | 2.90 | 2.83 | 0.95 | 1.82 | 87.0 | 85.3 | 99.5 | 81.6 |
| | 12 | Amorphous | 2.90 | 2.90 | 1.40 | 2.30 | 87.0 | 84.0 | 92.0 | 77.2 |

Tg of neat Phthalavin (PVAP) 120° C. and showed weight loss of 1.73% till 150° C.
Tg of neat CAP is 180° C. and showed weight loss of 2.77% till 150° C.

TABLE 2A-22
Summary of HPLC peak % area purity profile for
Elra:PVAP ASD samples monitored at 438 nm

| | | 5° C. | | 40° C./33% RH | | 40° C./75% RH | |
|---|---|---|---|---|---|---|---|
| | | 6 | 12 | 6 | 12 | 6 | 12 |
| RRT | Initial | week | week | week | week | week | week |
| 1.00 | 99.12 | 98.95 | 99.08 | 99.11 | 99.07 | 99.08 | 99.10 |
| 1.26 | 0.88 | 1.05 | 0.92 | 0.89 | 0.93 | 0.92 | 0.90 |

TABLE 2A-23
Summary of HPLC peak % area purity profile for
Elra:CAP ASD samples monitored at 438 nm

| | | 5° C. | | 40° C./33% RH | | 40° C./75% RH | |
|---|---|---|---|---|---|---|---|
| | | 6 | 12 | 6 | 12 | 6 | 12 |
| RRT | Initial | week | week | week | week | week | week |
| 1.00 | 99.14 | 99.11 | 99.13 | 99.02 | 99.10 | 99.1 | 99.095 |
| 1.26 | 0.86 | 0.89 | 0.87 | 0.98 | 0.90 | 0.9 | 0.91 |

TABLE 2A-24
Summary of HPLC peak % area purity profile for
Elra:PVAP ASD samples monitored at 210 nm

| | | 40° C./33% | 40° C./75% |
|---|---|---|---|
| RRT | 5° C. | RH | RH |
| 1.00 | 99.19 | 98.83 | 98.99 |
| 1.13 | 0.44 | — | — |
| 1.26 | 0.37 | 1.17 | 1.01 |

TABLE 2A-25
Summary of HPLC peak % area purity profile for
Elra:CAP ASD samples monitored at 210 nm

| | | 40° C. 33% | 40° C. 75% |
|---|---|---|---|
| RRT | 5° C. | RH | RH |
| 1.00 | 99.44 | 99.37 | 99.29 |
| 1.13 | 0.12 | 0.27 | — |
| 1.26 | 0.44 | 0.36 | 0.71 |

TABLE 2A-26
Summary of ssNMR signal to noise results
for first derivative spectra

| | | |
|---|---|---|
| | | Signal to Noise |
| ID | Sample | (87-96 ppm) |

| | |
|---|---|
| API Reference | 8.18 |
| 5° C. for 12 week | 1.86 |
| 40° C./75% RH for 12 week | 1.22 |
| 5° C. for 12 week | 1.21 |
| 40° C./75% RH for 12 week | 1.40 |

### Analytical Methods: Characterization of ASD Powders

HPLC Testing of Elra:CAP. Transferred 10.254 mg of Elra:CAP ASD from bottle 1 and 10.465 mg from bottle 2 into separate 50 mL volumetric flasks. Diluted to volume with acetonitrile and mixed well to dissolve. Filtered the solutions with 0.45-μm PVDF filters to remove small clear crystals from the solution. Transferred the solutions to HPLC vials and stored at 5° C. Expiration date was 5 days.

HPLC Testing of Elra:PVAP. Transferred 10.177 mg of 9-ING-41-Phth ASD from bottle 1 and 10.105 mg from bottle 2 into separate 50 mL volumetric flasks. Diluted to volume with acetonitrile and mixed well to dissolve. The solutions were sonicated to break up white clumps that had formed. Filtered the solutions with 0.45-μm PVDF filters to remove any insoluble particles from the solution. Transferred the solutions to HPLC vials and stored at 5° C. Expiration date was 5 days.

ATR FT-IR analysis of ASD powders: CAP polymer, PVAP polymer, Elra:CAP ASD, Elra:PVAP ASD, and elraglusibAPI were analyzed by FT-IR using the Attenuated Total Reflection (ATR) accessory. A background (16 scans) was run before each sample. A small sample was placed on the ATR crystal and the pressure plate was tightened. 16 scans were performed for each sample. The crystal was cleaned with isopropanol after each analysis.

XRPD Analysis: XRPD was performed with a Panalytical X'Pert3 Powder XRPD on a Si zero-background holder. The 20 position was calibrated against a Panalytical Si reference standard disc. XRPD parameters are listed in Table 2A-27.

TABLE 2A-27
Operating Parameters for XRPD

| Parameters | Reflection Mode |
|---|---|
| X-Ray wavelength | Cu, kα |
| | Kα1 (Å): 1.540598, Kα2 (Å): 1.544426 |
| | Kα2/Kα1 intensity ratio: 0.50 |
| X-Ray tube setting | 45 KV, 40 mA |
| Divergence slit | Fixed ⅛° |
| Scan mode | Continuous |
| Scan range | 3-40 |
| (°2θ) | |
| Scan step time [s] | 18.87 |
| Step size | 0.0131 |
| (°2θ) | |
| Test Time | 4 min 15 s |

TGA/DSC Analysis: TGA data was collected using a TA Discovery 550 TGA from TA Instrument. DSC was performed using a TA Q2000 DSC from TA Instrument. DSC was calibrated with Indium reference standard and the TGA was calibrated using nickel reference standard. Detailed parameters used are listed in Table 2A-28.

TABLE 2A-28
Operating Parameters for TGA and DSC

| Parameters | TGA DSC | DSC |
|---|---|---|
| Method | Ramp | Ramp |
| Sample pan | Platinum, open | Aluminum, crimped |

| | |
|---|---|
| Temperature | RT-380° C. |
| Heating rate | 10° C./min |
| Purge gas | N₂ |

mDSC analysis: mDSC was performed using a TA Q2000 DSC from TA Instrument. DSC was calibrated with Indium reference standard. Detailed parameters used are listed in Table 2A-29.

TABLE 2A-29
Operating Parameters for mDSC.

| Parameters | mDSC |
|---|---|
| Mode | Modulated |
| Method | Conventional mDSC |
| Sample pan | Aluminum, hermetic lid crimped |
| Purge gas | N₂ |
| Procedure | 1) Modulation temp amplitude +/− 1.000° C. |
| | 2) Modulate Period 60 s, 3) Ramp Rate 3.0° C./min 4) |
| | Start Temp 25° C. 5) Final Temp 300° C. |

PSD analysis: a Malvern (Mastersizer 3000E) Particle Size Analyzer with Hydro EV attachment was used. The parameters used were listed in Table 2A-30:

TABLE 2A-30
Operating Parameters for PSD test

| Parameters | Settings | |
|---|---|---|
| Instrument | Malvern 3000E | |
| Distribution | Volume | |
| Obscuration | 2-15% | |
| Particle Shape | Non-Spherical | |
| Particle RI | 1.60 | |
| Dispersant | Isopar G* | |
| Dispersant RI | 1.42 | |
| Run Time | 10 seconds/run | |
| Measurements | N = 3 | |
| Flow rate | 2500 | rpm |
| Sonication Power | 30 | W |
| Sonication Time | 30 | s |

ssNMR:

Solid-state NMR experiments were performed on a 2 channel Bruker NEO spectrometer (Bruker, Billerica, MA) operating at 100.52 MHz for 13C and 399.71 MHz for 1H. A Chemagnetics APEX probe, refitted with a 7 mm magic angle spinning module (Revolution NMR, Fort Collins, CO) was used to acquire the data. Each sample was packed into a 7 mm zirconia rotor with Kel-F spacers. The magic angle spinning (MAS) frequency that was used to acquire all of the data was 5 kHz. 13C chemical shifts are reported relative to the methyl peak of 3-methylglutaric acid at 18.84 ppm with an accuracy of ±0.4 ppm.

Saturation recovery was used to measure 1H T1 relaxation times. 13C spectra were collected using a pre-saturation period (1H π/2, 15 μs delay, loop 20×), variable delay, 1H π/2 pulse, and then the cross polarization total suppression of spinning sidebands (CPTOSS) sequence, and 1H SPINAL-64 decoupling. The T1T2 function in the Bruker Topspin 4.0.8 software package was used to determine the 1H T1 values of the samples. The 1H T1rho values were determined by 1H π/2 pulse, variable pulse (0.5-64 ms), and then the cross polarization total suppression of spinning sidebands (CPTOSS) sequence, and 1H SPINAL-64 decoupling. The T1T2 function in the Bruker Topspin 4.0.8 software package was used to determine the 1H T1rho values of the samples. High quality ¹³C CPTOSS spectra were acquired at 5 kHz MAS using the CPTOSS sequence, a 1.5 ms contact time, 3960 acquisition points (~50 ms acquisition time). Data collection was done at a nominal temperature of 18.5° C. 13C (KAS-RS-011, MGA) Information: ¹³C chemical shifts are reported relative to the methyl peak of 3-methylglutaric acid (MGA, KAS-RS-011) at 18.84 ppm with an accuracy of ±0.4 ppm. KAS-RS-011 (3-methylglutaric acid, Batch #0000062292) was purchased from Sigma Aldrich and used as is without further purification.

The data was processed using Topspin 4.0.8© software package from Bruker Biospin. The data was Fourier transformed using the full FID (1536 points) and 0 Hz line broadening, phased (apk), baseline corrected (abs). Manual phasing performed as needed.

The fully automated processing was performed by taking the unsubtracted ¹³C spectrum and then using the Topspin software package's “build first derivative” function. The first derivative spectrum was then processed using automatic phasing (apk) in the Topspin software. S/N calculations were then performed using the “sinocal” AU-program on Topsin with the following parameters: Left Limit=93 ppm, Right Limit=91.5 ppm, Left Limit of Noise Range=300 ppm, Right Limit of Noise Range=250 ppm, Noise Width=8 ppm.

The HPLC method 1 was used for solubility, stability monitoring at 438 nm, and drug loading measurements of the ASD is summarized in Table 2A-31 and the HPLC method 2 was used for stability monitoring at 210 nm, which is summarized in Table 2A-32.

TABLE 2A-31
HPLC method 1 for solubility, stability monitoring
at 438 nm, and drug loading measurements

| Parameter | Instrument Condition | | | |
|---|---|---|---|---|
| Column | Xbridge C18, 150 × 4.6 mm, 3.5 μm | | | |
| Instrumentation | Agilent 1100 HPLC | | | |
| Flow Rate | 1.0 mL/min | | | |
| Column Temperature | 30° C. | | | |
| Sample Temperature | Ambient | | | |
| Injection Volume | 10.0 μL | | | |
| Diluent | H₂O:ACN:THF (40:40:20) | | | |
| Detection Wavelength | UV 210 nm, 438 nm (no visible lamp) | | | |
| Mobile Phase A: | 0.02% TFA in H₂O | | | |
| Mobile Phase B: | 0.02% TFA in ACN | | | |
| Mobile Phase C: | H2O:ACN:THF (40:40:20) | | | |
| Gradient | Time (min) | % A | % B | % C |
| | 0.0 | 70 | 30 | 0 |
| | 15 | 10 | 90 | 0 |
| | 20 | 10 | 90 | 0 |
| | 20.1 | 0 | 0 | 100 |
| | 24 | 0 | 0 | 100 |
| | 24.1 | 70 | 30 | 0 |
| | 29 | 70 | 30 | 0 |

TABLE 2A-32
HPLC method 2 for stability monitoring at 210 nm

| Parameter | Instrument Condition | | | |
|---|---|---|---|---|
| Column | Xbridge C18, 150 × 4.6 mm, 3.5 μm | | | |
| Instrumentation | Agilent 1100 HPLC | | | |
| Flow Rate | 1.0 mL/min | | | |
| Column Temperature | 30° C. | | | |
| Sample Temperature | Ambient | | | |
| Injection Volume | 10.0 μL | | | |
| Diluent | H₂O:ACN: THF without stabilizer | | | |
| | (40:40:20) | | | |
| Detection Wavelength | UV 210 nm | | | |
| Mobile Phase A: | 0.05% TFA in H₂O | | | |
| Mobile Phase B: | 0.05% TFA in ACN | | | |
| Mobile Phase C: | H₂O:ACN:THF without stabilizer | | | |
| | (40:40:20) | | | |
| Gradient | Time (min) | % A | % B | % C |
| | 0.0 | 70 | 30 | 0 |
| | 15 | 10 | 90 | 0 |
| | 20 | 10 | 90 | 0 |
| | 20.1 | 0 | 0 | 100 |
| | 22.10 | 0 | 0 | 100 |
| | 24.0 | 5 | 95 | 0 |
| | 24.1 | 70 | 30 | 0 |
| | 29 | 70 | 30 | 0 |

### Formulation of Bulk ASD Powders for Encapsulation

Formulation of Elra:CAP Blend with Croscarmellose Sodium:Silica (90:10)

A croscarmellose sodium:silica (90:10) blend was prepared by mixing 45.02 g of croscarmellose sodium and 5.00 g of colloidal silica gel in a large mortar and mixed well with a pestle. In a humidity-controlled chamber, 1.00 g of the croscarmellose:silica (90:10) blend was added per 19.04 g of the Elra:CAP ASD and mixed together in a mortar/pestle to yield 20 g of formulation (ASD:croscarmellose sodium:silica 95:4.5:0.5) to fill into capsules.

Formulation of Elra:PVAP Blend with Croscarmellose Sodium:Silica (90:10)

In a humidity-controlled chamber, 1.00 g of the croscarmellose sodium:silica (90:10) blend was added per 19.00 g of the Elra:PVAP ASD and mixed together in a mortar/pestle to yield 20 g of formulation (ASD:croscarmellose sodium:silica 95:4.5:0.5) to fill into capsules.

### Fill of Hard Capsules

Both ASD powders were formulated (19 to 1 ratio) with a 90:10 blend of croscarmellose sodium:silica as a bulking agent to improve powder flow and capsule dissolution. Fill size #00 hard gelatin capsules, sized to fit 200 mg of ASD powder; target dose 90 mg of elraglusib (=200 mg of 45% w/w ASD). Two ASD formulations: Elra:CAP Elra:PVAP.

### Elra:CAP ASD Capsule Preparation

60 size #00 opaque hard gelatin capsules were filled at low humidity (≤20% RH) with approximately 235 mg of Elra:CAP blend with croscarmellose sodium:silica. Capsules were placed in HDPE screw cap bottles at low humidity (≤10% RH) with 12 capsules per bottle. The bottles were stored at ambient temperature protected from light. The appearance of Elra:CAP capsule contents was an orange, free-flowing flowing powder.

### Elra:PVAP ASD Capsule Preparation

60 size #00 opaque hard gelatin capsules were filled at low humidity (≤20% RH) with approximately 235 mg of Elra:PVAP blend with croscarmellose sodium:silica. Capsules were placed in HDPE screw cap bottles at low humidity (≤10% RH) with 12 capsules per bottle. The bottles were stored at ambient temperature protected from light. The appearance of Elra:PVAP capsule contents was an orange, free-flowing flowing powder.

### Characterization of Hard Capsules

Ten empty capsules were weighed and the mean and standard deviation determined. Three Elra:CAP capsules and three Elra:PVAP capsules were opened and the contents were weighed. The contents of three Elra:CAP capsules and three Elra:PVAP capsules were individually prepped for HPLC. HPLC results are summarized in Table 2A-33 to 2A-35.

TABLE 2A-33
HPLC Characterization of Elra:CAP and Elra:PVAP Powders

| | | Total % | |
|---|---|---|---|
| Sample Name | 9ING41% w/W | Impurities | % Purity |
| 9ING41-CAP ASD Bottle 1 | 48.0 | 0.86 | 99.14 |
| 9ING41-CAP ASD Bottle 2 | 48.2 | 0.86 | 99.14 |
| | Mean = 48.1 | | |
| 9ING41-Phth ASD Bottle 1 | 46.9 | 0.87 | 99.13 |
| 9ING41-Phth ASD Bottle 2 | 46.6 | 0.85 | 99.15 |
| | Mean = 46.75 | | |

TABLE 2A-34
HPLC Characterization of Elra:CAP Capsule Contents

| | Total ASD | 9ING41 | Total % | | 9ING41 per Capsule | 9ING41 |
|---|---|---|---|---|---|---|
| Sample Name | Powder (mg) | % w/w | Impurities | % Purity | mg* | Per 900 mL^{†}, mg |
| 9ING41-CAP #1 | 205.5 | 43.9 | 0.9 | 99.1 | 90.3 | 180.6 |
| 9ING41-CAP #2 | 216.8 | 44.1 | 0.9 | 99.1 | 95.6 | 191.2 |
| 9ING41-CAP #3 | 214.8 | 44.4 | 0.9 | 99.1 | 95.3 | 190.7 |
| Mean | 212.4 | 44.1 | 0.9 | 99.1 | 93.8 | 187.5 |
| % RSD | 2.8 | 0.5 | 0.7 | 0.0 | 3.2 | 3.2 |

*9ING41 mg per capsule = (Total ASD Powder, mg)*(9ING41 % w/w)
^{†}9ING41 mg per 900 mL dissolution medium = (2 capsules)*(9ING41 mg/capsule)

TABLE 2A-35
HPLC Characterization of Elra:PVAP Capsule Contents

| | Total ASD | 9ING41 | Total % | | 9ING41 per Capsule | 9ING41 |
|---|---|---|---|---|---|---|
| Sample Name | Powder (mg) | % w/w | Impurities | % Purity | mg* | Per 900 mL^{†}, mg |
| 9ING41-Phth #1 | 208.5 | 42.9 | 0.9 | 99.1 | 89.4 | 178.9 |
| 9ING41-Phth #2 | 176.8 | 42.6 | 0.9 | 99.1 | 75.4 | 150.7 |
| 9ING41-Phth #3 | 210.3 | 43.2 | 0.9 | 99.2 | 90.8 | 181.5 |
| Mean | 198.5 | 42.9 | 0.9 | 99.1 | 85.2 | 170.3 |
| % RSD | 9.5 | 0.6 | 1.8 | 0.0 | 10.0 | 10.0 |

*9ING41 mg per capsule = (Total ASD Powder, mg)*(9ING41 % w/w)
^{†}9ING41 mg per 900 mL dissolution medium = (2 capsules)*(9ING41 mg/capsule)

### Dissolution Bath Setup

A 3% w/v Sodium Dodecyl Sulfate (SDS) solution was prepared. Transferred 180.0239 g of SDS into a 6 L volumetric flask. Diluted to volume with milliQ water and mixed well. Stored at ambient temperature with a 1-month expiration date. Using a graduated cylinder, 900 mL of 3% SDS was added to each dissolution vessel (6 total). The bath temperature was set to 37.5° C. and the vessels were allowed to equilibrate for approximately 15 minutes with gentle stirring at 50 rpm. The average temperature of the vessels before the capsules were added was 37.4° C. Two capsules were added to each vessel. Vessels 1-3 contained the Elra:CAP capsules and vessels 4-6 contained the Elra:PVAP capsules. 5 mL samples were taken at 0.5-, 1-, 2-, and 4-hour time points. Each Elra:CAP dissolution sample pulled at the indicated time point was a hazy orange solution with no precipitation. Both the Elra:CAP and Elra:PVAP capsules dissolved completely within 0.5 hr in 3% aqueous SDS dissolution medium. The Elra:CAP dissolution samples were slightly hazy and the Elra:PVAP dissolution samples were clear based on visual inspection. Each Elra:PVAP dissolution sample pulled at the indicated time point was a clear orange solution with no precipitation. The samples were diluted 1:1 with acetonitrile and filtered with a 0.45-μm PVDF filter before being transferred to amber HPLC vials. HPLC sample were stored at 5° C. before analysis. Dissolution characteristics are summarized in Tables 2A-36 and 2A-37.

TABLE 2A-36
Elra:CAP Capsule Dissolution Rate Profile Results

| | 9-ING- | [9-ING- | |
|---|---|---|---|
| | 41% of | 41]_{obs}, | % |
| Sample Name | Max* | μg/mL^{†} | Purity |
| Elra:CAP Disso #1 T = 0.5 h | 72.5 | 151.1 | 99.49 |
| Elra:CAP Disso #2 T = 0.5 h | 72.7 | 151.4 | 99.33 |
| Elra:CAP Disso #3 T = 0.5 h | 75.1 | 156.6 | 99.32 |
| Elra:CAP Disso #1 T = 1 h | 90.1 | 187.6 | 99.25 |
| Elra:CAP Disso #2 T = 1 h | 85.9 | 178.9 | 99.41 |
| Elra:CAP Disso #3 T = 1 h | 89.7 | 186.7 | 99.29 |
| Elra:CAP Disso #1 T = 2 h | 95.4 | 198.7 | 99.25 |
| Elra:CAP Disso #2 T = 2 h | 92.6 | 192.7 | 99.43 |
| Elra:CAP Disso #3 T = 2 h | 94.9 | 197.6 | 99.42 |
| Elra:CAP Disso #1 T = 4 h | 94.3 | 196.4 | 99.42 |
| Elra:CAP Disso #2 T = 4 h | 98.1 | 204.3 | 99.38 |
| Elra:CAP Disso #3 T = 4 h | 97.8 | 203.6 | 99.38 |

^{†}Overall Mean = 184 μg/mL ± 11% RSD
*9-ING-41% of Max = 100*([9-ING-41]_{obs} ÷ (187,400 μg/900 mL)

TABLE 2A-37
Elra:PVAP Capsule Dissolution Rate Profile Results

| | | [9-ING- | |
|---|---|---|---|
| | 9-ING-41% | 41]_{obs}, | % |
| Sample Name | of Max* | μg/mL | Purity |
| Elra:PVAP Disso #1 T = 0.5 h | 68.7 | 130.0 | 99.55 |
| Elra:PVAP Disso #2 T = 0.5 h | 70.0 | 132.4 | 99.51 |
| Elra:PVAP Disso #3 T = 0.5 h | 53.9 | 102.0 | 99.50 |
| Elra:PVAP Disso #1 T = 1 h | 81.7 | 154.6 | 99.66 |
| Elra:PVAP Disso #2 T = 1 h | 80.0 | 151.3 | 99.29 |
| Elra:PVAP Disso #3 T = 1 h | 69.2 | 131.0 | 99.30 |
| Elra:PVAP Disso #1 T = 2 h | 90.2 | 170.6 | 99.57 |
| Elra:PVAP Disso #2 T = 2 h | 89.4 | 169.1 | 99.54 |
| Elra:PVAP Disso #3 T = 2 h | 83.4 | 157.7 | 99.30 |
| Elra:PVAP Disso #1 T = 4 h | 100.1 | 189.3 | 99.56 |
| Elra:PVAP Disso #2 T = 4 h | 97.8 | 185.0 | 99.31 |
| Elra:PVAP Disso #3 T = 4 h | 98.5 | 186.3 | 99.31 |

^{†} Overall Mean = 155 μg/mL ± 17% RSD
*9-ING-41% of Max = 100*([9-ING-41]_{obs} ÷ (170,300 μg/900 mL)

### Example 2B. Tablet Formulation of Elra:CAP ASD, Elraglusib and Micronized Elraglusib

Tablets comprising either Elra:CAP ASD of the disclosure, elraglusib API or micronized elraglusib API, were prepared as described in the following.

The materials used in Elra:CAP ASD tablet studies are summarized in Table 2B-1 (raw materials) and Table 2B-2 (analytical method reagents). Equipment used in Elra:CAP ASD tablet development studies are presented in Table 2B-3.

TABLE 2B-1
Materials used in Elra:CAP ASD Tablet development studies

| Reagent | Grade | Supplier | Lot |
|---|---|---|---|
| elraglusib | NA | Pharmaron | NB-9-ING- |
| | | | 41(NG)-A-1 |
| Cellulose acetate phthalate | NF | Eastman | P1579203, WA- |
| (CAP) | | | 3616 |
| Tetrahydrofuran (THF) | HPLC, Inhibitor | VWR | 21070220 |
| | free | | |
| Microcrystalline cellulose | Avicel PH 102 | Dupont | P218831679^{a} |
| (MCC) | | | P220834058^{b} |
| Mannitol | Pearlitol 100SD | Roquette | E022G |
| Croscarmellose Sodium | Ac-Di-Sol | Dupont | TN17831402^{c} |
| (CSS) | | | TN18832540^{d} |
| Magnesium stearate | Ligamed MF-2-K | Peter Greven | C8204046 |
| Sodium lauryl sulfate | Kolliphor SLS | BASF | 13370779 |
| | fine | | |
| Hydroxypropyl cellulose | Klucel EXF | Ashland | 191855 |
| Water for Injection | Sterile | Intermountain Life | 22007202 |
| | | Sciences | |

TABLE 2B-2
Material used in Elra:CAP Tableting analytical methods

| | | Supplier/Catalog | |
|---|---|---|---|
| Reagent | Grade | Number | Lot |
| Water | MilliQ | Millipore | 0042 |
| Acetonitrile | GC | Lab Depot | 21050055 |
| Trifluroacetic acid (TFA) | HPLC, 99%+ | Sigma-Aldrich | MKCL3567 |
| Water | Deionized | Signature Select | NV06439 |
| Hexadecyltrimethylammonium | Molecular | Sigma Life | BCBZ5729 |
| bromide (CTAB) | Biology, ≥99% | Science / H6269-1KG | |
| Sodium Chloride | ACS, ≥99.0% | Spectrum / S1240 | 2GD0136 |
| Hydranal CRM Water | Hydranal | Honeywell / | L0210 |
| Standard 10.0 | | 34425 | |
| Check P | N/A | Aquamicron / | I20088 |
| | | GCHP | |

TABLE 2B-3
Equipment used in Elra:CAP ASD Tablet development studies

| Equipment | Description |
|---|---|
| Spray Dryer | Buchi B-290 |
| Gerteis MiniPactor | Roller compactor |
| High shear wet granulator | GlobePharma High Shear 1-6 |
| Tablet press (manual) for | Globe Pharma MTCM-1 |
| compression profiles | |
| Fluidized bed dryer | SolidLab1 |
| Tablet hardness tester | Varian VK200 |
| Friability test apparatus | Sotax FT2 |
| Automatic disintegration tester | Sotax DT50 |
| Dissolution testing equipment | Sotax MD / Mettler Toledo UV |
| with in-line UV | |
| Karl Fischer titrator | Mettler Toledo V20S |
| Automated sample preparation | Sotax TPW |
| workstation | |
| HPLC | Agilent 1100 with Chemstation |
| | Software |
| HPLC | Agilent 1260 with OpenLab CDS |
| | Software |
| Scanning Electron Microscope | Phenom Pro in 10 kV |
| X-ray Powder Diffraction | Rigaku - Miniflex 6G |

### Testing Methods for Example 2B

HPLC testing was performed using an Agilent 1100 series HPLC equipped with CHEMSTATION data collection software.

Chromatographic separation was achieved using a Waters Atlantis T3 4.6×150 mm, 3 μm column and the parameters provided in Table 2B-4. Testing was performed using external standard prepared at 0.2 mg/mL elraglusib in diluent. Test samples for the tablet prototypes were prepared by serial dilution to a target concentration of 0.2 mg/mL elraglusib. All samples were diluted as necessary on the Sotax TPW automated sample preparation workstation. Confirmatory preparations were prepared using volumetric flasks on a case-by-case basis.

TABLE 2B-4
Analytical conditions for elraglusib ID, assay, and related substances
method

| Mobile phase | Mobile phase A: 0.02% v/v TFA in water | |
|---|---|---|
| | Mobile phase B: 0.02% v/v TFA in | |
| | acetonitrile | |
| Diluent | 75:25 Acetonitrile:Water | |
| Column | Waters Atlantis T3, 3.0 μm, 4.6 × 150 mm | |
| | (Part Number: 186003729, Serial Number: | |
| | 2003114513695) | |
| Flow rate (mL/min) | 1.0 | |
| Column temperature (° C.) | 30 | |
| Sample temperature (° C.) | Ambient | |
| Injection volume (μL) | 20 | |
| Detector wavelength (nm) | 438 | |
| Run time (min) | 27 | |
| Acquisition time (min) | 27 | |
| Time (min) | % A | % B |
| 0.0 | 70 | 30 |
| 15.0 | 10 | 90 |
| 20.0 | 10 | 90 |
| 20.1 | 70 | 30 |
| 27.0 | 70 | 30 |

### Dissolution Testing

Dissolution was performed on a Sotax MD automated dissolution apparatus with in-line ultraviolet detection. elraglusib standard was used for quantitation of percent dissolved using ultraviolet absorbance at 282 nm. Dissolution testing was performed using the parameters shown in Table 2B-5.

TABLE 2B-5
Dissolution method operating parameters

| | |
|---|---|
| Media^{a} | 2% w/w CTAB in water |
| | 1% w/w CTAB in 0.7M sodium chloride |
| | 6% w/w CTAB in 0.7M sodium chloride |
| Vessel volume | 900 mL |
| Timepoints | 0, 15, 30, 45, 60, 75, 90, 105 and |
| | 120 min (an infinity spin [250 rpm] was |
| | included for 15 min after the 120-min timepoint) |
| Paddle speed | 75 or 100 rpm^{b} |
| Filter | 0.45 μm PVDF Syringe Filters (Pall, Part Number 4500) |
| UV detector wavelength | 282 nm |

^{a}2% w/w CTAB in water used in 35044-018, 1% w/w CTAB in 0.7M sodium chloride used in 35044-026, and 6% w/w CTAB in 0.7 M sodium chloride used in 35044-021, 35044-023, 35044-025, and 35044-031
^{b}75 rpm used in 35044-018 and 35044-021 and 100 rpm used in 35044-023, 35044-025, 35044-026 and 35044-031

### Moisture Content by Karl Fischer

Water content was determined by performing a volumetric Karl Fischer (KF) titration using a Mettler Toledo V20S KF Titrator. Titration standardization was performed with Water Standard 10.0 and Check P solution. A 300 mg sample size was used for KF water titration.

### Bulk and Tapped Density

Bulk and tap density determinations were performed in accordance to USP <616> Method I. To determine bulk density, the test article powder was dispensed into a 50 mL graduated cylinder and the weight of test article recorded. The powder mass in the measuring vessel was determined and divided by the test article volume.

To determine tapped density, the graduated cylinder was tapped until the volume did not change. The final powder volume was recorded after tapping. Tapped density was calculated by dividing the initial mass by the final volume.

Hausner ratio and compressibility index were calculated with the experimental bulk and tap densities using Equation 1 and Equation 2, respectively.

$$Hausner ratio = \frac{bulk density}{tapped density} \qquad ( Eqn . 1 )$$

$$Compressibility index = \frac{tapped density - bulk density}{tapped density} \times 100 \qquad ( Eqn . 2 )$$

### Hardness

Tablet hardness was determined with a Varian VK200 Tablet Hardness Tester in accordance with USP <1217>.

### Disintegration

Disintegration testing was performed using a Sotax DT50 and distilled water in accordance with USP <701>.

### Friability

A Sotax FT2 friability test apparatus was used for friability testing by tumbling not-less-than 6.5 g of tablets for 4 minutes at 25 rpm for a total of 100 revolutions.

### Focused Beam Reflectance Measurement

A Focused Beam Reflectance Measurement (FBRM) probe (Model Lasentec® S400, Mettler Toledo, Columbia, MD) was used to measure particle size distribution. An overhead mixer on a fixed-beaker stand (Lasentec® S400 Mixer Controller) was used to keep particles in suspension during testing. The acquisition and analysis software was iC-FBRM™ version 4.3 (Mettler Toledo, Columbia, MD).

### Compression Profile

Compression profiles were generated on tablets prepared using a Globe Pharma manual tablet compaction machine model MTCM-1 with a 5/16-inch flat upper and lower punch and die.

### X-Ray Powder Diffraction

X-ray powder diffraction (XRPD) was performed on micronized and blended material using a Rigaku-Miniflex 6G benchtop x-ray diffractometer. Approximately 100 mg of sample was pressed onto a non-diffracting silicon substrate using a circular siliconized glass slide. The x-ray source was operated at 40 kV and 15 mA. Sample data was collected using a run speed of 0.4 degree/minute with a 0.02° step size from 3-40°. Sample spinning was turned on for all measurements.

### Scanning Electron Microscopy

Scanning electron microscopy (SEM) images were captured using a Phenom Pro in 10 kV high resolution mode. Carbon paper was used as the conductive substrate. A plastic spatula was rolled over a <100 mg sample then carefully rolled over double-sided adhesive carbon paper on top of the stage to prepare the sample. Three left-handed rotations of the sample holder were used to position the sample approximately 2 mm below the top of the stage for imaging.

### Preparation of Elra:CAP ASD and Elra:CAP Tablets for Example 2B

### Preparation of the Elra:CAP ASD:

Elraglusib and CAP were weighed and dispensed into a 1.0 L or 2.0 L graduated media storage bottle. The materials were dissolved in THF to produce about 8% weight by weight (w/w) total solids spray solution. The solution was stirred for 30 minutes prior to spraying to ensure full dissolution of the solids. The B-290 mini spray dryer was set up per the parameters summarized in Table 2B-6 and in Table 2B-7. After reaching the target oxygen percentage of not more than (NMT) 6% (actual value: lot 35044-004-4.6%, lot 35044-030-2.0%), the heater was turned on and the inlet temperature set to 100° C. Once the inlet temperature was reached, the outlet temperature was allowed to stabilize. Pure solvent was run until a steady state was reached. Upon reaching steady state, spraying of the elraglusib/CAP mixture in THF was initiated using a 30% pump flow rate and size 14 L/S tubing. After spraying as much of the product as possible and allowing the apparatus to cool, the product was collected and oven dried at 60° C. overnight.

TABLE 2B-6
Process parameters for the use of B-290 mini spray dryer

| | |
|---|---|
| Lot 35044-004 | Lot 35044-030 |

| | | |
|---|---|---|
| Loop configuration | Closed loop | Closed loop |

| | | | | |
|---|---|---|---|---|
| Cyclone volume | 1.1 | L | 1.1 | L |

| | | |
|---|---|---|
| B-295 loop system | Inert loop | Inert loop |
| Aspirator rate | 100% | 100% |

| | | | | |
|---|---|---|---|---|
| Nitrogen flow rate | 30 | mm | 30 | mm |
| Cooling temperature | −10° | C. | −10° | C. |

TABLE 2B-7
ASD processing target parameters for Elra:CAP

| Process Parameter | Target | Range |
|---|---|---|
| Inlet temperature (° C.) | 100 | 95-100 |
| Aspirator (%) | 97 | 97-100 |
| Pump rate (%) | 30 | 30-40 |
| Spray gas flowmeter (mm) | 30 | 30 |
| US nozzle temperature (° C.) | NA | NA |
| US nozzle power (W) | NA | NA |
| Nozzle cleans | 1 | 1 |

The spray-drying of Elra:CAP in tetrahydrofuran was successfully completed with a summary of the spray-drying presented in Table 2B-8 and Table 2B-9.

TABLE 2B-8
Elra:CAP Batch 35044-004Spray-drying parameter summary

| | Mass Lost | Inlet | Outlet | | Nitrogen | Spray |
|---|---|---|---|---|---|---|
| Time | from Spray | Temperature | Temperature | Pump | Flow | Rate |
| (mm:ss) | Solution (g) | (° C.) | (° C.) | Rate (%) | (g/min) | (g/min) |
| 00:00 | 0.00 | 97 | 74 | 30 | 30 | NA |
| 10:00 | 101.93 | 100 | 69 | 30 | 30 | 10.2 |
| 20:00 | 232.07 | 92 | 64 | 40 | 30 | 13.0 |
| 31:00 | 380.40 | 95 | 64 | 40 | 30 | 13.5 |
| 40:00 | 503.89 | 95 | 64 | 40 | 30 | 13.7 |
| 44:18 | 560.47 | 95 | 65 | 40 | 30 | 13.2 |

TABLE 2B-9
Elra:CAP Batch 35044-030 Spray-drying parameter summary

| | Mass Lost | Inlet | Outlet | | Nitrogen | Spray |
|---|---|---|---|---|---|---|
| Time | from Spray | Temperature | Temperature | Pump | Flow | Rate |
| (mm:ss) | Solution (g) | (° C.) | (° C.) | Rate (%) | (g/min) | (g/min) |
| 00:00 | 0.00 | 99 | 70 | 35 | 30 | NA |
| 10:00 | 55.28 | 100 | 70 | 35 | 30 | 5.5 |
| 20:00 | 113.17 | 100 | 69 | 40 | 30 | 5.8 |
| 30:00 | 186.99 | 100 | 70 | 45 | 35 | 7.4 |
| 40:00 | 247.19 | 100 | 70 | 45 | 35 | 6.0 |
| 50:00 | 318.21 | 100 | 70 | 45 | 35 | 7.1 |
| 62:00 | 394.73 | 100 | 70 | 45 | 35 | 6.4 |
| 70:00 | 466.78 | 100 | 70 | 45 | 35 | 9.0 |
| 80:00 | 530.60 | 100 | 69 | 45 | 35 | 6.4 |
| 93:00 | 621.95 | 100 | 68 | 50 | 35 | 7.0 |
| 100:00 | 681.66 | 100 | 67 | 50 | 35 | 8.5 |
| 110:00 | 751.40 | 100 | 67 | 50 | 35 | 7.0 |
| 130:00 | 906.40 | 100 | 67 | 50 | 35 | 7.8 |
| 150:00 | 1051.00 | 100 | 66 | 50 | 35 | 7.2 |
| 170:00 | 1203.30 | 100 | 66 | 50 | 35 | 7.6 |
| 190:00 | NA^{a} | 100 | 66 | 50 | 35 | NA^{a} |

^{a}Bottle tilted at the end of the run. Weight and spray rate unknown.

### Elra:ASD Characterization:

Elra:CAP batch 35044-004 was characterized by XRPD (FIG. 31) showing a monomodal distribution characteristic of an amorphous material. SEM imaging was used to examine the morphology of the ASD as shown in FIG. 32. The images showed the typical mixed morphology of hollow spheres, collapsed spheres, and shattered spheres for spray-dried material.

ASD manufacturing of 50:50 mixture of Elra:CAP batch 35044-004 was completed with an overall yield of 78%. XRPD confirmed the amorphous nature of the final product and SEM imaging showed mixed morphology typical of an ASD process.

### Elra:CAP ASD Granulation Batch 35044-014:

Prior to granulation, the Elra:CAP ASD was passed through a #20 mesh screen. All intragranular ingredients except magnesium stearate were then added to a 250 mL high density polyethylene (HDPE) bottle and blended for 50 revolutions. Magnesium stearate was added to the mixture and ingredients were blended for an additional 50 revolutions.

The preblend was roller compacted using the Gerteis compactor with parameters summarized in Table 2B-10. The blend was collected in a 500 mL HDPE bottle and weighed to calculate the adjustment factor for extragranular excipients. Extragranular excipients were added and the mixture was blended to produce the final blend.

TABLE 2B-10
Gerteis granulation parameters for
Elra:CAP ASD Batch 35044-014

| Process Parameter | 35044-014 |
|---|---|
| Roll type | Smooth die left, knurled |
| | punch right |
| Granulator type | Star |
| Gap control | On |
| Granulator angle clockwise (°) | 360 |
| Granulator angle counterclockwise | 330 |
| (°) | |
| Tamp to feed auger ratio (%) | 200 |
| Feed factor | 0.30 |
| Agitator speed | 3 |
| Roll speed (rpm) | 1 |
| Roll force (kN/cm) | 5 |
| Roll gap (mm) | 3.0 |
| Granulator speed clockwise (rpm) | 65 |
| Granulator speed counterclockwise | 65 |
| (rpm) | |
| Granulator screen (mm) | 1.25 |

### Elra:CAP ASD Granulation Batch 35044-019:

Compacts were prepared using approximately 1 g of preblend using the Manual Tablet Compression Machine (MTCM-1, Globe Pharma) with Gerteis compaction simulation (12.5 kN target force, which is equal to approximately 3200 psi on MTCM-1). The compacts were milled through a 1.25 mm screen to produce a free-flowing granulated blend. Extragranular excipients were added, and the mixture was blended for 50 revolutions to produce the final blend.

### Elra:CAP ASD Tableting:

Tablets were pressed using the MTCM-1 or Korsch XP-1.

### Non-Micronized API Dry Granulation—Feasibility:

Prior to granulation, the elraglusib non-micronized API was passed through a #20 mesh screen. All intragranular ingredients except magnesium stearate were then added to a 250 mL HDPE bottle and blended for 50 revolutions. Magnesium stearate was added to the mixture and ingredients were blended for an additional 50 revolutions. Compacts were prepared using approximately 1 g of preblend using the MTCM-1 with Gerteis compaction simulation (12.5 kN target force, which is equal to approximately 3200 psi on MTCM-1). The compacts were milled through a 1.25 mm screen to produce a free-flowing granulated blend. Extragranular excipients were added, and the mixture was blended for to produce the final blend.

### Non-Micronized API Dry Granulation—Prototype:

Prior to granulation the elraglusib non-micronized API was passed through a #20 mesh screen. All intragranular ingredients except magnesium stearate were then added to a 500 mL HDPE bottle and blended for 10 minutes at 25 revolutions per minute (rpm). Magnesium stearate was added to the mixture and ingredients were blended for an additional 5 minutes at 25 rpm. The preblend was roller compacted using the Gerteis compactor with parameters summarized in Table 2B-11. The blend was collected in a 500 mL HDPE bottle and weighed to calculate the adjustment factor for extragranular excipients. Extragranular microcrystalline cellulose (MCC) and croscarmellose sodium (CSS) were added, and the mixture was blended 5 minutes at 25 rpm. Magnesium stearate was sieved through a #20 mesh, added to the mixture, and blended for 5 minutes at 25 rpm to produce the final blend.

TABLE 2B-11
Gerteis parameters for non-micronized API Dry Granulation

| Process Parameter | |
|---|---|
| Roll type | Smooth die left, |
| | knurled punch right |
| Granulator type | Star |
| Gap control | On |
| Granulator angle clockwise (°) | 360 |
| Granulator angle counterclockwise | 330 |
| (°) | |
| Tamp to feed auger ratio (%) | 200 |
| Feed factor | 0.30 |
| Agitator speed | 3 |
| Roll speed (rpm) | 2 |
| Roll force (kN/cm) | 5 |
| Roll gap (mm) | 3.0 |
| Granulator speed clockwise (rpm) | 65 |
| Granulator speed counterclockwise | 65 |
| (rpm) | |
| Granulator screen (mm) | 1.25 |

### Non-Micronized API Wet Granulation—Feasibility:

Prior to granulation the elraglusib non-micronized API was passed through a #20 mesh screen. Intragranular ingredients were added and blended in a mortar for 1 minute. Water for Injection was added to 30% w/w at approximately 2.5 g/minute. Wet massing time of 1 minute was allowed for the blend prior to transfer of the blend to the oven at 60° C. for 6 hours. At the end of this time loss on drying was measured and a 1.00 mm mesh was used for granulation of the dried blend. The blend was weighed to calculate the adjustment factor for extragranular excipients. Extragranular MCC and CSS were added to the HPDE container, and the mixture was blended for 50 revolutions. Magnesium stearate was added to the mixture and blended for an additional 50 revolutions to produce the final blend.

### Non-Micronized API Wet Granulation—Prototype:

Intragranular ingredients were added to the high shear wet granulator adding half of the MCC prior to the non-micronized API and excipients and the second half of the MCC on top of the 1.0 L bowl. The ingredients were homogenized by dry mixing in the GlobePharma High Shear Granulator1-6 for 3 minutes with an impeller speed of 300 rpm and chopper speed of 1000 rpm. Wet granulation was initiated using an impeller speed of 300 rpm and chopper speed of 1000 rpm with a spray rate of approximately 10 mL/minute. After water was added to the target 30% w/w, the spray was turned off and 90 seconds of wet massing was performed to facilitate the distribution of water in the solids. Wet granules were then manually milled through a #6 screen (3.35 mm). The SolidLab1 equipped with a 0.5 L bowl was used for fluidized bed drying of the granules. The inlet air volume was set to 15 cubic feet per minute (CFM) at a temperature of 60.0° C. Once the product temperature reached 45° C. the fluid bed dryer was charged with the wet milled granules. After a 20-minute drying time, the observed loss on drying at 105° C. was less than 2.0% (lot 35044-027-0.49%). The dried granules were discharged and manually milled through a 1.00 mm screen. The granulated blend was transferred to a 500 mL polypropylene bottle and weighed to calculate the adjustment factor for extragranular excipients. Extragranular MCC and CSS were added, and the mixture was blended 5 minutes at 25 rpm. Magnesium stearate was sieved through a #30 mesh (0.595 mm), added to the mixture, and blended for 3 minutes at 25 rpm to produce the final blend.

### Elra:CAP ASD Tableting:

Tablets were pressed using the MTCM-1 or Korsch XP-1.

The spray-drying of Elra:CAP in tetrahydrofuran was successfully completed with a summary of the spray-drying presented in Table 2B-12 and Table 2B-13.

TABLE 2B-12
Elra:CAP Spray-drying parameter summary batch 35044-004

| | Mass Lost | Inlet | Outlet | | Nitrogen | Spray |
|---|---|---|---|---|---|---|
| Time | from Spray | Temperature | Temperature | Pump | Flow | Rate |
| (mm:ss) | Solution (g) | (° C.) | (° C.) | Rate (%) | (g/min) | (g/min) |
| 00:00 | 0.00 | 97 | 74 | 30 | 30 | NA |
| 10:00 | 101.93 | 100 | 69 | 30 | 30 | 10.2 |
| 20:00 | 232.07 | 92 | 64 | 40 | 30 | 13.0 |
| 31:00 | 380.40 | 95 | 64 | 40 | 30 | 13.5 |
| 40:00 | 503.89 | 95 | 64 | 40 | 30 | 13.7 |
| 44:18 | 560.47 | 95 | 65 | 40 | 30 | 13.2 |

TABLE 2B-13
Elra:CAP Spray-drying parameter summary batch 35044-030

| | Mass Lost | Inlet | Outlet | | Nitrogen | Spray |
|---|---|---|---|---|---|---|
| Time | from Spray | Temperature | Temperature | Pump | Flow | Rate |
| (mm:ss) | Solution (g) | (° C.) | (° C.) | Rate (%) | (g/min) | (g/min) |
| 00:00 | 0.00 | 99 | 70 | 35 | 30 | NA |
| 10:00 | 55.28 | 100 | 70 | 35 | 30 | 5.5 |
| 20:00 | 113.17 | 100 | 69 | 40 | 30 | 5.8 |
| 30:00 | 186.99 | 100 | 70 | 45 | 35 | 7.4 |
| 40:00 | 247.19 | 100 | 70 | 45 | 35 | 6.0 |
| 50:00 | 318.21 | 100 | 70 | 45 | 35 | 7.1 |
| 62:00 | 394.73 | 100 | 70 | 45 | 35 | 6.4 |
| 70:00 | 466.78 | 100 | 70 | 45 | 35 | 9.0 |
| 80:00 | 530.60 | 100 | 69 | 45 | 35 | 6.4 |
| 93:00 | 621.95 | 100 | 68 | 50 | 35 | 7.0 |
| 100:00 | 681.66 | 100 | 67 | 50 | 35 | 8.5 |
| 110:00 | 751.40 | 100 | 67 | 50 | 35 | 7.0 |
| 130:00 | 906.40 | 100 | 67 | 50 | 35 | 7.8 |
| 150:00 | 1051.00 | 100 | 66 | 50 | 35 | 7.2 |
| 170:00 | 1203.30 | 100 | 66 | 50 | 35 | 7.6 |
| 190:00 | NA^{a} | 100 | 66 | 50 | 35 | NA^{a} |

^{a}Bottle tilted at the end of the run. Weight and spray rate unknown.

### Elra:ASD Characterization:

Elra:CAP from batch 35044-004 was characterized by XRPD (FIG. 31) showing a monomodal distribution characteristic of an amorphous material. SEM imaging was used to examine the morphology of the ASD as shown in FIG. 32. The images showed the typical mixed morphology of hollow spheres, collapsed spheres, and shattered spheres for spray-dried material.

ASD manufacturing of 50:50 mixture of Elra:CAP was completed with an overall yield of 78% (batch 35044-004). XRPD confirmed the amorphous nature of the final product and SEM imaging showed mixed morphology typical of an ASD process.

### Micronized Elraglusib API:

Elraglusib API was micronized using a jet mill and evaluated for compressibility.

### Characterization of Micronized Elraglusib API:

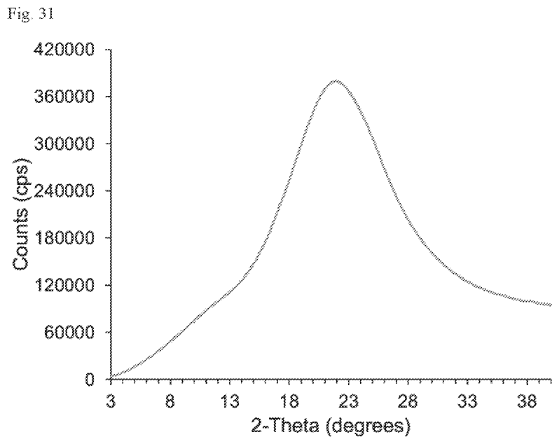

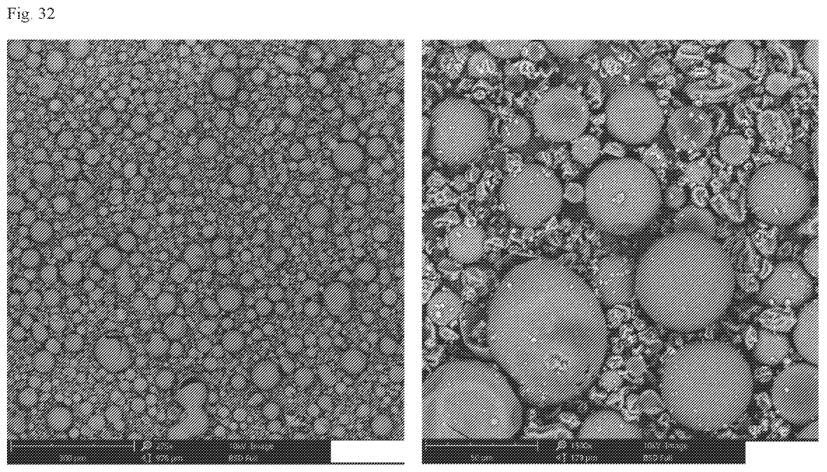

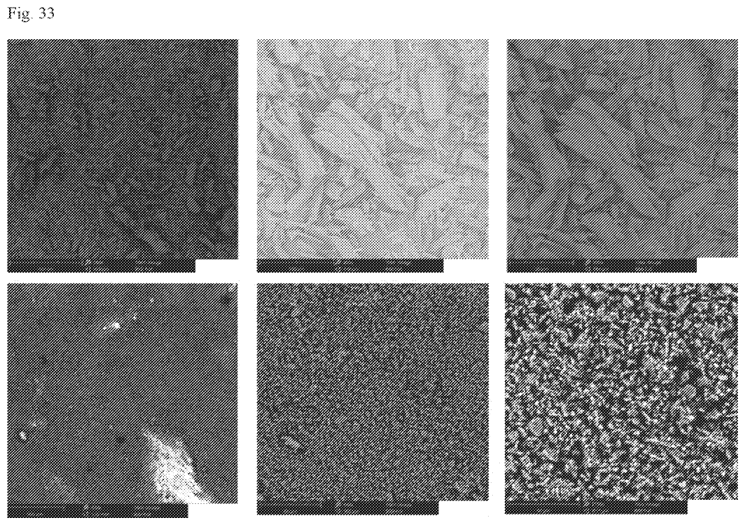

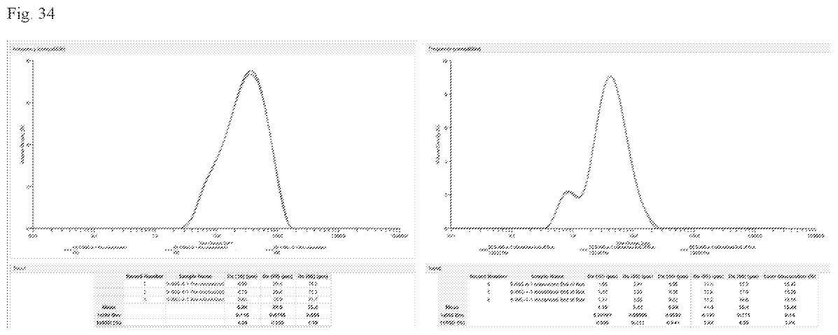

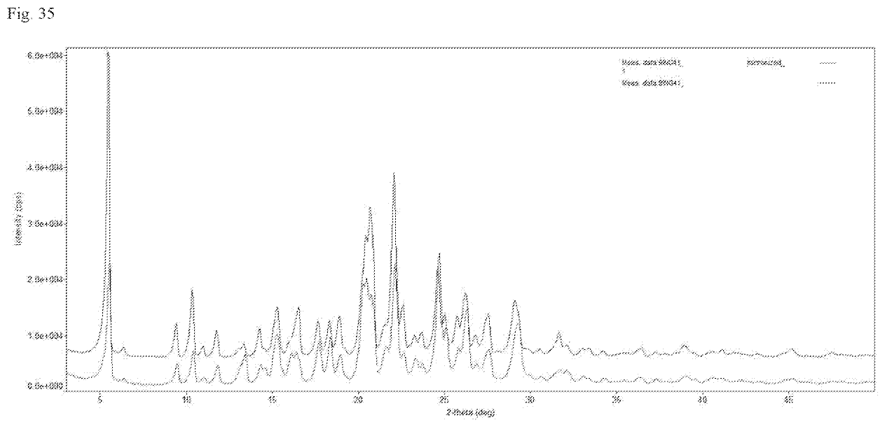

SEM images (FIG. 33), particle size distribution (PSD, FIG. 34) and XRPD (FIG. 35) were performed on elraglusib material before and after jet milling for evaluation of the micronization process. SEM, PSD, and XRPD data before and after milling were as expected. A bimodal particle size distribution was observed after milling which may be due to a single pass in the mill.

API micronization was successfully performed to produce material with improved compressibility over non-milled material. Particle size analysis displayed a bimodal distribution for the micronized material which could be evaluated and optimized further to produce a monomodal distribution.

### Micronized API Dry Granulation Formulation Development

Formulation development for the micronized API dry granulation process was designed to produce a tablet with the formulation compositions shown in Table 2B-14. Formulation 35044-008 was ultimately modified to utilize more extragranular microcrystalline cellulose (Formulation 35044-015).

TABLE 2B-14
Micronized API Dry granulation formulation composition

| | | | |
|---|---|---|---|
| | | 35044-008 | 35044-015 |
| Ingredient | Grade | (% w/w) | (% w/w) |

Intragranular

| | | | |
|---|---|---|---|
| elraglusib | Micronized | 42.70 | 42.70 |
| MCC | PH 102 | 26.05 | 23.05 |
| Mannitol | Pearlitol 100SD | 23.00 | 19.00 |
| CSS | Ac-Di-Sol | 3.50 | 3.50 |
| Sodium lauryl sulfate | Kolliphor SLS | NA | 2.00 |
| Magnesium stearate | Ligamed MF-2-K | 0.75 | 0.75 |

Extragranular

| | | | |
|---|---|---|---|
| MCC | PH 102 | NA | 5.00 |
| CSS | Ac-Di-Sol | 3.50 | 3.50 |
| Magnesium stearate | Ligamed MF-2-K | 0.50 | 0.50 |

| | | |
|---|---|---|
| Total | 100.00 | 100.00 |

### Micronized API Wet Granulation Formulation Development

Formulation development for the micronized API wet granulation was designed to produce a tablet with the formulation compositions shown in Table 2B-15.

TABLE 2B-15
Micronized API Wet granulation formulation compositions

| | | | |
|---|---|---|---|
| | | 35044-009 | 35044-027 |
| Ingredient | Grade | (% w/w) | (% w/w) |

Intragranular

| | | | |
|---|---|---|---|
| elraglusib | Micronized | 42.7 | 42.7 |
| MCC | PH 102 | 21.3 | 26.3 |
| Mannitol | Pearlitol 100SD | 19.0 | 13.0 |
| CSS | Ac-Di-Sol | 3.5 | 4.0 |
| Sodium lauryl sulfate | Kolliphor SLS | 2.0 | 2.0 |
| Hydroxypropyl | Klucel EXF | 2.0 | 2.0 |
| cellulose | | | |

Extragranular

| | | | |
|---|---|---|---|
| MCC | PH 102 | 5.0 | 5.0 |
| CSS | Ac-Di-Sol | 3.5 | 4.0 |
| Magnesium stearate | Ligamed MF-2-K | 1.0 | 1.0 |

| | | |
|---|---|---|
| Total | 100.0 | 100.0 |

The revised micronized API wet granulation formulation (35044-027) was processed using the GlobePharma high shear wet granulator with granulation data presented in Table 2B-16. The wet granules were manually milled through a #6 (3.35 mm) screen before charging material into the 0.5 L fluid processing bowl. Fluid-bed drying data is presented in Table 2B-17.

TABLE 2B-16
Micronized API Wet granulation processing data batch 35044-027

| | | | | | |
|---|---|---|---|---|---|
| | Weight of | Spray | Impeller | Chopper | |
| Time | Water | Rate | Speed (rpm) | Speed (rpm) | Power |
| (mm:ss) | (g) | (g/min) | Target: 300 | Target: 1000 | (amp) |

Dry Blending

| | | | | | |
|---|---|---|---|---|---|
| 00:00 | NA | NA | 300 | 1007 | 1.14 |
| 03:00 | NA | NA | 300 | 1007 | 1.15 |

Wet Granulation

| | | | | | |
|---|---|---|---|---|---|
| 00:00 | NA | NA | 300 | 1007 | 1.15 |
| 01:00 | 10.98 | 10.98 | 300 | 1007 | 1.15 |
| 02:00 | 21.09 | 10.11 | 300 | 1007 | 1.14 |
| 03:00 | 30.16 | 9.07 | 300 | 1007 | 1.14 |
| 04:00 | 39.49 | 9.33 | 300 | 1007 | 1.14 |
| 05:00 | 48.50 | 9.01 | 300 | 1007 | 1.14 |
| 05:36 | 54.18 | 9.47 | 300 | 1007 | 1.14 |

Wet Massing

| | | | | | |
|---|---|---|---|---|---|
| 00:00 | NA | NA | 301 | 1012 | 1.14 |
| 01:30 | NA | NA | 300 | 1007 | 1.14 |

TABLE 2B-17
Micronized API Fluid-bed drying data batch 35044-027

| | | | | | Pulse | |
|---|---|---|---|---|---|---|
| | | | | | Setting | Loss |
| | Inlet Air | Inlet Air | Product | Outlet | for Filter | on |
| Time | Volume | Temperature | Temperature | Temperature | Bag | Drying |
| (min) | (CMH) | (° C.) | (° C.) | (° C.) | (psi) | (%) |
| 0 | 18 | 59.3 | 28.9 | 29.1 | 30 psi | NA |
| 5 | 15 | 59.9 | 30.0 | 28.0 | every | NA |
| 10 | 15 | 60.2 | 37.2 | 29.0 | 3 sec | NA |
| 15 | 15 | 60.1 | 42.7 | 33.0 | | NA |
| 20 | 15 | 60.1 | 45.9 | 35.4 | | 0.49 |

Wet granulation of micronized elraglusib API was successfully completed with high shear wet granulation. Minimal increase in power was used during the granulation indicating that additional water could be used to granulate.

### Dissolution

Dissolution in 6% w/w CTAB in 0.7 M sodium chloride was assessed for wet and dry granulation and ASD materials at paddle speeds of 75 rpm and 100 rpm. FIG. 36.

The same conditions of 6% w/w CTAB in 0.7 M sodium chloride and paddle speed of 100 rpm were used to evaluate the impact of compaction pressure on the observed dissolution profile of the dry granulated material and wet granulated material.

The dissolution medium was revised to 1% w/w CTAB in 0.7 M sodium chloride for testing of the ASD material. The dissolution profile of the ASD tablets was improved with 1% w/w CTAB in 0.7 M sodium chloride compared to the 6% w/w CTAB in 0.7 M sodium chloride as the dip in dissolution between 45 minutes and 60 minutes was not observed. It is hypothesized that the increased concentration of CTAB may be disrupting the ASD polymer and releasing API out of the complex.

### Example 3. Bioavailability Studies

PK Study 1—Bioavailability of IV Formulation when Administered Intravenously Vs. Orally in Dogs

The objective of this study was to evaluate the pharmacokinetic profile of elraglusib following single oral and intravenous doses in Beagle dogs.

The test article (elraglusib in a vehicle of 85% PEG400, 10% ethanol, 5% water (all components are weight/weight/weight)) was administered orally and via intravenous infusion to dogs according to the tables below.

Male Beagle dogs were selected for use in this study. Animals were at least 6 months old and males weighed approximately 9.3-10.8 kilograms at the start of dosing.

The study animals received a single oral dose of elraglusib followed by an approximate 7-day washout period after which they received a single intravenous infusion of elraglusib. Animals underwent standard evaluations such as body weight and clinical observations, and were bled for pharmacokinetics following each dose. The study design is summarized in the following Table 3-1:

TABLE 3-1
Dog Bioavailability Study 1 Experimental Design

| Study | | Target | Dose | Dose | |
|---|---|---|---|---|---|
| Group/ | Compound | Dose | Conc. | Volume | Number of |
| Phase | Name | (mg/kg) | (mg/mL) | (mL/kg) | Dogs/Group |
| Oral | elraglusib | 20 | 10 | 2 | 3 |
| IV | | 20 | 10 | 2 | |

Notes:
After oral administration of elraglusib, dogs were re-used for intravenous infusion (approximately 15 minutes) of elraglusib after an approximate 7-day washout period.

Plasma concentrations of elraglusib were determined for dogs treated with 20 mg/kg elraglusib via oral administration on Day 1 and then again via intravenous infusion on Day 8. Systemic exposure was achieved in all animals following both oral and intravenous administration of elraglusib. Intravenous administration resulted in higher elraglusib plasma concentrations, compared to oral administration. Individual plasma concentrations were highest approximately 2-8 hours after oral administration (1,410-1,880 ng/mL), and highest at approximately 5 minutes following intravenous administration (11,800-13,600 ng/mL).

Overall, the half-life was similar by both the oral and IV routes of administration. Group mean Cₘₐₓ was approximately 7.5 fold greater for the IV versus the PO route and AUCₗₐₛₜ was approximately 3 fold greater for the IV versus the PO route. Comparing the plasma AUC values for dosing by oral gavage (21,300 hr*ng/mL) versus IV injection (68,900 hr*ng/mL), permits estimating oral percentage bioavailability (% BA) as 100*(21,300/68,900)=31% BA. Summary PK parameters by route are shown below:

TABLE 3-2
Summary of Individual and Group Mean PK Parameters
(Oral Route) for Test Article elraglusib on Day 1

| Animal | | Dose | t_{1/2b} | Tₘₐₓ | Cₘₐₓ^{b} | AUCₗₐₛₜ^{b} | AUC_{INF}^{b} |
|---|---|---|---|---|---|---|---|
| ID | Sex | (mg/kg) | (hr) | (hr) | (ng/mL) | (hr*ng/mL) | (hr*ng/mL) |
| 1 | M | 20 | 8.12 | 4 | 1,880 | 22,200 | 26,100 |
| 2 | M | 20 | a | 8 | 1,410 | 22,900 | a |
| 3 | M | 20 | a3.85 | 2 | 1,720 | 18,800 | 19,000 |
| Mean ± | M | 20 | 5.98 ± | 4.7 | 1,670 ± | 21,300 ± | 22,600 ± |
| SD | | | 3.02 | 3.1 | 239 | 2,220 | 4,500 |

aThere was insufficient data in the terminal elimination phase of elraglusib to calculate the parameter.
^{b}Values are reported to 3 significant figures.

TABLE 3-3
Summary of Individual and Group Mean PK Parameters
(IV Route) for Test Article elraglusib on Day 8

| Animal | | Dose | t_{1/2a} | Tₘₐₓ | Cₘₐₓ^{a} | AUCₗₐₛₜ^{a} | AUC_{INF}^{a} |
|---|---|---|---|---|---|---|---|
| ID | Sex | (mg/kg) | (hr) | (hr) | (ng/mL) | (hr*ng/mL) | (hr*ng/mL) |
| 1 | M | 20 | 7.26 | 0.083 | 12,900 | 61,200 | 66,800 |
| 2 | M | 20 | 6.73 | 0.083 | 13,600 | 69,200 | 74,400 |
| 3 | M | 20 | 4.17 | 0.083 | 11,800 | 76,300 | 77,200 |
| Mean ± | M | 20 | 6.05 ± | 0.083 ± | 12,800 ± | 68,900 ± | 72,800 ± |
| SD | | | 1.65 | 0 | 907 | 7,560 | 5,390 |

^{a}Values are reported to 3 significant figures.

### PK Study 2—Bioavailability of Oral Solution, Oral Suspension, and Elra:CAP and Elra:PVAP Capsule Dosage Forms in Dogs

A five-way cross-over study was conducted in which three animals were first dosed at 10 mg/kg by IV injection using 10 mg/mL elraglusib in PEG400:EtOH:Water (85:10:5% w/w).

Three male non-naïve and/or naïve beagle dogs are selected for use in this study. The animals are 7-12 months old and weigh 8-12 kilograms at the start of the quarantine period for this study. The actual body weights and ages may vary but will be recorded and maintained with the raw data.

Approximately 250-350 grams of Harlan Teklad Certified Canine Diet #2025 (or other suitable diet as documented in the study record) was provided to each animal on a daily basis. Each lot of diet is analyzed for contaminants to ensure that none are present at concentrations which would be expected to interfere with the conduct or purpose of this study. In addition, each dog receive d a commercially available canned dog food to ensure animals were in a fed-state prior to dosing. The canned dog food ½-1 can was provided at a minimum of 1 hour prior to each administration. The lot and brand of the dog food was documented in the study record.

The study was composed of a single group of 3 dogs each receiving 5 administrations of elraglusib dosage forms, each administration separated by a minimum of a 3-day treatment free period (wash-out). The first administration was a single intravenous (IV) infusion dose of elraglusib TA #1 (10 mg/mL in PEG400:EtOH:water, 85:10:5% w/w) followed by an oral dose of TA #2 (Elra:CAP capsules, 100 mg elraglusib), followed by an oral dose of TA #3 (Elra:PVAP capsules, 100 mg elraglusib), followed by an oral gavage dose of TA #4 (a 2 mg/mL oral suspension formulation), followed by an oral gavage dose of TA #5 (a 50 mg/mL oral solution). Animals underwent standard evaluations such as body weight and clinical observations, and were bled for pharmacokinetics preceding and following each dose. The Study 2 experimental design is summarized in Table 3-4.

TABLE 3-4

| | | Target | | | | | |
|---|---|---|---|---|---|---|---|
| | | Dose | Dose | | Dose Amount | | # of Dogs/ |
| Route | TA # | (mg/kg) | Strength | | Per Dog | | Administration |
| IV | 1 | 10 | 10 | mg/mL^{a} | 1 | mL/kg | 3 |
| Oral Elra:CAP | 2 | 20 | ~100 | mg/capsule | 2 | capsules | |
| Capsule | | | | | | | |
| Oral Elra:PVAP | 3 | 20 | ~100 | mg/capsule | 2 | capsules | |
| Capsule | | | | | | | |
| Oral | 4 | 10 | 2 | mg/mL^{b} | 5 | mL/kg | |
| Suspension, 2 | | | | | | | |
| mg/mL | | | | | | | |
| Oral Solution, | 5 | 20 | 50 | mg/mL | 0.4 | mL/kg | |
| 50 mg/mL in | | | | | | | |
| PEG400:EtOH:Tween80 | | | | | | | |
| (94:5:1% w/w) | | | | | | | |

^{a}9-ING-41 API was diluted by into Vehicle (PEG400:EtOH:water 85:10:5% w/w) and administered
^{b}TA #5 was diluted 25-fold into D5W and administered by gavage.
Notes:
After bolus IV administration of elraglusib, dogs were re-used for oral gavage and capsule administration of elraglusib after a minimum of 3 day washout period between each dose.

Dogs received a single 15-minute intravenous infusion of the test article into the cephalic vein (or other suitable vessel). The test article (TA #1) was infused into the cephalic vein (or other suitable vessel). After IV administration dogs received 4 additional oral (gavage or capsule) administrations of the test articles (TA #2, 3, 4, and 5) in the order of the Test Article #. For IV and oral gavage administration the doses were based on the most recent body weight. For capsule administration the pre-filled capsules were weighed prior to dosing, and the was back calculated based on the dogs weight.

Blood samples were collected from the jugular vein (or other suitable vessel) into 2 mL vacutainer tubes containing K2EDTA as anticoagulant from each dog at the following timepoints:
a) Intravenous administration: Prior to (0) and at 5, 15, 30, 60 minutes, as well as at 2, 4, 8, and 24 hours following the end of the injection. The zero hour (trough) was collected within approximately 24 hours prior to dosing.
b) Oral administration: Prior to (0) and at 15, 30, 60 minutes, as well as at 2, 4, 8, and 24 hours following dose administration. The zero hour (trough) was collected within approximately 24 hours prior to dosing.

These animals were maintained on a specified feeding regimen, and were also fed shortly before dosing to ensure that animals remained in a fed state during dose administration. After a washout period, the same three were dosed at either 20 mg/kg or at 10 mg/kg with each of four oral dosing formulations of the present disclosure:
1) 20 mg/kg dose using size 00 hard gelatin capsules with 100 mg Elra:CAP ASD;
2) 20 mg/kg dose using size 00 hard gelatin capsules with 100 mg Elra:PVAP ASD;
3) 20 mg/kg dose using elraglusib 50 mg/mL Oral Solution in Vehicle Variant 7 (PEG400:EtOH:Tween80 94:5:1% w/w);
4) 10 mg/kg using elraglusib 50 mg/mL in Vehicle Variant 7 after dilution to a 2-mg/mL suspension in 5% Dextrose Injection (D5W).

Blood samples were drawn at timed intervals plasma [elraglusib] determinations were made using a validated LC-MS/MS method.

TABLE 3-5
Mean Plasma Concentration Results of Dog PK Study 2

| | | | | Dose, | | Cₘₐₓ, | | |
|---|---|---|---|---|---|---|---|---|
| Description | Route | Strength | | mg/kg | Tₘₐₓ, h | ng/mL | AUC, ng-h/mL | % BA |
| Canine Vehicle | IV | 10 | mg/mL | 10 | 0.083 | 7,347 | 32,299 | 100* |
| Elra:CAP | Oral | 100 | mg/ | 20 | 8 | 4,223 | 62,611 | 97^{†} |
| Capsule | | | capsule | | | | | |
| Elra:PVAP | Oral | 100 | mg/ | 20 | 8 | 3,957 | 43,904 | 68^{†} |
| Capsule | | | capsule | | | | | |
| Oral Solution | Oral | 50 | mg/mL | 20 | 2 | 5,170 | 51,653 | 80^{†} |
| Oral Suspension | Oral | 2 | mg/mL | 10 | 4 | 1,802 | 11,161 | 35* |

*% BA = 100*(AUCₒᵣₐₗ ÷ AUC_{IV})
^{†}% BA = 100*(0.5*AUCₒᵣₐₗ ÷ AUC_{IV})

These results indicated high oral percentage bioavailability for the Elra:CAP solid oral dosage form (% BA=97) and for the 50 mg/ml Oral Solution in Vehicle Variant 7 (% BA=80). Compared with the two solid oral dosage forms, the 50 mg/mL Oral Solution in Vehicle Variant 7 demonstrated significantly shorter Tmax values (Tmax=8 hr for the solid dosage forms versus Tmax=2 hr for the oral solution).

### PK Study 3.—Bioavailability of Oral Solution and Tablet Dosage Forms in Dogs

Three male non-naïve beagle dogs were selected from an in-house pool of colony animals for use in this study. The animals were approximately 11-16 months old and weighed 10-12 kilograms at the start of the quarantine period for this study.

Approximately 250-350 grams of Harlan Teklad Certified Canine Diet #2025 (or other suitable diet as documented in the study record) was provided to each animal on a daily basis. Each lot of diet was analyzed for contaminants to ensure that none were present at concentrations which would be expected to interfere with the conduct or purpose of this study. In addition, each dog received a commercially available canned dog food to ensure animals were in a fed-state prior to dosing. The canned dog food ½-1 can was provided at a minimum of 1 hour prior to each administration.

City water was provided ad libitum to all animals by an automatic watering system. Supply water is analyzed for contaminants as defined by the U.S. EPA “National Primary Drinking Water Regulations” (Title 40, Code of Federal Regulations, Part 141). No known contaminants are expected to be present in the water that would interfere with the interpretation of the study.

Each dog was identified with an ear tattoo; the assigned animal were provided. Identifying numbers assigned were unique within the animal room used.

Controls were set to maintain a temperature range of approximately 22±3° C. (approximately 66 to 77° F.) and a relative humidity range of approximately 30 to 70%. Lighting controls are set to maintain a 12-hour light/12-hour dark cycle and were also continuously monitored. Temperature and relative humidity was monitored continuously and reviewed at least once daily to ensure the environmental system was functioning properly.

Upon arrival and during the study, the dogs were group-housed (when possible) by sex in kennels or were single-housed in tandem cages equipped with companion doors to allow for socialization/exercise. Comingling was confined to animals with the same sex and dose group. Animals were individually housed during the dosing and observation phases. Each kennel or cage was equipped with an automatic watering system.

Animal cages/kennels and rooms were cleaned and sanitized prior to placing animals in them, and periodically thereafter in accordance with accepted animal care practices and relevant standard operating procedures.

The test Articles were as follows:

TA #1 was 10 mg/mL elraglusib in Vehicle weight to volume solutions, micro-filtered using a syringe filter (or other suitable filtration system) and dispensed into sterile vials for use. The IV solution was prepared based on elraglusib assay value. The IV solution was administered by infusion at 10 mg/mL followed by a sufficient wash-out period. The vehicle is 85% PEG-400, 10% ethanol, 5% water (all components are weight/weight/weight).

Tablets (TA #2): Each dog received one tablet of TA #2 orally followed by a sufficient wash-out period.

Tablets (TA #3): Each dog received one tablet of TA #3 orally followed by a sufficient wash-out period.

Tablets (TA #4): Each dog received one tablet of TA #4 orally followed by a sufficient wash-out period.

The 50 mg/mL Solution for Oral Suspension (TA #5) was dosed dogs orally at 0.4 mL/kg.

Table 3-6 below summarizes the experimental design of Dog PK Study 3.

TABLE 3-6

| Test Article Description | TA # | Description |
|---|---|---|
| elraglusib Injection | 1 | 10 mg/mL solution in Vehicle |
| | | (PEG400:EtOH:water 85:10:5% w/w) |
| | | administered by IV infusion |
| elraglusib Dry Granulation | 2 | ~200 mg/ tablet administered orally |
| (Micronized API) Tablets | | |
| elraglusib Wet Granulation | 3 | ~200 mg/ tablet administered orally |
| (Micronized API) Tablets | | |
| Elra:CAP ASD Dry | 4 | ~200 mg/ tablet administered orally |
| Granulation Tablets | | |
| elraglusib Solution for Oral | 5 | elraglusib 50 mg/mL |
| Suspension | | Solution in Vehicle Variant 7 |
| | | (PEG-400:EtOH:Tween 80 94:5:1% |
| | | w/w) administered orally |

The study was composed of a single group of 3 dogs. Each received 5 administrations of elraglusib dosage forms, each administration was separated by a minimum of a 3-day treatment free period (wash-out). The first administration was a single intravenous (IV) infusion dose of elraglusib (TA #1) followed by an oral dose of TA #2 tablets, followed by an oral dose of TA #3 tablets, followed by an oral dose of TA #4 tablets, followed by an oral gavage dose of a 50 mg/mL oral solution (TA #5). Animals underwent standard evaluations such as body weight and clinical observations and were bled for pharmacokinetics preceding and following each dose. The Dog PK Study 3 experimental design is summarized in Table 3-7.

TABLE 3-7

| | | Target | | | Dose | |
|---|---|---|---|---|---|---|
| | | Dose | Dose | | Amount Per | |
| Route | TA # | (mg/kg) | Strength | | Dog | |
| IV | 1 | 10 | 10 | mg/mL^{a} | 1 | mL/kg |
| Oral | 2 | 20 | ~200 | mg/tablet | 1 | tablet |
| Oral | 3 | 20 | ~200 | mg/tablet | 1 | tablet |
| Oral | 4 | 20 | ~200 | mg/tablet | 1 | tablet |
| Oral | 5 | 20 | 50 | mg/mL | 0.4 | mL/kg |
| Solution | | | | | | |

^{a}elraglusib API was diluted into Vehicle and administered.
Notes:
After IV administration of elraglusib, dogs were re-used for tablet and oral gavage administration of elraglusib after a minimum of 3-day washout period between each dose.

Pharmacokinetics (PK): Blood samples were collected from the jugular vein (or other suitable vessel) into 2 mL vacutainer tubes containing K2EDTA as anticoagulant from each dog at the following timepoints:

Intravenous administration: Prior to (0) and at 5, 15, 30, 60 minutes, as well as at 2, 4, 8, and 24 hours following the end of the injection. The zero hour (trough) was be collected within approximately 24 hours prior to dosing.

Oral administration: Prior to (0) and at 15, 30, 60 minutes, as well as at 2, 4, 8, and 24 hours following dose administration. The zero hour (trough) was collected within approximately 24 hours prior to dosing.

Approximately 2.0 mL of whole blood was collected at each time-point. The blood samples were placed into tubes containing K2EDTA as anticoagulant. The tubes were inverted several times to disperse the anticoagulant and were placed in wet ice or a cryo-rack for cooling until centrifugation. The date and clock time of each blood sampling were recorded. Blood samples were centrifuged (at approximately 3,000 rpm for 10 minutes at 4±3° C.) using a refrigerated centrifuge. Plasma was harvested and transferred into pre-labeled plastic cryogenic tubes. Each sample tube was labeled with at least the following: study number, animal number, sex, treatment (defined as TA #1, 2, 3, 4, or 5), collection time (hour), date, and study day (dose). Processing of blood samples and collection of plasma was completed, and the plasma samples placed in dry ice or the freezer within approximately 1 hour of blood collection. Subsequently, all samples were stored in a freezer at approximately −70±15° C.

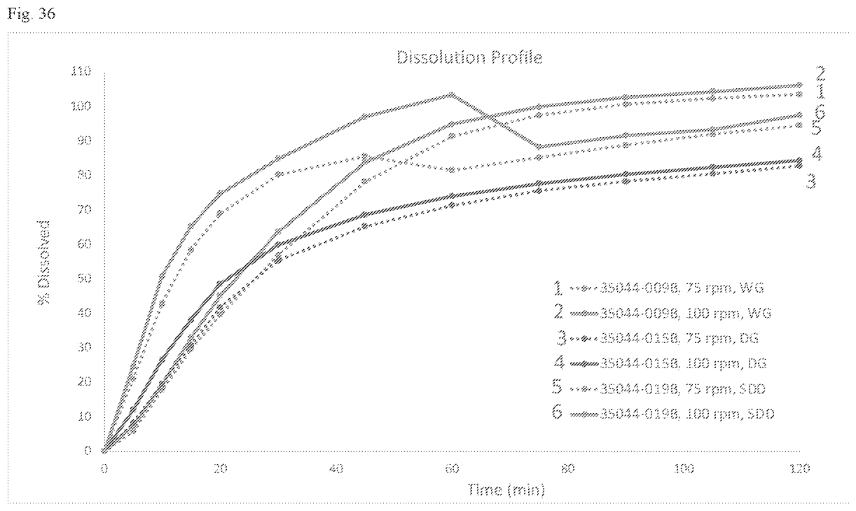

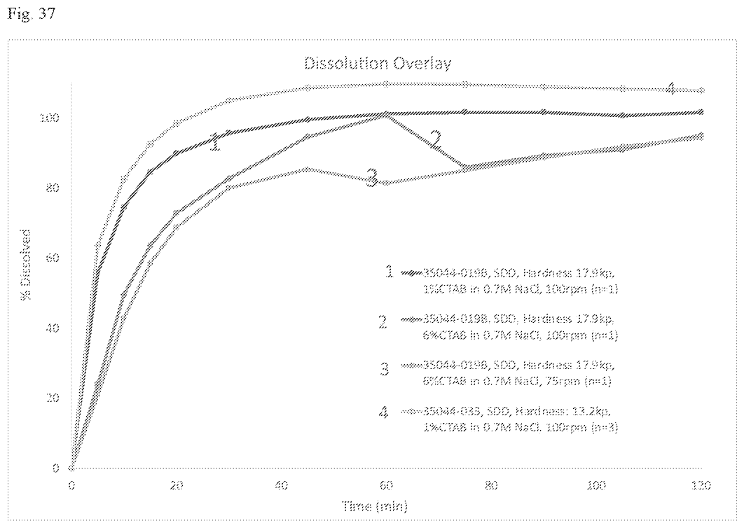

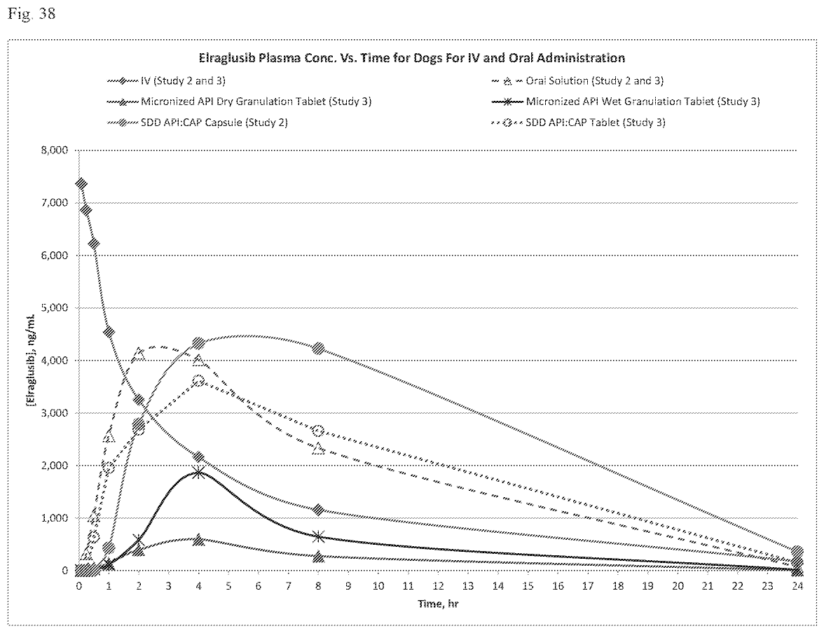

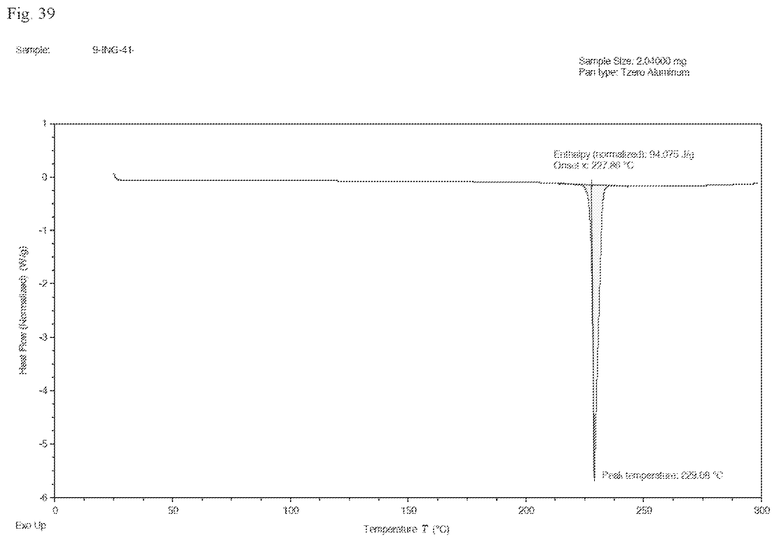

FIG. 38 shows the mean plasma profile of elraglusib observed in Study 3, as well as the plasma profile of elraglusib for the IV and oral solutions in studies 2 and 3, averaged.

Table 3-8 below summarizes the bioavailability data for each of the dog PK Studies 1, 2, and 3:

TABLE 3-8

| Dog | | | | | | | | | |
|---|---|---|---|---|---|---|---|---|---|
| PK | | Rt. | | | | | | AUC | |
| Study | | of | | Strength | Dose | | Cₘₐₓ | ng- | % |
| # | Descrpt. | Adm | Fed? | mg/mL | mg/kg | Tmax | ng/mL | h/mL | Bioavailable† |
| 1 | Canine | IV | No | 10 | 20 | 0.083 | 12,800 | 68,900 | 100% |
| | Vehicle | | | mg/mL | | | | | |
| 1 | Canine | Oral | No | 10 | 20 | 4.7 | 1,670 | 21,300 | 33% |
| | Vehicle | | | mg/mL | | | | | |
| 2 | Canine | IV | Yes | 10 | 10 | 0.083 | 7,347 | 32,299 | 100% |
| | Vehicle | | | mg/mL | | | | | |
| 2 | Elra:CAP | Oral | Yes | 100 | 20 | 8 | 4,323 | 62,611 | 97% |
| | powder | | | mg/capsule | | | | | |
| | capsule | | | | | | | | |
| 2 | Elra:PV | Oral | Yes | 100 | 20 | 8 | 3,957 | 43,904 | 68% |
| | AP | | | mg/capsule | | | | | |
| | Powder | | | | | | | | |
| | Capsule | | | | | | | | |
| 2 | Oral | Oral | Yes | 50 | 20 | 2 | 5,170 | 51,653 | 80% |
| | Solution | | | mg/mL | | | | | |
| 2 | Oral | Oral | Yes | 2 | 10 | 4 | 1,802 | 11,161 | 35% |
| | Suspension | | | mg/mL | | | | | |
| 2 and | IV | IV | Yes | 10 | 10 | ~0.083 | ~7800 | 32,433 | 100 |
| 3* | | | | mg/mL | | | | | |
| 2 and | Oral Soln | Oral | Yes | 50 | 20 | ~3 | ~3200 | 44,565 | 68.7 |
| 3* | | | | mg/mL | | | | | |
| 3 | Dry | Oral | Yes | 200 mg | 20 | ~4 | ~500 | 5,307 | 8.2 |
| | Granulation | | | | | | | | |
| | Tab | | | | | | | | |
| 3 | Wet | Oral | Yes | 200 mg | 20 | ~4 | ~1900 | 13,144 | 20.3 |
| | Granulation | | | | | | | | |
| | Tab | | | | | | | | |
| 3 | Elra:CAP | Oral | Yes | 200 mg | 20 | ~4 | ~3600 | 44,341 | 68.4 |
| | Tablet | | | | | | | | |

*Data of N = 6 animals from Study 2 and Study 3 combined were averaged for these groups
†% Bioavailable = 100*(AUC of Study Group)/(AUC of IV Group)

### PK Study 4—IV Versus Oral Solution in Normal Human Volunteers

An oral clinical proof of concept study with elraglusib is conducted as a companion study to an ongoing Phase 2 study of intravenous elraglusib. A subset of patients with recurrent or metastatic PDAC (Pancreatic ductal adenocarcinoma) previously untreated with systemic agents in the recurrent/metastatic setting receive a single oral dose of elraglusib 50 mg/mL Oral Solution drug product administered in combination with gemcitabine and nab-paclitaxel. Patients receive a single oral dose of elraglusib 50 mg/mL Oral Solution in place of the intravenous (IV) dose on Day 1 of Cycle 1 and receive IV dosing per the main protocol for all subsequent doses. The first three patients receive an oral dose of 9.3 mg/kg. In the absence of any significant toxicity, the subsequent groups of 3 patients receive 12.4 mg/kg, 15 mg/kg and 17.8 mg/kg oral doses to correspond with dose levels previously assessed in Phase 1 patients and to include one additional level with comparable exposure to 15 mg/kg based on the bioavailability of elraglusib 50 mg/mL Oral Solution (80% versus IV dosing) as determined in fed-state dogs.

| Investigational Product | elraglusib 50 mg/mL Oral Solution | |
|---|---|---|
| Phase | ½ Proof of Concept | |
| Rationale | The current elraglusib 10 mg/mL Injection drug product is | |
| | administered intravenously once or twice per week over multiple | |
| | hours which requires a significant amount of time and resource by | |
| | both patients and site personnel. An oral solution has been | |
| | developed and results of a single-dose study in fed-state dogs show | |
| | 80% dose-normalized bioavailability of the drug. | |
| | This companion proof of concept study is conducted, in a subset of | |
| | patients who are participating in the main study at select centers, in | |
| | order to compare the pharmacokinetic (PK) profiles of elraglusib | |
| | following a single dose of elraglusib 50 mg/mL Oral Solution to a | |
| | single dose administered intravenously. | |
| No. of Patients | Up to 15 | |
| Primary Objective | To determine the PK and local toxicity of elraglusib following a | |
| | single dose of 50 mg/mL oral solution administered in combination | |
| | with gemcitabine and nab-paclitaxel (GA) | |
| Secondary Objectives | To compare the PK of elraglusib following a single dose of 50 | |
| | mg/mL oral solution and a single intravenous infusion administered | |
| | in combination with gemcitabine and nab-paclitaxel (GA) | |
| Design | Open label, multicenter PK study done as a companion study to an | |
| | ongoing Phase 2 study | |
| Endpoints | Primary Endpoint: | |
| | AUC, Cₘₐₓ, Tₘₐₓ and t_{1/2} following a single oral dose of elraglusib | |
| | Adverse events will be monitored for the oral solution from the | |
| | time of dosing on Day 1 to receipt of the first dose of elraglusib | |
| | administered intravenously | |
| | Secondary Endpoints: | |
| | AUC, Cₘₐₓ, Tₘₐₓ and t_{1/2} following a single IV dose of elraglusib | |
| | compared to the single oral dose | |
| Population | Inclusion Criteria: Patient - | |
| | 1. Is able to understand and voluntarily sign a separate written | |
| | informed consent for this companion study and is willing and able to | |
| | comply with the companion protocol requirements | |
| | 2. Is able to swallow and retain oral medication | |
| | 3. Has been randomized to receive one of the treatment regimens | |
| | containing elraglusib in the main study | |
| | Patients who meet any of the following criteria are not eligible: | |
| | Exclusion Criteria: | |
| | 1. Has undergone significant surgery to the gastrointestinal tract | |
| | 2. Has any condition that would affect absorption of an oral drug | |
| Regimen | All patients who are eligible for this companion study receive GA | |
| | defined as a 30 to 40 minute intravenous infusion of nab-paclitaxel | |
| | at a dose of 125 mg per square meter, followed by an infusion of | |
| | gemcitabine according to the gemcitabine label at a dose of 1000 mg | |
| | per square meter, on Days 1, 8, and 15 of a 28-day cycle | |
| | Patients receive one of the following regimens: | |
| | If randomized to twice per week elraglusib in the main study: GA | |
| | followed by elraglusib given as an oral solution on Day 1 | |
| | On Day 4, patients continue in the full main study per protocol and | |
| | receive GA followed by elraglusib administered by IV infusion at a | |
| | dose of 9.3 mg/kg | |
| | If randomized to once per week elraglusib in the main study: GA | |
| | followed by elraglusib given as an oral solution of on Day 1 | |
| | On Day 8, patients continue in the full main study per protocol and | |
| | receive GA followed by elraglusib administered by IV infusion at a | |
| | dose of 9.3 mg/kg | |
| | Patients must consume one can of Ensure ® or eat a snack one hour | |
| | prior to taking the elraglusib 50 mg/mL Oral Solution. The oral | |
| | solution is prepared by the pharmacy and are administered using an | |
| | oral syringe to ensure accurate dosing. | |
| | Four dose levels are assessed in 3 patients each with PK and safety | |
| | assessed prior to each escalation: | |
| | 9.3 mg/kg | |
| | 12.4 mg/kg | |
| | 15.0 mg/kg | |
| | 17.8 mg/kg | |
| Duration of Therapy | Patients receive GA with a single dose of elraglusib 50 mg/ml Oral | |
| | Solution in this companion study. They then continue treatment with | |
| | additional IV infusions in the full study where they may continue on | |
| | study therapy with the combination as long as they do not have | |
| | clinically significant progressive disease and/or unacceptable | |
| | toxicity and as long as the investigator deems that the patient is | |
| | benefiting from treatment. | |
| Safety Evaluation | Safety of the elraglusib 50 mg/mL Oral Solution is assessed from the | |
| | time of dosing until receipt of elraglusib as an IV infusion by | |
| | recording and monitoring adverse events (AEs) and vital signs | |
| | (blood pressure, pulse, respiratory rate, and body temperature). | |
| Pharmacokinetic | Samples will be collected as part of this companion study for | |
| Evaluation | pharmacokinetic evaluation during Cycle 1 as follows: | |
| | Cycle 1 | Time Points |
| | Day 1 | elraglusib, Nab-Paclitaxel and Gemcitabine - Pre- |
| | (All patients - | dose (within 15 min prior to infusion start of Nab- |
| | elraglusib 50 | Paclitaxel) - Will assay for elraglusib, Gemcitabine |
| | mg/mL Oral | and Paclitaxel |
| | Solution) | Nab-Paclitaxel - End of infusion (+10 min) - Will |
| | | assay for Paclitaxel |
| | | Gemcitabine - End of infusion (+10 min) - Will |
| | | assay for Gemcitabine, dFdU, and Paclitaxel |
| | | elraglusib - 0.25, 0.5, 1, 2, 4, and 6 hours after |
| | | dosing - Will assay for elraglusib at all timepoints |
| | | and for Gemcitabine, dFdU, and Paclitaxel at 1, 2, 4 |
| | | and 6 hours after dosing |
| | | elraglusib, Nab-Paclitaxel, Gemcitabine, dFdU - 24 |
| | | 27 hours from start of the Nab-Paclitaxel infusion - |
| | | Will assay for elraglusib, Gemcitabine, dFdU, and |
| | | Paclitaxel |
| | Day 15 | elraglusib, Nab-Paclitaxel and Gemcitabine - Pre- |
| | (All patients - | dose (within 15 min prior to infusion start of Nab- |
| | IV elraglusib | Paclitaxel) - Will assay for elraglusib, Gemcitabine |
| | per the main | and Paclitaxel |
| | 1801 study) | Nab-Paclitaxel - End of infusion (+10 min) - Will |
| | | assay for Paclitaxel |
| | | Gemcitabine - End of infusion (+10 min) - Will |
| | | assay for Gemcitabine, dFdU, and Paclitaxel |
| | | elraglusib - End of Infusion End of Infusion and 1, |
| | | 2, 4 and 6 hours from end of infusion - Will assay |
| | | for elraglusib, Gemcitabine, dFdU, and Paclitaxel |
| | | elraglusib, Nab-Paclitaxel, Gemcitabine, and dFdU |
| | | 24-27 hours from start of the Nab-Paclitaxel |
| | | infusion - Will assay for elraglusib, Gemcitabine, |
| | | dFdU, and Paclitaxel |

| | * Note: Nab-Paclitaxel is given first, then Gemcitabine second |
|---|---|
| | elraglusib is administered after the chemo has finished. No |
| | observation period is required between infusions. |
| Statistical Considerations - | The primary objectives for this companion study to the 1801 Study |
| Methods Summary | Part 3 Arm B is to assess PK and local toxicity of GA plus a single |
| | dose of elraglusib given as an oral solution. |
| | The secondary objective is to compare the PK of a single dose of |
| | elraglusib given as an oral solution to those of an IV administration. |
| | AUC, Cmax, Tmax and t_{1/2} is calculated for each patient following a |
| | single IV dose and is compared to the data following a single PO |
| | dose. |
| | Sample Size: |
| | Up to 15 patients are enrolled in this companion study to ensure that |
| | there are 3 patients who are evaluable for PK assessment at each of |
| | the 4 planned dose levels. |
| | Statistical Methodology: |
| | The PK evaluable population for the primary objective is defined as |
| | all patients who receive a single dose of elraglusib 50 mg/mL Oral |
| | Solution and provide blood samples for PK analysis. The PK |
| | evaluable population for the secondary objective is defined as all |
| | patients who receive one dose of oral solution and one dose |
| | administered as an IV infusion and provide blood samples for PK |
| | analysis following both doses. |
| | For the primary endpoint, pharmacokinetic parameters including |
| | AUC, Cmax, Tmax and t½ are analyzed by non-compartmental |
| | methods using WinNonlin (Pharsight Corporation, CA). Adverse |
| | events and vital signs are recorded for the oral solution from the |
| | time of dosing to receipt of the next dose of elraglusib administered |
| | as an IV infusion. |
| | For the secondary endpoint, the same pharmacokinetic parameters |
| | are analyzed and are compared between oral and IV dosing. |
| | Safety: |
| | All safety assessments required in the full 1801 study are assessed |
| | for all patients in this companion study and results are included as |
| | part of the full 1801 study results. For the oral formulation |
| | specifically, the safety assessment is based on the frequency of AEs |
| | and vital signs during and following dosing until the patient receives |
| | his/her next dose of elraglusib as an IV infusion. |

This clinical study shows that administration of the oral administration of elraglusib 50 mg/mL Oral Solution provides AUC, Cₘₐₓ, Tₘₐₓ and t_{1/2} that enable therapeutic efficacy. This study also shows that administration of elraglusib 50 mg/mL Oral Solution is not associated with any significant adverse events.

The bioavailability studies described in Example 3 demonstrate the following points:
1) Oral administration of an IV solution formulation results in bioavailability that is approximately 31% of the bioavailability of the IV administered IV solution formulation. (PK Study 1)
2) The oral dosage forms of the disclosure provide elraglusib bioavailability that is comparable to obtained with IV administration. (PK Study 2, PK Study 3)
3) Oral administration of elraglusib with food provides increased bioavailability relative to administration without food. (PK Study 2, PK Study 3 vs. PK Study 1)
4) Oral administration of the dosage forms of the disclosure provide clinically meaningful elraglusib bioavailability. (PK Study 4).

### Example 4—Photostability Studies

### Background

Drug product development studies revealed occasional anomalous results with respect to [elraglusib], percentage label claim (% LC), elraglusib main peak Area %, and peak Area % values for related substances elraglusib RelS1 (HPLC RRT 0.96) and elraglusib RelS2 (HPLC RRT 0.97).

The apparently anomalous results suggested the possibility that elraglusib Injection, Solution drug product is more susceptible to light exposure than either solid elraglusib or elraglusib in acetonitrile solution.

Accordingly, elraglusib photo-sensitivity studies were conducted upon exposure to:
UV or visible light in a calibrated photo-chamber, and
Ambient light.

The photo-chamber and ambient light studies used a RP-HPLC method to monitor the following:
elraglusib concentration and % LC
elraglusib main peak Area %
elraglusib RelS1, elraglusib RelS2, and elraglusib Anhydride peak Area % values.

The photo-chamber studies included the following experimental variables:
Pharmaceutical Vehicles/Solvents
Formulation Vehicle PEG400:EtOH:Polysorbate 80 75:17:8% w/w (“FV”) Vehicle Variant 7 PEG400:EtOH:Polysorbate 80 94:5:1% w/w (“V7”)
PEG400 (“PEG-Only”)
PEG400:EtOH 95:5% w/w (“PEG-E”)
PEG400:Polysorbate 80 95:5% w/w (“PEG-T”)
Acetonitrile (“AN”)
elraglusib Concentration (2-, 10-, 15-, and 50-mg/mL)
Light Source (UV versus Visible)
Exposure duration (0, 25, 50, 75 and 100% of ICH Q1B conditions)
Container (clear glass vials versus cuvettes with and without foil over-wrap)
Dissolved gases (oxygen sparge versus nitrogen sparge versus no sparge)

The ambient light studies included the following experimental variables:
Primary Containers (clear versus amber glass vials)
Secondary Containers (translucent plastic divider boxes), and
Tertiary Containers (opaque cardboard boxes)

The results of the three photo-chamber studies support the following conclusions:
For elraglusib in various solvents and over the concentration range 10- to 50-mg/mL, elraglusib % LC and main peak Area % values decrease approximately linearly with increasing light exposure.
The elraglusib photo-exposure % LC loss rates exceed the main Peak Area % rates by a factor of approximately 6 (due to the elraglusib absorbance maximum shift from ~440 nm in elraglusib to ~330 nm for elraglusib RelS2.
elraglusib photo-exposure loss rates versus visible light exposure increase in the following order for the solvents studied: AN<<PEG-Only ~PEG-E<FV<V7<PEG-T
elraglusib photo-exposure loss rates versus visible light exposure are essentially independent of elraglusib concentration over the range 10- to 50-mg/mL
elraglusib photo-exposure loss rates are approximately 5 times faster upon exposure to visible light compared with UV light
elraglusib photo-exposure loss rates for samples exposed to visible light are essentially independent of dissolved gas (oxygen versus nitrogen)
elraglusib % LC losses are significant (approximately 20% loss) for elraglusib Injection, Solution upon exposure to 100% of 1200 klux-hr visible light
elraglusib RelS1 peak Area % changes for elraglusib Injection, Solution upon exposure to 100% of 1200 klux-hr visible light are not quantifiable owing to incomplete resolution between elraglusib RelS1 and elraglusib RelS2 using the RP-HPLC method.
elraglusib RelS2 peak Area % increases are significant (increase to approximately 3 to 5% peak Area %) for elraglusib Injection, Solution upon exposure to 100% of 1200 klux-hr visible light
elraglusib Anhydride peak Area % values are either unchanged or decrease slightly for elraglusib Injection, Solution upon exposure to 100% of 1200 klux-hr visible light

The results of the three ambient light studies support the following conclusions:
elraglusib Injection, Solution drug product in clear glass serum vials converts to elraglusib RelS2 upon exposure to ambient light over 28 days
Exposure to ambient light for 3 to 7 days has little impact, but elraglusib % LC and main peak Area % values decrease significantly after 14 to 28 days exposure while elraglusib RelS2 peak Area % values increase over the 7- to 28-day interval
Amber glass vials and the plastic divider cartons used as secondary packaging for the clear glass vials reduce, but do not completely protect against elraglusib photo-conversion upon ambient light exposure.
Placing clear glass vials in a plastic divider carton into an opaque cardboard box adequately reduces the rate of elraglusib photo-conversion upon ambient light exposure.

### Photo-Chamber Studies

### Photo-Chamber Study 1

The impact of light exposure on elraglusib in three solutions was examined, namely:
10FV (10 mg/mL elraglusib in Formulation Vehicle, PEG400:EtOH:Polysorbate80 75:17:8% w/w),
50FV (50 mg/mL elraglusib in Formulation Vehicle), and
50V7 (50 mg/mL elraglusib in Vehicle Variant 7, PEG400:EtOH:Polysorbate80 94:5:1% w/w).

The 10FV solution is identical to elraglusib Injection, Solution.

The three solutions were maintained in both 5-mL glass serum vials and UV-transparent plastic cuvettes (each with and without foil overwrap) and exposed to fractions (approximately 0, 25, 50, 75, and 100%) of ICH Q1B photostability conditions (1200 klux-hr in the visible region and 200 watt-h/m² in the UV region) using a calibrated photo-chamber.

Photo-exposure converted elraglusib to elraglusib RelS2. Solutions exposed to ≤25% of 1200 klux-hr visible light featured two partially-resolved peaks for elraglusib RelS1 and elraglusib RelS2 but the elraglusib RelS2 peak increased with increasing exposure interval and resolution between the two peaks decreased.

elraglusib % LC and main Peak Area % values decreased linearly with increasing visible and UV light exposure interval. The % LC loss rate was approximately 6 times faster than the main peak Area % loss rate.

% LC photo-conversion rates in vials (no foil) exposed to visible light were approximately 60% of the rates in cuvettes (no foil). Foil overwraps severely reduced the photo-conversion rates in both cuvettes and vials.

% LC and main peak Area % photo-conversion rates in both vials and cuvettes were significantly faster under visible light exposure compared with UV light exposure. Photo-conversion was approximately 5 times faster upon exposure to visible light compared with UV light.

For samples maintained in vials (no foil) and exposed to visible light, % LC and main peak Area % photo-conversion rates for 10FV were approximately 1.6 times higher than 50FV or 50V7 rates, whereas 50FV versus 50V7 photo-conversion rates were approximately equal. Table 4-1 below summarizes the photo-conversion rate comparisons.

TABLE 4-1
Photo-conversion Rate (Slope) Values for Vials Comparing
Formulations and % LC versus Main peak Area %

| | |
|---|---|
| Test Article | Slope Values* for Visible Light Exposure |

| Description | Main Peak Area %* | % LC^{†} |
|---|---|---|
| 10FV | −0.0433 | −0.250 |
| 50FV | −0.02741 | −0.154 |
| 50V7 | −0.02739 | −0.161 |
| 10FV/50FV | 1.6 | 1.6 |
| 10FV/50V7 | 1.6 | 1.6 |
| 50FV/50V7 | 1.0 | 1.0 |

*Slope = (Main Peak Area %)/(% of 1200 klux/hr)
^{†}Slope = (% LC)/(% of 1200 klux/hr)

For 10FV samples in vials exposed to 1200 klux-hr, elraglusib % LC losses were significant (approximately 20% loss). Under the same conditions, the elraglusib RelS2 peak Area % values increased to approximately 3 to 5%. elraglusib RelS1 peak Area % values were not reliably quantifiable owing to the incomplete resolution between the elraglusib RelS1 and elraglusib RelS2 peaks.

### Photo-Chamber Study 2

The impact of photo-chamber visible light exposure on 10 mg/mL elraglusib in four Vehicles, namely:
FV (PEG400:EtOH:Polysorbate 75:17:8% w/w, this is the elraglusib Injection Vehicle),
PEG-Only (100% PEG400),
PEG-E (PEG400:EtOH 95:5% w/w) and
PEG-T (PEG400:Polysorbate 95:5% w/w).

Visible light photo-exposure converted elraglusib to elraglusib RelS2 in each of the four Test Articles. The elraglusib Anhydride related substance peak Area % values decreased slightly with increasing photo-exposure in all Test Articles.

For all four Test Articles, elraglusib % LC and main Peak Area % values decreased linearly with increasing exposure interval.

Table 4-2 below shows that % LC and main peak Area % photo-conversion rate (slope) values increased in the order: PEG-Only ~PEG-E<FV<PEG-T.

TABLE 4-2
Slope Ratios for 10 mg/mL FV, PEG-Only, PEG-E, and
PEG-T Test Articles Exposed to Visible Light

| | | % LC | Main Peak | Main Peak |
|---|---|---|---|---|
| Test | % LC | Slope | Area % | Area % |
| Article | Slope^{†} | Ratio | Slope | Slope Ratio |
| FV | −0.305 | 1 | −0.057 | 1 |
| PEG-Only | −0.154 | 0.51 | −0.024 | 0.41 |
| PEG-E | −0.224 | 0.73 | −0.017 | 0.29 |
| PEG-T | −0.441 | 1.44 | −0.103 | 1.79 |

*Slope = (Main Peak Area %)/(% of 1200 klux/hr)
^{†}Slope = (% LC)/(% of 1200 klux/hr)

### Photo-Chamber Study 3

The impact of visible light exposure on elraglusib Test Articles that included the following:
10FV (10 mg/mL elraglusib in Formulation Vehicle PEG400:EtOH:Polysorbate 75:17:8% w/w),
2AN (2 mg/mL elraglusib in acetonitrile).

The 10FV Test Article is identical to elraglusib Injection, Solution drug product. The 2AN Test Article was included in this study to probe the effects of protic versus non-protic solvents on elraglusib photo-sensitivity and to supplement the prior photo-exposure study results with elraglusib in acetonitrile.

The 10FV Test Articles were initially sparged with either oxygen or nitrogen and the 2AN Test Article was not sparged.

The Test Articles were maintained in glass serum vials (25-mL fill in 50-mL vial) and exposed to fractions (0, 25, 50, 75, and 100%) 1200 klux-hr using a calibrated photo-chamber.

For all four Test Articles, elraglusib % LC and main Peak Area % values decreased linearly with increasing visible light exposure interval.

The % LC and elraglusib main peak Area % photo-conversion rates were essentially identical for both the oxygen-sparged and nitrogen-sparged samples.

The elraglusib photo-conversion rate was significantly slower for the 2AN Test Article compared with the 10FV Test Article.

For the 10FV Test Article, the elraglusib RelS2 peak Area % values increased approximately linearly to 4% peak Area % with increasing exposure interval, independent of sparging condition. elraglusib RelS2 peak Area % values remained below the method quantitation limit (0.08 peak Area %) at all visible light exposure intervals in the 2AN Test Article. The elraglusib Anhydride related substances peak Area % values either decreased slightly or did not change significantly with increasing visible light exposure interval.

### Ambient Light Study

This ambient light study used the same Test Articles, analytical methods, and procedures as Photo-chamber Study 1 except that Test Articles were exposed to ambient light for timed intervals (t=0, 1, 3, 7, 14, 21, and 28 days).

For all three Test Articles exposed to ambient light, elraglusib % LC and main peak Area % values remained essentially unchanged at the t=0-, 1-, and 3-d timepoints, but then decreased approximately linearly with increasing exposure interval. Foil overwraps severely reduced the photo-conversion rates in both cuvettes and vials.

Ambient light exposure converted elraglusib to elraglusib RelS2. The elraglusib RelS2 Area % values remained undetected at the t=0-, 1-, and 3-d timepoints, but increased approximately linearly with increasing ambient light exposure time thereafter. The elraglusib RelS2 Area % values increased significantly faster for the 10FV Test Article than for the 50FV and 50V7 Test Articles, but the rate of increase was approximately equal for the 50FV and 50V7 Test Articles.

### Example 5—Oral Solution Versus Intravenous Bioavailability in Human Volunteers

Example 5 sets forth the results of the PK study 4 in Example 3.

This pharmacokinetic (PK) study in healthy subjects was conducted to characterize and compare the PK parameters in plasma following a single intravenous (IV) dose of Elraglusib Injection, Elraglusib Oral Solution after fasting, and Elraglusib Oral Solution after a high-fat meal (fed). The study was intended to assess the potential feasibility of an oral formulation of elraglusib based on systemic exposure being within the biologically active range. In clinical studies conducted to date, elraglusib Injection (10 mg/mL) is administered IV once or twice per week over multiple hours, which requires a significant amount of time and resources by both patients and site personnel. An oral solution has been developed and PK Study 2 and PK Study 3 in fed-state dogs showed approximately ~70 to 80% dose-normalized bioavailability of the drug, which supports additional investigation. See Example 3 above.

This open label, single center, phase 1, randomized, single-dose, crossover, PK study of elraglusib administered as an oral solution in fed and fasted states and as an IV infusion in healthy subjects was conducted in 18 subjects to compare the PK profiles of Elraglusib Oral Solution to Elraglusib Injection.

The primary objectives of the study were: (1) Pharmacokinetics: to evaluate plasma pharmacokinetic (PK) of single dose elraglusib administered as an intravenous (IV) infusion and as an oral solution under both fasted and fed conditions in healthy subjects; and (2) Safety: to determine the tolerability of elraglusib administered as an IV infusion and as an oral solution.

Patient population: Eighteen subjects received at least 1 dose of elraglusib and are included in the safety population. Eighteen subjects had at least 1 blood sample providing evaluable PK data for elraglusib and are included in the PK Evaluable population. Sixteen subjects had sufficient data to calculate at least one of the PK parameters of Cmax, AUC, or λz and are included in the PK Analysis population for each of the treatments.

Inclusion criteria: Subjects were required to meet all the following criteria to be eligible for the study:
1. Was able to give signed informed consent of the protocol, which included compliance with the requirements and restrictions listed in the ICF
2. Was 18 to 55 years of age inclusive, at the time of signing the ICF
3. Was overtly healthy as determined by medical evaluation including medical history, physical examination, eye examination (ophthalmoscope and visual acuity), laboratory tests, and electrocardiogram (ECG)
4. Had systolic blood pressure (BP) 95-140 mmHg, diastolic BP 45-90 mmHg, and heart rate 45-100 beats per minute (bpm)
5. Had standard 12-lead ECG parameters that were normal unless the investigator considered the ECG tracing abnormality to be not clinically relevant. QTc had to be ≤450 milliseconds (ms) for both males and females.
6. Had laboratory parameters within the normal range unless the investigator considered an abnormality to be clinically irrelevant for healthy participants; however, serum creatinine, alkaline phosphatase (ALP), aspartate aminotransferase (AST), and alanine aminotransferase (ALT) were not to exceed 1.25 times the upper limit of normal (ULN). Total bilirubin value up to 1.5 times the ULN could be acceptable if associated with normal conjugated bilirubin value (unless the participant had documented Gilbert syndrome in which case bilirubin could be up to 3.0 times the ULN).
7. Had body weight between 50.0 and 100.0 kilograms (kg), inclusive, if male, and between 40.0 and 90.0 kg, inclusive, if female, with body mass index (BMI) between 18.0 and 32.0, inclusive
8. If female, was not pregnant, breastfeeding, lactating, or planning a pregnancy during the study period and had a negative urine pregnancy test within 24 hours prior to start of study. If postmenopausal, must have had a documented serum follicle stimulating hormone (FSH) level >40 mIU/mL to confirm.
   a. Women of childbearing potential (WOCBP) and males with partners of childbearing potential had to agree to use adequate contraception from the time of the first elraglusib dose until 4 months after the last elraglusib dose (unless exclusively in a same-sex relationship).
   b. Hormonal contraceptives were to begin at least 1 month prior to screening to ensure contraceptive was in full effect.
   c. Males with a WOCBP partner who was not currently pregnant had to agree to use a condom and be advised of the risk of a condom breaking or leaking during intercourse and the benefit for the WCOBP partner to use a highly effective method of contraception per protocol.
9. If male, must have agreed to refrain from donating sperm for 4 months after the last elraglusib dose.

### Exclusion Criteria:

Subject who met any of the following criteria was ineligible for the study:
1. At the time of screening, was symptomatic and had a clinically relevant cardiovascular, pulmonary, gastrointestinal, hepatic, renal, metabolic, hematological, neurological, osteomuscular, articular, psychiatric, systemic, ocular, or gynecologic (if female) history; infectious disease; or signs of acute illness
2. Had donated any volume of blood, including plasma, within 2 months before inclusion
3. Had symptomatic hypotension
4. Had presence or history of clinically significant drug hypersensitivity or allergic disease diagnosed and treated by a health care provider. Participants with known hypersensitivity to any component of the investigational product (IP) formulation were excluded.
5. Had a presence or history of drug abuse within the last year
6. Had history of heavy drinking within the past year [i.e., regular use of 21 drinks/week in males, 14 drinks/week in females]) with a risk of withdrawal symptoms arising during the study that may have confound the safety evaluation
   a. A drink was defined as 1 4 to 5-ounce glass of wine, 1 12-ounce beer, or 1 standard cocktail containing 1.5 ounces alcohol.
7. Regularly smoked more than 2 cigarettes per day (or vaping equivalent) and was unable to abstain during the confinement period of the study
8. Had used cannabinoids within 1 week before inclusion or did not agree to discontinue use of cannabinoids during the study
9. Excessively consumed beverages containing xanthine bases (more than 4 cups or glasses per day)
10. Had taken any prescription drugs or concomitant medications [other than contraceptives, non-live coronavirus disease (COVID) vaccines, supplements (e.g., vitamins)] within 3 days before the first elraglusib dose. Occasional acetaminophen and ibuprofen were allowed.
11. Had positive result on any of the following tests: hepatitis B surface antigen (HBs Ag), anti-hepatitis C virus antibodies (anti-HCV Ab), or anti-human immunodeficiency virus 1 and 2 antibodies (anti-HIV1 and anti-HIV2 Ab)
12. Had positive result on urine drug screen (amphetamines/methamphetamines, barbiturates, benzodiazepines, cannabinoids, cocaine, or opiates)
   a. If urine drug screen was positive for cannabinoids but subject stated they had not used cannabinoids within 1 week before inclusion and agreed to discontinue use of cannabinoids during the study, the subject could be included.
13. Had positive urine alcohol test
14. Had positive severe acute respiratory syndrome coronavirus 2 (SARS-CoV-2) test

### Randomization

Subjects were randomized to receive 1 of 6 dosing sequences:
1. oral fed-oral fasted-IV
2. oral fed-IV-oral fasted
3. oral fasted-oral fed-IV
4. oral fasted-IV-oral fed
5. IV-oral fed-oral fasted
6. IV-oral fasted-oral fed

The randomization followed a permuted block size of 6. There were no stratification factors to be implemented in the randomization schedule for this study. Subjects were randomized via a paper randomization schedule.

### Dosage Forms:

Elraglusib Injection is a is a light red to red clear solution of 10 mg/ml elraglusib in an inactive co-solvent mixture of polyethylene glycol 400, ethanol and Tween 80 (polysorbate 80) (PEG400:EtOH:Tween80; 75:17:8). The route of administration is by IV infusion after diluting elraglusib for injection with 0.9% Sodium Chloride Injection.

Elraglusib Oral Solution is 50 mg/mL elraglusib in Vehicle Variant 7, a co-solvent mixture of PEG400:EtOH:Tween80 (94:5:1%/w/w). It is a clear, red solution, free of visible particulate matter and a low bioburden liquid dosage form that is self-preserving against microbial growth.

### Dose Administration:

Elraglusib was administered on Day 1 of each of the 3 2-week study periods.

Subjects received a single 3.3 mg/kg dose of each of the following in a randomly assigned order separated by a 2-week period.

Elraglusib Oral Solution (50 mg/mL) in a fed state: Within a 30-minute period, a subject in a fed state consumed a high-fat meal (breakfast). The Elraglusib Oral Solution was also ingested within this 30-minute period. During the study, the high fat meal was administered over a period of under 30 minutes (in almost all cases, 28 minutes). The elraglusib dose was administered within 1 minute of the end of the meal, which in effect, resulted in the elraglusib oral dose being administered within 20 minutes of the start of the high fat meal.

Elraglusib Oral Solution (50 mg/mL) in a fasted state: Subjects fasted overnight (except water) for at least 8 hours before administration of the oral solution.

### NOTES for Oral Dosing:

Subjects were not to have their post-dose meal for at least 4 hours after the Elraglusib Oral Solution was ingested.

Subjects were not to eat or consume any liquid for 1 hour following ingestion of the Elraglusib Oral Solution. However, it was acceptable to consume a lozenge or hard candy or chew gum after dosing if the subject experienced any issues with the taste of the oral solution.

Elraglusib Injection (10 mg/mL) administered as an IV infusion over 60 minutes (concentration in mg/mL and infusion rate in mL/kg/hour could vary depending on the subject's body weight).

Subjects fasted overnight (except water) for at least 8 hours before administration of the IV infusion.

Subjects were not to have their post-dose meal for at least 4 hours after the end of the infusion.

The 3.3 mg/kg dose was based on the subject's Period 1 Day −1 weight (rounded to the nearest 0.1 kg) unless the subject's weight changed by more than 10%. In that instance, the dose was recalculated using the Day −1 weight (rounded to the nearest 0.1 kg) for Period 2 and/or 3, as indicated.

### Results:

PK parameters including AUC, Cₘₐₓ, Tₘₐₓ, and t_{1/2} were analyzed by standard non-compartmental analysis (NCA) methods following each of the elraglusib doses. Log transformed Cₘₐₓ and AUC parameters in subjects having adequate PK data following at least 1 of the oral and IV doses were used to assess the relative bioavailability of the oral dose (fed and/or fasted) compared to the IV dose.

Blood samples for PK analysis of elraglusib were to be drawn from all study subjects.

All efforts were made to obtain the PK samples at the scheduled nominal times relative to dosing. PK samples were assayed using validated analytical methods.

### Drug Dose, Drug Concentration

Subjects were targeted per protocol to receive 3.3 mg/kg of elraglusib. One subject (106) received only approximately 3% of the targeted dose in the IV infusion treatment after the infusion was interrupted in Period 2 due the adverse event of flushing (grade 2), which was assessed by the investigator as probably related to Elraglusib Injection. Oral doses were administered neat as an oral solution containing 50 mg/mL of elraglusib. The oral doses ranged from 3.6 to 6.3 mL of the oral solution depending on body weight. No additional liquid was allowed orally for at least an hour although subjects could consume a lozenge or hard candy or chew gum after dosing if taste was an issue for the subject.

TABLE 5-1
Summary of Dose Amounts, Volumes and Concentrations
Used for Elraglusib Administrations by Treatment
(PK Analysis Population)

| | Treatment | n | Mean | SD | CV(%) | Median | Min | Max |
|---|---|---|---|---|---|---|---|---|
| Dose | IV Infusion | 16 | 3.30 | 0.000 | 0.0 | 3.30 | 0.33 | 3.30 |
| (mg/kg) | Oral, | 16 | 3.30 | 0.000 | 0.0 | 3.30 | 0.33 | 3.30 |
| | Fasting | | | | | | | |
| | Oral, Fed | 16 | 3.30 | 0.000 | 0.0 | 3.30 | 0.33 | 3.30 |
| Or Dose | IV Infusion | 16 | 228.2 | 69.19 | 30.3 | 233 | 10.1 | 316 |
| (mg) | Oral, | 16 | 240.3 | 38.84 | 16.2 | 235 | 180 | 315 |
| | Fasting | | | | | | | |
| | Oral, Fed | 16 | 240.9 | 37.87 | 15.7 | 235 | 180 | 315 |
| Dose | IV Infusion | 16 | 22.82 | 6.919 | 30.3 | 23.3 | 1.01 | 31.6 |
| Volume^{a} | Oral, | 16 | 4.806 | 0.7767 | 16.2 | 4.70 | 3.60 | 6.30 |
| (mL) | Fasting | | | | | | | |
| | Oral, Fed | 16 | 4.819 | 0.7574 | 15.7 | 4.70 | 3.60 | 6.30 |
| Dose | IV Infusion | 16 | 10.00 | 0.000 | 0.0 | 10.0 | 10.0 | 10.0 |
| Concentration^{b} | Oral, | 16 | 50.0 | 0.00 | 0.0 | 50.0 | 50.0 | 50.0 |
| (mg/mL) | Fasting | | | | | | | |
| | Oral, Fed | 16 | 50.0 | 0.00 | 0.0 | 50.0 | 50.0 | 50.0 |
| Infusion | IV Infusion | 16 | 75.25 | 19.743 | 26.2 | 80.0 | 2.00 | 86.0 |
| Duration^{c} | Oral, | 16 | NA | NA | NA | NA | NA | NA |
| (minutes) | Fasting | | | | | | | |
| | Oral, Fed | 16 | NA | NA | NA | NA | NA | NA |

^{a}For the IV infusion, the volume of original drug product solution prior to adding the volume to an infusion bag containing 500 mL 0.9% normal saline
^{b}For IV infusion, the drug concentration in the drug product solution prior to being added to an infusion bag containing 500 mL of 0.9% normal saline
^{c}The infusion duration at the time the infusion bag was emptied of the original 500+ mL of the infused dose, but prior to flushing the infusion bag and infusion lines with approximately 25 mL of 0.9% normal saline at approximately 580 mL/hr to assure complete dose delivery.
The EOI PK sample was collected within 5 minutes after the EOI.
CV = coefficient of variation;
Min = minimum;
Max = maximum;
n = number of observations;
NA = not applicable;
SD = standard deviation;

### Elraglusib Mean PK Profiles by Treatment

Elraglusib plasma concentrations were collected for 96 hours post-dose for each of the 3 treatments. FIG. 41 presents the mean concentration time profiles for the 3 treatments using linear coordinates in the top panel and using semi-log coordinates in the bottom panel.

FIG. 41 shows that the IV infusion produced the highest peak mean concentration, while oral administration under fasted conditions produced the lowest peak mean values. The figure also indicates that ingesting the drug at the same time as a high fat meal resulted in a substantial increase in exposures and delayed the time to the peak mean concentration by a few hours compared to ingesting the drug under fasted conditions. All 3 mean profiles showed a similar rate of decline during the terminal washout phase. Twelve of 15 subjects PK Evaluable for all 3 treatments had elraglusib plasma concentrations above the LLOQ for at least 96 hours. Plasma concentrations had declined below the assay LLOQ (0.200 ng/ml) by 72 hours in 1 subject (118) with the IV infusion treatment, 1 subject (112) with the oral fasted treatment and 2 subjects (112, 118) with the oral fed treatment, and declined below the LLOQ in 1 subject (118) by 48 hours post-dose when on the oral fasted treatment. Only 1 subject (118) had a Cₗₐₛₜ of less than 96 hours for all 3 treatments. Quantifiable concentrations in the other subjects at 96 hours post-dose ranged from 0.273 ng/ml (109 in oral, fasted treatment) to 7.18 ng/ml (111 in IV infusion treatment).

There is a substantial between-subject variability in the elraglusib plasma concentrations. For the IV infusion the CVs over the initial 24 hours post-dose are generally in the 35% to 45%, but in the 20% to 40% range if the data from the subject that received only 3% of the planned dose (106) is ignored. However, following the oral treatments, the CVs are generally in the range of 60% to 100% when the drug is administered in the fasted state, or generally in the range of 45% to 150% when administered in the fed state.

### Elraglusib Mean PK Profiles by Treatment

Elraglusib Plasma PK parameters for elraglusib were determined for the 96-hour PK observations window for each of the 3 treatments. Table 5-2 summarizes the descriptive statistics following the IV infusion, Table 5-3 summarizes the descriptive statistics following the oral administration in the fasted state, and Table 5-4 summarizes the descriptive statistics following the oral administration in the fed state. Subject 106 was not evaluable for Cₘₐₓ- and AUC-related PK parameters after the IV infusion because of missed sample collections during the IV infusion, but PK parameters associated with the terminal elimination phase were calculable. Subject 114 was evaluable for PK parameters following the IV infusion treatment, but discontinued the study following that treatment so does not provide comparative information for the oral in the fasted or fed states. Both subjects are included in the descriptive statistics for the treatments and PK parameters for which they could be evaluated.

TABLE 5-2
Descriptive Statistics for Elraglusib PK Parameters after
Administration of Elraglusib as an IV Infusion, 3.3 mg/kg
(PK Evaluable Population)

| | | Arithmetic | | | | | | | Geometric | | | |
|---|---|---|---|---|---|---|---|---|---|---|---|---|
| | | | | | CV | Non-parametric | | | | GCV | LB | UB |
| Parameter | Units | n | Mean | SD | (%) | Median | Min | Max | GMean | (%) | 95% CI | 95% CI |
| Cₘₐₓ | ng/mL | 15 | 1601 | 217.0 | 13.6 | 1570 | 1230 | 1980 | 1588 | 13.7 | 1472 | 1712 |
| Cₘₐₓ/Dose | ng/mL/mg | 15 | 6.756 | 1.4110 | 20.9 | 7.02 | 4.59 | 9.50 | 6.615 | 21.8 | 5.870 | 7.454 |
| Tₘₐₓ | h | 15 | 1.218 | 0.4098 | 33.7 | 1.32 | 0.500 | 2.00 | 1.131 | 45.7 | 0.8886 | 1.440 |
| AUC₂₄ | ng · h/mL | 15 | 6848 | 1331.0 | 19.4 | 7200 | 4871 | 9902 | 6729 | 19.6 | 6043 | 7492 |
| AUC₄₈ | ng · h/mL | 15 | 7589 | 1511.6 | 19.9 | 7985 | 5158 | 10904 | 7448 | 20.4 | 6660 | 8329 |
| AUC₇₂ | ng · h/mL | 15 | 7845 | 1604.4 | 20.5 | 8194 | 5187 | 11346 | 7691 | 21.0 | 6855 | 8629 |
| AUC₉₆ | ng · h/mL | 15 | 7975 | 1656.2 | 20.8 | 8253 | 5195 | 11567 | 7813 | 21.4 | 6950 | 8783 |
| AUCₗₐₛₜ | ng · h/mL | 15 | 7974 | 1657.1 | 20.8 | 8253 | 5187 | 11567 | 7812 | 21.4 | 6948 | 8783 |
| AUC_{∞} | ng · h/mL | 15 | 8072 | 1707.1 | 21.1 | 8264 | 5194 | 11779 | 7903 | 21.8 | 7015 | 8903 |
| AUC_{∞}/Dose | h · ng/mL/mg | 15 | 33.79 | 8.057 | 23.8 | 32.7 | 23.5 | 49.7 | 32.93 | 23.7 | 28.93 | 37.48 |
| % extrap | % | 15 | 1.146 | 0.7747 | 67.6 | 1.04 | 0.102 | 2.52 | 0.7979 | 142.1 | 0.4458 | 1.428 |
| Cₗₐₛₜ | ng/mL | 16 | 3.045 | 2.1668 | 71.2 | 2.64 | 0.293 | 7.18 | 2.142 | 129.0 | 1.264 | 3.630 |
| t_{1/2} | h | 16 | 19.64 | 6.282 | 32.0 | 20.1 | 8.64 | 33.7 | 18.60 | 37.0 | 15.37 | 22.50 |
| λZ | 1/h | 16 | 0.0397 | 0.01635 | 41.1 | 0.0346 | 0.020 | 0.080 | 0.0372 | 37.0 | 0.0308 | 0.0451 |
| | | | 9 | 0 | | | 5 | 3 | 8 | | 0 | 1 |
| R² | # | 16 | 0.9904 | 0.01154 | 1.2 | 0.9964 | 0.965 | 0.999 | 0.9903 | 1.2 | 0.9842 | 0.9966 |
| | | | | | | | 2 | 9 | | | | |
| CL | L/h/kg | 15 | 0.4268 | 0.09445 | 22.1 | 0.399 | 0.280 | 0.635 | 0.4176 | 21.8 | 0.3707 | 0.4704 |
| CL | L/h | 15 | 31.14 | 7.095 | 22.8 | 30.6 | 20.1 | 42.6 | 30.37 | 23.7 | 26.68 | 34.56 |
| Vz | L/kg | 15 | 11.19 | 3.221 | 28.8 | 10.9 | 6.17 | 17.3 | 10.77 | 29.5 | 9.174 | 12.63 |
| Vz | L | 15 | 821.8 | 286.92 | 34.9 | 716 | 530 | 1618 | 783.0 | 31.9 | 658.9 | 930.3 |

AUCₜ = area under the curve from time = 0 to t;
CI = confidence interval;
CL = clearance;
CL/F = apparent clearance;
Cₗₐₛₜ = last quantifiable concentration;
Cₘₐₓ = maximum observed concentration;
CV = coefficient of variation;
F = fraction bioavailable;
GCV = geometric CV;
GMean = geometric mean;
LB = lower bound;
Max = maximum;
Min = minimum;
n = number of observations;
NC = not calculable;
SD = standard deviation;
R² = coefficient of determination;
t_{1/2} = terminal elimination half-life;
Tₘₐₓ = time of observed Cₘₐₓ;
UB = upper bound;
Vz = volume of distribution;
Vz/F = apparent volume of distribution;
λz = terminal elimination rate constant.

TABLE 5-3
Descriptive Statistics for Elraglusib PK Parameters
after Administration of Elraglusib Orally in a Fasted State
(PK Evaluable Population)

| | | Arithmetic | | | | Non-parametric | | |
|---|---|---|---|---|---|---|---|---|
| Parameter | Units | n | Mean | SD | CV(%) | Median | Min | Max |
| Cₘₐₓ | ng/mL | 15 | 171.7 | 123.71 | 72.1 | 169 | 32.2 | 473 |
| Cₘₐₓ/Dose | ng/mL/mg | 15 | 0.6705 | 0.42526 | 63.4 | 0.622 | 0.161 | 1.55 |
| Tₘₐₓ | h | 15 | 2.700 | 1.6345 | 60.5 | 2.00 | 1.50 | 8.00 |
| AUC₂₄ | ng · h/mL | 15 | 1226 | 871.6 | 71.1 | 1073 | 346 | 3400 |
| AUC₄₈ | ng · h/mL | 15 | 1620 | 1209.8 | 74.7 | 1425 | 584 | 4724 |
| AUC₇₂ | ng · h/mL | 15 | 1736 | 1309.5 | 75.5 | 1533 | 623 | 5126 |
| AUC₉₆ | ng · h/mL | 15 | 1781 | 1346.3 | 75.6 | 1560 | 633 | 5256 |
| AUCₗₐₛₜ | ng · h/mL | 15 | 1780 | 1346.9 | 75.7 | 1560 | 633 | 5256 |
| AUC_{∞} | ng · h/mL | 15 | 1811 | 1371.7 | 75.8 | 1591 | 640 | 5324 |
| AUC_{∞}/Dose | h · ng/mL/mg | 15 | 7.224 | 4.9740 | 68.9 | 5.77 | 2.78 | 21.3 |
| % extrap | % | 15 | 1.518 | 0.9419 | 62.0 | 1.45 | 0.322 | 3.29 |
| Cₗₐₛₜ | ng/mL | 15 | 1.158 | 1.0489 | 90.6 | 0.719 | 0.259 | 3.60 |
| t_{1/2} | h | 15 | 16.41 | 4.756 | 29.0 | 18.2 | 6.92 | 24.0 |
| λZ | 1/h | 15 | 0.04754 | 0.020905 | 44.0 | 0.0381 | 0.0288 | 0.100 |
| R² | # | 15 | 0.9926 | 0.01299 | 1.3 | 0.9962 | 0.9482 | 1.000 |
| CL | L/h/kg | 15 | 2.608 | 1.3976 | 53.6 | 2.07 | 0.620 | 5.16 |
| CL | L/h | 15 | 185.7 | 89.66 | 48.3 | 173 | 47.0 | 359 |
| Vz | L/kg | 15 | 56.64 | 26.906 | 47.5 | 52.0 | 14.1 | 109 |
| Vz | L | 15 | 4103 | 1867.8 | 45.5 | 3979 | 1068 | 7611 |

| | | Geometric | | | |
|---|---|---|---|---|---|
| | | | | LB | UB |
| Parameter | Units | GMean | GCV(%) | 95% CI | 95% CI |
| Cₘₐₓ | ng/mL | 132.6 | 91.5 | 86.08 | 204.2 |
| Cₘₐₓ/Dose | ng/mL/mg | 0.5482 | 77.2 | 0.3755 | 0.8005 |
| Tₘₐₓ | h | 2.417 | 46.8 | 1.889 | 3.092 |
| AUC₂₄ | ng · h/mL | 1014 | 68.4 | 719.5 | 1429 |
| AUC₄₈ | ng · h/mL | 1335 | 67.1 | 952.3 | 1872 |
| AUC₇₂ | ng · h/mL | 1426 | 67.6 | 1015 | 2003 |
| AUC₉₆ | ng · h/mL | 1461 | 68.1 | 1037.7 | 2056 |
| AUCₗₐₛₜ | ng · h/mL | 1459 | 68.3 | 1036 | 2055 |
| AUC_{∞} | ng · h/mL | 1482 | 68.7 | 1050 | 2091 |
| AUC_{∞}/Dose | h · ng/mL/mg | 6.128 | 61.0 | 4.488 | 8.365 |
| % extrap | % | 1.219 | 85.1 | 0.8097 | 1.834 |
| Cₗₐₛₜ | ng/mL | 0.8024 | 109.4 | 0.4910 | 1.311 |
| t_{1/2} | h | 15.60 | 36.7 | 12.81 | 18.99 |
| λZ | 1/h | 0.04443 | 36.7 | 0.03650 | 0.05409 |
| R² | # | 0.9925 | 1.3 | 0.9852 | 0.9999 |
| CL | L/h/kg | 2.227 | 68.7 | 1.578 | 3.142 |
| CL | L/h | 163.2 | 61.0 | 119.5 | 222.8 |
| Vz | L/kg | 50.12 | 59.0 | 37.03 | 67.83 |
| Vz | L | 3673 | 55.2 | 2760 | 4887 |

AUCₜ = area under the curve from time = 0 to t; CI = confidence interval; CL = clearance; CL/F = apparent clearance; Cₗₐₛₜ = last quantifiable concentration; Cₘₐₓ = maximum observed concentration; CV = coefficient of variation; F = fraction bioavailable; GCV = geometric CV; GMean = geometric mean; LB = lower bound; Max = maximum; Min = minimum; n = number of observations; NC = not calculable; SD = standard deviation; R² = coefficient of determination; t_{1/2} = terminal elimination half-life; Tₘₐₓ = time of observed Cmax; UB = upper bound; Vz = volume of distribution; Vz/F = apparent volume of distribution; λz = terminal elimination rate constant.

TABLE 5-4
Descriptive Statistics for Elraglusib PK Parameters
after Administration of Elraglusib Orally in a Fed State
(PK Evaluable Population)

| | | Arithmetic | | | | Non-parametric | | |
|---|---|---|---|---|---|---|---|---|
| Parameter | Units | n | Mean | SD | CV(%) | Median | Min | Max |
| Cₘₐₓ | ng/mL | 15 | 591.8 | 406.32 | 68.7 | 507 | 74.3 | 1670 |
| Cₘₐₓ/Dose | ng/mL/mg | 15 | 2.340 | 1.3667 | 58.4 | 2.11 | 0.338 | 5.48 |
| Tₘₐₓ | h | 15 | 5.338 | 2.1967 | 41.2 | 5.00 | 3.00 | 12.00 |
| AUC₂₄ | ng · h/mL | 15 | 3929 | 2071.8 | 52.7 | 3988 | 960 | 8372 |
| AUC₄₈ | ng · h/mL | 15 | 4549 | 2398.6 | 52.7 | 4445 | 1154 | 9792 |
| AUC₇₂ | ng · h/mL | 15 | 4780 | 2558.0 | 53.5 | 4597 | 1169 | 10350 |
| AUC₉₆ | ng · h/mL | 15 | 4894 | 2632.2 | 53.8 | 4727 | 1171 | 10572 |
| AUCₗₐₛₜ | ng · h/mL | 15 | 4893 | 2633.1 | 53.8 | 4727 | 1169 | 10572 |
| AUC_{∞}. | ng · h/mL | 15 | 5001 | 2712.5 | 54.2 | 5017 | 1171 | 10829 |
| AUC_{∞}/Dose | h · ng/mL/mg | 15 | 20.18 | 9.905 | 49.1 | 19.2 | 5.32 | 43.3 |
| % extrap | % | 15 | 1.865 | 1.4858 | 79.7 | 1.73 | 0.175 | 5.77 |
| Cₗₐₛₜ | ng/mL | 15 | 3.055 | 2.4488 | 80.2 | 2.03 | 0.200 | 7.74 |
| t_{1/2} | h | 15 | 20.83 | 9.799 | 47.0 | 21.2 | 7.12 | 50.5 |
| λZ | 1/h | 15 | 0.04026 | 0.020458 | 50.8 | 0.0327 | 0.0137 | 0.0974 |
| R² | # | 15 | 0.9928 | 0.00883 | 0.9 | 0.9963 | 0.9734 | 1.000 |
| CL/F | L/h/kg | 15 | 0.9052 | 0.63155 | 69.8 | 0.658 | 0.305 | 2.82 |
| CL/F | L/h | 15 | 64.49 | 40.995 | 63.6 | 52.1 | 23.1 | 188 |
| Vz/F | L/kg | 15 | 22.89 | 10.749 | 47.0 | 19.4 | 10.8 | 47.9 |
| Vz/F | L | 15 | 1686 | 880.7 | 52.2 | 1501 | 818 | 4212 |

| | | Geometric | | | |
|---|---|---|---|---|---|
| | | | | LB | UB |
| Parameter | Units | GMean | GCV(%) | 95% CI | 95% CI |
| Cₘₐₓ | ng/mL | 473.5 | 85.3 | 314 | 713.0 |
| Cₘₐₓ/Dose | ng/mL/mg | 1.958 | 75.7 | 1.349 | 2.842 |
| Tₘₐₓ | h | 5.023 | 35.3 | 4.155 | 6.074 |
| AUC₂₄ | ng · h/mL | 3422 | 61.9 | 2496 | 4691 |
| AUC₄₈ | ng · h/mL | 3975 | 60.6 | 2916 | 5417 |
| AUC₇₂ | ng · h/mL | 4159 | 61.8 | 3035 | 5700 |
| AUC₉₆ | ng · h/mL | 4248 | 62.5 | 3091 | 5840 |
| AUCₗₐₛₜ | ng · h/mL | 4247 | 62.6 | 3088 | 5839 |
| AUC_{∞}. | ng · h/mL | 4328 | 63.5 | 3135 | 5974 |
| AUC_{∞}/Dose | h · ng/mL/mg | 17.90 | 57.1 | 13.34 | 24.01 |
| % extrap | % | 1.301 | 125.1 | 0.7599 | 2.226 |
| Cₗₐₛₜ | ng/mL | 2.053 | 138.1 | 1.159 | 3.639 |
| t_{1/2} | h | 19.00 | 47.2 | 14.82 | 24.36 |
| λZ | 1/h | 0.03648 | 47.2 | 0.02846 | 0.04678 |
| R² | # | 0.9928 | 0.9 | 0.9879 | 0.9977 |
| CL/F | L/h/kg | 0.7625 | 63.5 | 0.5524 | 1.053 |
| CL/F | L/h | 55.88 | 57.1 | 41.64 | 74.98 |
| Vz/F | L/kg | 20.90 | 45.4 | 16.44 | 26.56 |
| Vz/F | L | 1532 | 44.9 | 1208 | 1942 |

AUCₜ = area under the curve from time = 0 to t;
CI = confidence interval;
CL = clearance;
CL/F = apparent clearance;
Cₗₐₛₜ = last quantifiable concentration;
Cₘₐₓ = maximum observed concentration;
CV = coefficient of variation;
F = fraction bioavailable;
GCV = geometric CV;
GMean = geometric mean;
LB = lower bound;
Max = maximum;
Min = minimum;
n = number of observations;
NC = not calculable;
SD = standard deviation;
R² = coefficient of determination;
t_{1/2} = terminal elimination half-life;
Tₘₐₓ = time of observed Cₘₐₓ;
UB = upper bound;
Vz = volume of distribution;
Vz/F = apparent volume of distribution;
λz = terminal elimination rate constant.

These summary tables indicate that mean Cₘₐₓ values (arithmetic mean values) were higher following the IV infusion (1601 ng/mL) than following either of the oral doses, and the oral administration in a fed state (591.8 ng/ml) gave a substantially higher Cₘₐₓ than was observed in the fasted state (171.7). A similar pattern was observed for the partial and total AUC estimates, with the mean AUC_{∞} values ranging from 8072 h·ng/mL following the IV administration to 1811 h·ng/mL following oral administration in the fasted state with oral administration in the fed state being intermediate at 5001 h·ng/mL. The 96-hour PK observation window was sufficient to capture between 98% and 99% of the estimated AUC_{∞} value after each of the 3 treatments.

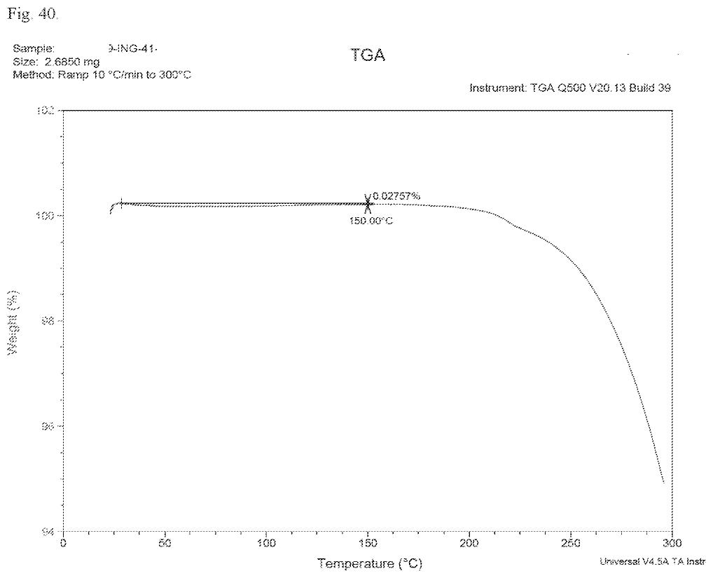

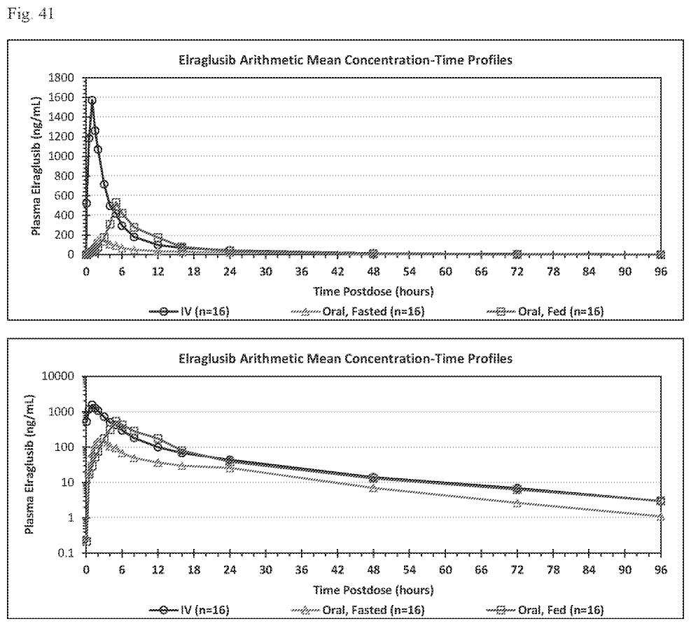

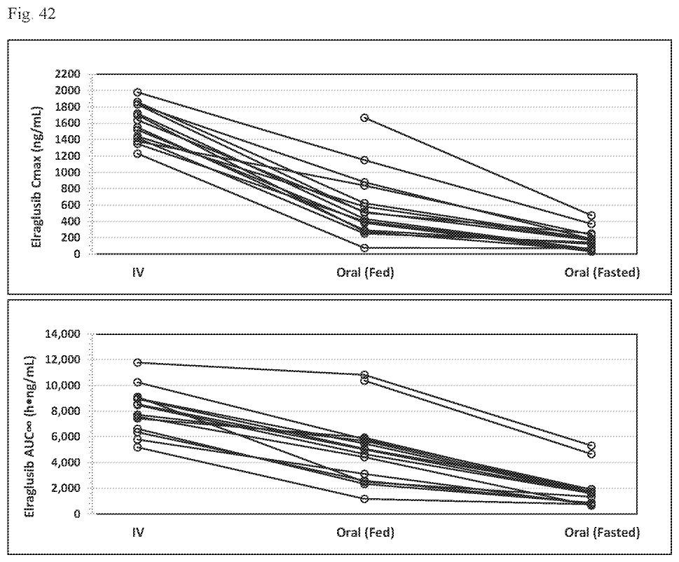

FIG. 42 provides a visual summary of how the various subjects participating in the study showed considerable consistency in the decreasing plasma exposure (as measured by both Cₘₐₓ and AUC_{∞}) as the subjects received the IV infusion, the oral dose in the fed state, and the oral dose in the fasted state. The transition plots indicate there was only 1 subject (118) where the Cₘₐₓ exposures following both fed and fasted were similar, and similarly only 1 subject (also 118) where the AUC_{∞} exposures were similar under both fed and fasted conditions. The transition plots also indicate there was only 1 subject (105) where the AUC_{∞} exposure following oral dosing in the fed state was relatively close to that of the IV infusion. A second patient (106) may also be in such a group, but that patient was not evaluable for Cₘₐₓ or AUC_{∞} exposures for the IV infusion because of missing data early in the infusion process and incomplete delivery of the targeted dose.

The mean terminal elimination half-lives were between 16 and 21 hours for all three treatments. The longest terminal half-life (50.5 h, 113) was observed after the oral treatment in the fed state, and the shortest half-life (6.92 h, 118) was observed after the oral administration in the fasted state. In general, it did not appear that the terminal elimination half-life was dependent on the treatment, with the majority of subjects having half-lives on all 3 treatments between 15 and 25 hours. Subjects with short half-lives (<11 hours) on 1 treatment showed a similar pattern on all 3 treatments. Except for the single instance in 1 subject with the 50.5-hour half-life, the longest half-lives were in the 24-27 hour range, not greatly dissimilar from the majority of the subjects, and not predominantly associated with any particular treatment.

Clearance of elraglusib averaged 0.4268 L/h/kg or 31.14 L/h among the subjects when they received the IV infusion treatment. This clearance value is greater than the nominal adult glomerular filtration rate of 7.2 L/h in humans, but less than nominal renal blood flow (~72 L/h) or nominal hepatic blood flow (~90 L/h), so consistent with renal and/or hepatic clearance mechanisms (Davies B and Morris T. Physiological Parameters in Laboratory Animals and Humans. Pharmaceutical Research 10 (7), 1093-1095 (1993) DOI: 10.1023/a: 1018943613122).

The mean elraglusib apparent (oral) clearance was 2.608 L/h/kg or 185.7 L/h following administration in the fasted state, but lower (0.9052 L/h/kg or 64.49 L/h) when administered in the fed state, both consistent with incomplete absorption from the gut. The difference in apparent clearances between fasting vs. fed states, without any change in the terminal elimination rate, indicates that, when compared to oral administration while fasting, the high fat breakfast in the fed state facilitates the extent of drug absorption from the gut.

The volume of distribution of elraglusib averaged 11.19 L/kg or 821.8 L among the subjects when they received the IV infusion treatment. This volume is substantially larger than the total body volume, indicating distribution into body tissues, probably at concentrations greater than observed in the plasma in several, or most, tissues. The mean apparent volume of distribution following oral administration was 2— to 5-times larger than the volume of distribution after IV administration, consistent with incomplete of absorption of elraglusib from the gut, as also noted for the apparent clearance. As noted from the apparent clearance values, the apparent volumes of distribution indicate less absorption from the gut occurred following administration under the fasted state than following administration in the fed state.

### Relative Bioavailability Comparisons for Elraglusib Treatments

The relative bioavailability of oral administration of elraglusib under fasted and fed states as compared to the absolute (100%) bioavailability following the IV infusion, and the relative bioavailability of the oral dose in the fed state compared to the oral dose in the fasted state are summarized in Table 5-5. Elraglusib administered in the fasted state has an oral bioavailability of approximately 5-11% when calculated using the geometric means of the Cₘₐₓ values of IV and oral in the fasted state but is 14-21% when based on the geometric means of the AUCₗₐₛₜ or AUC_{∞} values of the individual subjects. Administering elraglusib in the fed state results in an increased oral bioavailability. Elraglusib administered in the fed state results in an oral bioavailability of approximately 19% to 38% when calculated using the geometric means of the Cₘₐₓ values of IV and oral in the fasted state but is 41% to 63% when based on the geometric means of the AUCₗₐₛₜ or AUC_{∞} values of the individual subjects. Administration in the fed state results in an oral bioavailability based on Cₘₐₓ that is 2.5- to 5-times that observed when administered in the fasted state and 2.4- to 3.6-times than that observed in the fasted state when based on the AUC metrics.

TABLE 5-5
Assessments of Relative Bioavailability of Oral Elraglusib
Compared to IV Infusion and Comparison of Oral Bioavailability when
Administered in a Fed State as Compared to a Fasted State
(PK Analysis Population)

| | | LS Means | | Ratio (%) | 90% Confidence Interval | |
|---|---|---|---|---|---|---|
| | | IV | Fasted | Fasted/IV | Lower | Upper |
| Cₘₐₓ | ng/mL | 1588 | 132.6 | 7.62 | 5.16 | 11.27 |
| AUCₗₐₛₜ | h · ng/mL | 7812 | 1459 | 17.11 | 13.73 | 21.31 |
| AUC_{∞} | h · ng/mL | 7903 | 1482 | 17.15 | 13.78 | 21.34 |
| | | IV | Fed | Fed/IV | Lower | Upper |
| Cₘₐₓ | ng/mL | 1588 | 473.5 | 27.24 | 19.30 | 38.46 |
| AUCₗₐₛₜ | h · ng/mL | 7812 | 4247 | 50.75 | 40.73 | 63.24 |
| AUC_{∞} | h · ng/mL | 7903 | 4328 | 51.06 | 40.93 | 63.70 |
| | | Fasted | Fed | Fed/Fasted | Lower | Upper |
| Cₘₐₓ | ng/mL | 132.6 | 473.5 | 357.10 | 253.98 | 502.09 |
| AUCₗₐₛₜ | h · ng/mL | 1459 | 4247 | 291.00 | 238.82 | 354.58 |
| AUC_{∞} | h · ng/mL | 1482 | 4328 | 292.05 | 239.77 | 355.72 |

### Dose and Elraglusib Relative Bioavailability

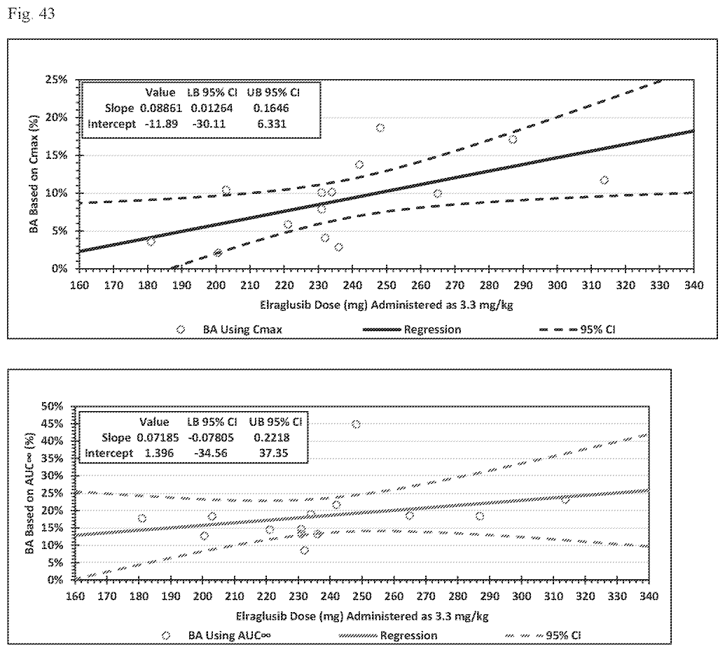

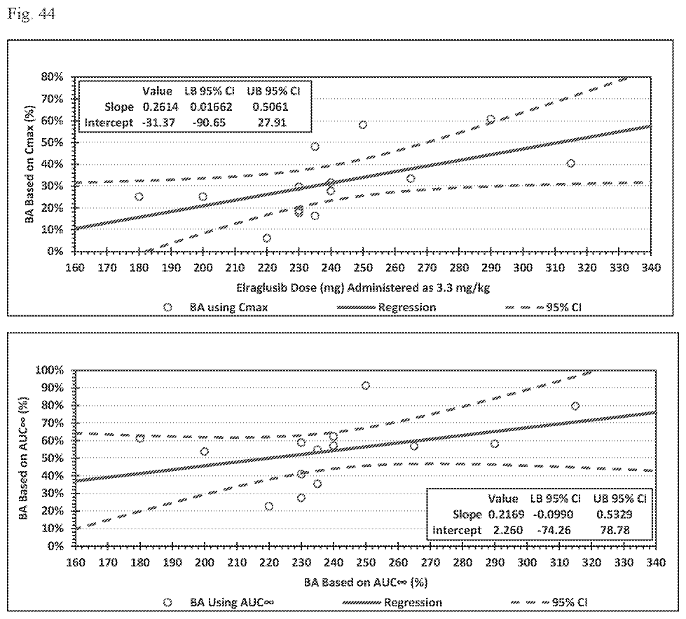

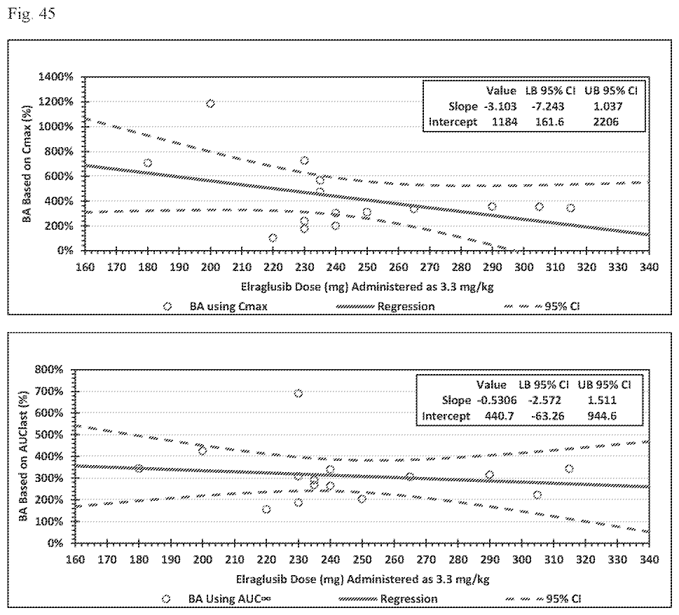

Although elraglusib was dosed at 3.3 mg/kg in the study subjects, the weights of the subjects varied by nearly 2-fold from 54.3 kg to 96.6 kg. Thus, total doses of administered elraglusib ranged from approximately 180 mg to 316 mg (with the exception of the incomplete IV infusion dose of approximately 10.1 mg administered to subject 106). Linear regressions of the relative bioavailability of elraglusib based on ratios of Cₘₐₓ or AUC_{∞} for the individual subjects are provided in FIG. 43 for the IV infusion, FIG. 44 for oral dosing in the fasted state, and FIG. 45 for oral dosing in the fed state. If the slopes of the relationships in each case are approximately 0.0 (i.e., a nearly horizontal line) then the bioavailability of elraglusib in that setting was approximately uniform over the 2-fold dosing range tested in this study. Confidence intervals (95% CI) are included on the plots to help estimate whether the regression line is statistically different from 0.0 given the limited number of data points available.

The bioavailability linear regressions of Fasted/IV vs. dose for elraglusib (FIG. 43) suggest that bioavailability increases with increasing dose. While the slope is statistically different from 0.0 when bioavailability is assessed using Cₘₐₓ (i.e., the 95% CI for the slope does not contain 0.0), it is not statistically different when comparing using AUC_{∞} values. The F_{Cmax} based regression estimates that the oral bioavailability under fasting conditions increases approximately from 4% to 16% as the dose increases from 180 mg to 320 mg, while the F_{AUC∞} based regression estimates that increase to be approximately from 14% to 24%.

The bioavailability linear regressions for Fed/IV vs. dose (FIG. 44) show a similar pattern of having a statistically significant increase in bioavailability with dose when based on Cₘₐₓ, but not when based on AUC_{∞}. The F_{Cmax} based regression estimates that the oral bioavailability in the fed state increases approximately from 16% to 52% as the dose increases from 180 mg to 320 mg, while the F_{AUC∞} based regression estimates that increase to be approximately from 41% to 71%. The trends indicate that the oral bioavailability of elraglusib may increase as drug is absorbed from the gut at a greater rate in subjects receiving higher doses, and the increase affects Cₘₐₓ slightly more than the AUC.

When the 2 oral dose treatments are compared using linear regression of Fed/Fasted vs. dose (FIG. 45) the slopes of the regressions are negative, but not statistically different from 0.0. If the trends are real, they would indicate that the divergence between oral bioavailability in fasted and fed states is greatest when the elraglusib dose is low and decreases at the higher elraglusib doses used in the study.

### Dose and Elraglusib Clearance and Volume of Distribution

Elraglusib clearance and apparent clearances were examined using linear regression for a dependence on the elraglusib dose administered. The slopes of the regression relationships for none of the 3 treatments were statistically different from 0.0. However, there was a trend for the slopes following both oral dose treatments to be slightly negative, consistent with the previously noted bioavailability findings that Cₘₐₓ and AUC_{∞} had regression relationships that increased with increasing dose. The regression line estimates that the apparent clearance after a 320 mg dose is a third to half of the apparent clearance after a 180 mg dose.

Elraglusib volume of distribution and apparent volume of distribution were also examined using linear regression for a dependence on the elraglusib dose administered. The slopes of the regression relationships for none of the 3 treatments were statistically different from 0.0. In addition, there was no apparent trend for the slopes following the oral dose treatments to be either consistently negative or consistently positive. There is little evidence of the volume of distribution or apparent volume of distribution being impacted by the elraglusib dose administered.

This study examined the plasma pharmacokinetics of elraglusib following administration of the drug as an IV infusion of approximately 80 minutes duration, as an oral dose administered in a fasted state, and as an oral dose administered in a fed state (i.e., high fat, high calorie breakfast). The pharmacokinetics objectives of the study included evaluating the pharmacokinetics of elraglusib when administered IV and orally and determining the relative bioavailability of elraglusib when administered orally in a fasted state or when n administered orally in a fed state compared to its bioavailability when administered as an IV infusion. Additionally, the impact of administering the oral dose in a fed state was to be compared to administration in a fasting state.

The study enrolled 18 healthy adult subjects in the 3 period, cross-over study, in which serial PK samples were collected for 96 hours following each of the single dose treatments. All the subjects were pharmacokinetically evaluable in that they had at least one post-dose PK sample collected, although only 16 of the subjects had 1 or more of their 3 concentration-time profiles with enough data available to calculate at least the pharmacokinetic parameters of Cₘₐₓ and AUC. Fourteen of the patients had sufficient pharmacokinetic information to make comparisons of drug exposure (i.e., Cₘₐₓ and AUC) between at least 2 of the 3 treatments.

### Concentrations and Exposures

Elraglusib plasma concentrations declined to near or below the assay LLOQ (0.200 ng/mL) by 96 hours post-dose with all 3 treatments, and the observed AUCₗₐₛₜ on average accounted for between 98% and 99% of the AUC. for all 3 treatments. The concentration-time profiles showed distinct differences between the amplitude and timing of the mean peak concentration, with the greatest exposure and most rapid rise in concentrations associated with the approximately 80-minute IV infusion. The least exposure (i.e., low Cₘₐₓ and low AUC) occurred when the oral dose was administered in the fasted state. Ingesting the dose in the fed state resulted in a considerable increase in exposures compared to the oral dose in the fasted state, the 95% CI indicating oral with meal exposures (i.e., fed state) being 2.5- to 5-fold the oral fasted state exposures, but taking the dose with the meal delayed the median time to reach Cₘₐₓ by 3 hours.

The terminal elimination half-lives appeared to be similar in most subjects and in the range of approximately 15 to 25 hours for all 3 treatments in the majority of the subjects. However, 2 or 3 of the 16 subjects with evaluable half-lives had elimination half-lives that were approximately a third to a half of the half-lives of the majority of the subjects, suggesting that the study population contained some subjects with a rapid clearance phenotype. This subset of subjects had terminal elimination half-lives ranging from 7 to 11 hours and tended to have low Cₘₐₓ and AUC values following oral dosing in the fed or fasted states. They also had among the lowest AUC_{∞} values following the IV infusion but were in the middle of the pack for Cₘₐₓ values following the IV infusion. The subset of subjects with short half-lives were among the subjects with the greatest systemic clearance values and smallest volumes of distribution following the IV infusion dosing, consistent with the shorter half-life observation.

### Bioavailability Assessments

Relative bioavailability assessments between the 3 treatments compared pairwise confirmed the differences visually apparent from their concentration time profiles. The IV infusion treatment is taken to have 100% bioavailability since the drug is infused directly into the systemic circulation. The oral route of administration is expected to have lower bioavailability either due to incomplete absorption from the gut or due to elimination of drug from the blood stream before the drug can get into the general systemic circulation due to one or more first pass effects. The oral bioavailability of elraglusib was considerably lower when administered in the fasted state than when administered in the fed state suggesting elraglusib preferentially partitions into fatty or lipid materials and can disperse more effectively and transfer across lipid interfaces more readily when administered in the fed state. This interpretation of high lipophilicity is consistent with the observed high volume of distribution, approximately 11 L/kg, observed for elraglusib following administration as an IV infusion.

The relative bioavailability of elraglusib following oral dosing also showed some evidence of dependence on the dose of elraglusib administered, the relative bioavailability increasing as the dose increased. This possibility hints at the presence of a saturable clearance pathway operating in parallel with non-saturable pathways, somewhere in the string of processes between reaching the gut wall and reaching the general circulation, such as a saturable metabolism pathway. This possibility would account for the observation of just a 3% to 11% (Cₘₐₓ based or AUC_{∞} based) oral bioavailability at the lowest doses utilized in the study but increasing by approximately 13% with doses approximately 75% higher, also used in the study. However, these differences in relative bioavailability are also associated with different subjects so it is possible that coincidently the higher doses were administered to individuals with a propensity for better absorption of elraglusib. This type of saturable drug elimination pathway would be expected to have a greater effect on Cₘₐₓ values than on AUC values since it would be most prominent when drug absorption is occurring most rapidly (i.e., in the time window before reaching and while at Cₘₐₓ).

The lesser impact of such a saturable drug elimination pathway being less on the elraglusib when administered in the fed state could be related to its higher lipophilicity allowing it to bypass that pathway, either because of location (the later Tₘₐₓ when administered with a high fat meal implies more of the absorption occurs further along the gut than when administered while fasting), or because of different uptake mechanisms than can occur with molecules dissolved in or carried along with fat particles.

Interestingly, the relative bioavailability following oral dosing in the fed state reached 91% in one subject (105). This case emphasizes the point that if some saturable pathway is operating to lower the oral bioavailability at low doses, it has less impact as oral doses are increased and will ultimately have a nearly undetectable effect as the relative bioavailability of the oral dose approaches 100% (i.e., producing essentially the same total exposure as if the dose was administered by IV infusion). This study used IV infusion and oral doses of 3.3 mg/kg; previous early phase studies in patient populations have used elraglusib dosed by IV infusion at doses of 1.0 to 15.0 mg/kg, nearly 3- to 5-times the dose utilized in this study, with only minor deviation from dose proportionality (a 15-fold dose change resulting in a 20-fold change in AUC₂₄. If that deviation is real, it is also consistent with the presence of a saturable drug elimination pathway working in parallel with other non-saturable pathways.

Drug Accumulation with Repeat Dosing

Accumulation with repeat dosing was estimated by identifying the extent of carryover from the first dosing interval (i.e., the single dose used in this current study to subsequent dosing intervals 1×tau, 2×tau, etc. to n×tau hours when the carryover would be less than 1%). For the IV infusion treatment, Cₘₐₓ occurs rapidly, and once the infusion is completed concentrations fall rapidly over the next approximately 10 to 12 hours because of a rapid distribution phase. Concentrations decline more than 10-fold over that time interval. Accumulation using a planned twice weekly dosing regimen is predicted to be in the 1% to 5% range and even lower if the adopted dosing interval is once weekly. This increase in concentrations between first dose and steady-state is similar to the precision of the current elraglusib assay and so would likely be undetectable unless first dose and steady-state data from a large number of patients were available.

The oral doses have lower Cₘₐₓ values but absorption of the elraglusib from the intestinal tract requires longer than typically used for the elraglusib infusions in this study. When taken with food, the terminal half-life becomes dominant after 18 to 24 hours post-dose, and concentrations are still approximately 10% to 20% of the Cₘₐₓ values 24 hours after administration of the first dose. Accumulation, using a planned twice weekly dosing regimen, is predicted to be in the 1% to 10% range and lower if the adopted dosing interval is once weekly. Substantial accumulation with repeated dosing (e.g., exceeding 30%) is unlikely unless dosing intervals of 24 hours or less are utilized. Patients with long elraglusib half-lives (i.e., 20+ hours) will generally show greater accumulation after reaching steady-state than will patients with short half-lives (i.e., 10 hours or less).

### Pharmacokinetic Conclusions

Elraglusib is systemically bioavailable when administered orally, with its oral bioavailability being enhanced by 2.5- to 5-fold when administered in a fed state (i.e., with a high fat breakfast). Doses greater than 3.3 mg/kg may show greater oral bioavailability than the approximately 5% to 21% observed when administered in a fasted state and the approximately 19% to 63% when administered in a fed state.

Elraglusib pharmacokinetics show substantial between-subject variability when the dose is administered orally. Concentrations and PK parameter estimates are substantially less variable if dosing is by IV infusion.

Elraglusib pharmacokinetics are in their terminal elimination phase after approximately 24 hours post-dose whether the dose is administered IV or oral. The terminal elimination phase has a half-life in the 15- to 25-hour range in the majority of individuals, although there may be a subset of individuals who clear the drug more rapidly exhibiting a terminal half-life in the 6- to 11-hour range.

Elraglusib has a total body clearance that exceeds the nominal glomerular filtration rate in healthy adults but is consistent with being either renally cleared if there are active secretion mechanisms or metabolically cleared in the liver. The apparent clearance observed after oral dosing appears to be a combination of incomplete absorption and first pass metabolism. Incomplete absorption is likely to play a larger role in the higher apparent clearance observed when dosed in the fasted state.

Elraglusib has a volume of distribution that exceeds body weight by approximately a factor of 11 indicating substantial distribution into tissues. Concentrations in some tissues are likely to substantially exceed plasma concentrations.

Significant accumulation of elraglusib with repeated dosing is unlikely with once or twice weekly dosing regimens. Significant accumulation is likely from first dose to steady state with dosing intervals of 24 hours or less.

### Example 6-Preparation and Evaluation of Elraglusib ASDs with Four Different Polymers Prepared by Spray Drying From Acetone:Water Solvent

Prior formulation development work compared micronized elraglusib formulated in both dry and wet granulated tablets as well as a spray-dried dispersion (ASD) in a dry granulated tablet. These formulations were dosed in dog PK Study 3. Based on that study an ASD composition of 1:1 elraglusib:cellulose acetate phthalate (CAP) dosed in a tablet was found to achieve approximately 68% oral bioavailability and nominated as the lead for further development. The lead ASD-tablet composition is shown in Table 6-1.

TABLE 6-1
Lead Elraglusib:CAP ASD tablet composition
identified from dog PK study

| | | Weight Percent |
|---|---|---|
| Ingredient | Brand Name | (% w/w) |
| 1:1 elraglusib:CAP ASD | | 41.67 |
| Microcrystalline Cellulose | PH 102 | 27.33 |
| Mannitol | Pearlitol 100SD | 25.00 |
| Croscarmellose Sodium | Ac-Di-Sol | 2.00 |
| Magnesium Stearate | Ligamed MF-2- | 1.00 |
| | K | |
| CCS | Ac-Di-Sol | 2.00 |
| Silicon Dioxide | Syloid 244FP | 0.50 |
| Magnesium Stearate | Ligamed MF-2- | 0.50 |
| | K | |

| | |
|---|---|
| Total | 100.00 |

Elraglusib ASD optimization and tableting evaluation was conducted to: (1) Evaluate feasibility of avoiding tetrahydrofuran (THF) as the spray-drying solvent by measuring active pharmaceutical ingredient (API) solubility in routine commercial spray-drying solvents and confirm solubility in presence of bioavailability enhancing polymers; (2) manufacture ASD prototypes and characterize these for dissolution performance and physicochemical stability; and (3) evaluate ASD prototypes in the lead tablet formulation.

To this end, a series of ASD prototypes were successfully prepared and characterized. Lead ASD formulations were selected based on initial biorelevant dissolution and physical state characterization data. These lead ASD formulations were then progressed into tablet formulation evaluations and development stability studies.

### Materials

Materials used in elraglusib ASD development studies are summarized in Table 6-2 (raw materials) and Table 6-3 (analytical method reagents). Equipment used in elraglusib ASD development studies are presented in Table 6-4.

TABLE 6-2
Materials used in Elraglusib ASD development studies

| Reagent | Brand Name/Grade | Supplier | Lot |
|---|---|---|---|
| elraglusib | NA | Pharmaron | NB-elraglusib(NG)- |
| | | | A-1 |
| Acetone | ACS | LabChem | L014-16 |
| Cellulose acetate phthalate (CAP) | NF | Eastman | P157920 3-WA-3616 |
| Hydroxypropyl methyl cellulose | AQOAT AS-MG | Shin-Etsu | 1083255 |
| acetate succinate (HPMCAS-M) | | | |
| Hydroxypropyl methyl cellulose | AQOAT AS-HG | Shin-Etsu | 2043114 |
| acetate succinate (HPMCAS-H) | | | |
| Eudragit ® L100 | L100 | Evonik | b210603207 |
| Tetrahydrofuran (THF) | HPLC, Inhibitor | VWR/BDH29559 | 21070220 |
| | free | | |
| Microcrystalline cellulose (MCC) | Avicel PH 102 | Dupont | P220934058 |
| Mannitol | Pearlitol 100SD | Roquette | E121G |
| Croscarmellose sodium (CCS) | Ac-Di-Sol SD711 | Dupont | 2174052814 |
| Magnesium stearate | Ligamed MF-2-K | Peter Greven | C949145 |
| Silicon dioxide | Aerosil 200 Pharma | Evonik | 300053100 |

TABLE 6-3
Material used in Elraglusib analytical methods

| | Brand | | |
|---|---|---|---|
| Reagent | Name/Grade | Supplier/Catalog Number | Lot |
| Acetonitrile | GC | LabSolv/LSG1120 | 22030045 |
| Trifluoroacetic acid (TFA) | HPLC | Sigma Aldrich/302031- | MKCR0683 |
| | | 10x1ML | 100344973 |
| Hexadecyltrimethylammonium | Molecular | Sigma/H6269 | BCCF0998 |
| bromide (CTAB) | biology, ≥99% | | |
| Sodium chloride | ACS | Spectrum/S1240 | 2JL0397 |
| Hydranal Water Standard 10.0 | Hydranal | Honeywell Fluka/34425 | K2220 |
| Check P Solution | NA | Aquamicron/GCHP | A20086 |
| CompoSolver E | Hydranal | Honeywell/34734-1L | L0210 |
| Hydranal Composite 2 | Hydranal | Honeywell/34806 | L2110 |
| Acetone | ACS | LabChem/LC104204 | L014-16 |
| Fasted state simulated | | Biorelevant/FASBUF | FASBUF-1121-A |
| intestinal fluid (FaSSIF) buffer | NA | | |
| concentrate, pH 6.5 | | | |
| Fed state simulated intestinal | NA | Biorelevant/FESBUF | FESBUF-0122-A |
| fluid (FeSSIF) buffer | | | |
| concentrate, pH 5.0 | | | |
| FaSSIF/FeSSIF/fasted state | | Biorelevant/FFF03 | FFF-1121-A |
| simulated gastrointestinal fluid | NA | | |
| (FaSSGF) powde , pH 1.6 | | | |

TABLE 6-4
Equipment used in Elraglusib ASD development studies

| Equipment | Brand Name or Description |
|---|---|
| Balance | Analytical balance |
| | Top load balance |
| Positive displacement pipette | Gilson 1000 μL pipette |
| | Gilson 5 mL pipette |
| | Gilson 1 mL pipette |
| | Gilson M1000 pipette |
| | Gilson M100 pipette |
| Timer | Durac timer |
| Spray dryer | Buchi B-290 |
| Tablet press (manual) for | Globe Pharma MTCM-1 |
| compression profiles | |
| Dissolution testing equipment | Sotax MD |
| Karl Fischer titrato | Mettler Toledo V20S |
| HPLC | Agilent 1100 |
| Scanning electron microscope | Phenom Pro in 10 kV |
| X-ray powder diffractometer | Rigaku Miniflex 6G |

### Manufacturing Methods

### Spray Dried Dispersion (ASD) Preparation

Four different elraglusib ASD formulations were prepared using four unique polymers (refer to Table 6-5). Elraglusib and polymer were weighed and dissolved in a 90:10 acetone:water mixture to produce spray solutions containing 4.0% weight by weight (w/w) total solids. The solution was stirred until full dissolution of the solids was visually confirmed. The B-290 mini spray dryer was set up per the configuration summarized in Table 6-6. As the apparatus was heating, the pump was calibrated with solvent to achieve a spray rate of 10-15 g/min (actual rate in Table 6-6). Once the inlet temperature was reached, the outlet temperature was allowed to stabilize. Solvent was run until a steady outlet temperature of 40-45° C. was reached. Upon reaching steady state conditions, spraying of the elraglusib/polymer solution was initiated using the calibrated pump flow rate and size 14 L/S tubing. Following spray-drying, the product was collected, and oven dried at 40° C. for not less than (NLT) 20 hours. Full process parameters are provided in Table 6-7.

TABLE 6-5
Polymer usage by lot in ASD prototypes

| Lot | Polymer |
|---|---|
| 35044-040 | HPCMAS-M |
| 35044-041 | HPCMAS-H |
| 35044-042 | Eudragit ® L100 |
| 35044-043 | Eastman CAP |

TABLE 6-6
B-290 mini spray dryer configuration

| | Lots 35044-040, 35044-041, | |
|---|---|---|
| Parameter | 35044-042, and 35044-043 | |
| Loop | Closed | |
| configuration | loop | |
| Cyclone volume | 1.1 | L |
| B-295 loop system | Inert loop | |
| Aspirator rate | 100% | |
| Nitrogen flow rate | 30 | m |
| Cooling temperature | −20° | C. |

TABLE 6-7
ASD processing parameters

| Process | | | | Lot #65044- |
|---|---|---|---|---|
| Parameter | Lot #35044-040 | Lot #35044-041 | Lot #35044-042 | 043 |
| Nozzle | Two-fluid, 0.7 mm | Two-fluid, 0.7 mm | Two-fluid, 0.7 mm | Two-fluid, 0.7 mm |
| | Liquid Tip, | Liquid Tip, | Liquid Tip, | Liquid Tip, |
| | 1.5 mm Air Cap | 1.5 mm Air Cap | 1.5 mm Air Cap | 1.5 mm Air Cap |
| Inlet temperature | 64-68 | 68-69 | 68-70 | 68-71 |
| (° C.) | | | | |
| Outlet temperature | 40-45 | 40-45 | 40-45 | 40-45 |
| (° C.) | | | | |
| Aspirator (%) | 100 | 100 | 100 | 100 |
| Solution | 10-15 | 10-15 | 10-15 | 10-15 |
| flowrate | | | | |
| (g/min) | | | | |
| Condenser | −20 | −20 | −20 | −20 |
| temperature (° C.) | | | | |
| Spray gas | 30 | 30 | 30 | 30 |
| flowmeter (mm) ^{(a)} | | | | |
| Secondary | 40 | 40 | 40 | 40 |
| drying | | | | |
| temperature | | | | |
| (° C.) | | | | |
| Secondary drying | NLT 20 | NLT 20 | NLT 20 | NLT 20 |
| time (hours) | | | | |

^{(a)} The 30 mm height on the rotameter corresponds to 439 L/hour actual volume flow at standard temperature and pressure.

### Granulation

The microcrystalline cellulose (MCC), mannitol, and croscarmellose sodium (CCS) were sieved through #20 mesh screen (0.85 mm). The sieved ingredients and elraglusib ASD material were transferred into a 500 mL polypropylene bottle and blended for 10 minutes at 25 revolutions per minute (rpm). Magnesium stearate was sieved through a #30 mesh (0.595 mm) and added to the mixture. The intragranular ingredients were blended for an additional 5 minutes at 25 rpm.

The preblend was compacted using the MCTM-1 manual press (roller compaction simulator) with a simulated roll force of 4-5 kN/cm and milled through a 1.25 mm screen. The blend was collected in a 250 mL polypropylene bottle and weighed to calculate the adjustment factor for extragranular excipients. The extragranular CCS was added to the milled granules and blended for 5 minutes at 25 rpm. The extragranular silicon dioxide was sieved through a #20 mesh (0.85 mm) into the mixture which was blended for 5 minutes at 25 rpm. The extragranular magnesium stearate was then sieved through a #30 mesh (0.595 mm) into the mixture which was blended for 5 minutes at 25 rpm to produce the final blend.

### Tableting

Tablets were pressed using the MTCM-1 using 9.5×21.0 mm plain-faced modified oval tooling.

### Analytical Methods

### ID, Assay, and Related Substances by HPLC

High performance liquid chromatography (HPLC) testing was performed using an Agilent 1100 series HPLC equipped with Chemstation data collection software or Agilent 1260 series HPLC equipped with OpenLab data collection software.

Chromatographic separation was achieved using a Waters Atlantis T3, 4.6×150 mm, 3 μm column and the parameters provided in Table 6-8. Testing was performed using external standard prepared at 0.2 mg/mL elraglusib (lot NB-elraglusib (NG)-A-1) in diluent. Test samples for the tablet prototypes were prepared by serial dilution to a target concentration of 0.2 mg/mL elraglusib. All samples were diluted using volumetric flasks.

TABLE 6-8
Analytical conditions for Elraglusib ID,
assay, and related substances method.

| Mobile phase | Mobile phase A: 0.02% v/v | |
|---|---|---|
| | TFA in water Mobile phase | |
| | B: 0.02% v/v TFA in | |
| | acetonitrile | |
| Diluent | 90:10 Acetonitrile:Water | |
| Column | Waters Atlantis T3, 3.0 μm, 4.6 × 150 mm | |
| | (Part number: 186003729) | |
| Flow rate (mL/min) 1.0 | 1.0 | |
| Column temperature (° C.) 30 | 30 | |
| Sample temperature (° C.) 5 | 5 | |
| Injection volume (μL) 20 | 20 | |
| Peak width | >0.05 min (1 second response time, 5 Hz) | |
| Detector wavelength (nm) | UV at 438 nm, 4 nm bandwidth | |
| Needle wash | 50:50 acetonitrile:water | |
| Run time (min) | 27 | |
| Acquisition time (min) | 27 | |
| Time (min) | % A | % B |
| 0.0 | 70 | 30 |
| 15.0 | 10 | 90 |
| 20.0 | 10 | 90 |
| 20.1 | 70 | 30 |
| 27.0 | 70 | 30 |

### Sink Dissolution

Sink dissolution experiments were performed on a Sotax MD automated dissolution apparatus with in-line ultraviolet detection. Elraglusib standard was used for quantitation of percent dissolved using ultraviolet absorbance at 282 nm. Sink dissolution testing was performed using the parameters shown in Table 6-9.

TABLE 6-9
Sink dissolution method parameters

| Dissolution media | 1% w/w CTAB in 0.7M sodium chloride | |
|---|---|---|
| Diluent | 6% w/w CTAB in 0.7M sodium chloride | |
| Medium volume | 900 | mL |
| Medium temperature | 37° | C. |
| Timepoints | 0, 5, 10, 15, 20, 30, 45, 60, 75, 90, 105 | |
| | minutes and infinity (15 min at 250 rpm] | |
| Speed | 100 | rpm |
| Filter | 0.45-μm PVDF Syringe Filters (Pall, Part | |
| | Number 4500) | |
| UV detector wavelength | 282 | nm |

### Non-Sink Dissolution

Non-sink dissolution testing was performed in duplicate using manual sampling (Table 6-10), and a R&D HPLC method developed by Core Rx with the same column as the assay and related substances method (Table 6-11). Approximately 15 mg of material was weighed into scintillation vials equipped with cross-shaped stir bars. To initiate the non-sink dissolution test, 15 mL of either fasted state (FaSSIF), or fed state (FeSSIF), simulated intestinal fluid was then added to each vial while stirring at 125 rpm. Sampling of 1.0 mL was performed at the time intervals shown in table 10. The aliquots were microcentrifuged at 15800×g for two minutes, and the supernatant diluted using a 5× dilution factor directly into amber HPLC vials. For the 10- and 90-minute time points after microcentrifugation, an additional sample of the supernatant was passed through a 0.2 μm microcentrifuge cut-off filter, using 15800×g for two minutes centrifuge parameters, in an attempt to extract unbound “free” drug from solubilized colloidal species without the use of an ultracentrifuge.

TABLE 6-10
Non-sink dissolution method parameters

| Dissolution media | Fasted state (FaSSIF) or Fed State | |
|---|---|---|
| | (FeSSIF) simulated intestinal fluid | |
| Diluent | 25:75 water:acetonitrile | |
| Medium volume | 15 | mL |
| Medium temperature | 37° | C. |

| | |
|---|---|
| Timepoints | 0, 5, 10, 20, 30, 60, 90 minutes |
| Speed | 125 rpm |
| Detector wavelength (nm) | UV at 438 nm, 4 nm bandwidth |

TABLE 6-11
Analytical HPLC parameters for Elraglusib
R&D non-sink dissolution method

| Mobile phase | Mobile phase | |
|---|---|---|
| | A: water | |
| | Mobile phase B: | |
| | acetonitrile | |
| Diluent | 25:75 water:acetonitrile | |
| Column | Waters Atlantis T3, 3.0 μm, 4.6 × 150 mm | |
| | (Part number: 186003729) | |
| Flow rate (mL/min) | 1.0 | |
| Column temperature (° C.) | 30 | |
| Sample temperature (° C.) | Ambient | |
| Injection volume (μL) | 20 | |
| Peak width | >0.05 min (1 second response time, 5 Hz) | |
| Detector wavelength (nm) | UV at 438 nm, 4 nm bandwidth | |
| Needle wash | 25:75 water:acetonitrile | |
| Run time (min) | 5 | |
| Acquisition time (min) | 5 | |
| Time (min) | % A | % B |
| 0.0 | 25 | 75 |
| 5.0 | 25 | 75 |

### Moisture Content by Karl Fischer

Water content was determined by performing a volumetric Karl Fischer (KF) titration using a Mettler Toledo V20S KF Titrator. Titration standardization was performed with Hydranal Composite 2 and Check P solution. A 300 mg sample was dissolved in CompoSolver E and used for KF water titration.

### Bulk and Tapped Density

To determine bulk density, the test article powder was dispensed into a 25 mL graduated cylinder and the weight of test article recorded. The powder mass in the measuring vessel was determined and divided by the test article volume.

To determine tapped density, the graduated cylinder was tapped until the volume did not change. The final powder volume was recorded after tapping. Tapped density was calculated by dividing the initial mass by the final volume.

The compressibility index and Hausner ratio were calculated as described in USP <616>.

### Hardness

Tablet hardness was determined with a Varian VK200 Tablet Hardness Tester in accordance with USP <1062>.

### Friability

A friability test apparatus (Sotax FT2) was used for friability testing by tumbling 10 tablets for 4 minutes at 25 rpm (100 revolutions) in accordance with USP <1216>.

### Compression Profile

Compression profiles were generated on tablets prepared using a Globe Pharma manual tablet compaction machine model MTCM-1 with a 5/16-inch flat upper and lower TSM-B punch and die.

### X-Ray Powder Diffraction

X-ray powder diffraction (XRPD) was performed using a Rigaku-Miniflex 6G benchtop x-ray diffractometer and 2 mm deep aluminum cup. The sample was pressed into the cup well using a glass slide. The x-ray source was operated at 40 kV and 15 mA. Sample data was collected using a run speed of 10 degree/minute for elraglusib and 2.5 degree/min for ASD samples with a 0.02° step size from 3-40°. Sample spinning was turned on for all measurements.

### Scanning Electron Microscopy

Scanning electron microscopy (SEM) images were captured using a Phenom Pro in 10 kV high resolution mode. Carbon paper was used as the conductive substrate. A plastic spatula was rolled over a <100 mg sample then carefully rolled over double-sided adhesive carbon paper on top of the stage to prepare the sample. Three left-handed 45° rotations of the sample holder were used to position the sample approximately 2 mm below the top of the stage for imaging.

### Differential Scanning Calorimetry

A modulated differential scanning calorimeter was used in conjunction with a controlled humidity chamber at <5% relative humidity (RH) to test the glass transition temperature. Samples were prepared in differential scanning calorimetry (DSC) pans and lids. Approximately 5-10 mg of sample was weighed into each pan which was placed into the appropriate equilibration chamber for at least 10 hours. Samples were removed from the chamber and the lids were crimped as appropriate based on study design. Samples were reweighed and placed on the DSC autosampler. Samples were then run with the method parameters described in Table 6-12.

TABLE 6-12
DSC method parameters

| Method Parameter | Target |
|---|---|
| Pan | Tzero w/ hole |
| Tstart (° C.) | −20 |
| Tend (° C.) | 180 |
| Scan rate (C./min) | 2.5 |
| Modulated (yes/no) | Yes |
| Modulation Period (sec) | 60 |
| Modulation Amplitude (° C.) | 1.5 |
| Drying Temp (° C.) | 40 |
| Drying Time (min) | 30 |

### Solubility of Elraglusib in Acetone:Water and in Acetone

The solubility of elraglusib API with and without polymers of interest was evaluated in acetone. Elraglusib was soluble up to 4% w/w; however, it was observed to crystallize at room temperature when left at static conditions.

Tests performed with 4% w/w elraglusib, and 4% w/w polymer indicate that HPMCAS-MG, Eudragit® L100, and Eastman cellulose acetate phthalate (CAP) were feasible for preliminary spray drying at a solids loading of 8% w/w, if the solutions were used immediately. The HPMCAS-MG and Eastman CAP samples were visually observed to contain some undissolved solids, which are commonly observed for these polymer types, due to the presence of residual levels of insoluble high molecular weight fractions of polymer.

Prior to ASD prototype manufacture additional solvent and polymer combinations were evaluated to determine which mixtures should undergo further development, and range-find the appropriate elraglusib and polymer loadings that would result in suitable solution stability for a reasonable preliminary spray solution hold-time (NLT 24 h). Samples were prepared according to Table 6-13.

TABLE 6-13

| | | | | | | | Total |
|---|---|---|---|---|---|---|---|
| | | API | Polymer | Solvent | | | Solids |
| | | Amount | Amount | Amount | API Concentration | | Loading |
| Sample Description | | (g) | (g) | (g) | (mg/mL) | (% w/w) | (% w/w) |
| 1 | Acetone | 1.0042 | 0.0 | 23.95 | 33 | 4.02 | 4.02 |
| 2 | Tetrahydrofuran | 1.0058 | 0.0 | 24.03 | 37 | 4.02 | 4.02 |
| 3 | 95:5 Acetone:Water | 1.0045 | 0.0 | 24.00 | 33 | 4.02 | 4.02 |
| 4 | Acetone with | 1.0023 | 1.0090 | 23.27 | 34 | 3.96 | 7.96 |
| | HPMCAS- MG | | | | | | |
| 5 | Acetone with | 0.0 | 1.0087 | 23.94 | NA | NA | 4.04 |
| | HPMCAS- MG | | | | | | |
| | Control | | | | | | |

The samples were stirred and observed visually over 24 hours with observations summarized in Table 6-14. At 2 hours precipitation of bright orange fines was observed in both acetone (Sample 1) and 95:5 acetone:water (Sample 3) while the elraglusib with HPMCAS-MG in acetone (Sample 4) took approximately 4 hours for precipitation to occur. The elraglusib in tetrahydrofuran (THF) remained soluble for over 24 hours and no crystallization occurred overnight when stored at 0° C.

TABLE 6-14
Polymer and elraglusib solvent solubility study visual observation results

| Sample | | Duration | | | | | |
|---|---|---|---|---|---|---|---|
| Description | | 30 min | 1 hour | 2 hours | 4 hours | 8 hours | 24 hours |
| 1 | Acetone | Translucent | Translucent | Precipitation | No change | No change | No change |
| | | solution | solution | of bright | | | |
| | | | | orange fines | | | |
| 2 | THF | Translucent | Translucent | Translucent | Translucent | Translucent | Translucent |
| | | solution | solution | solution | solution | solution | solution |
| 3 | 95:5 | Translucent | Translucent | Precipitation | No change | No change | No change |
| | Acetone:Water | solution | solution | of bright | | | |
| | | | | orange fines | | | |
| 4 | Acetone | Hazy | Hazy | Hazy but no | Precipitation | No change | No change |
| | with | | | significant | of bright | | |
| | HPMCAS- | | | precipitation | orange fines | | |
| | MG | | | | | | |
| 5 | Acetone | Hazy | Hazy | Hazy | Hazy | Hazy | Hazy |
| | with | | | | | | |
| | HPMCAS- | | | | | | |
| | MG | | | | | | |
| | Control | | | | | | |

Following the completion of the solubility study at 24 hours the precipitated mixtures were allowed to settle and an aliquot was taken and centrifuged. The supernatant was then diluted and tested by HPLC to determine the solubility of elraglusib as shown in Table 6-15.

TABLE 6-15
Polymer and elraglusib Solvent solubility
study elraglusib concentration by HPLC

| | Concentration at 24 hours (n = 4) | |
|---|---|---|
| Sample | (mg/mL) | (% w/w) |
| elraglusib in acetone | 13.08 | 1.67 |
| elraglusib in THF | 34.68 | 3.90 |
| elraglusib in 95:5 acetone:water | 16.12 | 2.06 |
| 1:1 elraglusib:HPMCAS-MG in | 13.95 | 1.78 |
| acetone | | |

The measured solubility of elraglusib in acetone was 13.08 mg/mL. The THF solution measured 34.68 mg/mL elraglusib, which nearly matches the theoretical concentration (i.e., 37 mg/mL) based on the dispensed weights of elraglusib and THF, which is consistent with product remaining in solution after 24 hours. The 1:1 elraglusib:HPMCAS-MG samples did take longer to precipitate solids, but the measured concentration is close to that of the acetone sample after 24 hours.

In addition to analyzing the supernatant of the solubility study samples, the precipitate was collected where applicable and analyzed via XRPD. The spectra indicated that a form conversion to the acetone solvate form of elraglusib occurred.

Additional studies were performed to determine if an increased water content would prevent formation of the solvate precipitate and more dilute solutions were tested for potential ASD processing. The sample details are summarized in Table 6-16.

TABLE 6-16
Elraglusib Acetone: Water solubility study sample summary

| | | API | Polymer | Solvent | | | Total Solids |
|---|---|---|---|---|---|---|---|
| Sample | | Amount | Amount | Amount | API Concentration | | Loading |
| Description | | (g) | (g) | (g) | (mg/mL) | (% w/w) | (% w/w) |
| 1 | 90:10 | 0.4985 | 0.0 | 12.006 | 33.5 | 3.99 | 3.99 |
| | acetone:water | | | | | | |
| 2 | 95:5 acetone:water | 0.4992 | 0.0 | 12.0075 | 33.2 | 3.99 | 3.99 |
| 3 | 95:5 acetone:water | 0.2499 | 0.0 | 12.003 | 16.6 | 2.04 | 2.04 |
| 4 | 95:5 acetone:water | 0.2251 | 0.0 | 12.003 | 15.0 | 1.84 | 1.84 |
| 5 | 95:5 acetone:water | 0.1875 | 0.0 | 12.0032 | 12.5 | 1.54 | 1.54 |

The samples were stirred and observed visually over the course of 24 hours with observations summarized in Table 6-17.

TABLE 6-17
Elraglusib and Acetone:water solubility study visual observation results

| Sample | | Duration | | | | | |
|---|---|---|---|---|---|---|---|
| Description | | 30 min | 1 hour | 2 hours | 3.5 hours | 4 hours | 21.5 hours |
| 1 | 90:10 | Translucent | Translucent | Translucent | Precipitation | No change | No change |
| | acetone:water | solution | solution | solution | of bright | | |
| | | | | | orange fines | | |
| 2 | 95:5 | Translucent | Translucent | Precipitation | No change | No change | No change |
| | acetone:water | solution | solution | of bright | | | |
| | | | | orange fines | | | |
| 3 | 95:5 | Translucent | No change | No change | No change | No change | No change |
| | acetone:water | solution | | | | | |
| 4 | 95:5 | Translucent | No change | No change | No change | No change | No change |
| | acetone:water | solution | | | | | |
| 5 | 95:5 | Translucent | No change | No change | No change | No change | No change |
| | acetone:water | solution | | | | | |

The 95:5 acetone:water and 90:10 acetone:water mixtures with ~4% w/w solids loadings both precipitated bright orange solids when agitated for 2 hours and 3.5 hours, respectively. Elraglusib at 1.5-2.0% w/w in 95:5 acetone:water remained soluble for over 24 hours. These results support spray-drying the 95:5 acetone:water solution with drug substance loading of not more than 2.0% w/w.

The 90:10 acetone water sample was sampled and centrifuged after 24 hours to determine concentration of elraglusib by HPLC as summarized in Table 6-18. The measured concentration aligned to the initial 95:5 acetone:water sample indicating that the additional water did not result in improved solubility.

TABLE 6-18
90:10 acetone:water solubility study
elraglusib concentration results

| | Concentration at 24 hours | |
|---|---|---|
| | (n = 4) | |
| Sample | (mg/mL) | (% w/w) |
| 4.0% w/w elraglusib in 95:5 acetone:water | 16.12 | 2.06 |
| 4.0% w/w elraglusib in 90:10 acetone:water | 15.7 | 2.00 |

Solubility of elraglusib in simulated intestinal fluid with varying levels of SIF powder was measured to support formulation development, facilitate interpretation of in vitro dissolution, and support potential modeling and interpretation of in vivo pharmacokinetic data. The obtained solubility data are shown in Table 6-19 and is generally aligned with data previously collected for elraglusib. As anticipated for a compound with few readily ionizable sites, equilibrium solubility was essentially independent of pH.

TABLE 6-19
Elraglusib solubility data in simulated intestinal fluid

| | | | Average Concentration | |
|---|---|---|---|---|
| | Initial | [NaTC] | (μg/mL) | |
| Media | pH | (mM) | 2 hour | 24 hour |
| 0.01N HCl | 2 | 0 | 2.1 | 1.4 |
| Blank FeSSIF | 5 | 0 | 2.4 | 2.6 |
| Blank FaSSIF | 6.5 | 0 | 2.5 | 2.6 |
| FaSSIF | 6.5 | 3 | 5.2 | 5.7 |
| 0.5% Simulated Intestinal | 6.5 | 6.7 | 6.0 | 4.7 |
| Fluid (SIF) | | | | |
| FeSSIF | 5 | 15 | 8.4 | 11.6 |
| 2% SIF | 6.5 | 26.8 | 17.0 | 17.3 |

### ASD Development

### Manufacture

Four elraglusib/polymer mixture lots were successfully spray dried with 4% w/w total solids loading and 90:10 acetone:water solvent. The lots used various polymers and were completed with no significant interruptions or parameters outside of the desired ranges. The spray-drying parameters for these lots is presented in Table 6-20-Table 6-23. A summary of the manufacturing parameters for these lots is provided in Table 6-24.

TABLE 6-20
Detailed spray drying parameters for 50%
elraglusib:HPMCAS-M ASD, lot 35044-040

| | | | | | Mass Lost | |
|---|---|---|---|---|---|---|
| | Rotameter | Inlet | Outlet | Condenser | from Spray | Spray |
| Time | Reading | Temp. | Temp. | Temp. | Solution | Rate |
| (hh:mm:ss) | (mm) | (° C.) | (° C.) | (° C.) | (g) | (g/min) |
| 00:00:00 | 30 | 64 | 43 | −19 | 0.00 | NA |
| 00:05:00 | 30 | 65 | 39 | −19 | 68.12 | 13.624 |
| 00:10:10 | 30 | 66 | 40 | −18 | 138.69 | 13.659 |
| 00:15:00 | 30 | 67 | 41 | −17 | 203.75 | 13.461 |
| 00:20:30 | 30 | 68 | 41 | −20 | 278.32 | 13.558 |
| 00:31:00 | 30 | 68 | 42 | −19 | 421.38 | 13.625 |
| 00:40:30 | 30 | 67 | 42 | −18 | 552.2 | 13.771 |
| 00:50:15 | 30 | 68 | 42 | −17 | 684.31 | 13.550 |
| 01:00:00 | 30 | 68 | 42 | −17 | 815.45 | 13.450 |
| 01:15:00 | 30 | 68 | 42 | −17 | 1014.68 | 13.282 |
| 01:25:00 | 30 | 68 | 42 | −19 | 1146.81 | 13.213 |
| 01:34:24 | 30 | 68 | 42 | −20 | NA | NA |
| Minimum | — | 64 | 39 | −20 | — | 13.213 |
| Maximum | — | 68 | 43 | −17 | — | 13.771 |
| Average | — | 67 | 42 | −18 | — | 13.493 |

TABLE 6-21
Detailed spray drying parameters for 50% elraglusib:HPMCAS-H, lot 35044-041

| | | | | | Mass Lost | |
|---|---|---|---|---|---|---|
| | | | | | from | |
| | Rotameter | Inlet | Outlet | Condenser | Spray | Spray |
| Time | Reading | Temp. | Temp | Temp. | Solution | Rate |
| (hh:mm:ss) | (mm) | (° C.) | (° C.) | (° C.) | (g) | (g/min) |
| 00:00:00 | 30 | 68 | 44 | −21 | 0.00 | NA |
| 00:05:00 | 30 | 68 | 43 | −20 | 65.93 | 13.186 |
| 00:10:00 | 30 | 68 | 42 | −17 | 127.45 | 12.304 |
| 00:20:00 | 30 | 68 | 42 | −19 | 260.62 | 13.317 |
| 00:30:00 | 30 | 68 | 42 | −20 | 398.41 | 13.123 |
| 00:40:00 | 30 | 68 | 42 | −21 | 532.45 | 13.404 |
| 00:50:10 | 30 | 68 | 42 | −18 | 669.32 | 13.463 |
| 01:00:15 | 30 | 68 | 42 | −21 | 803.81 | 13.338 |
| 01:10:00 | 30 | 68 | 42 | −21 | 937.98 | 12.78 |
| 01:20:00 | 30 | 69 | 42 | −21 | 1063.80 | 12.582 |
| 01:25:40 | 30 | 68 | 42 | −17 | 1140.44 | 13.525 |
| 01:30:00 | 30 | 68 | 41 | −17 | 1201.55 | 14.102 |
| 01:33:00 | 30 | 68 | 42 | −21 | NA | NA |
| Minimum | — | 68 | 41 | −21 | — | 12.304 |
| Maximum | — | 69 | 44 | −17 | — | 14.102 |
| Average | — | 68 | 42 | −19 | — | 13.193 |

TABLE 6-22
Detailed spray drying parameters for 50% elraglusib:EL-100*, lot 35044-042

| | | | | | Mass Lost | |
|---|---|---|---|---|---|---|
| | | | | | from | |
| | Rotameter | Inlet | Outlet | Condenser | Spray | Spray |
| Time | Reading | Temp. | Temp. | Temp. | Solution | Rate |
| (hh:mm:ss) | (mm) | (° C.) | (C) | (° C.) | (g) | (g/min) |
| 00:00:00 | 30 | 68 | 44 | −18 | 0.00 | NA |
| 00:05:00 | 30 | 68 | 42 | −20 | 66.16 | 13.232 |
| 00:10:00 | 30 | 68 | 41 | −19 | 131.36 | 13.040 |
| 00:20:00 | 30 | 68 | 41 | −20 | 260.85 | 12.949 |
| 00:32:30 | 30 | 69 | 42 | −21 | 425.58 | 13.178 |
| 00:40:00 | 30 | 70 | 42 | −18 | 526.04 | 13.395 |
| 00:50:50 | 30 | 69 | 42 | −21 | 670.10 | 13.298 |
| 01:01:40 | 30 | 69 | 42 | −17 | 811.86 | 13.086 |
| 01:10:00 | 30 | 69 | 42 | −18 | 923.5 | 13.397 |
| 01:21:00 | 30 | 69 | 41 | −21 | 1067.98 | 13.135 |
| 01:30:00 | 30 | 69 | 41 | −18 | 1189.25 | 13.474 |
| 01:32:28 | 30 | 70 | 42 | −19 | NA | NA |
| Minimum | — | 68 | 41 | −21 | — | 12.949 |
| Maximum | — | 70 | 44 | −17 | — | 13.474 |
| Average | — | 69 | 42 | −19 | — | 13.218 |

*“EL100” is Eudragit ® L100 polymer

TABLE 6-23
Detailed spray drying parameters for 50% elraglusib:CAP, lot 35044-043

| | | | | | Mass Lost | |
|---|---|---|---|---|---|---|
| | Rotameter | Inlet | Outlet | Condenser | from Spray | Spray |
| Time | Reading | Temp. | Temp. | Temp. | Solution | Rate |
| (hh:mm:ss) | (mm) | (° C.) | (° C.) | (° C.) | (g) | (g/min) |
| 00:00:00 | 30 | 68 | 44 | −18 | 0.00 | NA |
| 00:05:00 | 30 | 68 | 40 | −18 | 64.32 | 12.864 |
| 00:10:00 | 30 | 69 | 41 | −18 | 133.87 | 13.910 |
| 00:20:00 | 30 | 70 | 41 | −19 | 263.05 | 12.918 |
| 00:30:00 | 30 | 70 | 41 | −20 | 391.75 | 12.870 |
| 00:40:00 | 30 | 70 | 41 | −18 | 526.83 | 13.508 |
| 00:50:00 | 30 | 70 | 40 | −19 | 661.31 | 13.448 |
| 01:00:00 | 30 | 71 | 41 | −18 | 790.45 | 12.914 |
| 01:10:00 | 30 | 71 | 41 | −20 | 923.72 | 13.327 |
| 01:20:00 | 30 | 71 | 41 | −19 | 1056.00 | 13.228 |
| 01:30:00 | 30 | 71 | 41 | −18 | 1189.28 | 14.809 |
| 01:34:53 | 30 | 71 | 41 | −20 | NA | NA |
| Minimum | — | 68 | 40 | −20 | — | 12.864 |
| Maximum | — | 71 | 44 | −18 | — | 14.809 |
| Average | — | 70 | 41 | −19 | — | 13.380 |

TABLE 6-24
ASD manufacturing summary for 90:10 acetone:water and 4% w/w solids loading

| | | Outlet | Average | | | Total | |
|---|---|---|---|---|---|---|---|
| | | Temp. | Spray | Drying | Drying | Dry | Loss on |
| | | Range | Rate | Temp. | Time | Yield | Drying |
| ASD Description | Lot Number | (° C.) | (g/min) | (° C.) | (mm:ss) | (%) | (%) |
| 50% | 35044-040 | 39-42 | 13.5 | 40 | 24:00 | 65.7 | 1.6 |
| elraglusib:HPMCAS-M | | | | | | | |
| 50% | 35044-041 | 41-43 | 13.1 | 40 | 24:00 | 68.7 | 0.5 |
| elraglusib:HPMCAS-H | | | | | | | |
| 50% elraglusib:L-100 | 35044-042 | 41-42 | 13.2 | 40 | 25:10 | 66.1 | 1.2 |
| 50% elraglusib:CAP | 35044-043 | 40-41 | 13.4 | 40 | 46:19 | 63.5 | 1.7 |

Yields were greater than 60% for all lots, which was expected for the scale and equipment used.

The ASD prototypes were tested for assay and related substances as shown in Table 6-25. Assay values were within the expected range.

TABLE 6-25
ASD prototypes assay and related substances

| | elraglusib | RRT 1.21 Related |
|---|---|---|
| Sample | (%) | Substance ^{(a)} |
| 50% elraglusib:HPMCAS-M | 99.7% | 0.97% |
| 50% elraglusib:HPMCAS-H | 99.6% | 0.96% |
| 50% elraglusib:L-100 | 99.2% | 0.95% |
| 50% elraglusib:CAP | 100.5% | 0.96% |

^{(a)} API lot NB-elraglusib(NG)-A-1 used in formulation has 0.75% RRT 1.21 related substance. Per Sponsor, RRT 1.21 related substance is the elraglusib anhydride analog.

The ASD prototypes were also characterized via SEM and XRPD to assess ASD particle morphology and whether any crystallization could be detected as shown in FIG. 46 and FIG. 47.

It was observed with the SEM that all ASD lots had a mixed morphology of intact, collapsed, and shattered spheres. There was no surface crystallinity observed and particle sizes appeared to be fairly consistent across the lots. See FIG. 46.

The elraglusib ASD lots showed an amorphous halo with no detectable crystalline peaks. The observed difference in the amorphous halo is expected due to inclusion of different polymers. The aluminum background holder used for the ASD material resulted in the observed peak at 37° 2-theta. See FIG. 47.

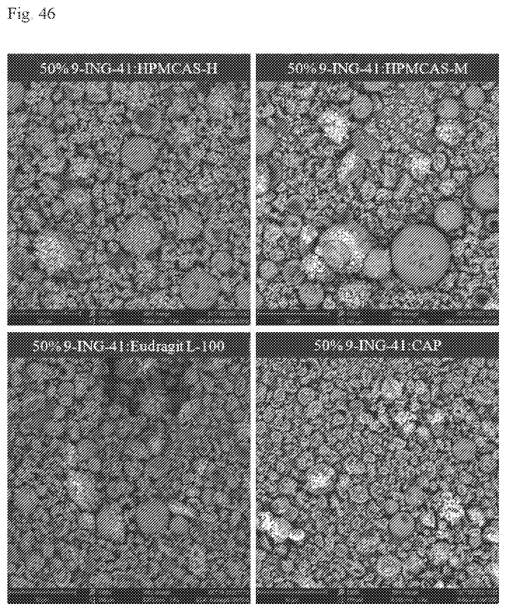

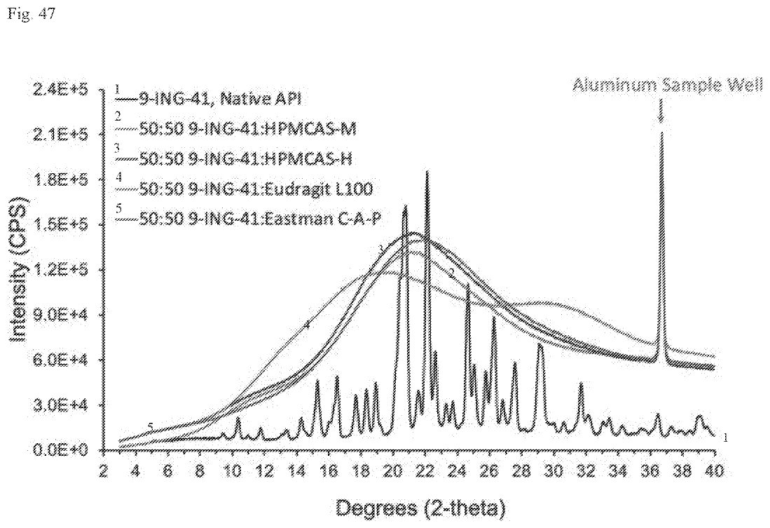

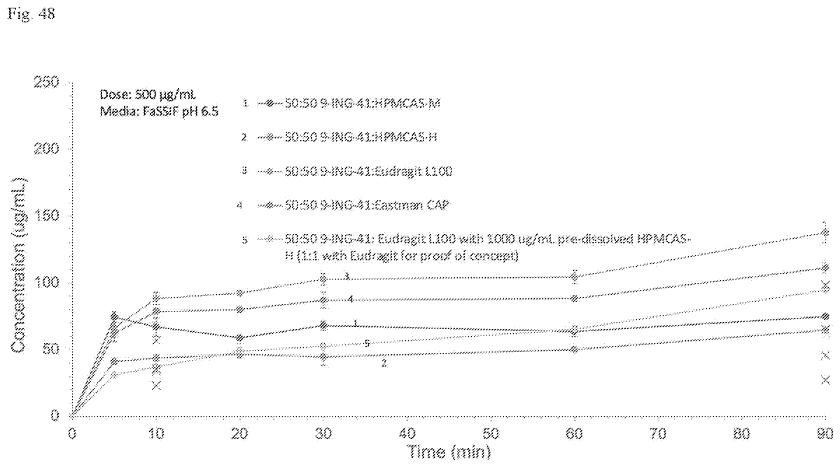

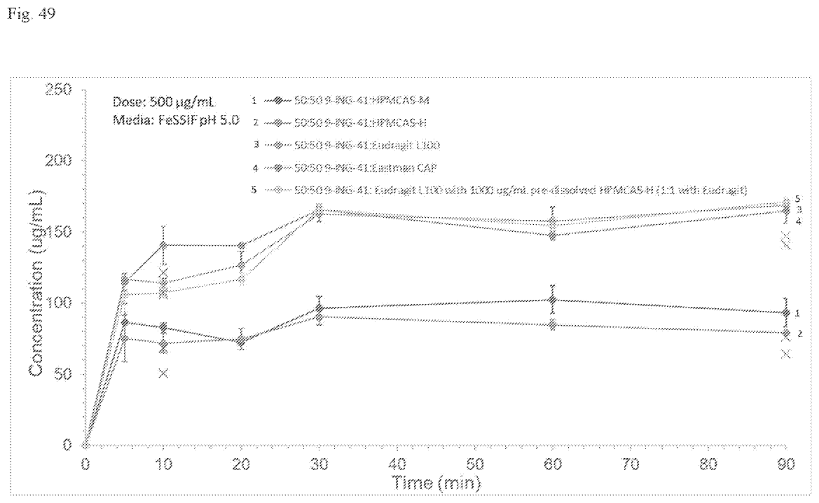

Non-sink biorelevant dissolution of the ASD prototypes was evaluated in both FaSSIF pH 6.5 (FIG. 48) and FeSSIF pH 5.0 (FIG. 49).

The ASD prototypes showed approximately 10 times greater solubility than the measured solubility of crystalline elraglusib in FaSSIF (5.7 μg/mL). The HPCMAS-M ASD sample exhibited the fastest dissolution rate; however, the Eudragit® L100 and Eastman CAP ASDs sustained a higher concentration of the drug throughout the test. It was also noted that the 50:50 elraglusib:EL100 with 500 μg/mL pre-dissolved HPMCAS-H displayed poor wettability which may have contributed to the sub-optimal performance. Full concentration data is provided in Table 6-26 with “free” indicating the concentration of drug that remains after filtration of >200 nm in size colloidal species.

TABLE 6-26
Dissolution concentration versus time profiles for Elraglusib ASDs in FaSSIF pH 6.5

| | [Elraglusib], μg/mL at Timepoints (min)= | | | | | | | | |
|---|---|---|---|---|---|---|---|---|---|
| | | | | 10 - | | | | | 90 - |
| Sample | 0 | 5 | 10 | Free | 20 | 30 | 60 | 90 | Free |
| 50:50 elraglusib:HPMCAS-M | 0 | 74.4 | 66.9 | 35.4 | 58.8 | 68.0 | 63.6 | 74.8 | 45.4 |
| 50:50 elraglusib:HPMCAS-H | 0 | 41.1 | 43.5 | 23.2 | 46.4 | 44.5 | 50.0 | 64.5 | 27.0 |
| 50:50 elraglusib:EL100 | 0 | 66.3 | 88.2 | 68.4 | 92.3 | 102.8 | 104.3 | 137.6 | 98.4 |
| 50:50 elraglusib:Eastman CAP | 0 | 62.4 | 78.6 | 57.3 | 80.1 | 87.1 | 88.2 | 111.2 | 65.4 |
| 50:50 elraglusib:EL100 with | 0 | 30.9 | 37.1 | 33.6 | 48.8 | 52.6 | 65.3 | 94.7 | 61.9 |
| 1000 μg/mL pre-dissolved | | | | | | | | | |
| HPMCAS- H (1:1 with | | | | | | | | | |
| Eudragit ®) | | | | | | | | | |

The elraglusib:CAP and elraglusib:EL 100 ASD prototypes displayed similar performance for the solubilized drug and free drug species. The HPMCAS-based formulations had lower total solubilized drug and free drug relative to the elraglusib:EL100 and elraglusib:CAP dispersions. Also, adding HPMCAS-H to the elraglusib:EL 100 ASD did not result in a significant difference in dissolution relative to the elraglusib:EL100 ASD alone. Typically, HPMCAS-H can be added as a concentration sustainment polymer. Related data is provided in Table 6-27 with “free” indicating the concentration of drug that remains after filtration of >200 nm in size colloidal species.

TABLE 6-27
Dissolution concentration versus time profiles for Elraglusib ASDs in FeSSIF pH 5.0

| | [Elraglusib], μg/mL at Timepoints (min)= | | | | | | | | |
|---|---|---|---|---|---|---|---|---|---|
| | | | | 10 - | | | | | 90 - |
| Sample | 0 | 5 | 10 | Free | 20 | 30 | 60 | 90 | Free |
| 50:50 elraglusib:HPMCAS-M | 0 | 86.6 | 82.8 | 68.3 | 72.5 | 96.5 | 102.5 | 93.3 | 75.8 |
| 50:50 elraglusib:HPMCAS-H | 0 | 75.2 | 71.8 | 50.6 | 74.9 | 90.7 | 84.8 | 79.4 | 64.3 |
| 50:50 elraglusib:EL100 | 0 | 116.9 | 114.2 | 107.2 | 126.9 | 162.6 | 157.5 | 169.0 | 147.2 |
| 50:50 elraglusib:Eastman | 0 | 114.9 | 140.6 | 121.5 | 140.4 | 165.6 | 147.5 | 164.9 | 140.9 |
| CAP | | | | | | | | | |
| 50:50 elraglusib:EL100 | 0 | 106.2 | 107.5 | 105.8 | 116.7 | 165.7 | 154.3 | 171.2 | 147.0 |
| with 500 μg/mL pre- | | | | | | | | | |
| dissolved HPMCAS-H | | | | | | | | | |
| (1:1 with Eudragit ®) | | | | | | | | | |

A final characterization was performed to evaluate the glass transition temperature (Tg) by DSC of the ASD prototypes under dry conditions. Results of the DSC study are presented in Table 6-28.

TABLE 6-28
ASD prototype DSC results

| Material | Tg (° C.) |
|---|---|
| 50:50 elraglusib:HPMCAS-M | 95 |
| 50:50 elraglusib:HPMCAS-H | 95 |
| 50:50 elraglusib:EL100 | 106 |
| 50:50 elraglusib:Eastman CAP | 114 |

All ASD prototypes were observed to have a single Tg event, indicative of a single amorphous phase and a uniform amorphous dispersion. Dry ASD Tg values ranged from 95-114° C. amongst the different ASD formulations with elraglusib:CAP ASD having the highest Tg and the elraglusib:EL100 ASD having Tg=106° C. Higher Tg values tend to be indicative of reduced risk for physical state changes on stability.

### Stability Testing

After three months of exposure to 40° C./75% RH packaged with desiccant, the elraglusib ASDs showed no significant evidence of changes to physical appearance, physical state, or chemical state. There has not been a decrease in performance when evaluated in the non-sink dissolution conditions.

Assay of the ASD formulations trended approximately 1% lower than previous timepoints. In the CAP based ASD, no change in phthalic acid content has been observed after 3 months at 40° C./75% RH closed with desiccant.

### Conclusion

Manufacturing of four ASD prototypes was successfully completed with overall yields ranging from 63.5-68.7% and assay values ranging from 99.2-100.5%. XRPD confirmed the amorphous nature of the final product and SEM imaging showed mixed morphology typical of an ASD process. All ASD prototypes showed significant solubility enhancement in SIF relative to crystalline elraglusib. The elraglusib:CAP and elraglusib:EL100 ASD prototypes were selected for further development into tablet prototypes.

### Granulation and Blending

The elraglusib:CAP and elraglusib:EL100 ASDs were granulated and blended according to the formulations presented in Table 6-29.

TABLE 6-29
Elraglusib ASD granule formulations (35044-052)

| | | | |
|---|---|---|---|
| | | | Eudragit ® L100 |
| | | Eastman CAP ASD- | ASD- Based |
| | | Based Formulation, | Formulation, |
| Ingredient | Brand Name/Grade | Material (% w/w) | Material (% w/w) |

Intragranular

| | | | |
|---|---|---|---|
| 50:50 elraglusib:CAP ASD | Lot: 35044-043 | 41.67 | NA |
| 50:50 elraglusib:EL100 | Lot: 35044-042 | NA | 41.67 |
| ASD | | | |
| MCC | PH 102 | 27.33 | 27.33 |
| Mannitol | Pearlitol 100SD | 25.00 | 25.00 |
| CCS | Ac-Di-Sol | 2.00 | 2.00 |
| Magnesium stearate | Ligamed MF-2-K | 1.00 | 1.00 |

Extragranular

| | | | |
|---|---|---|---|
| CCS | Ac-Di-Sol | 2.00 | 2.00 |
| Silicon Dioxide | Aerosil 200 Pharma | 0.50 | 0.50 |
| Magnesium stearate | Ligamed MF-2-K | 0.50 | 0.50 |
| Total | | 100.00 | 100.00 |

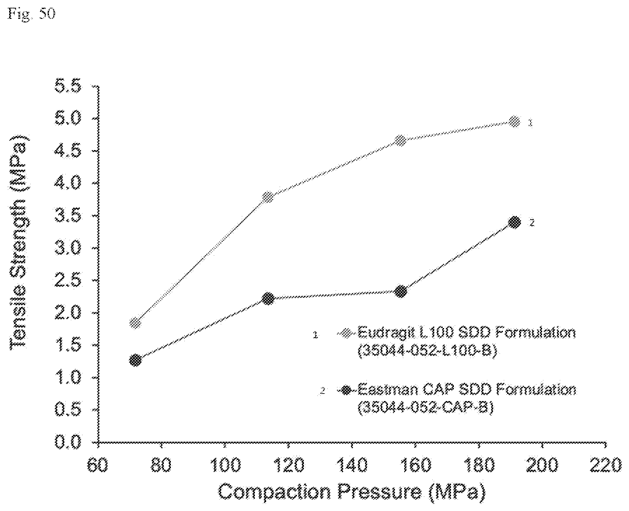

The final blends for the elraglusib:CAP and elraglusib:EL100 ASDs were weighed and characterized. A final blend yield of 96.3% was obtained for the Eastman CAP formulation and 96.0% for the Eudragit® L100 formulation. Both blending yields are within the expected range. Compression profiles were measured using 8 mm round, flat-faced tooling for both formulations with data presented in Table 6-30 and Table 6-31. The elraglusib:EL100 formulation showed significantly increased compressibility compared to the elraglusib:CAP formulation as shown in FIG. 50, which is likely inherent to the polymer structure.

TABLE 6-30
Elraglusib:CAP ASD final blend formulation compression profile, lot 35044-052- CAP-B

| | | | | | | | Compaction | Tensile |
|---|---|---|---|---|---|---|---|---|
| Compaction | Compaction | Weight | Breaking Force | | Thickness | Diameter | Pressure | Strength |
| Pressure (psi) | Force (kN) | (mg) | (kP) | (N) | (mm) | (mm) | (MPa) | (MPa) |
| 1200 | 4.732 | 200.4 | 5.5 | 53.9366 | 3.41 | 7.9375 | 95.63 | 1.27 |
| 1900 | 7.492 | 200.1 | 9.0 | 88.2599 | 3.19 | 7.9375 | 151.41 | 2.22 |
| 2600 | 10.253 | 201.5 | 9.2 | 90.2212 | 3.10 | 7.9375 | 207.20 | 2.33 |
| 3200 | 12.619 | 200.7 | 13.1 | 128.4671 | 3.03 | 7.9375 | 255.01 | 3.40 |

TABLE 6-31
Elraglusib:EL100 ASD Final blend formulation compression profile, lot 35044-052- L100-B

| Compaction | | | | | | | Compaction | Tensile |
|---|---|---|---|---|---|---|---|---|
| Pressure | Compaction | Weight | Breaking Force | | Thickness | Diameter | Pressure | Strength |
| (psi) | Force (kN) | (mg) | (kP) | (N) | (mm) | (mm) | (MPa) | (MPa) |
| 1200 | 4.732 | 201.9 | 8.1 | 79.4339 | 3.46 | 7.9375 | 95.63 | 1.84 |
| 1900 | 7.492 | 201.0 | 15.4 | 151.0224 | 3.20 | 7.9375 | 151.41 | 3.79 |
| 2600 | 10.253 | 202.4 | 18.6 | 182.4037 | 3.14 | 7.9375 | 207.20 | 4.66 |
| 3200 | 12.619 | 200.2 | 19.2 | 188.2877 | 3.05 | 7.9375 | 255.01 | 4.95 |

The elraglusib:EL 100 bulk blend was also tested for bulk and tapped density with 3 replicates as shown in Table 6-32. The CI and HR values indicate that the final blend should have good flow properties.

TABLE 6-32
ASD final blend bulk and tapped density

| | Bulk | Tap | | Hausner |
|---|---|---|---|---|
| | Density | Density | Compressibility | Ratio |
| Sample | (g/mL) | (g/mL) | Index (CI) | (HR) |
| Elraglusib:EL100 Final | 0.53 | 0.59 | 11 | 1.12 |
| Blend | | | | |
| 35044-052-L100-B | | | | |

### Tableting

Tablets were manufactured using the MCTM-1 tablet press according to the previously defined tablet formulation to compare performance of the formulations. Compression of the unit dose targeting at compression pressure of 4,000 psi was performed using a 9.5×21.0 mm plain-faced modified oval tooling.

Physical characterization testing was performed on the tablet prototypes. Tablet characterization results are shown in Table 6-33.

TABLE 6-33
ASD prototype tablet characterization data

| | | Weight | | | Tensile | Friability |
|---|---|---|---|---|---|---|
| | Compression | (n = 20) | Thickness | Hardness | Strength | (n = 10) |
| Lot | Force (psi) | (mg) | (mm) | (kP) | (MPa) | (%) |
| elraglusib:CAP | 4000 | 1200.3 | 7.15 | 19.2 | 1.40 | 0.18 |
| 35044-052-CAP-B | | | | | | |
| Elraglusib:EL100 | 4000 | 1202.2 | 7.18 | 26.4 | 1.90 | 0.07 |
| 35044-052-L100-B | | | | | | |

Visually, the core tablets compressed with 9.5×21.0 mm tooling exhibited acceptable sideband and cup depth (approximately 24% of the total thickness) as shown in FIG. 51.

At equal compaction pressures (4000 psi) the elraglusib:EL100 formulation was approximately 1.9 MPa. The elraglusib:CAP formulation had an approximate tensile strength of 1.4 MPa, but had higher friability, which may result in issues during coating.

Tablets were also evaluated for appearance, water content, assay, and related substances as summarized in Table 6-34.

TABLE 6-34
Tablet characterization for elraglusib:CAP and elraglusib:EL 100 formulations

| | | | | | IRRT 1.21 |
|---|---|---|---|---|---|
| | | Water | | elraglusib | Related |
| | | Content (%) | | Assay (% | Substance^{(a)} |
| Lot | Appearance | Replicates | Average | w/w) | (Area %) |
| Elraglusib:CAP | Large dark orange | 1.8522 | 1.8 | 97.2 | 0.84 |
| 35044-052-CAP-B | capsule shaped | 1.8236 | | | |
| | tablets with white | 1.8347 | | | |
| | spots | | | | |
| Elraglusib:EL100 | Large dark orange | 2.0976 | 2.1 | 96.8 | 0.84 |
| 35044-052-L100-B | capsule shaped | 2.0444 | | | |
| | tablets | 2.0437 | | | |

^{(a)}API lot NB-elraglusib(NG)-A-I used in formulation has 0.75% RRT 1.21 related substance.
Sponsor-provided information indicates that RRT 1.21 related substance is the elraglusib anhydride analog.

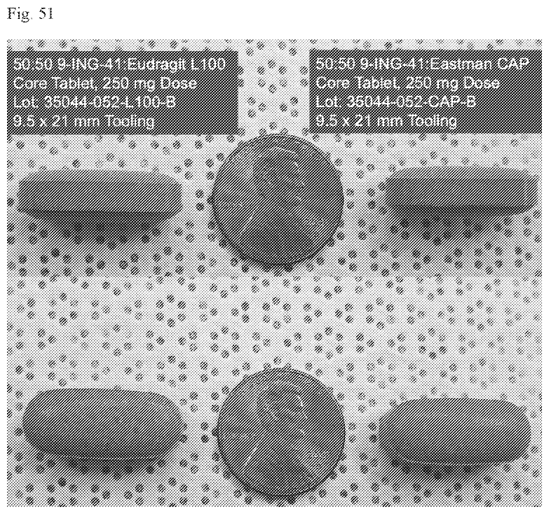

Dissolution was measured on both ASD formulation lots and compared to previous elraglusib:CAP prototypes as shown in FIG. 52. The tablet elraglusib:CAP prototype (35044-052-CAP-B) was comparable to previous data generated for the large-scale CAP tablet lot 35044-033. However, the elraglusib:EL100 tablet prototype (35044-052-L100-B) had a slower dissolution release than the CAP tablet prototypes. By T=15 mins, the CAP prototype is >80% dissolved versus the slower dissolving elraglusib:EL 100 tablet prototype (>80% at T=60 minutes). The slower release profile observed for the elraglusib:EL100 tablet prototype could be related to the higher tensile strength (1.9 MPa) of these tablets relative to the tensile strength (1.4 MPa) for the elraglusib:CAP tablet prototypes.

### Conclusions

The solubility of elraglusib in commercially viable spray-drying solvents was successfully evaluated with and without the inclusion of various polymers. Switching solvents from THF to acetone reduced drug substance solubility, however, elraglusib was still soluble in the presence of dispersion polymers at commercially relevant spray solution weight-by-weight percents in acetone:water mixtures.

Micelle partition coefficients were experimentally determined for elraglusib to inform in vitro non-sink biorelevant dissolution testing, and in vivo performance. This pre-formulation knowledge was leveraged to manufacture spray-dried dispersions of elraglusib for subsequent characterization.

Two ASD prototypes were identified as leads-50:50 elraglusib:CAP and 50:50 elraglusib:EL100. These were successfully dry granulated, final blended, and pressed into 250 mg strength tablets. The tablets were characterized. The elraglusib:CAP ASD tablets demonstrated performance consistent with previous tablet prototypes. The elraglusib:CAP tablets demonstrated more rapid dissolution than the elraglusib:EL 100 tablet prototypes, which may be related to the reduced tensile strength of the elraglusib:CAP tablets compared to the elraglusib:EL100 tablets.

### Stability Testing

This protocol documents the stability study of packaged Elraglusib ASD Tablets non-GMP lots: 35044-052-CAP-B (250 mg) and 35044-052-L100-B (250 mg). This stability study will evaluate changes, if any, to product quality over time under the following conditions: storage (25° C./60% RH) and accelerated (40° C./75% RH).

Elraglusib ASD Tablets, 250 mg, were packaged in 5-count configurations. The packaging for the 5-count configuration was in 60 cc, white HDPE plastic round packer bottle (33/400) closed with a 33 mm white child resistant polypropylene cap with activated heat induction seal foil inner foam liner and 1-gram desiccant sachet. The packaged active tablet product was stored at 25° C./60% RH, and 40° C./75% RH.

3 Month tablet samples were pulled and analyzed for appearance, assay and related substances, and sink dissolution.

At 3 months, appearance remained unchanged for both formulations and all conditions.

Sink dissolution at the 3 month timepoint was slower for both formulations relative to the initial dissolution profile when compared as normalized to the final percent dissolved. The normalized results are presented based on the initial and 1 month timepoints having percent dissolved greater than 100%.

Sink dissolution for the 3 month timepoint was analyzed using HPLC rather than the previously used in line UV analysis. Additionally, the samples were analyzed using a standard prepared in 6% CTAB and a separate standard prepared in ACN. This was performed to evaluate the hypothesis that the API standard in CTAB was not fully dissolved and at least partially responsible for the higher than 100% dissolved results that were collected at initial and 1 month timepoints. The CTAB standard was observed to have a low response relative to the ACN standard and to precipitate over time. During the 3 month analysis it was monitored closely and injected soon after preparation to ensure it was dissolved during analysis. The data presented here is based on the CTAB standard, which closely matched the ACN standard and did not result in greater than 100% dissolved like was observed in previous timepoints.

The 3 month assay and related substances results for the Eastman CAP tablets were comparable to initial results. The 3 month Eudragit® L100 tablets results were aligned with the initial result (97%).

The impurity profile of both tablet samples at 3 months stored at 25 C/60% RH and 40 C/75% RH was in line with previous data generated at T=0.

The sample preparation for the tablets was updated to include a 30 min shake in water to facilitate complete disintegration of the tablets. The samples were also placed on a shaker and sonicated after diluent was added to approximately 75% fill. These updates to the sample preparation appear to be more robust for stability samples and are recommended for future testing.

### Tablet Formulation Optimization

### Prototype Tablet Manufacture

Prototype tablets were manufactured based on the formulation compositions included in Table 6-35. The previous formulation is included for reference. The goal of the tablet optimization was to minimize tablet weight while increasing processing robustness. Two composition tables are included for each prototype, the first is in wt % and the second is in mg/tablet.

TABLE 6-35

| | Formulation Optimization | | | | Formulation Optimization | | | |
|---|---|---|---|---|---|---|---|---|
| | Common | Prototype | Prototype | Prototype | Common | Prototype | Prototype | Prototype |
| | Blend | 1 | 2 | 3 | Blend | 1 | 2 | 3 |
| Component | (wt. %) | (wt. %) | (wt. %) | (wt. %) | (mg) | (mg) | (mg) | (mg) |
| Dose (mg) | 250 | 250 | 250 | 250 | 250 | 250 | 250 | 250 |
| Intragranular (%) | | | | | Intragranular (mg) | | | |
| 50% ELRA:CAP | 41.7 | | | | 500.0 | | | |
| 50% | | 50.0 | 55.6 | 58.8 | | 500.0 | 500.0 | 500.0 |
| ELRA:Eudragit ® | | | | | | | | |
| L100 | | | | | | | | |
| MCC (Avicel | 27.33 | 21.5 | 24.9 | 20.8 | 328.0 | 215.0 | 224.1 | 176.8 |
| Ph-102) | | | | | | | | |
| Mannitol | 25 | 21.5 | 12.5 | 10.4 | 300.0 | 215.0 | 112.5 | 88.4 |
| (Perlitol 100SD) | | | | | | | | |
| Ac-Di-Sol | 2 | 3 | 3 | 5 | 24.0 | 30.0 | 27.0 | 42.5 |
| Syloid 244 FP | | 0.5 | 0.5 | 0.5 | | 5.0 | 4.5 | 4.3 |
| Magnesium | 1 | 0.5 | 0.5 | 0.5 | 12.0 | 5.0 | 4.5 | 4.3 |
| Stearate | | | | | | | | |
| Intragranular | 97.0 | 97.0 | 97.0 | 96.0 | 1164.0 | 970.0 | 872.6 | 816.2 |
| Total | | | | | | | | |
| Extragranular (%) | | | | | Extragranular (mg) | | | |
| MCC (PH-102) | 2 | | | | 24.0 | | | |
| Ac-Di-Sol | | 2 | 2 | 3 | | 20.0 | 18.0 | 25.5 |
| Cabosil | 0.5 | | | | 6.0 | | | |
| Syloid 244 FP | | 0.5 | 0.5 | 0.5 | | 5.0 | 4.5 | 4.3 |
| Magnesium | 0.5 | 0.5 | 0.5 | 0.5 | 6.0 | 5.0 | 4.5 | 4.3 |
| Stearate | | | | | | | | |
| Total | 100.0 | 100.0 | 100.0 | 100.0 | 1200.0 | 1000.0 | 899.6 | 850.2 |
| Tablet Weight | 1200 | 1000 | 900 | 850 | 1200 | 1000 | 900 | 850 |

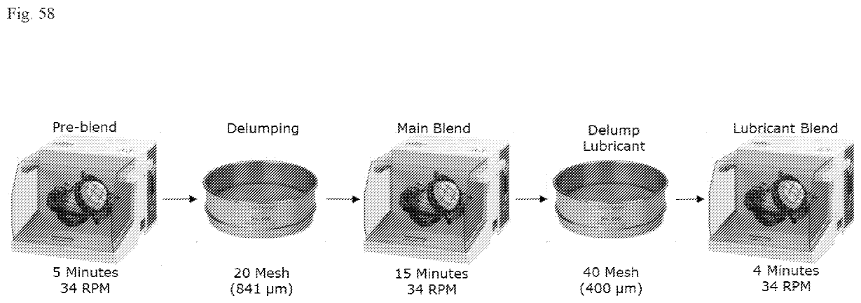

A Turbula T2 F blender was used to facilitate a blend-delump-blend procedure for the pre-granulated blend. This process is depicted in FIG. 58 with additional details.

### Slugging and Milling

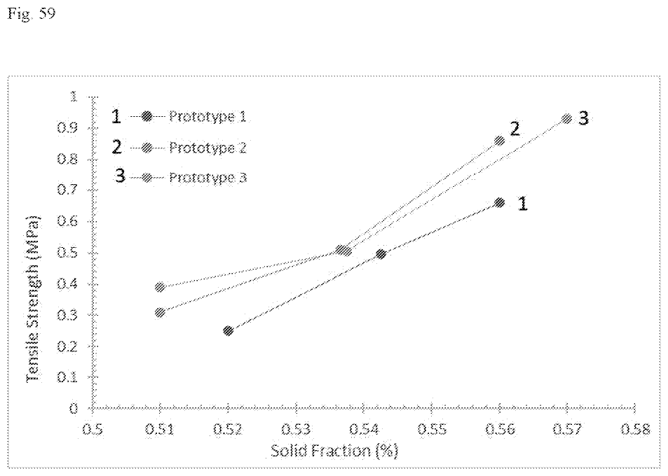

Compaction profiles were generated on the MTCM-1 hydraulic single station press with 0.9735″ round flat faced tooling. A summary of this data is presented in FIG. 59. All formulations had very good compactability at reasonable pressures.

Slugging was performed on an MTCM-1 hydraulic single station press with 0.9735″ round flat faced tooling. Slugs were manufactured targeting a 0.5 MPa tensile strength. Summary data is included in Table 6-36.

TABLE 6-36

| | Average Tensile | Average Solid |
|---|---|---|
| Formulation | Strength (MPa) | Fraction (%) |
| Prototype 1 | 0.49 | 0.54 |
| Prototype 2 | 0.51 | 0.54 |
| Prototype 3 | 0.51 | 0.54 |

Slugs were milled using the small scale Gerteis mill with a 1.00 mm square wire mesh screen and a 1.0 mm gap. Representative images of the granules show there is no evidence of extruding material through the screen that would result in high aspect ratio granules.

### Final Blend

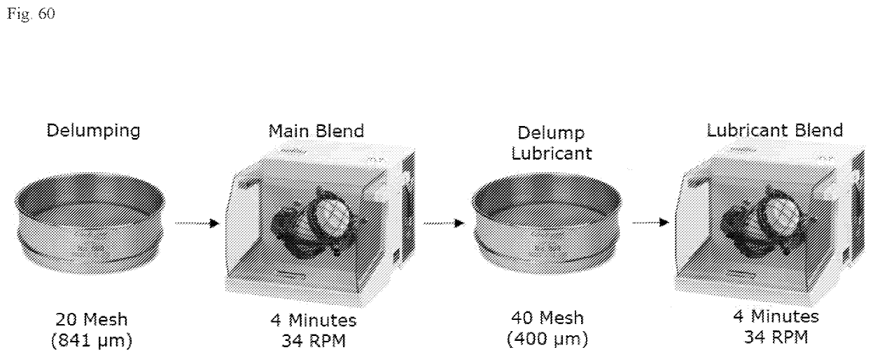

Final blends were prepared on the Turbula T2 F Blender with additional details included in FIG. 60.

### Compression

Tablets were compressed on an MTCM-1 hydraulic tablet press with 9.5×21.5 mm modified oval tooling (1000 mg) and 8.5×19.0 mm modified oval tooling (900 and 850 mg). The tablets were manufactured targeting a tensile strength of approximately 2.0 MPa to ensure robust tablet integrity for potential future coating activities. Summary data from the tablet manufactures can be found below in Table 6-37.

TABLE 6-37

| | | Average Tensile | Average Solid |
|---|---|---|---|
| Formulation | Hardness (kP) | Strength (MPa) | Fraction (%) |
| Prototype 1 | 25 | 2.09 | 0.71 |
| Prototype 2 | 28 | 2.05 | 0.73 |
| Prototype 3 | 27 | 2.07 | 0.72 |

Disintegration of the prototype tablet formulations was performed in 0.01 N HCl pH 2.0. Summary data is included below in Table 6-38 with all formulation disintegrating in less than 10 minutes.

TABLE 6-38

| | Disintegration | Individual Disintegration |
|---|---|---|
| Formulation | Time (Min:Sec) | Time (Min:Sec) |
| Prototype 1* | 6:49 | 5:38, 5:54, 8:56 |
| Prototype 2 | 7:41 | 7:10, 7:32, 8:22 |
| Prototype 3 | 8:05 | 7:24, 7:42, 9:08 |

*Due to the large dimensions of the tablets, inconsistent tablet agitation was observed during the disintegration test.

There was an observation that for the prototype 1 tablets, the tablet dimensions were relatively large compared to the tubes used for disintegration. This likely caused variability in the end point determination by the disintegration apparatus and higher than expected variability in the results.

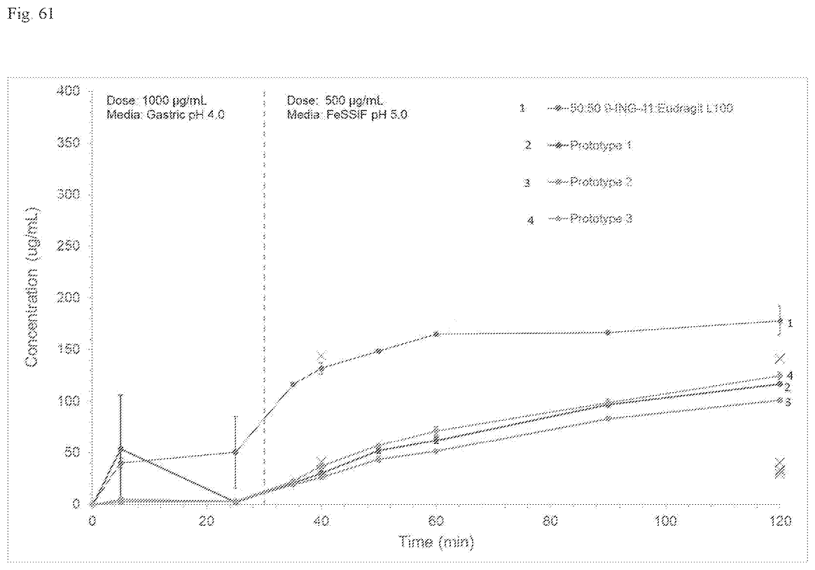

Non-sink dissolution was performed on the three tablet formulations relative to the ingoing ASD formulation (Conditions: USP Type II, IL Vessels, large paddles, 50 RPM, pH 4.0 HCl 1000 μg/mL to pH 5.0 FeSSIF 500 μg/mL). The tablets were significantly slower than the ASD as shown in FIG. 61 and Table 6-39.

TABLE 6-39

| | | | AUC-IB35- | | C40 | | C120 |
|---|---|---|---|---|---|---|---|
| | Cmax-G₂₋₂₅ | Cmax-IB₃₅₋₁₂₀ | 120 | C40 | Ultra | C120 | Ultra |
| Formulation | (μg/mL) | (μg/mL) | (μg/mL*min) | (μg/mL) | (μg/mL) | (μg/mL) | (μg/mL) |
| 50:50 Elra:EL100 | 51 | 178 | 13751 | 132 | 144 | 178 | 142 |
| Prototype 1 | 54 | 117 | 6718 | 30 | 33 | 117 | 33 |
| Prototype 2 | 3 | 101 | 5752 | 27 | 29 | 101 | 30 |
| Prototype 3 | 5 | 125 | 7200 | 37 | 41 | 125 | 41 |

It was noted during the dissolution experiment that coning was present for all formulations and there was material that was not being agitated.

### Drug Product Performance Investigation

Based on slow dissolution of the prototype 1-3 tablets relative to the ASD formulation, a second round of dissolution testing was performed to better understand the potential sources for slow dissolution in the non-sink biorelevant test.

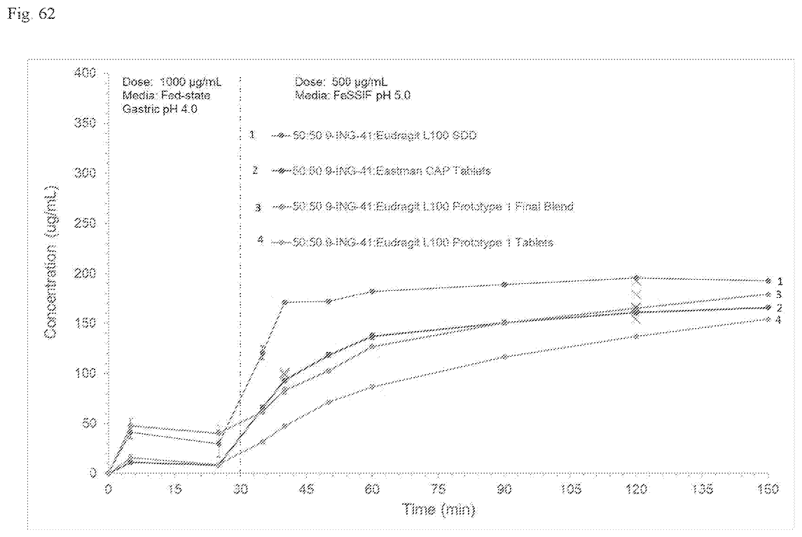

In this experiment, the prototype 1 tablets were compared to final blend to determine if the formulation dissolves rapidly after granulation/final blending and prior to compression. See Table 6-40. The final blend dissolved to a higher maximum concentration and had higher concentrations at each timepoint relative to the tablet formulation. This indicates that the compression step in the process is slowing down the dissolution and extent during non-sink biorelevant testing. See FIG. 62 (Conditions: USP Type II, IL Vessels, large paddles, 75 RPM, pH 4.0 HCl 1000 μg/mL to pH 5.0 FeSSIF 500 μg/mL).

TABLE 6-40

| | | Cmax-IB35- | AUC-IB35- | | C40 | | C120 |
|---|---|---|---|---|---|---|---|
| | Cmax-G2-25 | 120 | 120 | C40 | Ultra* | C120 | Ultra |
| Formulation | (μg/mL) | (μg/mL) | (μg/mL*min) | (μg/mL) | (μg/mL) | (μg/mL) | (μg/mL) |
| 50:50 9-ING- | 42 | 196 | 15567 | 171 | NT | 196 | 193 |
| 41:Eudragit ® | | | | | | | |
| L100 ASD | | | | | | | |
| 50:50 9-ING- | 11 | 161 | 11733 | 93 | 99 | 161 | 166 |
| 41:Eastman CAP | | | | | | | |
| Tablets | | | | | | | |
| 50:50 9-ING- | 48 | 165 | 11353 | 83 | 101 | 165 | 179 |
| 41:Eudragit ® | | | | | | | |
| L100 Prototype 1 | | | | | | | |
| Final Blend | | | | | | | |
| 50:50 9-ING- | 16 | 137 | 8452 | 48 | NT | 137 | 154 |
| 41:Eudragit ® | | | | | | | |
| L100 Prototype 1 | | | | | | | |
| Tablets | | | | | | | |

*Ultra data for Eastman CAP tablets and L100 Final blend was taken from n = 1 replicates. Data for the ASD and prototype tablets was not collected - supernatant from microcentrifugation was inadvertently spilled inside of the rotor during ultracentrifugation

The experiment also compared the tablet and final blend for prototype 1 to the CAP tablet formulation which has been dosed in-vivo with acceptable performance. The final blend for prototype 1 matched the CAP tablet formulation reasonably well and the prototype 1 tablets were slower to dissolve and dissolved to a lower maximum concentration.

One note for the comparison is that the CAP tablet formulation was only compressed to a tensile strength of 1.4 MPa while the prototype 1 tablets were compressed to a tensile strength of 2.1 MPa. The tensile strength of the tablets is an important attribute for downstream processing if a cosmetic coating is to be applied.

### Drug Product Formulations and Performance Optimization

Based on the findings from the prototype 1-3 tablets performance investigation, a second round of tablet formulations were manufactured. The goal of the second round was to increase disintegrant to improve dissolution performance and assess different disintegrants to provide a more robust formulation that is not sensitive to hardness/tensile strength and to ensure acceptable attributes for downstream processing in a pan coating operation.

Tablet Prototypes 4 through 7 formulations manufactured as part of the second round of prototypes are summarized below in Table 6-41. Manufacturing data is summarized in Table 6-42.

TABLE 6-41

| | Formulation Optimization | | | | |
|---|---|---|---|---|---|
| Component | Common Blend | Prototype 4 | Prototype 5 | Prototype 6 | Prototype 7 |
| Dose (mg) | 250 | 250 | 250 | 250 | 250 |

Intragranular (%)

| | | | | | |
|---|---|---|---|---|---|
| 50% ELRA:CAP | 41.7 | | | | |
| 50% | | 50.0 | 50.0 | 50.0 | 48.5 |
| ELRA:Eudragit ® | | | | | |
| L100 | | | | | |
| MCC (Avicel Ph- | 27.33 | 19.5 | 19.5 | 19.5 | 18.9 |
| 102) | | | | | |
| Mannitol (Perlitol | 25 | 19.5 | 19.5 | 19.5 | 18.9 |
| 100SD) | | | | | |
| Ac-Di-Sol | 2 | 7 | | | 8.7 |
| PVP XL | | | 7 | | |
| PVP XL 10 | | | | 9 | |
| Syloid 244 FP | | 0.5 | 0.5 | 0.5 | 0.5 |
| Magnesium Stearate | 1 | 0.5 | 0.5 | 0.5 | 0.5 |
| Intragranular Total | 97.0 | 97.0 | 97.0 | 99.0 | 96.0 |

Extragranular (%)

| | | | | | |
|---|---|---|---|---|---|
| MCC (PH-102) | 2 | | | | |
| Ac-Di-Sol | | 2 | | | 3 |
| PVP XL | | | 2 | | |
| Cabosil | 0.5 | | | | |
| Syloid 244 FP | | 0.5 | 0.5 | 0.5 | 0.5 |
| Magnesium Stearate | 0.5 | 0.5 | 0.5 | 0.5 | 0.5 |
| Total | 100.0 | 100.0 | 100.0 | 100.0 | 100.0 |
| Tablet Weight | 1200 | 1000 | 1000 | 1000 | 1000 |

TABLE 6-42

| | | Average | Average |
|---|---|---|---|
| | | Tensile | Solid |
| | Hardness | Strength | Fraction |
| Formulation | (kP) | (MPa) | (%) |
| Prototype 4 | 24 | 1.71 | 0.66 |
| Prototype 5 | 26 | 1.81 | 0.66 |
| Prototype 6 | 26 | 1.81 | 0.66 |

Prototype 6 included an additional manufacturing step to commingle the ASD and PVP XL10 to see if a small particle size disintegrant could be used to disintegrate the granules more effectively into small particles that would have rapid dissolution.

Tablet formulations were tested for disintegration and results are presented in Table 6-43. Prototype 4 tablets had the fastest disintegration time and had good reproducibility.

TABLE 6-43
Average Disintegration Results for Elraglusib
Prototype Formulations (Ref: BBS0002-041)

| | Average | Minimum | Maximum |
|---|---|---|---|
| | Disintegration | Disintegration | Disintegration |
| | Time | Time | Time |
| Formulation | (min:sec) | (min:sec) | (min:sec) |
| Prototype 4 | 6:18 | 6:08 | 6:33 |
| Prototype 5 | <7:00* | <7:00 | <7:00 |
| Prototype 6 | >40:00 | >40:00 | >40:00 |

*Prototype 5 tablets disintegrated into large agglomerates that prevented the ST50 from registering disintegration time, however, time is estimated visually to be less than 7 minutes

Based on the tooling size used for the 1 g tablets, they did not fit in the disintegration apparatus as designed. The tablets were too large to fall flat in the tube and this resulted in the tablets not being agitated as designed in the apparatus. The tablets were all stuck in the bottom of the tube and were not floating up and down with the same agitation as expected for this apparatus.

Tablet formulations were tested for friability and results are presented in Table 6-44.

TABLE 6-44
Friability Results for Elraglusib Prototype
Formulations (Ref: BBS0002-036, -039, -040)

| | Single Tablet | 10 Tablet |
|---|---|---|
| Formulation | Friability (%) | Friability (%) |
| Prototype 4 | 0.01 | 0.06 |
| Prototype 5 | 0.02 | NT |
| Prototype 6 | 0.04 | 0.04 |

Single tablet friability was used as an in process check to provide confidence in proceeding with manufacture of 10-20 tablets at a tensile strength of approximately 1.8 MPa. All formulations compressed to 1.7-1.8 MPa had low friability and are anticipated to be acceptable for downstream processing.

Based on the poor disintegration of prototype 6, 3% extragranular disintegrant was added to the formulation for evaluation in dissolution (prototype 7-composition presented in Table 6-41 above). The intent of this evaluation was to add disintegrant to break the tablet apart to see if the commingling of the disintegrant with ASD had a positive impact on dissolution. This formulation was also intentionally manufactured at a tensile strength of 1.5 MPa to gather friability data and evaluate performance.

Friability and disintegration were tested on the prototype 7 formulation. This data along with the manufacturing data are included below in Table 6-45.

TABLE 6-45
Manufacturing, Disintegration, and Friability Data for Prototype 7

| | | | | | Single |
|---|---|---|---|---|---|
| | | Tensile | Solid | Disintegration | Tablet |
| | Hardness | Strength | Fraction | (min:sec) | Friability |
| Formulation | (kP) | (MPa) | (%) | n = 1 | (%) |
| Prototype 7 | 21.5 | 1.49 | 0.64 | 6:09 | 0.05 |

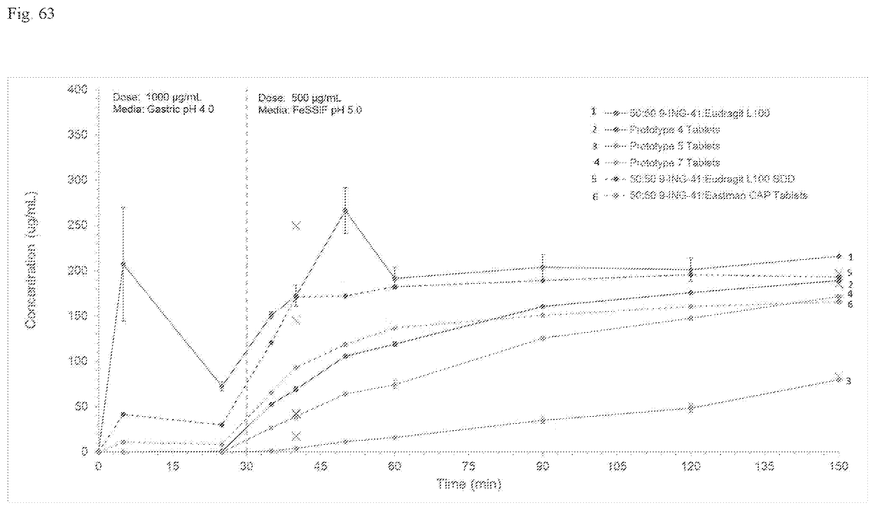

Dissolution in non-sink biorelevant conditions was carried out on prototype formulations 4, 5, and 7. (Conditions: USP Type II, IL Vessels, large paddles, 75 RPM, pH 4.0 HCl 1000 μg/mL to pH 5.0 FeSSIF 500 μg/mL) Data is presented below in FIG. 63 and Table 6-50.

TABLE 6-50
Tabulated Non-sink Dissolution of Elraglusib Prototype
Formulations Compared to Ingoing ASD (Ref: BBS0002-044)

| | | | AUC-IB35- | | C40 | | C150 |
|---|---|---|---|---|---|---|---|
| | Cmax-G2-25 | Cmax-IB₃₅₋₁₅₀ | 150 | C40 | Ultra | C150 | Ultra |
| Formulation | (μg/mL) | (μg/mL) | (μg/mL*min) | (μg/mL) | (μg/mL) | (μg/mL) | (μg/mL) |
| 50:50 9-ING- | 207 | 267 | 23579 | 172 | 145 | 216 | 197 |
| 41:Eudragit ® | | | | | | | |
| L100 | | | | | | | |
| Prototype 4 | 1 | 189 | 17034 | 69 | 42 | 189 | 186 |
| Tablets | | | | | | | |
| Prototype 5 | 0 | 80 | 4180 | 4 | 17 | 80 | 83 |
| Tablets | | | | | | | |
| Prototype 7 | 0 | 172 | 13258 | 39 | 250* | 172 | 169 |
| Tablets | | | | | | | |
| 50:50 9-ING- | 42 | 196 | 15567 | 171 | NT | 196 | NT |
| 41:Eudragit ® | | | | | | | |
| L100 ASD** | | | | | | | |
| 50:50 9-ING- | 11 | 161 | 16632 | 93 | 99 | 161 | NT |
| 41:Eastman CAP | | | | | | | |
| Tablets** | | | | | | | |

*Value is unreasonably high and likely related to sampling error.
**Formulations tested in BBS0002-030 and included for comparison purposes only.

The dissolution of the prototype 4 formulation compressed to a tensile strength of 1.71 MPa matched closely to the previously tested CAP formulation that had good in-vivo performance. Prototype 7 formulation had performance like the first round of prototype formulations and prototype 5 was slow to dissolve.

It is worth noting that prototype formulations 4, 5, and 7 all floated during dissolution testing. Sinkers should be evaluated and used if necessary to keep future iterations of these formulations from floating in dissolution.

### Example 7—Scaled up Preparation of Elra:EL100 ASD by Spray Drying Lead ASD Supply for Tablet Manufacture

The lead ASD formulation was manufactured to supply a larger tablet batch to support stability and analytical method development. The batch had a target batch size of 720 g and was split into 5 equal sub-batches due to limitations on the tank size utilized to support the manufacture. The batch utilized a SD40 spray dryer.

Spray drying parameters were maintained for all 5 batches within reasonable ranges relative to targets. The atomization pressure was increased from a target of 130 Psig to approximately 145 Psig to help mitigate atomizer build-up. Some nozzle fouling was observed on each sub-batch and the time it took to need to clean the nozzle improved with higher atomization pressure. This is expected to be scale-dependent and should be less of an issue upon scale-up where a larger atomization pressure and available nozzle geometries are available.

TABLE 7-1
Spray Drying Manufacturing Process Summary

| | BBS0002-028 | | | | |
|---|---|---|---|---|---|
| Batch Reference | #1 | #2 | #3 | #4 | #5 |
| ASD Formulation | 50/50 9-ING-41/Eudragit ® L100 | | | | |
| Solvent Blend (wt % | 90/10 Acetone/Water | | | | |
| ASD Batch Size (g) | 720 (5 sub-batches of 144 g) | | | | |
| Solids Loading (wt %) | 3.0 | | | | |
| Atomizer | Schlick 2.0 Pressure-Swirl | | | | |
| | Atomizer | | | | |
| Atomization Pressure (Psig) | 136 | 145 | 145 | 142 | 144 |
| Liquid Feed Rate (g/min) | 26 | 24 | 25 | 23 | 27 |
| Gas Flow Rate (g/min) | 501 | 504 | 504 | 504 | 507 |
| Inlet Temperature (° C.) | 99 | 99 | 98 | 101 | 102 |
| Outlet Temperature (° C.) | 45 | 45 | 45 | 45 | 44 |

| | |
|---|---|
| Calculated outlet RS (% | 3.0/7.8 |
| Acetone/Water) | |
| Dew Point (° C.) | 5.4 |
| Yield After Secondary | 90.2 |
| Drying (%) | |

TABLE 7-2
Secondary drying conditions and Final Residual Solvent
Concentration at the Conclusion of Drying

| Dried ASD Lot | BBS0002-018 |
|---|---|
| Vacuum level (inHg) | −20 |
| Nitrogen Sweep Rate (SCFH) | 10 |
| Drying Temperature (° C.) | 30 (2 Hr)/50 (2 Hr)/80 (22 Hr) |
| Final Acetone Concentration (wt %) | 0.01 |

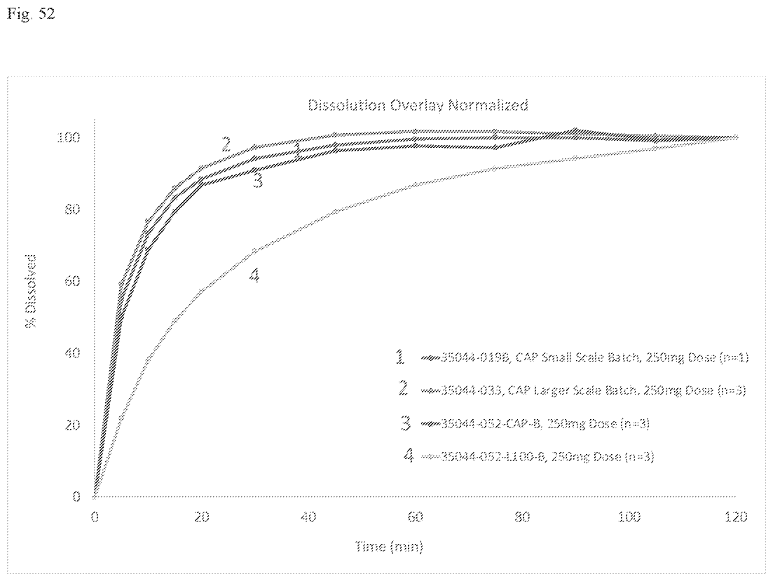

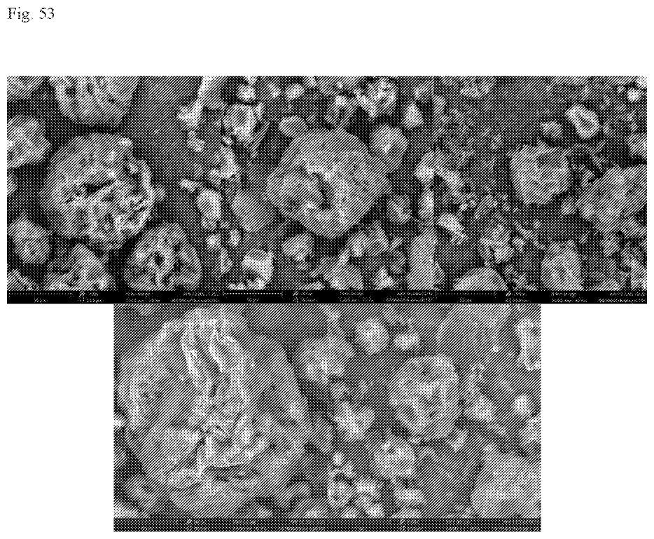

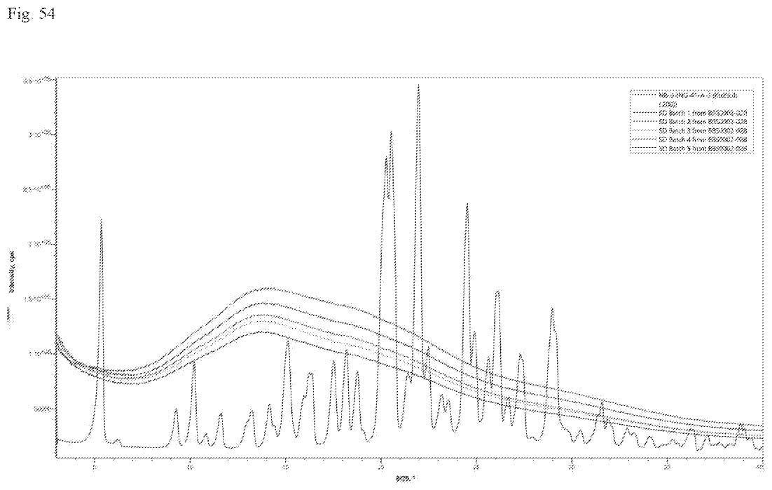

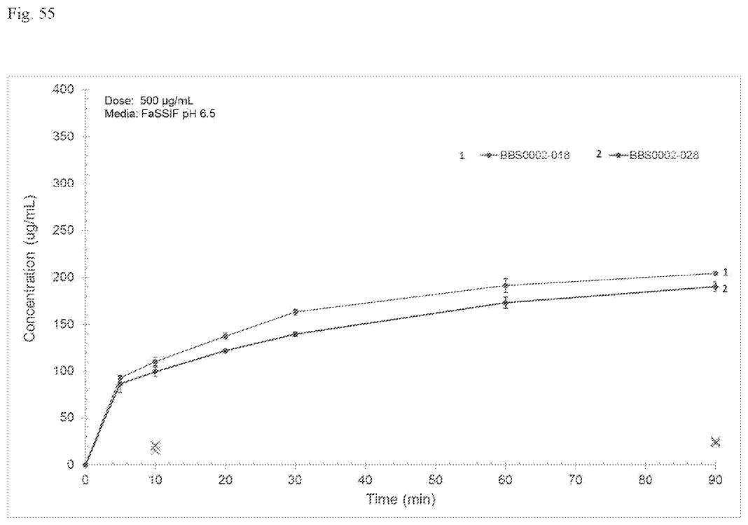

The 5 sub-lots of ASD were analyzed by SEM and PXRD to confirm morphology and initial physical state were similar across all lots. SEM images are presented below in FIG. 53. The PXRD testing resulted in amorphous halos with no evidence of crystallinity (FIG. 54). All sub-lots were deemed to be similar by SEM and PXRD and were combined by bag blending to generate a single lot of BBS0002-028 for further characterization and downstream processing.

The composite ASD (Lot BBS0002-028) has a Tg of 112.3±0.1 C with no indication of phase separation or crystallization. This is slightly higher than the Tg measured for BBS0002-005 at 107.4 C (Test Ref. BBS0002-017), indicating that this lot was likely more completely dried of acetone than the previous batch, which was shown to contain a significant amount of residual acetone.

Non-sink biorelevant (conditions: 50 ml beaker, 30 mL FaSSIF pH=6.5) dissolution was performed to assess concentration vs. time profile for the lead ASD. Data in FIG. 55 and Table 7-3 show that the composite ASD preforms similar to the previously manufactured formulation.

TABLE 7-3

| | Cmax-IB₅₋₉₀ | AUC-IB₅₋₉₀ | C10 | C10 Ultra | C90 | C90 Ultra |
|---|---|---|---|---|---|---|
| Formulation | (μg/mL) | (μg/mL*min) | (μg/mL) | (μg/mL) | (μg/mL) | (μg/mL) |
| BBS0002-018 | 204 | 14492 | 111 | 15 | 204 | 23 |
| BBS0002-028 | 190 | 13005 | 100 | 21 | 190 | 25 |

Non-sink dissolution was performed at a slightly different volume relative to previous tests to aid in the wettability of the ASD when testing at the smaller scale.

Ultracentrifugation data continues to be variable for the lead elraglusib ASD formulation. The control formulation (BBS0002-018) has had higher ultra values in the past, indicating that the variability is not anticipated to have any impact and is related to sampling and analytical variability.

50:50 Elra:Eudragit® L100 ASD (BBS0002-028) did not show signs of significant degradation in the form of related peaks. 50:50 Elra:Eudragit® L100 (BBS0002-028) was found to be 48.7% 9-ING-41 and had average impurities of 1.13 area %. Assay and related substances data is presented below in Table 7-4.

TABLE 7-4
Assay and Related Substances Data

| | | | | | | | AVG | |
|---|---|---|---|---|---|---|---|---|
| | | | | | | | % | AVG |
| Injection | | | | | Area | | Assay | Impurities |
| Name | Prep | RT (min) | RRT | Component | (mAu*s) | Result | (w/w) | (% area) |
| API | 1 | 11.073 | 1.000 | 9-ING-41 | 4424.1873 | 99.7782% | n/a | 0.74% |
| Injection | | | | | | | | |
| (STD-1) | | 13.355 | 1.206 | Impurity RRT | 33.1701 | 0.7442% | | |
| | | | | 1.21 | | | | |
| 50:50 | 1 | 11.073 | 1.000 | 9-ING-41 | 4446.2214 | 48.7626% | 48.7% | 1.13% |
| Elra/EL100 | | 13.352 | 1.206 | Impurity RRT | 50.7737 | 1.1291% | | |
| BBS0002- | | | | 1.21 | | | | |
| 028 | 2 | 11.069 | 1.000 | 9-ING-41 | 4607.2519 | 48.7145% | | |
| | | 13.352 | 1.206 | Impurity RRT | 52.6092 | 1.1290% | | |
| | | | | 1.21 | | | | |

### Example 8—Scaled up Preparation of Elra:EL100 Tablets

Blending and dry granulation were executed at 1131.52 g batch size to furnish the 50% ASD (25% Elraglusib) loaded final blend. A blend-sieve-blend process was used during intragranular blending to delump the components and eliminate any potential foreign contaminants (i.e. HDPE) that may have inadvertently been left in excipient bags. The intragranular blend was cohesive; gentle tapping of the 8 qt tote was required to discharge the blend. The intragranular blend was at approximately 50% fill in the 8 qt tote during blending. A 50 g sample of the IG blend was taken for future FloDex, bulk/tap density, and CTC profiling.

TABLE 8-1

| | | | | | | | |
|---|---|---|---|---|---|---|---|
| | | | | | Amount | Amount | |
| | | Compendial | | | Theoretical | Actual | Lot |
| Component | Grade | Listing | % w/w | mg/tablet | (g) | (g) | Number |

Intragranular

| | | | | | | | |
|---|---|---|---|---|---|---|---|
| 50% 9-ING- | Spray dried | USP, NF, | 50 | 500 | 568.26 | 568.26 | BBS0002- |
| 41:Eudragit ® L | Intermediate | JP, Ph Eur | | | | | 028 |
| 100 | | (EP) | | | | | |
| Microcrystalline | Avicel PH- | USP, NF, | 19.5 | 195 | 221.6214 | 221.63 | 21740481 |
| Cellulose (PH | 102 | JP, Ph Eur | | | | | 83 |
| 102) | | (EP) | | | | | |
| Mannitol (100 | Pearlitol 100 | USP, NF, | 19.5 | 195 | 221.6214 | 221.62 | E121G |
| SD) | SD | JP, Ph Eur | | | | | |
| | | (EP) | | | | | |
| Croscarmellose | Ac-Di-Sol | USP, NF, | 7.0 | 70 | 79.5564 | 79.57 | 21740528 |
| Sodium | SD-711 | JP, Ph Eur | | | | | 14 |
| | | (EP) | | | | | |
| Silicon Dioxide | Syloid 244FP | USP, NF, | 0.5 | 5 | 5.6826 | 5.68 | 52102374 |
| | | JP, Ph Eur | | | | | 32 |
| | | (EP) | | | | | |
| Magnesium | Ligamed MF- | USP, NF, | 0.5 | 5 | 5.6826 | 5.68 | C238300 |
| Stearate | 2-V-MB | JP, Ph Eur | | | | | |
| | | (EP) | | | | | |
| Total | | | 97 | 970 | 1102.4244 | | |

Extragranular

| | | | | | | | |
|---|---|---|---|---|---|---|---|
| Croscarmellose | Ac-Di-Sol | USP, NF, | 2 | 20 | 22.7304 | 19.91* | 21740528 |
| Sodium | SD-711 | JP, Ph Eur | | | | | 14 |
| | | (EP) | | | | | |
| Silicon Dioxide | Syloid 244FP | USP, NF, | 0.5 | 5 | 5.6826 | 4.97* | 52102374 |
| | | JP, Ph Eur | | | | | 63 |
| | | (EP) | | | | | |
| Magnesium | Ligamed MF- | USP, NF, | 0.5 | 5 | 5.6826 | 4.97* | C238300 |
| Stearate | 2-V-MB | JP, Ph Eur | | | | | |
| | | (EP) | | | | | |
| Total | | | 100 | 1000 | 1136.52 | | |

*Extragranular amounts were adjusted based on a batch factor of 0.8753.

### Roller Compaction and Granulation

Dry granulation was performed on a Gerteis Mini-pactor with the parameters specified in Table 8-2. Minimal sticking to the feed/tamp augers and rollers was observed, and material was readily conveyed into the compaction zone. Set gap was reached approximately 1.5 minutes into the run. Close to 2 g of ribbons were collected for solid fraction determination via the GeoPyc, an additional NLT 5 g sample of granules were collected for SEM imaging and sieve analysis.

Gerteis Parameters for Manufacture of Elraglusib Lead Tablet Formulation (Data Ref: BBS0002-025)

TABLE 8-2

| | | Value Initial |
|---|---|---|
| Process Parameter | Set Point | (t = 0) |
| Roll Type | Knurled/ | Knurled/ |
| | Knurled | Knurled |
| Granulator Type | Pocket | Pocket |
| Gap Control | On | On |
| Granulator Angle Clockwise | 360 | 360 |
| Granulator Angle Counterclockwise | 360 | 360 |
| Tamp to Feed Auger Ratio (%) | 180 | 180 |
| Feed Factor | 0.5 | 0.5 |
| Agitator Speed | 5.0 | 5.0 |
| Roll Speed (rpm) | 2.0 | 0.5 then ramp |
| | | to 2.0 |
| Roll Force (kN/cm) | 2.0 | 2.0 |
| Roll Gap (mm) | 2.0 | 2.0 |
| Granulator speed-Clockwise (rpm) | 60 | 60 |
| Granulator speed-Counterclockwise | 60 | 60 |
| (rpm) | | |
| Square Wire Mesh Granulator | 1.0 | 1.0 |
| Screen (mm) | | |
| Screen Gap Setting (mm) | 1.0 | 1.0 |

Ribbon solid fraction was determined via measurement of the envelope density using a GeoPyc and the calculated true density of the formulation. The average solid fraction from n=3 measurements on varying ribbon sample quantities was determined to be 0.49, consistent with successful dry granulation without over-compaction of the blend (Table 8-3).

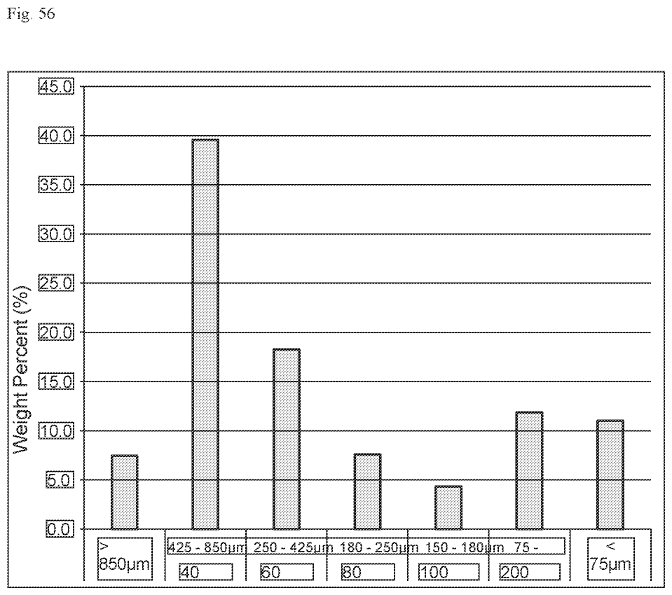

Images were taken of the granules from the manufacture to understand granule morphology and ensure the granules are not being extruded through the screen resulting in high aspect ratio granules. Visually it is estimated that less than 20% of the granules have aspect ratios larger than 1:2, and the majority of particles are below an aspect ratio of 1:1.5. Sieve analysis was performed on the granulation to determine the particle size distribution. Results indicate nearly half of the granules are above 425 um in size (47.0%). Minimum fines (<15%) were observed. Data from the analysis can be found below in FIG. 56.

After batch factor adjustment and final blending, a 50 g sample of the final blend was collected for Flodex, bulk/tap density, and CTC profiling. Ultimately the final blend was produced in an overall 83.3% yield (940.63 g) and total accountable yield of 93.3%. No observable process issues were encountered during the dry granulation process.

Average (n=3) bulk and tap density values were measured for both the intragranular and final blend in-process samples. Bulk density of the intragranular blend was low (0.28 g/cc), consistent with an approximately 1100 g batch size filling 50% of the 8 quart tote. Hausner ratios calculated for the intragranular and final blends indicates successful improvement in flowability via roller compaction; Hausner ratio decreased from 1.55 g/cc for the intragranular blend, to 1.13 g/cc for the final blend.

TABLE 8-3

| | Ribbon | | True | Average | | |
|---|---|---|---|---|---|---|
| | Sample | Measurement | Density | Envelope | Porosity | Solid |
| Test # | Quantity (g) | Cycles | (g/cm3) | Density (g/cm3) | (%) | Fraction |
| 1 | 0.2982 | 5 | 1.4012 | 0.6401 | 54 | 0.46 |
| 2 | 0.4559 | 5 | 1.4012 | 0.7218 | 48 | 0.52 |
| 3 | 0.2602 | 10 | 1.4012 | 0.6959 | 50 | 0.50 |
| Average | | | | 0.6859 | 51 | 0.49 |

Powder flowability was also evaluated via Flodex on the intragranular and final blend. Results are consistent with improved flowability after granulation, albeit minimal reduction in Flow index was observed. Values of 18 and 16 were determined for the intragranular and final blend, respectfully. The minimal change in Flow index could be due to inherent cohesiveness of the powder blend before and after granulation (Table 8-4).

TABLE 8-4
Bulk/Tap Density and Flodex of Intragranular and Final
Blends (Data Ref: BBS0002-059 and BBS0002-060)

| | Average | Average | Average | | |
|---|---|---|---|---|---|
| | Bulk | Tap | Compress- | Average | |
| | Density | Density | ibility | Hausner | Flow |
| Blend | (n = 3, | (n = 3, | Index | Ratio | Index |
| Identity | g/mL) | g/mL) | (n = 3) | (n = 3) | (mm) |
| Intra- | 0.28 ± 0.01 | 0.42 ± 0.00 | 34 ± 2 | 1.51 ± 0.04 | 18 |
| granular | | | | | |
| Blend | | | | | |
| Final | 0.42 ± 0.01 | 0.48 ± 0.01 | 11 ± 0 | 1.13 ± 0.00 | 16 |
| Blend | | | | | |

Tabletability, Compressibility, and Compactability profiles were determined for the intragranular (BBS0002-025 IG) and final blends (BBS0002-025 FB). The IG blend shows higher tabletability and compactability relative to the FB, as interparticulate binding sites were removed during the roller compaction process. Compressibility of both the IG and FB are comparable, indicating dry granulation had minimal effect on the blends ability to reduce in volume as a result of applied pressure.

Collectively all data collected on the intragranular blend, granules, and final blend indicate minimal process refinement is necessary: 2 kN/cm roll force, 1.0 mm screen size, and the parameters listed in Table 8-2 are suitable for scale-up.

### Compression

1000 mg core (250 mg dose) Elraglusib tablets were pressed using the TPR200 rotary tablet press equipped with 0.3740×0.8465 mod. oval TSM-D tooling, a 14 mm fill cam, and the paddle feeder. Initial compression parameters were set based on previous compression parameters from the initial Prototype 4 tablet production on the STYL'One Nano (BBS0002-034). Parameters were then adjusted to achieve targets listed above. The collection container was switched every ten minutes. After verifying in-process samples were within range, collection containers were combined to form the bulk tablet supply. Overall approximately 758 tablets with an average (n=30) tensile strength of 1.59 MPa were produced. Both the initial (t=0, beginning) and final (t=20) ten tablet samples were below 0.02% friability, indicating the slightly lower (<2 MPa) tablets are suitable for shipment and coating.

TABLE 8-5
Target Compression Parameters for Elraglusib Lead
Tablet Manufacture (Data Ref: BBS0002- 027)

| Press Used | TPR 200 | |
|---|---|---|
| Fill Cam | 14 | |
| Tooling | .3740 × | 4 D-tooling sets |
| | .8465 in | mod ovalultracoat |
| | Target Value* | Target Range* |
| Paddle Speed (RPM) | 15 | 10-20 |
| Pre-compression Force (kN) | 1 | 0.5-1.5 |
| Compression Force (kN) | 10 | 9-11 |
| Individual Weight (mg) [+−5%] | 1000 | 950-1050 |
| Average Weight (mg, n = 10) | 1000 | 970-1030 |
| [+−3%] | | |
| Thickness (mm) [+−5%] | 7.22 | 6.87-7.59 |
| Hardness (kp) [+−16%] | 22.5 | 18.5-26.5 |
| Tensile Strength ((MPa) | 1.6 | 1.4-1.8 |

*Parameters are guideline only, adjust as necessary to meet target core weight and tensile strength

TABLE 8-6
In Process Manufacturing Summary for In-
process Checks (Data Ref: BBS0002-027)

| | Core Tablet | | | Tensile | Solid |
|---|---|---|---|---|---|
| | Weight | Thickness | Hardness | Strength | Fraction |
| | (mg) | (mm) | (kP) | (MPa) | (%) |
| Average | 998.05 | 7.28 | 22.6 | 1.59 | 0.64 |
| Min | 971.9 | 7.24 | 17.3 | 1.21 | 0.62 |
| Max | 1029.4 | 7.34 | 28.2 | 2 | 0.66 |
| Std | 19.8 | 0.04 | 3.1 | 0.22 | 0.02 |
| Deviation | | | | | |
| % RSD | 2.0 | 0.49 | 13.6 | 14.10 | 2.60 |

TABLE 8-7
Batch Summary for Lead Elraglusib Tablet
Manufacture (Data Ref: BBS0002-027)

| | | Average tablet |
|---|---|---|
| | Weight of 100 | weight from |
| Sample # | Tablets (g) | compression (g) |
| 100 Tablet Sample | 101.17 | 1.0117 |

| | Net Weight (g) |
|---|---|
| Bulk Tablets Collected | 766.39 |
| Start Up/Sampled | 110.72 |
| Tablets | |
| Content Uniformity | 10.038 |
| Friability | 20.1209 |
| Blend Recovered | 23.6 |

| Tablet Bulk | Accountable Yield | Actual Yield |
|---|---|---|
| Quantity | (%) | (%) |
| 758 | 99.0 | 81.5 |

### Tablet Characterization

Characterization was performed on the lead elraglusib tablets to evaluate the performance of the tablet relative to prototype 4 manufactured at small scale and in comparison to the ingoing ASD formulation.

Friability was measured at the beginning of the batch as an in process control and at the end of the batch. Data is presented below in Table 8-8.

TABLE 8-8
Friability for Elraglusib Lead Tablet
Manufacture (Data Ref: BBS0002-027)

| | | |
|---|---|---|
| Initial Weight (g) | Final Weight (g) | Friability (%) |

Beginning (n = 10)

| | | |
|---|---|---|
| 10.1737 | 10.1731 | 0.006 |

End (n = 10)

| | | |
|---|---|---|
| 9.9472 | 9.9459 | 0.013 |

Disintegration was performed on the tablets in 0.01 N pH=2.0. Data is presented below in Table 8-9.

TABLE 8-9
Disintegration for Elraglusib Lead
Tablets (Data Ref: BBS0002-054)

| Basket | Disintegration Time |
|---|---|
| Position | (00:00 min:sec) |
| 1 | 06:14 |
| 2 | 03:58 |
| 3 | 04:00 |

It should be noted that the tablets are large enough that they do not sit flat in the disintegration tubes. This results in the tablets either sticking in the tube and not floating during agitation or the tablets can get stuck to the float as well.

Appearance of the tablets was performed for future reference during stability studies and can be found in Table 8-10.

TABLE 8-10
Appearance Elraglusib Lead Tablets (Data Ref: BBS0002-057)

| Sample | Lot | Appearance |
|---|---|---|
| 250 mg 9-ING- | BBS0002- | Large Capsule Shaped tablets. |
| 41 Eudragit ® | 027 | Orange (#7253 Colorcon |
| L100 tablets | | General Color Wheel V.4) |

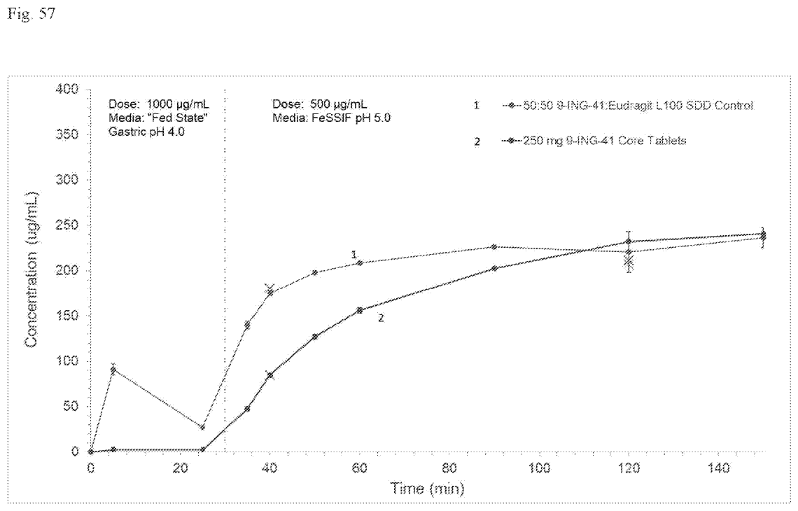

Non-sink dissolution testing was performed on the lead tablet formulation compared to the ingoing ASD. Under the non-sink conditions tested, the tablet matched the ASD reasonably well with similar concentrations as early as 90 minutes. Non-sink dissolution results are presented below in FIG. 57 (Conditions: USP Type II, IL Vessels, large paddles, 50 RPM, pH 4.0 HCl 1000 μg/mL to pH 5.0 FeSSIF 500 μg/mL) and Table 8-11.

TABLE 8-11
Tabulated Non-sink Dissolution of Elraglusib Lead Formulation
Compared to Ingoing SDD (Ref: BBS0002-056)

| | | | AUC- | | | | |
|---|---|---|---|---|---|---|---|
| | Cmax- | Cmax- | IB35-120 | | C40 | | C120 |
| | G2-25 | IB35-120 | (μg/mL* | C40 | Ultra | C120 | Ultra |
| Formulation | (μg/mL) | (μg/mL) | min) | (μg/mL) | (μg/mL) | (μg/mL) | (μg/mL) |
| 50:50 | 91 | 227 | 17927 | 175 | 180 | 221 | 207 |
| Elra:EL100 | | | | | | | |
| ASD Control | | | | | | | |
| 250 mg | 3 | 232 | 14707 | 85 | 85 | 232 | 212 |
| Elraglusib | | | | | | | |
| CoreTablets | | | | | | | |

Content uniformity was assessed on the lead tablet formulation manufactured on the TPR-200 tablet press, resulting in passing results with an acceptance value of 4.6. Data is presented below in Table 8-12.

TABLE 8-12
Content Uniformity Summary for Elraglusib
Lead Tablets (Data Ref: BBS0002-054)
AV Calculation

| min | 95.5% | M | 98.48% | AV = \|M − X\| + ks | |
|---|---|---|---|---|---|
| max | 100.6% | X (mean of | 98.48% | \|M − X\| | 0.0000% |
| | | individual strength) | | | |
| mean | 98.5% | k (n = 10) | 2.4 | ks | 4.5551% |
| std dev | 1.9% | s(sample std dev) | 1.90% | AV | 4.5551% |
| % RSD | 1.9% | | | | |

| Criteria | Result |
|---|---|
| Report Acceptance Value (AV) | 4.6 |
| Meets USP <905> Requirement: L1 NMT 15.0. | Conforms |

Assay and related substances data resulted in similar data to the ingoing ASD (Table 7-4), as expected. The assay of the tablets with no correction for the ASD potency (Table 7-4) was acceptable at 98.7%. Assay and related substances data is summarized in Table 8-13.

TABLE 8-13
Tabulated Assay and Related Substances of Lead
Elraglusib Tablets (Data Ref: BBS0002-054)

| | | | | | | AVG | |
|---|---|---|---|---|---|---|---|
| Injection | | | | Area | | 9-ING-41 | Avg |
| Name | Prep | RRT | Component | (mAu*s) | Result | (% w/w) | Impurities |
| Composite | 1 | 1.000 | 9-ING-41 | 4477.2371 | 99.4397% | 98.67% | 1.11% |
| | | 1.206 | Impurity RRT | 50.4624 | 1.1145% | | |
| | | | 1.21 | | | | |
| | 2 | 1.000 | 9-ING-41 | 4407.8997 | 97.8997% | | |
| | | 1.206 | Impurity RRT | 49.2963 | 1.1060% | | |
| | | | 1.21 | | | | |

Nothing in this specification should be considered as limiting the Scope of this disclosure. All examples presented are representative and non-limiting. The above-described embodiments can be modified or varied, as appreciated by those skilled in the art in light of the above teachings. It is therefore to be understood that, within the scope of the claims and their equivalents, the embodiments disclosed herein can be practiced otherwise than as specifically described.

What is claimed:

1. A solid dispersion comprising amorphous 3-(5-fluorobenzofuran-3-yl)-4-(5-methyl-5H-[1,3]dioxolo[4,5-f]indol-7-yl)-1H-pyrrole-2,5-dione and a stabilizing polymer.

2. The solid dispersion according to claim 1, wherein said stabilizing polymer is N-vinyl-2-pyrrolidone-vinyl acetate copolymer (copovidone), cellulose acetate phthalate, hydroxypropyl methylcellulose phthalate, hydroxypropyl methylcellulose acetate succinate, polyvinyl acetate phthalate, polyvinylpyrrolidone, poly(methyl methacrylate-co-methacrylic acid), or a miscible mixture thereof.

3. The solid dispersion according to claim 1, wherein said stabilizing polymer is poly(methylmethacrylate-co-methacrylic acid) (1:1), Eudragit® L100 polymer (“EL100”), or Eudragit® L100-55 polymer.

4. The solid dispersion according to claim 1, wherein said stabilizing polymer is soluble at a pH above 5, above 5.5, or above 6.

5. The solid dispersion according to claim 1, wherein the 3-(5-fluorobenzofuran-3-yl)-4-(5-methyl-5H-[1,3]dioxolo[4,5-f]indol-7-yl)-1H-pyrrole-2,5-dione is present in an amount of: (a) from about 10% to about 70% by weight relative to the weight of the stabilizing polymer; (b) from about 20% to about 60% by weight relative to the weight of the stabilizing polymer; and/or (c) from about 25% to about 50% by weight relative to the weight of the stabilizing polymer.

6. The solid dispersion according to claim 1, wherein the weight ratio of amorphous 3-(5-fluorobenzofuran-3-yl)-4-(5-methyl-5H-[1,3]dioxolo[4,5-f]indol-7-yl)-1H-pyrrole-2,5-dione to stabilizing polymer ranges from about 30:70 to about 50:50.

7. The solid dispersion according to claim 1, wherein said solid dispersion: (a) has a single glass transition temperature; (b) is stable for at least 48 hours at 60° C. and 75% relative humidity; (c) is stable for at least 4 weeks at 40° C. and 75% relative humidity and/or (d) is stable for at least 12 weeks at 40° C. and 75% relative humidity.

8. The solid dispersion according to claim 1, wherein oral administration of the solid dispersion to a patient results in an elraglusib AUC∞ that is: (a) at least 40% of the elraglusib AUC∞ resulting from IV administration to the patient of an equivalent dose (on a mg/kg basis) of elraglusib; (b) is at least 68% of the elraglusib AUC∞ resulting from IV administration to the patient of an equivalent dose (on a mg/kg basis) of elraglusib; or (c) at least 97% of the elraglusib AUC∞ resulting from IV administration to the patient of an equivalent dose (on a mg/kg basis) of elraglusib.

9. A pharmaceutical composition comprising a therapeutically effective amount of a solid dispersion according to claim 1, and a pharmaceutically acceptable excipient.

10. A tablet for oral administration, comprising:
(i) an ASD comprising elraglusib and a stabilizing polymer;
(ii) a binder;
(iii) a filler;
(iv) a disintegrant; and
(v) a lubricant.

11. The tablet of claim 10, comprising:
(a) (i) about 40-60% by weight of an ASD comprising elraglusib and a stabilizing polymer;
(ii) about 19-27% by weight of a binder;
(iii) about 10-25% by weight of a filler;
(iv) about 4-9% by weight of a disintegrant; and
(v) about 1-3% of a lubricant; or
(b) (i) about 50% by weight of an ASD comprising elraglusib and a stabilizing polymer; (ii) about 19.5% by weight of a binder;
(iii) about 19.5% by weight of a filler;
(iv) about 9% by weight of a disintegrant; and
(v) about 2% of a lubricant.

12. The tablet of claim 10, wherein: (a) the stabilizing polymer is poly(methyl methacrylate-co-methacrylic acid); (b) wherein the ASD comprises about 50% by weight elraglusib and about 50% by weight poly(methyl methacrylate-co-methacrylic acid); (c) the stabilizing polymer is CAP; (d) the binder is microcrystalline cellulose; (e) the filler is mannitol; (f) the disintegrant is croscarmellose sodium or a polyvinyl pyrrolidone; and/or (g) the lubricant is one or more of a silicon dioxide or magnesium stearate.

13. The tablet of claim 10, comprising:
(a) (i) an ASD comprising about 50% by weight elraglusib and about 50% by weight poly(methyl methacrylate-co-methacrylic acid);
(ii) microcrystalline cellulose;
(iii) mannitol;
(iv) croscarmellose sodium;
(v) silicon dioxide; and
(vi) magnesium stearate; or
(b) (i) about 50% by weight of an ASD comprising about 50% by weight elraglusib and about 50% by weight poly(methyl methacrylate-co-methacrylic acid);
(ii) about 19.5% by weight microcrystalline cellulose;
(iii) about 19.5% by weight mannitol;
(iv) about 9% by weight croscarmellose sodium;
(v) about 1% by weight silicon dioxide; and
(vi) about 1% by weight magnesium stearate.

14. The tablet of claim 10, wherein oral administration of the tablet to a subject results in: (a) a plasma elraglusib Cₘₐₓ (fed) that is at least 250% of the corresponding plasma elraglusib Cₘₐₓ (fasted); (b) a plasma elraglusib AUC∞ (fed) that is at least 240% of the corresponding plasma elraglusib AUC∞ (fasted); and/or (c) an elraglusib AUC∞ that is at least 40% of the elraglusib AUC∞ resulting from IV administration to the subject of an equivalent dose (on a mg/kg basis) of elraglusib.
